(19) United States
(12) Patent Application Publication
(10) Pub. No.: US 2003/0236257 A1
(43) Pub. Date: Dec. 25, 2003

(54) Polycyclic aryl and heteroaryl substituted 4-pyridones useful for selective inhibition of the cogulation cascade

(76) Inventors: Michael S. South, St. Louis, MO (US); Chun C. Ma, Chesterfield, MO (US); Kevin J. Koeller, Richmond Heights, MO (US); Hayat K. Rahman, Belleville, IL (US); William L. Neumann, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/240,817

(22) PCT Filed: Apr. 4, 2001
(86) PCT No.: PCT/US01/11014

(51) Int. Cl. A61K 31/53; A61K 31/506; A61K 31/501; A61K 31/497; A61K 31/444; A61K 31/4439; C 07D 4 1/02; C 07D 4 3/02; C07D 413/02; C07D 417/02

(52) U.S. Cl. 514/241; 514/252.03; 514/255.05; 514/256; 514/340; 514/341; 514/342; 544/212; 544/238; 544/333; 544/405; 546/255; 546/269.7; 546/271.4; 546/272.7

(57) ABSTRACT

The invention relates to polycyclic aryl and heteroaryl substituted 4-pyridone compounds useful as inhibitors of serine proteases of the coagulation cascade and compounds, compositions and methods for anticoagulant therapy for the treatment and prevention of a variety of thrombotic conditions including coronary artery and cerebrovascular diseases.

### FIELD OF THE INVENTION

This invention is in the field of anticoagulant therapy, and specifically relates to compounds, compositions and methods for preventing and treating thrombotic conditions such as coronary artery and cerebrovascular disease. More particularly, the invention relates to polycyclic aryl and heteroaryl substituted 4-pyridone compounds that inhibit serine proteases of the coagulation cascade.

### BACKGROUND OF THE INVENTION

Physiological systems control the fluidity of blood in mammals [Majerus, P. W. et al: Anticoagulant, Thrombolytic, and Antiplatelet Drugs. In Hardman, J. G. and Limbird, L. E, editors: Goodman & Gilman's The Pharmacological Basis of Therapeutics. 9th edition. New York, McGraw-Hill Book Co., 1996, pp. 1341-1343]. Blood must remain fluid within the vascular systems and yet be able to undergo hemostasis, cessation of blood loss from a damaged vessel, quickly. Hemostasis or clotting begins when platelets first adhere to macromolecules in subendothelian regions of an injured and/or damaged vessels. These platelets aggregate to form the primary hemostatic plug and stimulate local activation of plasma coagulation factors leading to generation of a fibrin clot that reinforces the aggregated platelets.

Plasma coagulation factors include factors II, V, VII, VIII, IX, X, XI, and XII; these are also called protease zymogens. These coagulation factors or protease zymogens are activated by serine proteases leading to coagulation in a so called “coagulation cascade” or chain reaction [Handin, R. I.: Bleeding and Thrombosis. In Wilson, J., et al. editors: Harrison's Principles of Internal Medicine. 12th Edition, New York, McGraw-Hill Book Co., 1991, p350]. Coagulation or clotting occurs in two ways through different pathways. An intrinsic or contact pathway leads from XII to XIIa to XIa to IXa and to the conversion of X to Xa. Xa with factor Va converts prothrombin (II) to thrombin (IIa) leading to conversion of fibrinogen to fibrin. Polymerization of fibrin leads to a fibrin clol An extrinsic pathway is initiated by the conversion of coagulation factor VII to VIIa by Xa. The presence of Tissue Factor and Vila accelerates formation of Xa in the presence of calcium ion and phospholipids. Formation of Xa leads to thrombin, fibrin, and a fibrin clot as described above. The presence of one or more of these many different coagulation factors and two distinct pathways of clotting could enable the efficacious, selective control and better understanding of parts of the coagulation or clotting process.

While clotting as a result of an injury to a blood vessel is a critical physiological process for mammals such as man, clotting can also lead to disease states. A pathological process called thrombosis results when platelet aggregation and/or a fibrin clot blocks (i.e., occludes) a blood vessel. Arterial thrombosis may result in ischemic necrosis of the tissue supplied by the artery. When the thrombosis occurs in a coronary artery, a myocardial infarction or heart attack can result. A thrombosis occurring in a vein may cause tissues drained by the vein to become edematous and inflamed. Thrombosis of a deep vein may be complicated by a pulmonary embolism. Preventing or treating clots in a blood vessel may be therapeutically useful by inhibiting formation of blood platelet aggregates, inhibiting formation of fibrin, inhibiting thombus formation, inhibiting embolus formation, and for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels.

There have been several reports of non-peptidic and peptidic 2-pyridone compounds that act as an inhibitor of a coagulation factor present in the coagulation cascade or clotting process. In U.S. Pat. No. 5,668,289, Sanderson et al. describe 6-alkyl, &cycloalkyl, and 6-triiluoromethyl 2-pyridones unsubstituted at the 4 and 5 positions and reported to inhibit thrombin. In PCT Patent Application WO 97/01338, Sanderson et al. describe 6-alkyl, 6-cycloalkyl, and &trifluoromethyl 2-pyridones unsubstituted at the 4 and 5 positions and reported to inhibit thrombin. In U.S. Pat. No. 5,792,779, Sanderson et al. describe substituted 4,6-alkyl, 4,6-cycloalkyl, and 4,6-trifluoromethyl 2-pyridones having utility as thrombin inhibitors. In PCr Patent Application WO 97/30708, Sanderson et al. describe additional substituted 4,6-alkyl, 4,6-cycloalkyl, and 4,6-trifluoromethyl 2-pyridones having utility as thrombin inhibitors. In U.S. Pat. No. 5,869,487, Coburn et al. describe pyrido[3,4-B]pyrazines containing a fused 6-methyl-2-pyridone functionality and having utility as thrombin inhibitors. In PCI Patent Application WO 98/47876, Van Boeckel et al. describe 6-alkyl-2-pyridones as anti-thrombotic compounds. In PCT Patent Application WO 98116547, Zhu and Scarborough describe 4,5,6-substituted-3-amino-2-pyridonylacetamides containing amide substituents having a heteroaroyl functions and having activity against mammalian factor Xa In U.S. Pat. No. 5,656,645, Tamura et al. describe 4,5,6-substituted-3-amino-2-pyridonyl-acetamides containing amide substituents having a formyl function and having activity against thrombin. In U.S. Pat. No. 5,658,930, Tamura et al. again describe 4,5,6-substituted-3-amino-2-pyridonyl-acetamides containing amide substituents having a formyl function and having activity against thrombin. In PCT Patent Applications 96118644, Tamura et al. further describe 4,5,6-substituted-3-amino-2-pyridonylacetamides containing amide substituents having a formyl function and having activity against thrombin. In PCT Patent Application WO 98131670, Sanderson et al. describe additional 4-substituted 6-alkyl, 6-cycloalkyl, and 6-trifluoromethylpyridones having utility as thrombin inhibitors. In PCT Patent Application WO 98/17274, Coburn et al. disclose substituted 3,4-diamino-6-methyl-2-pyridones having utility as human thrombin inhibitors. In PCT Patent Application WO 98142342, Isaacs et al. describe additional 6-alkyl, cycloalkyl, and trifluoromethyl substituted 2-pyridones reported to inhibit human thrombin. In PCI Patent Applications WO 00/039102, Wexler et al. describe certain 4,6-disubstituted-2-pyridonylacetamides that are further substituted at the 3-position by several groups including several substituted aminos and that are reported to be inhibitors of trysin-like serine protease enzymes, especially factor Xa and thrombin.

In contrast to the disclosures that some-2-pyridone derivatives may function as thrombin inhibitors, 4-pyridonyl compounds, compositions thereof, their use for preventing and treating thrombotic conditions such as coronary artery and cerebrovascular disease, and their ability to inhibit serine proteases of the coagulation cascade have not been previously disclosed.

### SUMMARY OF THE INVENTION

It is an object of the present invention to provide novel compounds that are beneficial in anticoagulant therapy and that have a general structure:

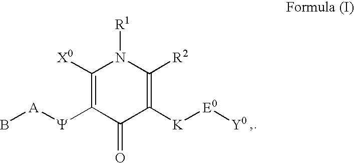

It is another object of the present invention to provide methods for preventing and treating thrombotic conditions, such as coronary artery disease, cerebrovascular disease, and other coagulation related disorders. Such thrombotic conditions are prevented and treated by administering to a patient in need thereof an effective amount of compounds of Formula (I).

Various other objects and advantages of the present invention will become apparent from the following description of the invention.

### DESCRIPTION OF THE INVENTION

The present invention relates to a class of compounds comprising Polycyclic Aryl and Heteroaryl Substituted 4-pyridones, which are beneficial in anticoagulant therapy for the treatment and prevention of a variety of thrombotic conditions including coronary artery and cerebrovascular disease, as given in Formula (I):

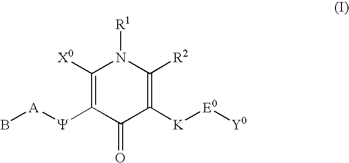

or a pharmaceutically acceptable salt thereof, wherein;

B is formula (V):

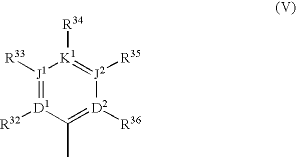

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D¹, J¹, J² and K¹ are O and S, and no more than four of D¹, D2 J J2 and K can be N with the proviso that R³², R³³, R³⁴, R³⁵, and R³⁶ are each independently selected to maintain the tetravalent nature of carbon, trivalent nature of nitrogen, the divalent nature of sulfur, and the divalent nature of oxygen;

R³², R³³, R³⁴, R³⁵ and R³⁶ can independently be Q^{b};

R⁹, R¹⁰, R¹¹, R¹², R¹³, R¹⁶, R¹⁷, R¹⁸, R¹⁹, R³², R³³, R³⁴, R³⁵, and R³⁶ are independently selected from the group consisting of hydrido, amidino, guanidino, dialkylsulfonium, trialkylphosphonium, dialkylsulfoniumalkyl, carboxy, heteroaralkylthio, heteroaralkoxy, cycloalkylamino, acylalkyl, acylalkoxy, aryloylalkoxy, heterocyclyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsufinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyallyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, alkoxyamino, thio, nitro, lower alkylamino, alkylthio, alkylthioalkyl, arylamino, aralkylamino, arylthio, axylthioalkyl, heteroaralkoxyalkyl, alkylsulfinyl, alkylsulfinylalkyl, arylsulfinylalkyl, alkylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, aminoalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl;

R³² and R³³, R³³ and R³⁴, R³⁴ and R³⁵, and R³⁵ and R³⁶ can be independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R³² and R³³, R³³ and R³⁴, R³⁴ and R³⁵, and R³⁵ and R³⁶ can be used at the same time;

R⁹ and R¹⁰, R¹⁰ and R¹¹, R¹¹ and R¹², and R¹² and R¹³ can be independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R⁹ and R¹⁰, R¹⁰ and R¹¹, R¹¹ and R¹², and R¹² and R¹³ can be used at the same time;

B can be formula (VI):

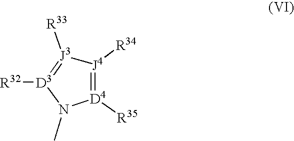

wherein D³, D⁴, J³, and J⁴ are independently selected from the group consisting of C, N, O, and S, no more than one of D³, D⁴, J³, and J⁴ can be O, no more than one of D³, D⁴, J³, and J⁴ can be S, and no more than three of D¹, D², J¹, and J² can be N with the proviso that R³², R³³, R³⁴, and R³⁵ are each independently selected to maintain the tetravalent nature of carbon, trivalent nature of nitrogen, the divalent nature of sulfur, and the divalent nature of oxygen;

B can be selected from the group consisting of C3-C8 alkyl, C3-C8 alkenyl, C3-C8 alkynyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;

B can be selected from the group consisting of C3-C10 cycloalkyl, C5 C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;

A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, Se(O), Se(O)₂, Se(O)₂N(R⁷), (R⁷)NSe(O)₂, P(O)(R⁸), N(R⁷)P(O)(R⁸), P(O)(Re)N(R⁷), C(NR⁷)N(R⁷), (R⁷)NC(NR⁷), and N(R⁷);

R⁷ and R⁸ are independently selected from the group consisting of hydrido, hydroxy, alkyl, alkenyl, aryl, aralkyl, aryloxy, alkoxy, alkenyloxy, alkylthio, alkylamino, arylthio, arylamino, acyl, aroyl, heteroaroyl, aralkoxyalkyl, heteroaralkoxyalkyl, aryloxyalkyl, alkoxyalkyl, alkenyloxyalkyl, alkylthioalkyl, arylthioalkyl, aralkoxyalkyl, heteroaralkoxyalkyl, alkylsulfinylalkyl, alkylsulfonylalkyl, heteroaryl, heteroaryloxy, heteroarylamino, heteroaralkyl, heteroaralkyloxy, heteroaralkylamino, and heteroaryloxyalkyl;

R^{14,} R¹⁵, R³⁷, R³⁸, R³⁹, R⁴⁰, R⁴¹ and R⁴² are independently selected from the group consisting of hydrido, hydroxy, halo, cyano, aryloxy, amino, alkylamino, dialkylamino, hydroxyalkyl, aminoalkyl, acyl, aroyl, heteroaroyl, heteroaryloxyalkyl, sulfhydryl, acylamido, alkoxy, alkylthio, arylthio, alkyl, alkenyl, alkynyl, aryl, aralkyl, aryloxyalkyl, aralkoxyalkylalkoxy, alkylsulfinylalkyl, alkylsulfonylalkyl, aralkylthioalkyl, heteroaralkoxythioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, saturated heterocyclyl, partially saturated heterocyclyl, heteroaryl, heteroarylalkyl, heteroarylthioalkyl, heteroaralcylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, aralkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, trialkylsilyl, dialkoxyphosphono, diaralkoxyphosphono, dialkoxyphosphonoalkyl, and diaralkoxyphosphonoalkyl;

R¹⁴ and R¹⁴, when bonded to different carbons, can be taken together to form a group selected from the group consisting of covalent bond, alkylene, haloalkylene, and a linear moiety spacer selected to form a ring selected from the group consisting of cycloalkyl ring having from 5 through 8 contiguous members, cycloalkenyl ring having from 5 through 8 contiguous members, and a heterocyclyl having from 5 through 8 contiguous members;

R¹⁴ and R¹⁵, when bonded to different carbons, can be taken together to form a group selected from the group consisting of covalent bond, alkylene, haloalkylene, and a linear moiety spacer selected to form a ring selected from the group consisting of a cycloalkyl ring having from 5 through 8 contiguous members, a cycloalkenyl ring having from 5 through 8 contiguous members, and a heterocyclyl having from 5 through 8 contiguous members;

R¹⁵ and R¹⁵, when bonded to different carbons, can be taken together to form a group selected from the group consisting of covalent bond, alkylene, haloalkylene, and a linear moiety spacer selected to form a ring selected from the group consisting of cycloalkyl ring having from 5 through 8 contiguous members, cycloalkenyl ring having from 5 through 8 contiguous members, and a heterocyclyl having from 5 through 8 contiguous members;

Ψ is selected from the group consisting of NR⁵, O, C(O), C(S), S, S(O), S(O)₂, ON(R⁵), P(O)(R⁸), and CR³⁹R⁴⁰;

R⁵ is selected from the group consisting of hydrido, alkyl, alkenyl, alkynyl, aryl, aralkyl, aryloxy, alkoxy, alkenyloxy, alkylthio, arylthio, aralkoxyalkyl, heteroaralkoxyalkyl, aryloxyalkyl, alkoxyalkyl, alkenyloxyalkyl, alkylthioalkyl, arylthioalkyl, aralkoxyalkyl, heteroaralkoxyalkyl, alkylsulfinylalkyl, alkylsulfonylalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxyalkyl, halocycloalkenyloxyalkyl, heteroaryl, heteroarylalkyl, monocarboalkoxyalkyl, monocarboalkoxy, dicarboalkoxyalkyl, monocarboxamido, monocyanoalkyl, dicyanoalkyl, carboalkoxycyanoalkyl, acyl, aroyl, heteroaroyl, heteroaryloxyalkyl, and dialkoxyphosphonoalkyl;

R³⁹ and R⁴⁰, when bonded to the same carbon, can be taken together to form a group selected from a group consisting of oxo, thiono, R⁵—N, alkylene, haloalkylene, and a linear moiety spacer having from 2 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

R¹ is selected from the group consisting of hydrido, alkyl, alkenyl, haloalkyl, and alkoxyalkyl;

X⁰ and R² are independently selected from the group consisting of Z⁰-Q, hydrido, alkyl, alkenyl, and halo;

X⁰ and R² can be independently selected from the group consisting of amidino, guanidino, dialkylsulfonium, trialkylphosphonium, dialkylsulfoniumalkyl, heteroarylamino, amino, nitro, alkylamino, arylamino, aralkylamino, alkanoyl, alkenoyl, aroyl, heteroaroyl, aralkanoyl, heteroaralkanoyl, haloalkanoyl, hydroxyhaloalkyl, cyano, and phosphono;

X⁰ and R¹ can be taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X⁰ and R¹ and spacer pair R² and R¹ can be used at the same time;

R² and R¹ can be taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X⁰ and R¹ and spacer pair R² and R¹ can be used at the same time;

X₀ and R⁵ can be taken together to form a spacer pair wherein the spacer pair forms a linear spacer moiety having from 2 through 5 contiguous atoms connecting the points of bonding of said spacer pair members to form a heterocyclyl ring having from 5 through 8 contiguous members;

X⁰ and R³⁹ can be taken together to form a spacer pair wherein the spacer pair forms a linear spacer moiety having from 2 through 5 contiguous atoms connecting the points of bonding of said spacer pair members to form a heterocyclyl ring having from 5 through 8 contiguous members;

X⁰ and R⁴⁰ can be taken together to form a spacer pair wherein the spacer pair forms a linear spacer moiety having from 2 through 5 contiguous atoms connecting the points of bonding of said spacer pair members to form a heterocyclyl ring having from 5 through 8 contiguous members;

X⁰ can be independently selected to form a linear moiety having from 2 through 5 contiguous atoms linked to the points of bonding of both R³⁹ and R⁴⁰ to form a heterocyclyl ring having from 5 through 8 contiguous members;

R² and R^{4a}, R² and R^{4b}, R² and R¹⁴, and R² and R¹⁵ can be independently selected to form spacer pairs wherein a spacer pair is taken together to form a linear moiety having from 2 through 5 contiguous atoms connecting the points of bonding of said spacer pair members to form a heterocyclyl ring having from 5 through 8 contiguous members with the proviso that no more than one of the group of spacer pairs consisting of R² and R^{4a}, R² and R^{4b}, R² and R¹⁴, and R² and R¹⁵ can be used at the same time;

R² can be independently selected to form a linear moiety having from 2 through 5 contiguous atoms linked to the points of bonding of both R^{4a} and R^{4b} to form a heterocyclyl ring having from 5 through 8 contiguous members;

Z⁰ is selected from the group consisting of covalent single bond, (CR⁴¹R⁴²)_{q} wherein q is an integer selected from 1 through 6, (CH(R⁴¹))_{g}—W⁰—(CH(R⁴²)), wherein g and p are integers independently selected from 0 through 3 and W⁰ is selected from the group consisting of O, S, C(O), C(S), C(O)O, C(S)O, C(O)S, C(S)S, C(O)N(R⁴¹), (R⁴¹)NC(O), C(S)N(R⁴¹), (R⁴¹)NC(S), OC(O)N(R⁴¹), (R⁴¹)NC(O)O, SC(S)N(R⁴¹), (R⁴¹)NC(S)S, SC(O)N(R⁴¹), (R⁴¹)NC(O)S, OC(S)N(R⁴¹), (R⁴¹)NC(S)O, N(R⁴²)C(O)N(R⁴¹), (R⁴¹)NC(O)N(R⁴²), N(R⁴²)C(S)N(R⁴¹) (R⁴¹)NC(S)N(R⁴²), S(O), S(O)₂, S(O)₂N(R⁴¹), N(R⁴¹)S(O)₂, Se, Se(O), Se(O)₂, Se(O)₂N(R⁴¹), N(R⁴¹)Se(O)₂, P(O)(R⁸), N(R⁷)P(O)(R⁸), P(O)(R⁸)N(R⁷), N(R⁴¹), ON(R⁴¹), and SiR²⁸R²⁹, and (CH(R⁴¹))ₑ—W²—(CH(R⁴²))ₕ wherein e and h are integers independently selected from 0 through 2 and W² is selected from the group consisting of CR⁴¹═CR⁴² CR⁴¹R⁴²C; vinylidene), and ethynylidene (C—C; 1,2-ethynyl), with the proviso that R and R are selected from other than halo and cyano when directly bonded to N and Z⁰ is directly bonded to the pyridone ring;

Q^{b} is formula (II):

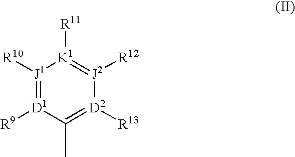

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four of D¹, D², J¹, J² and K¹ can be N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;

Q can be formula (III):

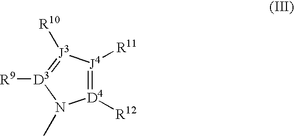

wherein D³, D⁴, J³, and J⁴ are independently selected from the group consisting of C, N, O, and S, no more than one of D³, D⁴, J³, and J⁴ can be O, no more than one of D³, D⁴, J³, and J⁴ can be S, and no more than three of D¹, D², J¹, and J² can be N with the proviso that R⁹, R¹⁰, R¹¹, and R¹² are each independently selected to maintain the tetravalent nature of carbon, trivalent nature of nitrogen, the divalent nature of sulfur, and the divalent nature of oxygen;

Q can be selected from the group consisting of alkyl, alkoxy, alkylamino, alkylthio, haloalkylthio, alkenyl, alkynyl, saturated heterocyclyl, partially saturated heterocyclyl, acyl, aroyl, heteroaroyl, cycloalkyl, cycloalkylalkyl, cycloalkenyl, cycloalkenylalkyl, cycloalkylalkenyl, haloalkyl, haloalkoxy, haloalkenyl, halocycloalkyl, halocycloalkenyl, haloalkoxyalkyl, haloalkenyloxyalkyl, halocycloalkoxyalkyl, and halocycloalkenyloxyalkyl;

K is (CR^{4a}R^{4b})ₙ wherein n is an integer selected from 1 through 4;

R^{4a} and R^{4b} are independently selected from the group consisting of halo, hydrido, hydroxy, cyano, hydroxyalkyl, alkyl, alkenyl, aryl, aralkyl, aralkoxyalkyl, aryloxyalkyl, alkoxyalkyl, heteroaryloxyalkyl, alkenyloxyalkyl, alkylthioalkyl, aralkylthioalkyl, arylthioalkyl, cycloalkyl, cycloalkylalkyl, haloalkyl, haloalkenyl, heteroaryl, heteroarylalkyl, heteroarylthioalkyl, heteroaralkylthioalkyl, cyanoalkyl, alkylsulfinylalkyl, alkylsulfonylalkyl, haloalkylsulfinyl, arylsulfinylalkyl, arylsulfonylalkyl, heteroarylsulfonylalkyl, heteroarylsulfinylalkyl, aralkylsulfinylalkyl, and aralkylsulfonylalkyl;

R^{4a} and R^{4b}, when bonded to the same carbon, can be taken together to form a group selected from the group consisting of oxo, thiono, and a linear spacer moiety having from 2 through 7 contiguous atoms connected to form a ring selected from the group consisting of a cycloalkyl ring having 3 through 8 contiguous members, a cycloalkenyl ring having 5 through 8 contiguous members, and a heterocyclyl ring having 5 through 8 contiguous members;

E⁰ is E¹, when K is (CR^{4a}R^{4b})ₙ, wherein E is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, S(O)₂N(R⁷)C(O), C(O)N(R⁷)S(O)₂, Se, Se(O), Se(O)₂, Se(O)₂N(R⁷), N(R⁷)Se(O)₂, P(O)(R⁸), N(R⁷)P(O)(R⁸), P(O)R⁸)N(R⁷), N(R⁷), ON(R⁷), SiR²⁸R²⁹, CR^{4a}═CR^{4b}, ethynylidene (CRC; 1,2-ethynyl), and C═CR^{4a}R^{4b};

R²⁸ and R²⁹ are independently selected from the group consisting of hydrido, hydroxyalkyl, alkyl, alkenyl, alkynyl, aryl, aralkyl, aryloxyalkyl, acyl, aroyl, aralkanoyl, heteroaroyl, aralkoxyalkyl, alkylsulfinylalkyl, alkylsulfonylalkyl, aralkylthioalkyl, heteroararlylthioalkyl, alkoxyalkyl, heteroaryloxyalkyl, alkenyloxyalkyl, alkylthioalkyl, arylthioalkyl, cycloagly cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, arylsulfinylalkyl, arylsulfonylalkyl, aralkylsulfinyl, cycloalkylsulfinylalkyl, cycloalkylsufonylalkyl, heteroarylsulfonylalkyl, heteroarylsulfinylalkyl, aralkylsulfinylalkyl, aralkylsulfonylalkyl, carboxy, dialkoxyphosphono, diaralkoxyphosphono, dialkoxyphosphonoalkyl and diaralkoxyphosphonoalkyl;

R²⁸ and R²⁹ can be taken together to form a linear moiety spacer having from 2 through 7 contiguous atoms and forming a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

K can be (CH(R¹⁴))ⱼ-T wherein j is selected from a integer from 0 through 3 and T is selected from the group consisting of single covalent bond, O, S, and N(R⁷) with the proviso that (CH(R¹⁴))ⱼ is bonded to the pyridone ring;

E⁰ is E², when K is (CH(R¹⁴))ⱼ-T, wherein B is selected from the group consisting of a covalent single bond, C(O), C(S), C(O)O, C(S)O, C(O)S, C(S)S, C(O)N(R⁷), (R⁷)NC(O), C(S)N(R⁷), (R⁷)NC(S), (R⁷)NC(O)O, (R⁷)NC(S)S, (R⁷)NC(O)S, (R⁷)NC(S)O, N(R⁷)C(O)N(R⁷), (R⁷)NC(O)N(R⁸), N(R⁷)C(S)N(R⁷), (R⁷)NC(S)N(R⁸), S(O), S(O)₂, S(O)₂N(R⁷), N(R⁷)S(O)₂, S(O)₂N(H)C(O), C(O)N(H)S(O)₂, Se(O), Se(O)₂, Se(O)₂N(R⁷), N(R⁷)Se(O)₂, P(O)(R⁸), N(R⁷)P(O)(R⁸), P(O)(R⁸)N(R⁷), and N(R⁷);

K can be G-(CH(R¹⁵))ₖ wherein k is selected from an integer from 1 through 3 and G is selected from the group consisting of O, S, and N(R⁷) with the proviso that R¹⁵ is other than hydroxy, cyano, halo, amino, alkylamino, dialkylamino, and sulfhydryl when k is 1;

E⁰ is E³ when K is G-(CH(R¹⁵))ₖ wherein E³ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R ⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, Se, Se(O), Se(O)₂, Se(O)₂N(R⁷), N(R^{7)Se(O)}₂, P(O)(R⁸), N(R⁷)P(O)(R⁸), P(O)(R⁸)N(R⁷), N(R⁷), ON(R⁷), SiR²⁸R²⁹, CR^{4a}═CR^{4b} ethynylidene (C═C; 1,2-ethynyl), and C═CR^{4a}R^{4b};

Y⁰ is formula (IV):

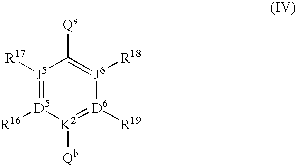

wherein D⁵, D⁶, J⁵, and J⁶ are independently selected from the group consisting of C, N, O, S and a covalent bond with the provisos that no more than one can be a covalent bond, K² is independently selected from the group consisting of C, and N⁺, no more than one of D ⁵, D⁶, J⁵, and J⁶ can be O, no more than one of D⁵, D⁶, J⁵, and J⁶ can be S, one of D⁵, D⁶, J⁵, and J⁶ must be a covalent bond when two of D⁵, D⁶, J⁵, and J⁶ are and S, no more than three of D⁵, D⁶, J⁵, and J⁶ can be N when K² is N⁺, and no more than four of D⁵, D⁶, J⁵, and J⁶ can be N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;

R¹⁶ and R¹⁷ can be independently taken together to form a linear moiety spacer having from 3 through 6 contiguous atoms connected to form a ring selected from the group consisting of a cycloalkenyl ring having from 5 through 8 contiguous members, a partially saturated heterocyclyl ring having from 5 through 8 contiguous members, a heteroaryl having from 5 through 6 contiguous members, and an aryl;

R¹⁸ and R¹⁹ can be independently taken together to form a linear moiety spacer having from 3 through 6 contiguous atoms connected to form a ring selected from the group consisting of a cycloalkenyl ring having from 5 through 8 contiguous members, a partially saturated heterocyclyl ring having from 5 through 8 contiguous members, a heteroaryl having from 5 through 6 contiguous members, and an aryl;

Q^{b} is selected from the group consisting of NR²⁰R²¹, ⁺NR²⁰R²¹R²², oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² can be hydroxy, alkoxy, alkylamino, amino, and dialkylamino and that R²⁰, R²¹, and R²² must be other than be hydroxy, alkoxy, alkylamino, amino, and dialkylamino when K² is N⁺;

R²⁰ and R²¹, R²⁰ and R²², and R²¹ and R²² can be independently selected to form a spacer pair wherein a spacer pair is taken together to form a linear moiety having from 4 through 7 contiguous atoms connecting the points of bonding of said spacer pair members to form a heterocyclyl ring having 5 through 8 contiguous members with the proviso that no more than one of the group consisting of spacer pairs R²⁰ and R²¹, R²⁰ and R²², and R²¹ and R²² can be used at the same time;

Q^{b} can be selected from the group consisting of N(R²⁶)SO₂N(R²³)(R²⁴). N(R²⁴)C(O)OR⁵, N(R²⁶)C(O)SR⁵, N(R²⁶)C(S)OR⁵ and N(R²⁶)C(S)SR⁵ with the proviso that no more than one of R²³, R²⁴, and R²⁶ can be hydroxy, alkoxy, alkylamino, amino, or dialkylamino when two of the group consisting of R²³, R²⁴, and R²⁶ are bonded to the same atom;

Q^{b} can be selected from the group consisting of dialkylsulfonium, trialkylphosphonium,C(NR²⁵)NR²³, R²⁴,N(R²⁶)C(NR²⁵)N(R²³)(R²⁴), N(R²⁶)C(O)N(R²³)(R²⁴), N(R²⁶)C(S)N(R²³)(R²⁴), C(NR²⁵)OR⁵, C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁴), N(O)N(R²⁶)SO₂N(R²³)(R²⁴), C(NR²⁵)SR⁵, C(O)NR²³R²⁴, and C(O)NR²³R²⁴ with the provisos that no more than one of R²³, R²⁴, and R²⁶ can be hydroxy, alkoxy, alkylamino, amino, or dialkylamino when two of the group consisting of R²³, R²⁴, and R²⁶ are bonded to the same atom and that said Q group is bonded directly to a carbon atom;

R²³, R²⁴, R²⁵, and R²⁶ are independently selected from the group consisting of hydrido, alkyl, hydroxy, alkoxy, alkylamino, dialkylamino, aminoalkyl, and hydroxyalkyl;

R²³ and R²⁴ can be taken together to form a linear spacer moiety having from 4 through 7 contiguous atoms connecting the points of bonding to form a heterocyclyl ring having 5 through 8 contiguous members;

R²³ and R²⁵, R²⁴ and R²⁵, R²⁵ and R²⁶, R²⁴ and R²⁶, and R²³ and R²⁶ can be independently selected to form a spacer pair wherein a spacer pair is taken together from the points of bonding of selected spacer pair members to form the group L-U-V wherein L, U, and V are independently selected from the group consisting of O, S, C(O), C(S), C(J_{H})₂ S(O), SO₂, OP(OR³¹)R³⁰, P(O)R³⁰, P(S)R³⁰ C(R³⁰)R³¹, C═C(R³⁰)R³¹, (O)₂POP(O)₂, R(O)POP(O)R³⁰ Si(R²⁹)R²⁸ Si(R²⁹)R²⁸Si(R²⁹)R²⁸, Si(R²⁹)R²⁸OSi(R²⁹)R²⁸ (R²⁸)R²⁹COC(R²⁸)R²⁹, R²⁸)(R²⁹CSC(R²⁸)R²⁹, C(O)C(R³⁰)=C(R³¹), C(S)C(R³⁰)=C(R³¹), S(O)C(R³⁰)c(R³¹), SO₂C(R³⁰)=C(R³¹), PR³⁰C(R³¹)═C(R³¹), P(O)R³⁰C(R³⁰)═C(³¹), P(s)R³⁰C(R³⁰)=C(R³¹), DC(R³⁰)(R³¹)D, OP(OR³¹)R³⁰, P(O)R³⁰P(S)R³⁰, Si(R²⁸)R²⁹ and N(R³⁰), and a covalent bond with the proviso that no more than any two of L, U and V are simultaneously covalent bonds and the heterocyclyl comprised of by L, U, and V has from 5 through 10 contiguous member;

D is selected from the group consisting of oxygen, C═O, C═S, S(O)ₘ wherein m is an integer selected from 0 through 2;

J_{H} is independently selected from the group consisting of OR²⁷, S²⁷ and N(R²⁰)R²¹;

R²⁷ is selected from the group consisting of hydrido, alkyl, alkenyl, alkynyl, aralkyl, aryloxyalkyl, aralkoxyalkyl, alkylsulfinylalkyl, alkylsulfonylalkyl, aralkylthioalkyl, heteroaralkylthioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxyalkyl, halocycloalkenyloxyalkyl, perhaloaryloxyalkyl, heteroaryl, heteroarylalkyl, heteroarylthioalkyl, heteroarlkylthioalkyl, arylsulfinylalkyl, arylsulfonylalkyl, cycloalkylsulfinylalkyl, cycloalkylsufonylalkyl, heteroarylsulfonylalkyl, heteroarylsulfinylalkyl, aralkylsulfinylalkyl and aralkylsulfonylalkyl;

R³⁰ and R³¹ are independently selected from hydrido, hydroxy, thiol, aryloxy, amino, alkylamino, dialkylamino, hydroxyalkyl, heteroaryloxyalkyl, alkoxy, alkylthio, arylthio, alkyl, alkenyl, alkynyl, aryl, aralkyl, aryloxyalkyl, aralkoxyalkyl, alkylsulfinylalkyl, alkylsulfonylalkyl, aralkylthioalkyl, heteroaralkoxythioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, haloaralkylsulfinylalkyl, aralkylsulfonylalkyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, sulfonylalkyl, alkoxysulfonylalkyl, aralkoxysulfonylalkyl, alkoxysulfonylalkoxy, aralkoxysulfonylalkoxy, sulfonylalkoxy, alkoxysulfonylalkylamino, aralkoxysulfonylalkylamino, and sulfonylalkylamino;

R³⁰ and R³¹ can be taken to form a linear moiety spacer group having from 2 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

R²³ and R²⁵, R²⁴ and R²⁵, R²⁵ and R²⁴, R²⁴ and R²⁶ and R²³ and R²⁶ can be independently selected to form a spacer pair wherein a spacer pair is taken together from the points of bonding of selected spacer pair members to form the group L-U-V wherein L, U, and V are independently selected from the group of 1,2, disubstituted radicals consisting of a cycloalkyl radical, a cycloalkenyl radical wherein cycloalkyl and cycloalkenyl radicals are substituted with one or more groups selected from R³⁰ and R³¹, an aryl radical, an heteroaryl radical, a saturated heterocyclic radical and a partially saturated heterocyclic radical wherein said 1,2-substitutents are independently selected from C═O, C═S, C(R²⁸)R³², SO), S(O)₂ OP(OR³¹)R³⁰, P(O)R³⁰, P(S)R³⁰ and Si(R²⁸)R²⁹;

R²³ and R²⁵, R²⁴ and R²⁵, R²⁵ and R²⁶, R²⁴ and R²⁶, and R²³ and R²⁶ can be independently selected to form a spacer pair wherein a spacer pair is taken together from the points of bonding of selected spacer pair members to form the group L-U-V wherein L, U, and V are independently selected from the group of radicals consisting of 1,2-disubstituted alkylene radicals and 1,2-disubstituted alkenylene radical wherein said 1,2-substitutents are independently selected from C═O, C═S, C(R²⁸)R²⁹, S(O), S(O)₂, OP(OR³¹)R³⁰, P(O)R³⁰, P(S)R³⁰, and Si(R²⁸)R²⁹ and said alkylene and alkenylene radical are substituted with one or more R³⁰ or R³¹ substituents;

Q^{s} is selected from the group consisting of a single covalent bond, (CR³⁷R³⁸)_{b}—(W⁰)_{az} wherein az is an integer selected from 0 through 1, b is an integer selected from 1 through 4, and W⁰ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), SC(S)N(R¹⁴), SC(O)N(R¹⁴), OC(S)N(R¹⁴), N(R¹⁵)C(O)N(R¹⁴), (R¹⁴)NC(O)N(R¹⁵ N(R¹⁵)C(S)N(R¹⁴), (R¹⁴)NC(S)N(R¹⁵), S(O), S(O)₂, S(O)₂N(R¹⁴), N(R¹⁴)S(O)₂, Se, Se(O), Se(O)₂, Se(O)₂N(R¹⁷), N(R¹⁴)Se(O)₂, P(O)(R⁸), N(R⁷)P(O)(R⁸), P(O)(R⁸)N(R⁷), N(R¹⁴), ON(R¹⁴), and SiR²⁸R²⁹, (CH(R¹⁴))_{c}—W¹—(CH(R¹⁵))_{d} wherein c and d are integers independently selected from 1 through 4, and W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁵), (R¹⁴)NC(S)O, N(R¹⁵)C(O)N(R¹⁴), (R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, Se, Se(O), Se(O)₂, Se(O)₂N(R¹⁴), N(R¹⁴)Se(O)₂, P(O)(R⁸), N(R⁷)P(O)(R⁸), P(O)(R⁸)N(R⁷), N(R¹⁴), ON(R¹⁴), SiR²⁸R²⁹ and (CH(R¹⁴))ₑ—W²—(CH(R¹⁵))ₕ wherein e and h are integers independently selected from 0 through 2 and W² is selected from the group consisting of CR^{4a}═CR^{4b}, ethynylidene (C═C; 1,2-ethynyl), and C═CR^{4a}R^{4b} with the provisos that R¹⁴ and R¹⁵ are selected from other than halo and cyano when directly bonded to N and that (CR³⁷R³⁸)_{b}, (CH(R¹⁴))_{c}, (CH(R¹⁴))ₑ and are bonded to E⁰;

R³⁷ and R³⁷, when bonded to different carbons, can be taken together to form a linear moiety spacer having from 1 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

R³⁷ and R³⁸, when bonded to different carbons, can be taken together to form a linear moiety spacer having from 1 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

R³⁸ and R³⁸, when bonded to different carbons, can be taken together to form a linear moiety spacer having from 1 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

R³⁷ and R³⁸, when bonded to the same carbon, can be taken together to form a group selected from a group consisting of oxo, thiono, alltylene, haloalkylene, and a linear moiety spacer having from 2 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;

Y⁰ can be Q^{b} Q^{ss} wherein Q^{ss} is selected from the group consisting of (CR³⁷R³⁸)_{f} wherein f is an integer selected from 1 through 6, (CH(R¹⁴))_{c}—W¹—(CH(R¹⁵))_{d} wherein c and d are integers independently selected from 1 through 4, and W¹ is selected from the group consisting of W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, Se, Se(O), Se(O)₂, Se(O)₂N(R¹⁴), N(R¹⁴)Se(O)₂, P(O)(R⁸), N(R⁷)P(O)(R), P(O)e)N(R), N(R¹⁴), ON(R⁴), SiR²⁸R²⁹, and (CH(R¹⁴))ₑ—W²—(CH(R¹⁵))ₕ wherein e and h are integers independently selected from 0 through 2 and W² is selected from the group consisting of CR^{4a}═CR^{4b}, ethynylidene (C≡C; 1,2-ethynyl), and C═CR^{4a}R^{4b} with the provisos that R¹⁴ and R¹⁵ are selected from other than halo and cyano when directly bonded to N and that (CR³⁷R³⁸)_{f}, (CH(R¹⁵))_{c}, and (CH(R¹⁵))ₑ are bonded to E⁰;

Y⁰ can be Q^{b}-Q^{sss} wherein Q^{sss} is (CH(R³⁸))ᵣ—W³, r is an integer selected from 1 through 3, and W³ is selected from the group consisting of 1,1-cyclopropyl, 1,2-cyclopropyl, 1,1-cyclobutyl, 1,2-cyclobutyl, 1,2-cyclohexyl, 1,3-cyclohexyl, 1,4-cyclohexyl, 1,5-cyclopentyl, 1,3-cyclopentyl, 2,3-morpholinyl, 2,4-morpholinyl, 2,5-morpbolinyl, 2,6-morpholinyl, 3,4-morpholinyl, 3,5-morpholinyl, 1,2-piperazinyl, 1,3-piperazinyl, 1,4-piperazinyl, 2,3-piperazinyl, 2,5-piperazinyl, 2,6-piperazinyl, 1,2-piperidinyl, 1,3-piperidinyl, 1,4-piperidinyl, 2,3-pipenidinyl, 2,4-pipenidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4,5-yl, 4H-pyranone-2,3-yl, 2,3-tetrahydrofuranyl, 2,4-tetrahydrofuranyl, 2,5-tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the proviso that (CH(R³⁸))ᵣ is bonded to E⁰ and Q^{b} is bonded to lowest numbered substituent position of each W³;

Y⁰ can be Q^{b}-Q^{sssr} wherein Q^{b} is (CH(R³⁸))ᵣ—W⁴, r is an integer selected from 1 through 3, and W is selected from the group consisting of 1,2-cyclobutyl, 1,2-cyclohexyl, 1,3-cyclohexyl, 1,4-cyclohexyl, 1,2-cyclopentyl, 1,3-cyclopentyl, 2,3-morpholinyl, 2,4-morpholinyl, 2,;morpholinyl, 2,6-morpholinyl, 3,4-morpholinyl, 3,5-morpholinyl, 1,2-piperazinyl, 1,3-piperazinyl, 1,4-piperazinyl, 2,3-piperazinyl, 2,5-piperazinyl, 2,6-piperazinyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, ²H-pyran-2-one-4,5-yl, 4H-pyran-ne-2,3-yl, 2,3-tetrahydrofuranyl, 2,4-tetrahydrofuranyl, 2,5-tetrahydrofuranyl, 3,4-tethydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the provisos that (CH(R³⁸))ᵣ is bonded to E⁰ and Q^{b} is bonded to highest number substituent position of each W⁴;

Y⁰ can be Q^{b}-Q^{SSSS} wherein Q^{ssss} (CH(R³⁸))ᵣ—W⁵, r is an integer selected from 1 through 3, and W is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,7-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-beiizisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2.5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to lowest number substituent position of each W⁵ and that (CH(R³⁸))ᵣ is bonded to E;

Y⁰ can be Q^{b}-Q^{ssssr} wherein Q^{ssssr} is (CH(R³⁸))ᵣ—W⁶, r is an integer selected from 1 through 3, and W is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofumrnyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-sbenzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyi, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,&indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,5-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,5-qtquinolinyl, 3,7-quinolinyl, 3,5-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to highest number substituent position of each W⁶ and that (CH(R³⁸))ᵣ is bonded to E⁰.

In an embodiment of compounds of Formula I or a pharmaceutically acceptable salt thereof,

B is formula (V):

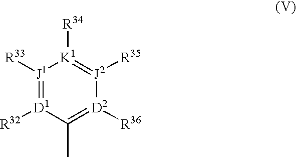

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four of D¹, D², J¹, J² and K¹ can be N with the proviso that R³², R³³, R³⁴, R³⁵, and R³⁶ are each independently selected to maintain the tetravalent nature of carbon, trivalent nature of nitrogen, the divalent nature of sulfur, and the divalent nature of oxygen;

R³², R³³, R³⁴, R³⁵, and R³⁶ can independently be Q^{b};

R⁹, R¹⁰, R¹¹, R¹², R¹³, R¹⁶, R¹⁷, R¹⁸, R¹⁹, R³², R³³, R³⁴, R³⁵, and R³⁶ are independently selected from the group consisting of hydrido, amidino, guanidino, dialkylsulfonium, trialkylphosphonium, dialkylsulfoniumalkyl, carboxy, heteroaralkylthio, heteroaralkoxy, cycloalkylamino, acylalkyl, acylalkoxy, aryloylalkoxy, heterocydyloxy, aralkylaryl, aralkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyaglyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, alkoxyamino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosuifonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, aminoalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, axylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl;

R³² and R³³, R³³ and R³⁴, R³⁴ and R³⁵, and R³⁵ and R³⁶ can be independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R³² and R³³, R³³ and R³⁴, R³⁴ and R³⁵, and R³⁵ and R³⁶ can be used at the same time;

R⁹ and R¹⁰, R¹⁰ and R¹¹, R¹¹ and R¹², and R¹² and R¹³ can be independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R⁹ and R¹⁰, R¹⁰ and R¹¹, R¹¹ and R¹², and R¹² and R¹³ can be used at the same time;

B can be selected from the group consisting of C3-C8 alkyl, C3-C8 alkenyl, C3-C8 alkynyl, C3-CS haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;

B can be selected from the group consisting of C3-C10 cycloalkyl, C5-C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;

A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, P(O)(R⁸), N(R⁷)P(O)(R⁸), P(O)(R⁸)N(R⁷), C(NR⁷)N(R⁷), (R⁷)NC(NR⁷),and N(R⁷);

R⁷ and R⁸ are independently selected from the group consisting of hydrido, hydroxy, alkyl, acyl, aroyl, heteroaroyl, and alkoxyalkyl;

R¹⁴, R¹⁵, R³⁷, and R³⁸ are independently selected from the group consisting of hydrido, hydroxy, halo, cyano, hydroxyalkyl, alkoxy, alkyl, alkoxyalkyl, cycloalkyl, cycloalkylalkyl, cycloalkenyl, cycloalkenylalkyl, haloallyl, haloalkenyl, haloalkoxy, haloalkoxyalkyl, haloalkenyloxyalkyl, halocycloalkoxy, halocycloalkoxyalkyl, halocycloalkenyloxyalkyl, carboxy, carboxyalkyl, carboalkoxy, carboxamide, and carboxamidoalkyl;

Ψ is selected from the group consisting of NR⁵, O, C(O), C(S), S, S(O), S(O)₂, ON(R⁵), P(O)(R⁸), and CR³⁹R⁴⁰;

R⁵ is selected from the group consisting of hydrido, alkyl, alkoxy, alkoxyalkyl, haloalkyl, acyl, aroyl, and heteroaroyl;

R³⁹ and R⁴⁰ are independently selected from the group consisting of hydrido, hydroxy, halo, cyano, hydroxyalkyl, acyl, aroyl, heteroaroyl, acylamido, alkoxy, alkyl, alkoxyalkyl, haloalkyl, haloalkoxy, haloalkoxyallyl, alkylsulfonyl, haloalkylsulfonyl, carboxy, carboxyalkyl, carboalkoxy, carboxamide, and carboxamidoalkyl;

R¹ is selected from the group consisting of hydrido, alkyl, alkenyl, haloalkyl, and alkoxyalkyl;

X⁰ and R² are independently selected from the group consisting of Z⁰-Q, hydrido, alkyl, alkenyl, and halo;

X⁰ and R² can be independently selected from the group consisting of amidino, guanidino, dialkylsulfonium, trialkylphosphonium, dialkylsulfoniumalkyl, heteroarylamino, amino, nitro, alkylamino, arylamino, aralkylamino, alkanoyl, alkenoyl, aroyl, heteroaroyl, aralkanoyl, heteroaralkanoyl, haloalkanoyl, hydroxyhaloalkyl, cyano, and phosphono;

X⁰ and R¹ can be taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X⁰ and R¹ and spacer pair R² and R¹ can be used at the same time;

R² and R¹ can be taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X⁰ and R¹ and spacer pair R² and R¹ can be used at the same time;

Z⁰ is selected from the group consisting of covalent single bond, (CR⁴¹R⁴²)_{q} wherein q is an integer selected from 1 through 6, (CH(R⁴¹))_{g}-W⁰—(CH(R⁴²))ₚ wherein g and p are integers independently selected from 0 through 3 and W⁰ is selected from the group consisting of O, S, C(O), C(S), C(O)O, C(S)O, C(O)S, C(S)S, C(O)N(R⁴¹), (R⁴¹)NC(O), C(S)N(R⁴¹), (R⁴¹)NC(S), OC(O)N(R⁴¹), (R⁴¹)NC(O)O, SC(S)N(R⁴¹), (R⁴¹)NC(S)S, SC(O)N(R⁴¹), (R⁴¹)NC(O)S, OC(S)N(R⁴¹) (R⁴¹)NC(S)O, N(R⁴²)C(O)N(R⁴¹), (R⁴¹)NC(O)N(R⁴²), N(R⁴¹)C(S)N(R⁴¹), (R⁴¹)NC(S)N(R⁴²), S(O), S(O)₂, S(O)₂N(R⁴¹), N(R⁴¹)S(O)₂, P(O)(R⁸), N(R⁷)P(O)(R⁸), P(O)(R⁸)N(R⁷), N(R⁴¹), ON(R⁴¹), and (CH(R⁴¹))ₑ—W²—(CH(R⁴²))ₕ wherein e and h are integers independently selected from 0 through 2 and W is selected from the group consisting of CR═CR CR⁴¹R⁴²═C; vinylidene), and ethynylidene (C≡C; 1,2-ethynyl), with the proviso that R⁴¹ and R⁴² are selected from other than halo and cyano when directly bonded to N and Z⁰ is directly bonded to the pyridone ring;

R⁴¹ and R⁴² are independently selected from the group consisting of hydrido, hydroxy, halo, cyano, aryloxy, hydroxyalkyl, acyl, aroyl, heteroaroyl, heteroaryloxyalkyl, alkoxy, alkyl, aryl, aralkyl, aryloxyalkyl, aralkoxyalkylalkoxy, alkoxyalkyl, heteroaryloxyalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, saturated heterocyclyl, partially saturated heterocyclyl, heteroaryl, heteroaralkyl, heteroarylthioalkyl, heteroaralkylthioalkyl, alkylsulfonyl, haloalkylsulfonyl, arylsulfonyl, arylsulfonylalkyl, aralkylsulfonyl, cycloalkylsulfonyl, cycloalkylsufonylalkyl, heteroarylsulfonylalkyl, heteroarylsulfonyl, and aralkylsulfonylalkyl;

Q is formula (II):

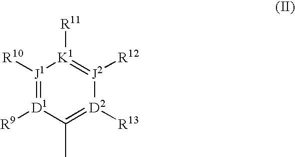

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four of D¹, D², J¹, J² and K¹ can be N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;

Q can be formula (III):

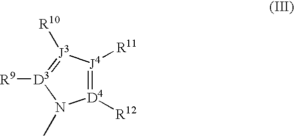

wherein D³, D⁴, J³, and J⁴ are independently selected from the group consisting of C, N, O, and S, no more than one of D³,D⁴, J³, and J⁴ can be O, no more than one of D³, D⁴, J³, and J⁴ can be S, and no more than three of D¹, D², J¹ and J² can be N with the proviso that R⁹, R¹⁰, R¹¹, R¹², and R¹² are each independently selected to maintain the tetravalent nature of carbon, trivalent nature of nitrogen, the divalent nature of sulfur, and the divalent nature of oxygen;

Q can be selected from the group consisting of alkyl, alkoxy, alkylamino, alkylthio, haloalkylthio, alkenyl, alkynyl, saturated heterocycyl, partially saturated heterocyclyl, acyl, aroyl, heteroaroyl, cycloalkyl, cycloalkylalkyl, cycloalkenyl, cycloalkenylalkyl, cycloalkylalkenyl, haloalkyl, haloalkoxy, haloalkenyl, halocycloalkyl, halocycloalkenyl, haloalkoxyalkyl, haloalkenyloxyalkyl, halocycloalkoxyalkyl, and halocycloalkenyloxyalkyl;

K is (CR^{4a}R^{4b})ₙ wherein n is an integer selected from 1 through 2;

R^{4a} and R^{4b} are independently selected from the group consisting of halo, hydrido, hydroxy, cyano, hydroxyalkyl, alkyl, alkenyl, alkoxyalkyl, haloalkyl, haloalkenyl, and cyanoalkyl;

R^{4a} and R^{4b}, when bonded to the same carbon, can be taken together to form a group selected from the group consisting of oxo, and a linear spacer moiety having from 2 through 7 contiguous atoms connected to form a ring selected from the group consisting of a cycloalkyl ring having 3 through 8 contiguous members, a cycloalkenyl ring having 5 through 8 contiguous members, and a heterocyclyl ring having 5 through 8 contiguous members;

E⁰ is a E¹, when K is (CR^{4a}R^{4b})ₙ wherein E¹ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, S(O)₂N(R⁷)C(O), C(O)N(R⁷)S(O)₂, P(O)(R⁸), N(R⁷)P(O)(R⁸), P(O)(R⁸)N(R⁷), N(R⁷), ON(R⁷), CR^{4a}═CR^{4b}, ethynylidene (C≡C; 1,2-ethynyl), and C—R^{4a}R^{4b};

K can be (CH(R¹⁴))ⱼ-T wherein j is selected from a integer from 0 through 2 and T is selected from the group consisting of single covalent bond, O, S, and N(R⁷) with the proviso that (CH(R⁷))ⱼ is bonded to the pyridone ring;

E⁰ is E², when K is (CH(R¹⁴))ⱼ-T, wherein E² is selected from the group consisting of a covalent single bond, C(O), C(S), C(O)O, C(S)O, C(O)S, C(S)S, C(O)N(R⁷), (R⁷)NC(O), C(S)N(R⁷), (R⁷)NC(S), (R⁷)NC(O)O, (R⁷)NC(S)S, (R⁸)NC(O)S, (R⁸)NC(S)O, N(R⁸)C(O)N(R⁷), (R⁷)NC(O)N(R⁸), N(R⁸)C(S)N(R⁷), R⁷)NC(S)N(R⁸), S(O), S(O), S(O)₂N(R⁷), N(R⁷)S(O)₂, S(O)₂N(H)C(O), C(O)N(H)S(O)₂, P(O)(R⁸), N(R⁷)P(O)(R⁸), P(O)(R⁸)N(R⁷), and N(R⁷);

K can be G-(CH(R¹⁵))ₖ wherein k is selected from an integer from 1 through 2 and G is selected from the group consisting of O, S, and N(R⁷) with the proviso that R¹⁵ is other than hydroxy, cyano, halo, amino, alkylamino, dialkylamino, and sulfhydryl when k is 1;

E⁰ is E³ when K is G-(CH(R¹⁵))ₖ wherein E³ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, P(O)(R⁸), N(R⁷)P(O)(R⁸), P(O)(R⁸)N(R⁷), N(R⁷), ON(R⁷), CR^{4a}═CR^{4b}, ethynylidene (C≡C; 1,2-ethynyl), and C═CR^{4a}R^{4b};

Y⁰ is formula (IV):

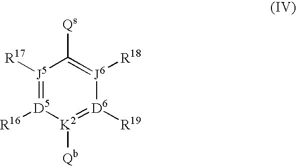

wherein D⁵, D⁶, J⁵, and J⁶ are independently selected from the group consisting of C, N, O, S and a covalent bond with the provisos that no more than one can be a covalent bond, K² is independently selected from the group consisting of C, and N⁺, no more than one of D⁵, D⁶, J⁵, and J⁶ can be O, no more than one of D⁵, D⁶, J5, and J⁶ can be S, one of D⁵, D⁶, J⁵, and J⁶ must be a covalent bond when two of D⁵, D⁶, J⁵, and J⁶ are O and S, no more than three of D⁵, D⁶, J⁵, and J⁶ can be N when K² is N⁺, and no more than four of D⁵, D⁶, J⁵, and J⁶ can be N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;

R¹⁶ and R¹⁷ can be independently taken together to form a linear moiety spacer having from 3 through 6 contiguous atoms connected to form a ring selected from the group consisting of a cycloalkenyl ring having from 5 through 8 contiguous members, a partially saturated heterocyclyl ring having from 5 through 8 contiguous members, a heteroaryl having from 5 through 6 contiguous members, and an aryl;

Q^{b} is selected from the group consisting of NR²⁰R²¹, ⁺NR²⁰R²¹R²², oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R R²¹, and R²² can be hydroxy, alkoxy, alkylamino, amino, and dialkylamino and that R²⁰, R²¹, and R²² must be other than be hydroxy, alkoxy, alkylamino, amino, and dialkylamino when K is N⁺,

R²⁰ and R²¹, R²⁰ and R²², and R²¹ and R²² can be independently selected to form a spacer pair wherein a spacer pair is taken together to form a linear moiety having from 4 through 7 contiguous atoms connecting the points of bonding of said spacer pair members to form a heterocyclyl ring having 5 through 8 contiguous members with the proviso that no more than one of the group consisting of spacer pairs R²⁰ and R²¹, R²⁰ and R²², and R²¹ and R²² can be used at the same time;

Q^{b} can be selected from the group consisting of N(R²⁶)SO₂N(R²³)(R²⁴),N(R²⁶)C(O)OR⁵, N(R²⁶)C(O)SR⁵, N(R²⁶)C(S)OR⁵ and N(R²⁶)C(S)SR⁵ with the proviso that no more than one of R²³, R²⁴, and R²⁶ can be hydroxy, alkoxy, alkylamino, amino, or dialkylamino when two of the group consisting of R²³, R²⁴, and R²⁶ are bonded to the same atom;

Q^{b} can be selected from the group consisting of dialkylsulfonium, trialkylphosphonium, C(NR²⁵)NR²³R²⁴, N(R²⁶)C(NR²⁵)N(R²³)(R²⁴), N(R²⁶)C(O)N(R²³)(R²⁴), N(R²⁶)C(S)N(R²³)(R²⁴), C(NR²⁵)OR⁵, C(O)N(R²⁶)C(NR²⁶)N(R²³)(R²⁴), C(S)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(NR²⁵)SR⁵, C(O)NR²³R²⁴, and C(O)NR²³R²⁴ with the provisos that no more than one of R²³, R²⁴, and R²⁶ can be hydroxy, alkoxy, alkylamino, amino, or dialkylamino when two of the group consisting of R²³, R²⁴, and R²⁶ are bonded to the same atom and that said Q^{b} group is bonded directly to a carbon atom;

R²³, R²⁴, R²⁵, and R²⁶ are independently selected from the group consisting of hydrido, alkyl, hydroxy, alkoxy, alkylamino, dialkylamino, aminoalkyl, and hydroxyalkyl;

R²³ and R²⁴ can be taken-together to form a linear spacer moiety having from 4 through 7 contiguous atoms connecting the points of bonding to form a heterocyclyl ring having 5 through 8 contiguous members;

Q^{s} is selected from the group consisting of a single covalent bond, (CR³⁷R³⁸)_{b}—(W⁰)_{az} wherein az is an integer selected from 0 through 1, b is an integer selected from 1 through 4, and W⁰ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), SC(S)N(R¹⁴), SC(O)N(R¹⁴), OC(S)N(R¹⁴), N(R¹⁵)C(O)N(R¹⁴), (R¹⁴)NC(O)N(R¹⁵), N(R¹⁵)C(S)N(R¹⁴) (R¹⁴)NC(S)N(R¹⁵), S(O), S(O)₂, S(O)₂N(R¹⁴), N(R¹⁴)S(O)₂, P(O)(R⁸), N(R⁷)P(O)(R⁸), P(O)(R⁸)N(R⁷), N(R¹⁴),

ON(R¹⁴), (CH(R¹⁴))_{c}—W¹—(CH(R¹⁵))_{d} wherein c and d are integers independently selected from 1 through 4, and W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁵), N(R¹⁴)C(S)N(R⁸), (R⁷)NC(S)N(R¹⁵), S(O), S(O)₂, S(O)₂N(R¹⁴), N(R¹⁴)S(O)₂, P(O)(R⁸), N(R⁷)P(O)(R⁸), P(O)(R⁸)N(R⁷), N(R¹⁴), ON(R¹⁴), and (CH(R¹⁴))ₑ—W²—(CH(R¹⁵))ₕ wherein e and h are integers independently selected from 0 through 2 and W² is selected from the group consisting of CR^{4a}═CR^{4b}, ethynylidene (C≡C; 1,2-ethynyl), and C—CR^{4a}R^{4b} with the provisos that R¹⁴ and R¹⁵ are selected from other than halo and cyano when directly bonded to N and that (CR³⁷R³⁸)_{b}, (CH(R¹⁴))_{c}, (CH(R¹⁴))ₑ and are bonded to E;

Y⁰ can be Q^{b}-Q^{ss} wherein Q^{ss} is selected from the group consisting of (CR³⁷R³⁸)_{f} wherein f is an integer selected from 1 through 6, (CH(R¹⁴))_{c}—W¹—(CH(R¹⁵))_{d} wherein c and d are integers independently selected from 1 through 4, and W¹ is selected from the group consisting of W¹ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴)(R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, P(O)(R⁸), N(R⁷)P(O)(R⁸), P(O))N(R⁷), N(R¹⁴), ON(R¹⁴), and (CH(R¹⁴))ₑ—W²—(CH(R¹⁵))ₕ wherein e and h are integers independently selected from 0 through 2 and W² is selected from the group consisting of CR^{4a}—CR^{4b}, ethynylidene (C≡C; 1,2-ethynyl), and —C═CR^{4a}R^{4b} with the provisos that R¹⁴ and R¹⁵ are selected from other than halo and cyano when directly bonded to N and that (CR³⁷R³⁸)_{f}, (CH(R¹⁵))_{c}, and (CH(R¹⁵))ₑ are bonded to E⁰;

Y⁰ can be Q^{b}-Q^{sss} wherein Q^{sss} is (CH(R³⁸))ᵣ—W³, r is an integer selected from 1 through 3, and W³ is selected from the group consisting of 1,1-cyclopropyl, 1,2-cyclopropyl, 1,1-cyclobutyl, 1,2-yclobutyl, 1,2-cyclohexyl, 1,3-cyclohexyl, 1,4-cyclohexyl, 1,2-cyclopentyl, 1,3-cyclopentyl, 2,3-morpholinyl, 2,4-morpholinyl, 2,5-morpholinyl, 2,6-morpholinyl, 3,4-morpholinyl, 3, morpholinyl, 1,2-piperazinyl, 1,3-piperazinyl, 1,4-piperazinyl, 2,3-piperazinyl, 2,5-piperazinyl, 2,6-piperazinyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4-yl, 4H-pyran-one-2,3-yl, 2,3-tetrahydrofuranyl, 2,4-tetrahydrofuranyl, 2, tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the proviso that (CH(R³⁸))ᵣ is bonded to E⁰ and Q^{b} is bonded to lowest numbered substituent position of each W³;

Y⁰ can be Q^{b}-Q^{sssr} wherein Q^{sssr} is (CH(R))ᵣ—W⁴, r is an integer selected from 1 through 3, and W is selected from the group consisting of 1,2-cyclobutyl, 1,2-cyclohexyl, 1,3-cyclohexyl, 1,4-cyclohexyl, 1,2-cyclopentyl, 1,3-cyclopentyl, 2,3-morpholinyl, 2,4-morpholinyl, 2,5-morpholinyl, 2,6-morpholinyl, 3,4-morpholinyl, 3,5-morphohnyl, 1,2-piperazinyl, 1,3-piperazinyl, 1,4-piperazinyl, 2,3-piperazinyl, 2,5-piperazinyl, 2,6-piperazinyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4,5-yl, 4H-pyran-4-one-2,3-yl, 2,3-tetrahydrofuranyl, 2,4-tetrahydrofuranyl, 2,5-tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the provisos that (CH(R³⁸))ᵣ is bonded to E⁰ and Q^{b} is bonded to highest number substituent position of each W⁴;

Y⁰ can be Q^{b}-Q^{ssss} wherein Q^{ssss} is (CH(R³⁸))ᵣ—W⁵, r is an integer selected from 1 through 3, and W⁵ is selected from the group consisting of 1,4-indenyl, 1,5-indenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-ininolinyl, 3,6-quinolinyl, 3,7-quinolinyl, 3,8-quinolinyl, 4,5-quinolinyl, 4,6-quinolinyl, 4,7-quinolinyl, 4,8-quinoliiyl, 1,4-isoquinolinyl, 1, isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to lowest number substituent position of each W⁵ and that (CH(R³⁸))ᵣ is bonded to E⁰;

Y⁰ can be Q^{b}-Q^{ssssr} wherein Q^{ssssr} is (CH(R³⁸))ᵣ—W⁶, r is an integer selected from 1 through 3, and W is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofurayl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5;benzthiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-qiinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,6-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cirmolinyl, 4,7-cinnolinyl, and 4,8-cinnolinyl with the proviso that Q^{b} is bonded to highest number substituent position of each W⁶ and that (CH(R³⁸))ᵣ is bonded to E⁰.

In another embodiment of compounds of Formula I or a pharmaceutically acceptable salt thereof,

B is formula (V):

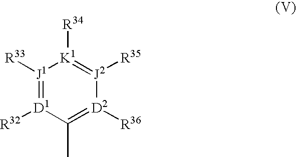

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four of D¹, D², J¹, J² and K¹ can be N;

R³² R³³ R³⁴ R³⁵, and R³⁶ can independently be Q^{b};

R⁹, R¹⁰, R¹¹, R¹², R¹³, R¹⁶, R¹⁷ R¹⁸, R¹⁹, R³², R³³, R³⁴, R³⁵ and R³⁶ are independently selected from the group consisting of hydrido, amidino, guanidino, dialkylsulfonium, trialkylphosphonium, dialkylsulfoniumalkyl, carboxy, heteroaaalkylthio, heteroaralkoxy, cycloalkylamino, acylalkyl, acylalkoxy, aryloylalkoxy, heterocyclyloxy, aralkylaryl, arlkyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloallylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroamLkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, alkoxyamino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylaniidosulfonyl, monoalkyl monoaryl amidosulfonyl, arylsulfinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyamlkyl, hydroxyalkyl, aminoalkyl, hydoxyheteroarakyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl;

B can be selected from the group consisting of C3-C8 alkyl, C3-C8 alkennyl, C3-C8 aknyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;

B can be selected from the group consisting of C3-C10 cycloalkyl, C5-C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;

A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, C(NR⁷)N(R⁷), (R⁷)NC(NR⁷), and N(R⁷);

R⁷ and R⁸ are independently selected from the group consisting of hydrido, hydroxy, alkyl, and alkoxyalkyl;

R¹⁴, R¹⁵, R³⁷, and R³⁸ are independently selected from the group consisting of hydrido, hydroxy, halo, alkyl, alkoxyalkyl, haloalkyl, haloalkoxy, and haloalkoxyalkyl;

Ψ is selected from the group consisting of NR⁵, O, C(O), C(S), S, S(O), S(O)₂, and CR³⁹R⁴⁰;

R⁵ is selected from the group consisting of hydrido, alkyl, and alkoxy;

R³⁹ and R⁴⁰ are independently selected from the group consisting of hydrido, hydroxy, halo, hydroxyalkyl, alkyl, alkoxyalkyl, haloalkyl, haloalkoxy, and haloalkoxyalkyl;

R¹ is selected from the group consisting of hydrido, alkyl, alkenyl, haloalkyl, and alkoxyalkyl;

X⁰ and R² are independently selected from the group consisting of Z⁰-Q, hydrido, alkyl, alkenyl, and halo;

X⁰ and R² can be independently selected from the group consisting of amidino, guanidino, dialkylsulfonium, trialkylphosphonium, dialkylsulfoniumalkyl, heteroarylamino, amino, nitro, alkylamino, arylamino, aralkylamino, alkanoyl, alkenoyl, aroyl, heteroaroyl, aralkanoyl, heteroaralkanoyl, haloalkanoyl, hydroxyhaloalkyl, cyano, and phosphono;

Z⁰ is selected from the group consisting of covalent single bond, (CR⁴¹R⁴²)_{q} wherein q is an integer selected from 1 through 2, (CH(R⁴¹))_{g}—W⁰—(CH(R⁴²))ₚ wherein g and p are integers independently selected from 0 through 2 and W° is selected from the group consisting of O, S, C(O), C(S), C(O)O, C(S)O, C(O)S, C(S)S, C(O)N(R⁴¹), (R⁴¹)NC(O), C(S)N(R⁴¹), (R⁴¹)NC(S), OC(O)N(R⁴¹), (R⁴¹)NC(O)O, SC(S)N(R⁴¹), (R⁴¹)NC(S)S, SC(O)N(R⁴¹), (R⁴¹)NC(O)S, OC(S)N(R⁴¹), (R⁴¹)NC(S)O, NR⁴²)C(O)N(R⁴¹), (R⁴¹)NC(O)N(R⁴²), N(R⁴²)C(S)N(R⁴¹), (R⁴¹)NC(S)N(R⁴²), S(O), S(O)₂, S(O)₂N(R⁴¹), N(R⁴¹)S(O)₂, N(R⁴¹), ON(R⁴¹), and (CH⁴¹))ₑ—W²—(CH(R⁴²))^{h} wherein e and h are integers independently selected from 0 through 2 and W² is selected from the group consisting of CR⁴¹═CR⁴², CR⁴¹R⁴²═C; vinylidene), and ethynylidene (C≡C; 1,2-ethynyl), with the proviso that R⁴¹ and R⁴² are selected from other than halo and cyano when directly bonded to N and Z⁰ is directly bonded to the pyridone ring;

R⁴¹ and R⁴² are independently selected from the group consisting of hydrido, hydroxy, halo, cyano, aryloxy, hydroxyalkyl, acyl, aroyl, heteroaroyl, heteroaryloxyalkyl, alkoxy, alkyl, aryl, aralkyl, aryloxyalkyl, aralkoxyalkylalkoxy, alkoxyalkyl, heteroaryloxyalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, saturated heterocyclyl, partially saturated heterocyclyl, heteroaryl, and heteroaralkyl;

Q^{b} is formula (II):

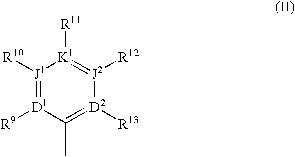

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four of D¹, D², J¹, J² and K¹ can be N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;

Q can be selected from the group consisting of alkyl, alkoxy, alkylamino, alkylthio, haloalkylthio, saturated heterocyclyl, alkyl, partially saturated heterocyclyl, acyl, aroyl, heteroaroyl, cycloalkyl, cycloalkylkyl, cycloalkenyl, cycloalkenylalkyl, cycloalkylalkenyl, haloalkyl, haloalkoxy, haloalkenyl, halocycloalkyl, halocycloalkenyl, haloalkoxyalkyl, haloalkenyloxyalkyl, halocycloalkoxyalkyl, and halocycloalkenyloxyalkyl;

K is (CR^{4a}R^{4b})ₙ wherein n is the integer 1;

R^{4a} and R^{4b} are independently selected from the group consisting of halo, hydrido, hydroxy, hydroxyalkyl, alkyl, alkoxyalkyl, and haloalkyl;

is E⁰ is E¹, when K is (CR^{4a}R^{4b})ₙ, wherein E is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, S(O)₂N(R⁷)C(O), C(O)N(R⁷)S(O)₂, N(R⁷), ON(R⁷), CR^{4a}═CR^{4b}, ethynylidene (CRC; 1,2-ethynyl), and C═CR^{4a}R^{4b};

K can be (CH(R¹⁴))ⱼ-T wherein j is selected from a integer from 0 through 1 and T is selected from the group consisting of single covalent bond, O, S, and N(R⁷) with the proviso that (CH(R⁷))ⱼ is bonded to the pyridone ring;

E⁰ is E², when K is (CH(R¹⁴))ⱼ-T, wherein E is selected from the group consisting of a covalent single bond, C(O), C(S), C(O)O, C(S)O, C(O)S, C(S)S, C(O)N(R⁷), (R⁷)NC(O), C(S)N(R⁷), (R⁷)NC(S), (R⁷)NC(O)O, (R⁷)NC(S)S, (R⁷)NC(O)S, (R⁷)NC(S)O, N(R⁸)C(O)N(R⁷), (R⁷)NC(O)N(R^{N}), N(R⁸)C(S)N(R⁷), (R⁷)NC(S)N(R⁸), S(O), S(O)₂, S(O)₂N(R⁷), N(R⁷)S(O)₂, S(O)₂N(H)C(O), C(O)N(H)S(O)₂, and N(R⁷);

K can be G-(CH(R¹⁵))ₖ wherein k is the integer 1 and G is selected from the group consisting of O, S, and N(R⁷);

E⁰ is E³ when K is G-(CH(R¹⁵)), wherein k is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, N(R⁷), ON(R⁷), CR^{4a}═CR^{4b}, ethynylidene (C≡C; 1,2-ethynyl), and C═CR^{4a}R^{4b};

Y⁰ is formula (IV):

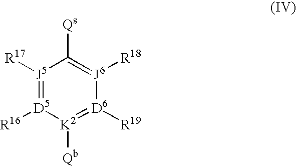

wherein D⁵, D⁶, J⁵, and J⁶ are independently selected from the group consisting of C, N, O, S and a covalent bond with the provisos that no more than one can be a covalent bond, K² is independently selected from the group consisting of C, and N⁺, no more than one of D⁵, D⁶, J⁵, and J⁶ can be O, no more than one of D⁵, D⁶, J⁵, and J⁶ can be S. one of D5, D⁶, J5, and J⁶ must be a covalent bond when two of D, D⁶, J5, and P are O and S, no more than three of D⁵, D⁶, J5, and J3 can be N when K² is N⁺, and no more than four of D⁵, D⁶, J5, and 3 can be N when K² is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;

Q^{b} is selected from the group consisting of NR²⁰R²¹, ⁺NR²⁰R²¹R²², oxy, alkyl, alIylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² can be hydroxy, alkoxy, alkylamino, amino, and dialkylamino and that R²⁰, R²¹, and R²² must be other than be hydroxy, alkoxy, alkylamino, amino, and dialkylamino when K is N⁺;

Q^{b} can be selected from the group consisting of N(R²⁶)SON(R²³)(R²⁴), N(O)C(O)OR⁵, N(R²⁶)C(O)SR⁵, N(R²⁶)C(S)OR⁵ and N(R²⁶)C(S)SR⁵ with the proviso that no more than one of R²³, R²⁴, and R²⁶ can be hydroxy, alkoxy, alkylamino, amino, or dialkylamino when two of the group consisting of R²³, R²⁴, and R²⁶ are bonded to the same atom;

Q^{b} can be selected from the group consisting of dialkylsulfonium, trialkylphosphonium, C(NR²⁵)NR²³R²⁴, N(R²⁶)C(NR²⁵)N(R²³)(R²⁴), N(R²⁶)C(O)N(R²³)(R²⁴), N(R²⁶)C(S)N(R²³)(R²⁴), C(NR²⁵)OR⁵, C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁶), N(R²⁶)N(R²⁶)SO₂N(R²³)(R²⁴), C(NR²⁵)SR⁵, C(O)NR²³R²⁴, and C(O)NR²³R²⁴ with the provisos that no more than one of R²³, R²⁴, and R²⁶ can be hydroxy, alkoxy, alkylamino, amino, or dialkylamino when two of the group consisting of R²³, R²⁴, and R²⁶ are bonded to the same atom and that said Q group is bonded directly to a carbon atom;

R²³, R²⁴, R²⁵, and R²⁶ are independently selected from the group consisting of hydrido, alkyl, hydroxy, alkoxy, alkylamino, dialkylamino, aminoalkyl, and hydroxyalkyl;

Q^{b} is selected from the group consisting of a single covalent bond, (CR³⁷R³⁸)_{b}—(W⁰)_{az} wherein az is an integer selected from 0 through 1, b is an integer selected from 1 through 2, and W⁰ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), SC(S)N(R¹⁴), SC(O)N(R¹⁴), OC(S)N(R¹⁴), N(R¹⁵)C(O)N(R¹⁴), (R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, N(R¹⁴), ON(R¹⁴), and (CH(R¹⁴))_{c}—W¹— (CH(R¹⁵))_{d} wherein c and d are integers independently selected from 1 through 2, and W¹ is selected from the group consisting of O, S, C(O), C(S), C(O)O, C(S)O, C(O)S, C(S)S, C(O)N(R¹⁴), (R¹⁴)NC(O), C(S)N(R¹⁴) (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁵), N(R¹⁵)C(S)N(R¹⁴) (R¹⁴)NC(S)N(R¹⁵), S(O), S(O)₂, S(O)₂N(R¹⁴), N(R¹⁴)S(O)₂, N(R¹⁴), ON(R¹⁴), and (CH(R¹⁴))ₑ—W²—(CH(R¹⁵))ₕ wherein e and h are integers independently selected from 0 through 2 and W² is selected from the group consisting of CR^{4a}═CR^{4b}, ethynylidene (C≡C; 1,2-ethynyl), and C═CR^{4a}R^{4b} with the provisos that R¹⁴ and R¹⁵ are selected from other than halo and cyano when directly bonded to N and that (CR³⁷R³⁸)_{b}, (CH(R¹⁴))_{c}, (CH(R¹⁴))ₑ and are bonded to E⁰;

Y⁰ can be Q^{b}-Q^{ss} wherein Q^{ss} is selected from the group consisting of (CR³⁷R³⁸)_{f} wherein f is an integer selected from 1 through 4, (CH(R¹⁴))_{c}—W¹—(CH(R¹⁵))_{d} wherein c and d are integers independently selected from 1 through 2, and W¹ is selected from the group consisting of W¹ is selected from the group consisting of O, S, C(O), C(S), C(O)O, C(S)O, C(O)S, C(S)S, C(O)N(R¹⁴) (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁵), N(R¹⁵)C(S)N(R¹⁴) (R¹⁴)NC(S)NR¹⁵), S(O), S(O)₂, S(O)₂N(R¹⁴), N(R¹⁴)S(O)₂, N(R¹⁴), ON(R¹⁴), and (CH(R¹⁴))ₑ—W²—(CH(R¹⁵))ₕ wherein e and h are integers independently selected from 0 through 2 and W² is selected from the group consisting of CR^{4a}═CR^{4b}, ethynylidene (C≡C; 1,2-ethynyl), and C—CR^{4a}R^{4b} with the provisos that R¹⁴ and R¹⁵ are selected from other than halo when directly bonded to N and that (CR³⁷R³⁸)_{f}, (CH(R¹⁵))_{c}, and (CH(R¹⁵))ₑ are bonded to E⁰;

Y⁰ can be Q^{b}-Q^{sss} wherein Q^{sss} is (CH(R³⁸))ᵣ—W³, r is an integer selected from 1 through 2, and W is selected from the group consisting of 1,1-cyclopropyl, 1,2-cyclopropyl, 1,1-cyclobutyl, 1,2-cyclobutyl, 1,2-cyclohexyl, 1,3-cyclohexyl, 1,4-cyclohexyl, 1,2-cyclopentyl, 1,3-cyclopentyl, 2,3-morpholinyl, 2,4-morpholinyl, 2,5Lmorpholinyl, 2,6-morpholinyl, 3,4-morpholinyl, 3,5-morpholinyl, 1,2-piperazinyl, 1,3-piperazinyl, 1,4-piperznyl, 2,3-piperazinyl, 2,5-piperazinyl, 2,6-piperazinyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrmlidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4,5-yl, 4H-pyran-4-one-2,3-yl, 2,3-tetrahydrofuranyl, 2,4-tetrahydrofuranyl, 2, tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the proviso that (CH(R³⁸))ᵣ is bonded to E⁰ and Q^{b} is bonded to lowest numbered substituent position of each W³;

Y⁰ can be Q^{b}-Q^{sssr} wherein Q^{sssr} is (CH(R³⁸))ᵣ—W⁴, r is an integer selected from 1 through 2, and W is selected from the group consisting of 1,2-cyclobutyl, 1,2-cyclohexyl, 1,3-cyclohexyl, 1,4-cyclohexyl, 1,2-lyclopentyl, 1,3-cyclopentyl, 2,3-morpholinyl, 2,4-morpholinyl, 2,5-morpholinyl, 2,6-morpholinyl, 3,4-morpholinyl, 3,5-morpholinyl, 1,2-piperazinyl, 1,3-piperziyl, 1,4-piperziyl, 2,3-piperazinyl, 2,5-piperazinyl, 2,6-pipeazinyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidiyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4,5-yl, 4H-pyran-one-2,3-yl, 2,3-tetrahydrofuranyl, 2,4-tetrahydrofuranyl, 2,5-tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the provisos that (CH(R³⁸))ᵣ is bonded to E⁰ and Q^{b} is bonded to highest number substituent position of each W⁴;

Y⁰ can be Q^{b}-Q^{ssss} wherein Q^{ssss} is (CH(R³⁸))ᵣ—W⁵, r is an integer selected from 1 through 2, and W is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,6-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to lowest number substituent position of each W⁵ and that (CH(R³⁸))ᵣ is bonded to E⁰;

Y⁰ can be Q^{b}-Q^{ssssr} wherein Q^{ssssr} is (CH(R³⁸))ᵣW⁶, r is an integer selected from 1 through 2, and W⁶ is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,%benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1, isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to highest number substituent position of each W⁶ and that (CH(R³⁸))ᵣ is bonded to E⁰.

In a preferred embodiment of compounds of Formula I or a pharmaceutically acceptable salt thereof,

B is formula (V):

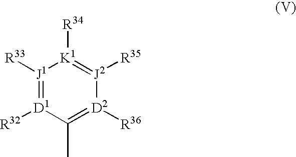

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four D¹, D², J¹, J² and K¹ can be N;

R³², R³³, R³⁴ R³⁵ and R³⁶ can independently be Q^{b};

R⁹, R¹⁰, R¹¹, R¹², R¹³, R³², R³³, R³⁴, R³⁵, and R³⁶ are independently selected from the group consisting of hydrido, amidino, guanidino, dialkylsulfonium, carboxy, haloalkylthio, alkanoyloxy, alkoxy, alkoxyalkyl, haloalkoxylalkyl, hydroxy, amino, alkoxyamino, thio, nitro, lower alkylamino, alkylthio, alkylthioalkyl, alkylsulfinyl, alkylsulfinylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, alkanoyl, alkenoyl, haloalkanoyl, alkyl, alkenyl, alkenyloxy, alkenyloxyalky, halo, haloalkyl, haloalkenyl, haloalkoxy, hydroxyhaloalkyl, hydroxyalkyl, aminoalkyl, haloalkoxyalkyl, carboxyalkyl, carboalkoxy, alkoxycarboxamido, alkylamidocarbonylamido, carboalkoxyalkyl, carboalkoxyalkenyl, carboxamido, carboxamidoalkyl, and cyano;

B can be selected from the group consisting of C3-C8 alkyl, C3-C8 alkenyl, C3-C8 alkynyl, C3-C8 haloalayl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;

B can be selected from the group consisting of C3-C10 cycloalkyl, C5-C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;

A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, C(NR⁷)N(R⁷), (R⁷)NC(NR⁷), and N(R⁷);

R⁷ and R⁸ are independently selected from the group consisting of hydrido, hydroxy, alkyl, and alkoxyalkyl;

R¹⁵ is selected from the group consisting of hydrido, hydroxy, halo, alkyl, and haloalkyl;

v is NH;

X⁰ is hydrido;

R¹ is selected from the group consisting of hydrido, alkyl, and haloalkyl;

R² is selected from the group consisting of Z⁰-Q, hydrido, alkyl, alkenyl, and halo;

Z⁰ is a covalent single bond;

Q is formula (II):

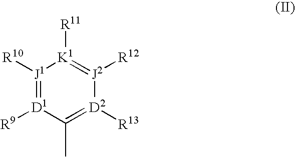

wherein D¹, D², J¹, J² and K¹ are independently selected from the group consisting of C, N, O, S and a covalent bond with the provisos that no more than one can be a covalent bond, no more than one of D¹, D², J¹, J² and K¹ can be O, no more than one of D¹, D², J¹, J² and K¹ can be S, one of D¹, D², J¹, J² and K¹ must be a covalent bond when two of D¹, D², J¹, J² and K¹ are O and S, and no more than four of D¹, D², J¹, J² and K¹ can be N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;

K is CR^{4a}R^{4b};

R^{4a} and R^{4b} are independently selected from the group consisting of halo, hydrido, hydroxy, alkyl, and haloalkyl;

E⁰ is E¹, when K is CR^{4a}R^{4b}, wherein E¹ is selected from the group consisting of a covalent single bond, (O)N(H), (H)NC(O), C(S)N(H), (H)NC(S), S(O)₂N(H), N(H)S(O)₂, S(O)₂N(H)C(O), and C(O)N(H)S(O)₂;

K can be (CH(R¹⁴))ⱼ-T wherein j is selected from an integer from 0 through 1 and T is selected from the group consisting of single covalent bond and N(R 7) with the proviso that (CH(R¹⁴))ⱼ is bonded to the pyridone ring;

E⁰ is E², when K is (CH(R¹⁴))ⱼ-T, wherein E² is selected from the group consisting of C(O)N(H), (H)NC(O), C(S)N(H), (H)NC(S), S(O)₂N(H), N(H)S(O)₂, S(O)₂N(H)C(O), and C(O)N(H)S(O)₂;

R¹⁴ is selected from the group consisting of hydrido, halo, alkyl, and haloalkyl;

Y⁰ is formula (IV):

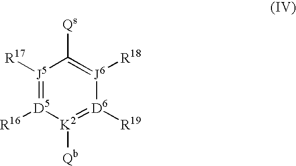

wherein D⁵, D⁶, J⁵, and J⁶ are independently selected from the group consisting of C, N, O, S and a covalent bond with the provisos that no more than one can be a covalent bond, K² is independently selected from the group consisting of C, and N⁺, no more than one of D⁵, D⁶, J⁵, and J⁶ can be O, no more than one of D⁵, D⁶, J⁵, and J⁶ can be S, one of D⁵, D⁶, J⁵, and J⁶ must be a covalent bond when two of D⁵, D⁶, J5, and J6 are O and S, no more than three of D⁵, D⁶, J⁵ and J⁶ can be N when K is N⁺, and no more than four of D⁵, D6,J⁵, and J⁶ can be N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;

R¹⁶, R¹⁷, R¹⁸, and R¹⁹ are independently selected from the group consisting of hydrido, amidino, guanidino, dialkylsulfonium, carboxy, haloalkylthio, alkoxy, hydroxy, amino, thio, nitro, lower alkylamino, alkylthio, alkylsulfinyl, alkylsulfonyl, alkanoyl, alkenoyl, haloalkanoyl, alkyl, alkenyl, halo, haloalkyl, haloalkenyl, haloalkoxy, hydroxyalkyl, aminoalkyl, haloalkoxyalkyl, carboalkoxy, carboalkoxyalkyl, and cyano;

Q^{b} is selected from the group consisting of NR²⁰R²¹, NR²⁰R²¹R²² oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² can be hydroxy, alkoxy, alkylamino, amino, and dialkylamino and that R²⁰, R²¹, and R²² must be other than be hydroxy, alkoxy, alkylamino, amino, and dialkylamino when K² is N⁺;

Q can be N(R²⁶)SO₂N(R²³)(R²⁴) with the proviso that no more than one of R²³, R²⁴, and R²⁶ can be hydroxy, alkoxy, alkylamino, amino, or dialkylamino when two of the group consisting of R²³, R²⁴, and R²⁶ are bonded to the same atom;

Q^{b} can be selected from the group consisting of dialkylsulfonium, trialkylphosphonium, C(NR²⁵)NR²³R²⁴, N(R²⁶)C(NR²⁵)N(R²³)(R²⁴), N(R²⁶)C(O)N(R²³)(R²⁴), N(R²⁶)C(S)N(R²³)(R²⁴), C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²³, R²⁴, and R²⁶ can be hydroxy, alkoxy, alkylamino, amino, or dialkylamino when two of the group consisting of R²³, R²⁴, and R²⁶ are bonded to the same atom and that said Q group is bonded directly to a carbon atom;

R²³, R²⁴, R²⁵, and R²⁶ are independently selected from the group consisting of hydrido, alkyl, hydroxy, alkoxy, alkylamino, dialkylamino, aminoalkyl, and hydroxyalkyl;

Q^{b} is selected from the group consisting of a single covalent bond and (CR³⁷R³⁸)_{b}—(W⁰)_{az} wherein az is an integer selected from 0 through 1, b is an integer selected from 1 through 2, and W⁰ is selected from the group consisting of O, S, C(O), S(O)₂, N(R¹⁴), and ON(R¹⁴) with the proviso that R¹⁴ is selected from other than halo when directly bonded to N and that (CR³⁷R³⁸) is bonded to E⁰;

R³⁷ and R³⁸ are independently selected from the group consisting of hydrido, halo, alkyl, and haloalkyl;

Y⁰ can be Q^{b}-Q^{ssss} wherein Q^{ssss} is (CH(R³⁸))ᵣ—W⁵, r is an integer selected from 1 through 2, and W⁵ is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1, isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to lowest number substituent position of each W⁵ and that (CH(R³⁸)ᵣ is bonded to E⁰;

Y⁰ can be Q^{b}-Q^{sssr} wherein Q^{sssr} is (CH(R³⁸))ᵣ—W⁶, r is an integer selected from 1 through 2, and W⁶ is selected from the group consisting of 1,4-indenyl, 1,5-indenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-sbenzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-bcnzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1, isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to highest number substituent position of each W⁶ and that (CH(R³⁸))ᵣ is bonded to E⁰.

In a more preferred embodiment of compounds of Formula I or a pharmaceutically acceptable salt thereof,

B is selected from the group consisting of aryl and heteroaryl wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;

R³², R³³, R³⁴, R³⁵, and R³⁶ are independently selected from the group consisting of hydrido, amidino, guanidino, dialkylsulfonium, carboxy, haloalkylthio, alkoxy, hydroxy, amino, alkoxyamino, thio, nitro, lower alkylamino, alkylthio, alkylsulfinyl, alkylsulfonyl, amidosulfonyl, alkanoyl, haloalkanoyl, alkyl, halo, haloalkyl, haloalkenyl, haloalkoxy, hydroxyhaloalkyl, hydroxyalkyl, aminoalkyl, carboxyalkyl, carboalkoxy, carboxamido, and cyano;

R³², R³³, R³⁴, R³⁵ and R³⁶ can independently be Q^{b};

B can be selected from the group consisting of C3-C8 alkyl, C3-C8 alkenyl, C3-C8 haloalkyl, and C3 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;

B can be selected from the group consisting of C3-C10 cycloalkyl, C5-C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R¹⁰ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;

R⁹, R¹⁰, R¹¹, R¹², and R¹³ are independently selected from the group consisting of hydrido, amidino, guanidino, dialkylsulfonium, carboxy, haloalkylthio, alkoxy, hydroxy, amino, alkoxyamino, thio, nitro, lower alkylamino, alkylthio, alkylsulfinyl, alkylsulfonyl, amidosulfonyl, alkanoyl, haloalkanoyl, alkyl, halo, haloalkyl, haloalkenyl, haloalkoxy, hydroxyhaloalkyl, hydroxyalkyl, aminoalkyl, carboxyalkyl, carboalkoxy, carboxamido, and cyano;

A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, and C(O);

R¹⁵ is selected from the group consisting of hydrido, hydroxy, halo, alkyl, and haloalkyl;

Ψ is NH;

X⁰ is hydrido;

R¹ is selected from the group consisting of hydrido, alkyl, and haloalkyl;

R² is Q, wherein Q is selected from the group consisting of aryl and heteroaryl wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;

K is CR^{4a}R^{4b} wherein R^{4a} and R^{4b} are independently selected from the group consisting of halo and hydrido;

E⁰ is E¹, when K is CR^{4a}R^{4b}, wherein E¹ is selected from the group consisting of a covalent single bond, (O)N(H), (H)NC(O), S(O)₂N(H), N(H)S(O)₂, S(O)₂N(H)C(O), and C(O)N(H)S(O)₂;

K can be (CH(R¹⁴))ⱼ-T wherein j is selected from an integer from 0 through 1 and T is selected from the group consisting of single covalent bond and N(R⁷) with the proviso that (CH(R¹⁴))ⱼ is bonded to the pyridone ring;

R⁷ is selected from the group consisting of hydrido, hydroxy, alkyl, and alkoxyalkyl;

R¹⁴ is selected from the group consisting of hydrido and halo;

E⁰ is E², when K is (CH(R¹⁴))ⱼ-T, wherein E² is selected from the group consisting of C(O)N(H), (H)NC(O), S(O)₂N(H), N(H)S(O)₂, S(O)₂N(H)C(O), and C(O)N(H)S(O)₂;

Y⁰ is formula (IV):

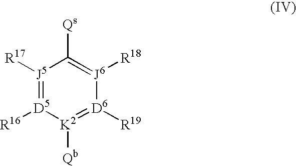

wherein D⁵, D⁶, J⁵, and J⁶ are independently selected from the group consisting of C, N, O, S and a covalent bond with the provisos that no more than one can be a covalent bond, K is independently selected from the group consisting of C, and N⁺, no more than one of D⁵, D⁶,J⁵, and J⁶ is O, no more than one of D⁵,D⁶, J⁵, and J⁶ is S, one of D⁵, D⁶, J⁵, and J⁶ must be a covalent bond when two of D⁵, D⁶, J⁵, and J⁶ are O and S, no more than three of D⁵, D⁶, J⁵, and J⁶ can be N when K is N⁺, and no more than four of D⁵, D⁶, J⁵, and J⁶ can be N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;

R¹⁶, R¹⁷, R¹⁸, and R¹⁹ are independently selected from the group consisting of hydrido, amidino, guanidino, dialkylsulfonium, carboxy, haloalkylthio, alkoxy, hydroxy, amino, thio, nitro, lower alkylamino, alkylthio, alkylsulfinyl, alkylsulfonyl, alkanoyl, alkenoyl, haloalkanoyl, alkyl, alkenyl, halo, haloalkyl, haloalkenyl, haloalkoxy, hydroxyalkyl, aminoalkyl, haloalkoxyalkyl, carboalkoxy, carboalkoxyalkyl, and cyano;

Q^{b} is selected from the group consisting of NR²⁰R²¹, ⁺NR²⁰R²¹R²², oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² can be hydroxy, alkoxy, alkylamino, amino, and dialkylamino and that R²⁰, R²¹, and R²² must be other than be hydroxy, alkoxy, alkylamino, amino, and dialkylamino when K is N⁺;

Q^{b} can be selected from the group consisting of dialkylsulfonium, ttialkylphosphonium, C(NR²⁵)NR²³R²⁴, N(R²⁶)C(NR²⁵)N(R²³)(R²⁴), C(O)N(R²⁶)C(NR²⁵)N(R²³)R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²³, R²⁴, and R²⁶ can be hydroxy, alkoxy, alkylamino, amino, or dialkylamino when two of the group consisting of R²³, R²⁴, and R²⁶ are bonded to the same atom and that said Q group is bonded directly to a carbon atom; R²³, R²⁴, R²⁵, and R²⁶ are independently selected from the group consisting of hydrido, alkyl, hydroxy, alkoxy, alkylamino, dialkylamino, aminoalkyl, and hydroxyalkyl;

Q⁸ is selected from the group consisting of a single covalent bond and (CR³⁷R³⁸)_{b}—(W⁰)_{az} wherein az is an integer selected from 0 through 1, b is the integer 1, and W⁰ is selected from the group consisting of O, S, and C(O) with the proviso that (CR³⁷R³⁸)_{b} is bonded to E⁰;

R³⁷ and R³⁸ are independently selected from the group consisting of hydrido, halo, alkyl, and haloalkyl.

In a specific embodiment of Formula I, compounds have the Formula I-S:

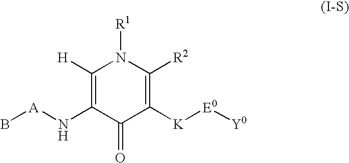

or a pharmaceutically acceptable salt thereof, wherein;

B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrrolyl, 4-pyrazolyl, 1,2,5-triazol-3-yl, 1,2,5-thiazol-5-yl, 1,2,4-triazol-3-yl, 1,2,4-oxadiazol-5-yl, 1,3,4-oxadiazol-3-yl, 1,3,4-oxadiawol-yl, 3-isothiazolyl, 5 isothiazolyl, 2-oxazolyl, 2-thiazolyl, 3-isoxazolyl, 5-isoxazolyl, 2-pyridyl, 3-pyridyl, 4-pyridyl, 2-pyrazinyl, 2-pyrimidinyl, 4-pyrimidinyl, 5-pyrimidinyl, 3-pyridazinyl, 4-pyridazinyl, 1,3,5-triazin-2-yl, 1,2,4-triazin-3-yl, 1,2,4-triazin-5-yl, 1,2,4-triazin-4-yl, and 1,2,5-triazin-4-yl, wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;

R³², R³³, R³⁴, R³⁵, and R³⁶ are independenfly selected from the group consisting of hydrido, amidino, guanidino, dimethylsulfonium, carboxy, methoxy, ethoxy, isopropoxy, propoxy, hydroxy, amino, methoxyamino, ethoxyamino, thio, nitro, aminomethyl, 1-aminoethyl, 2-aminoethyl, N,N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-etrafluoroethoxy, fluoro, chloro, bromo, amidosulfonyl, N-methylamidosulfonyl, N,N-dimethylamidosulfonyl, acetyl, propanoyl, trifluoroacetyl, pentafluoropropanoyl, hydroxymethyl, 1-hydroxyethyl, 2-hydroxyethyl, 2,2,2-triuoro-1-hydroxyethyl, 2,2,2-trifluoro-1-trifluoromethyl-1-hydroxyethyl, carboxymethyl, methoxycarbonyl, ethoxycarbonyl, amidocarbonyl, N-methylamidocarbonyl, N,N-dimethylamidocarbonyl, cyano, and Q^{b};

B can be selected from the group consisting of 1-propenyl, propyl, isopropyl, butyl, 2-butenyl, 3-butenyl, 2-butynyl, sec-butyl, isobutyl, 2-methylpropenyl, 1-pentyl, 2-pentenyl, 3-pentenyl, 4-pentenyl, 2-pentynyl, 3-pentynyl, 2-pentyl, 1-methyl-2-butenyl, 1-methyl-3-butenyl, 1-methyl-2-butynyl, 3-pentyl, 1-ethyl-2-propenyl, 2-methylbutyl, 2-methyl-2-butenyl, 2-methyl-3-butenyl, 2-methyl-3-butynyl, 3-methylbutyl, 3-methyl-2-butenyl, 3-methyl-3-butenyl, 1-hexyl, 2-hexenyl, 3-hexenyl, 4-hexenyl, 5-hexenyl, 2-hexynyl, 3-hexynyl, 4-hexynyl, 2-hexyl, 1-methyl-2-pentenyl, 1-methyl-3-pentenyl, 1-methyl-4-pentenyl, 1-methyl-2-pentynyl, 1-methyl-3-pentynyl, 3-hexyl, 1-ethyl-2-butenyl, 1-ethyl-3-butenyl, 1-propyl-2-propenyl, 1-ethyl-2-butynyl, 1-heptyl, 2-heptenyl, 3-heptenyl, 4-heptenyl, 5-heptenyl, 6-heptenyl, 2-heptynyl, 3-heptynyl, 4-heptynyl, 5-heptynyl, Z-heptyl, 1-methyl-2-hexenyl, 1-methyl-3-hexenyl, 1-methyl-hexenyl, 1-methyl-5-hexenyl, 1-methyl-2-hexynyl, 1-methyl-3-hexynyl, 1-methylhexynyl, 3-heptyl, 1-ethyl-2-pentenyl, 1-ethyl-3-pentenyl, 1-ethyl-4-pentenyl, 1-butyl-2-propenyl, 1-ethyl-2-pentynyl, 1-ethyl-3-pentynyl, 1-octyl, 2-octenyl, 3-octenyl, 4-octenyl, S-octenyl, 6-octenyl, 7-octenyl, 2-octynyl, 3-octynyl, 4-octynyl, 5-octynyl, 6-octynyl, 2-octyl, 1-methyl-2-heptenyl, 1-methyl-3-heptenyl, 1-methyl-heptenyl, 1-methyl-5-heptenyl, 1-methyl-6-heptenyl, 1-methyl-2-heptynyl, 1-methyl-3-heptynyl, 1-methylheptenyl, 1-methyl-5-heptenyl, 1-methylheptenyl, 1-methyl-2 heptenyl, 1-methyl-3-heptynyl, 1-methyl-4-heptynyl, 1-methyl-5-heptynyl, 3-octyl, 1-ethyl-2-hexenyl, 1-ethyl-3-hexenyl, 1-ethylhexenyl, 1-ethyl-2-hexynyl, 1-ethyl-3-hexynyl, 1-ethylhexynyl, 1-ethyl-S-hexenyl, 1-pentyl-2-propenyl, 4-octyl, 1-propyl-2-pentenyl, 1-propyl-3-pentenyl, 1-propyl pentenyl, 1-butyl-2-butenyl, 1-propyl-2-pentynyl, 1-propyl-3-pentynyl, 1-butyl-2-butynyl, 1-butyl-3-butenyl, 2,2-difluoropropyl, 4-trifluoromethyl-5,5, trifluoropentyl, 4-trifluoromethylpentyl, 5,5,6,6,6-pentafluorohexyl, and 3,3,3-trifluoropropyl, wherein each member of group B may be optionally substituted at any carbon up to and including 5 atoms from the point of attachment of B to A with one or more of the group consisting of R₃₂, R₃₃, R₃₄, R₃₅, and R₃₆;

B can be selected from the group consisting of cyclopropyl, cyclobutyl, oxetan-2-yl, oxetan-3-yl, azetidin-1-yl, azetidin-2-yl, azetidin-3-yl, thiaetan-2-yl, thiaetan-3-yl, cyclopentyl, cyclopent-2-enyl, cyclopent-3-enyl, cyclohexyl, 4 methylcyclohexyl, 4-chloro-3-ethylphenoxycyclohexyl, 3-trifluoromethoxyphenoxycyclohexyl, 3-trifluoromethylcyclohexyl, 4 trifluoromethylcyclohexyl, 3,5-bis-txifluoromethylcyclohexyl, adamantyl, 3-trifluoromethyladamantyl, norbornyl, 3-trifluoromethylnorbornyl, norbornenyl, 7-oxabicyclo[2.2.1]heptan-2-yl, bicyclo[3.1.0]hexan-6-yl, cyclohex-2-enyl, cyclohex-3-enyl, cycloheptyl, cyclohept-2-enyl, cyclohept-3-enyl, cyclooctyl, cyclooct-2-enyl, cyclooct-3-enyl, cyclooctaenyl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 2H-2-pyranyl, 2H-3-pyranyl, 2HApyranyl, 4H-2-pyranyl, 4H-3-pyranyl, 4H-4-pyranyl, 2H-pyran-2-one-3-yl, 2H-pyran-2-one-4-yl, 2H-pyran-2-one-5-yl, 4H-pyrantone-2-yl, 4H-pyran-4-one-3-yl, 2-tetrahydrofuranyl, 3-tetrahydrofuranyl, 2-tetrahydropyranyl, 3-tetrahydropyranyl, 4-tetrahydropyranyl, 2-tetrahydrothienyl, and 3-tetrahydrothienyl, wherein each ring carbon may be optionally substituted with R₃₃, a ring carbon and nitrogen atoms adjacent to the carbon atom at the point of attachment may be optionally substituted with R₉ or R₁₃, a Ting carbon or nitrogen atom adjacent to the R₉ position and two atoms from the point of attachment may be substituted with R₁₀, and a ring carbon or nitrogen atom adjacent to the R₁₃ position and two atoms from the point of attachment may be substituted with R₁₂;

R⁹, R¹⁰, R¹¹, R¹², and R¹³ are independently selected from the group consisting of amidino, guanidino, dimethylsulfonium, methylethylsulfonium, carboxy, methoxy, ethoxy, isopropoxy, propoxy, butoxy, hydroxy, amino, methoxyamino, ethoxyamino, aminomethyl, 1-aminoethyl, 2-aminoethyl, N,N-dimethylamino, N-methylamino, N-ethylamino, methylsulfinyl, ethylsulfinyl, methylsulfonyl, ethylsulfonyl, amidosulfonyl, N-methylamidosulfonyl, N,N-dimethylamidosulfonyl, acetyl, propanoyl, butanoyl, trifluoroacetyl, pentafluoropropanoyl, hydroxymethyl, 1-hydroxyethyl, 2-hydroxyethyl, 2,2,2-trifluoro-1-hydroxyethyl, 2,2,2-trifluoro-1-trifluoromethyl-1-hydroxyethyl, carboxymethyl, 2-carboxyethyl, methoxycarbonyl, ethoxycarbonyl, amidocarbonyl, N-methylamidocarbonyl, N,N-dimethylamidocarbonyl, and cyano;

A is selected from the group consisting of single covalent bond, O, C(O), CH₂, CH₃CH, CF₃CH, CH₃CC(O), CF₃CC(O), C(O)CCH₃, C(O)CCF₃, CH₂C(O), (O)CCH₂, CH₂CH₂, CH₂CH₂CH₂, CH₃CCH₂, CF₃CCH₂, CH₃CC(O)CH₂, CF₃CC(O)CH₂, CH₂C(O)CCH₃, CH₂C(O)CCF₃, CH₂CH₂C(O), and CH₂(O)CCH₂;

R¹ is selected from the group consisting of hydrido, methyl, ethyl, propyl, butyl, 2,2,2-bifluoroethyl, and 2,2,3,3,3-pentafluoropropyl;

R² is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 1,2,4-triazol-3-yl, 1,2,4-triazol-5-yl, 1,2,4-oxadiazol-3-yl, 1,2,4-oxadiazol-5-yl, 1,3,4-oxadiazol-3-yl, 1,3,4-oxadiazol-5-yl, 3-isothiazolyl, 5-isothiazolyl, 2-oxazolyl, 2-thiazolyl, 3-isoxazolyl, 5-isoxazolyl, 2-pyridyl, 3-pyridyl, 4-pyridyl, 2-pyrazinyl, 2-pytimidinyl, 4-pyrimidinyl, 5-pyjijmidinyl, 3-pyridazinyl, 4-pyridazinyl, 1,3,5-triazin-2-yl, 1,2,4-triazin-3-yl, 1,2,4-triazin-5-yl, 1,2,4-triazin-6-yl, and 1,2,3-triazin-4-yl, wherein a carbon adjacent to the carbon at the point of attachment may be substituted by R⁹, the other carbon adjacent to the carbon at the point of attachment may be substituted by R^{13,}, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;

K is CR^{4a}R^{4b} wherein R^{4a} and R^{4b} are independently selected from the group consisting of chloro, fluoro, and hydrido;

E⁰ is E¹, when K is CR^{4a}R^{4b}, wherein E¹ is selected from the group consisting of a covalent single bond, C(O)N(H), (H)NC(O), S(O)₂N(H), N(H)S(O)₂, S(O)₂N(H)C(O), and C(O)N(H)S(O)₂;

K can be N(H) and CH₂N(H);

E⁰ is E², when K is N(H) and CH₂N(H), wherein E² is selected from the group consisting of C(O)N(H), (H)NC(O), S(O)₂N(H), N(H)S(O)₂, S(O)₂N(H)C(O), and C(O)N(H)S(O)₂;

Y⁰ is selected from the group of formulas consisting of:

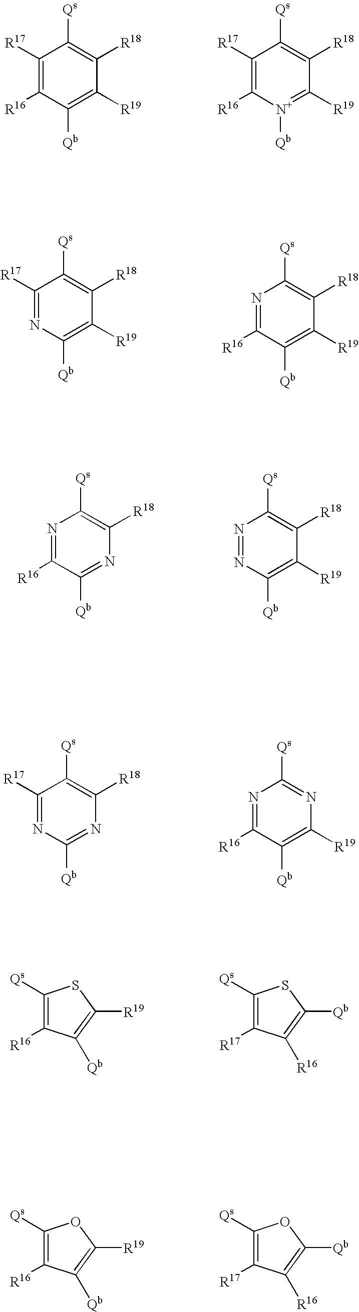

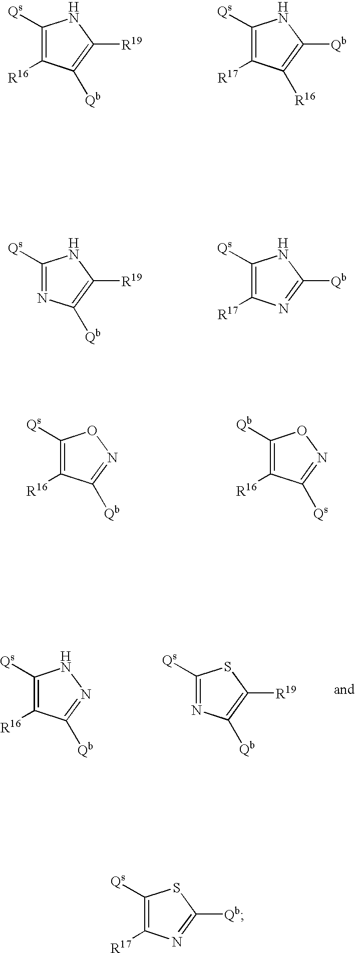

R¹⁶, R¹⁷, R¹⁸, and R¹⁹ are independently selected from the group consisting of hydrido, amidino, guanidino, dimethylsulfonium, carboxy, methoxy, ethoxy, isopropoxy, propoxy, hydroxy, amino, methoxyamino, ethoxyamino, thio, nitro, aminomethyl, 1-aminoethyl, 2-aminoethyl, N,N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-tetiafluoroethoxy, fluoro, chloro, bromo, amidosulfonyl, N-methylamidosulfonyl, N,N-dimethylamidosulfonyl, acetyl, propanoyl, trfluoroacetyl, pentafluoropropanoyl, hydroxymethyl, 1-hydroxyethyl, 2-hydroxyethyl, 2,2,2-trifuoro-1-hydroxyethyl, 2,2,2-trifluoro-1-trifluoromnethyl-1-hydroxyethyl, carboxymethyl, methoxycarbonyl, ethoxycarbonyl, amidocarbonyl, N-methylamidocarbonyl, N,N-dimethylamidocarbonyl, and cyano;

Q^{b} is selected, when bonded to a carbon, from the group consisting of NR²⁰R²¹, ⁺NR²⁰R²¹R²², dimethylsulfonium, methylethylsulfonium, diethylsulfonium, trimethylphosphonium, C(NR²⁵)NR²³R²⁴, N(R²⁶)C(NR²⁵)NR²³)(R²⁴), C(O)N(R²⁶)C(NR²⁵)N(R²³)(R²⁴), N(R²⁶)N(R²⁶)C(NR²⁵)N(R²³)(R²⁴) N(R²⁶)N(R²⁶)SO₂N(R²³)(R²⁴), C(O)NR²³R²⁴, and C(O)NR³R²⁴ with the provisos that no more than one of R²⁰, R²¹, and R²² can be hydroxy, methoxy, ethoxy, N-methylamino, N,N-dimethylamino, and N,N,N-trimethylamino, and amino and that no more than one of R²³, R²⁴, and R²⁶ can be hydroxy, methoxy, ethoxy, N-methylamino, N,N-dimethylamino, N,N,N-trimethylamino, or amino when two of the group consisting of R²³, R²⁴, and R²⁶ are bonded to the same atom and that said Q group is bonded directly to a carbon atom;

R²⁰, R²¹, R²², R²³, R²⁴, R²⁵ and R²⁶ are independently selected from the group consisting of hydrido, methyl, ethyl, propyl, butyl, isopropyl, hydroxy, methoxy, ethoxy, isopropoxy, propoxy, 2-aminoethyl, 2-(N-methylamino)ethyl, 2-(N,N-dimethylamino)ethyl, 2-(N,N,N-trimethylamino)ethyl, N-(2-hydroxyethyl)amino, N,N-bis-(2-hydroxyethyl)amino, N-(2-hydroxyethyl)-N-(2-aminoethyl)amino, N-methylamino, N-ethylamino, N,N-dimethylamino, N,N-diethylamino, and N,N,N-trimethylamino;

Q^{b} is selected, when bonded to a nitrogen, from the group consisting of oxy, methyl, ethyl, 2,5-aminoethyl, 2(N-methylamino)ethyl, N,N-dimethylaio)ethyl, 2-N,N,N-trimethylamino)ethyl, N-(2-hydroxyethyl)amino, N,N-bis-(2-hydroxyethyl)amino, amino, hydroxylamino, N-methoxyamino, N-methylamino, N,N-limethylamino, and N,N,N-trimethylamino;

Q^{s} is selected from the group consisting of a single covalent bond, CH₂, CH₃CH, CF₂, CF₃CH, CH₂O, CH₃C(H)O, CF₃C(H)O, CH₂S, CH₃C(H)S, CF₃C(H)S, CH₂C(O), CH₃C(H)C(O), CF₃C(H)C(O), and CF₂C(O) with the proviso that Q^{s} is bonded to E⁰ through a carbon atom.

In a more preferred specific embodiment of Formula I, compounds have the Formula I-MPS:

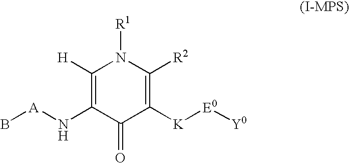

or a pharmaceutically acceptable salt thereof, wherein;

B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, 5-isoxazolyl, 2-pyridyl, 3-pyridyl, 4 pyridyl, 2-pyrazinyl, 2-pyrimidinyl, 4-pyrimidinyl, 5-pyrimidinyl, 3-pyridazinyl, 4-pyridazinyl, and 1,3,5-triazin-2-yl, wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;

R³², R³³, R³⁴, R³⁵, and R³⁶ are independently selected from the group consisting of hydrido, amidino, guanidino, dimethylsulfonium, carboxy, methoxy, ethoxy, isopropoxy, propoxy, hydroxy, amino, methoxyamino, ethoxyamino, thio, nitro, aminomethyl, 1-aminoethyl, 2-aminoethyl, N,N-methylamino, dimethylamino, N-thylamino, methylthio, ethylthio, trifluoromethylthio, methylsulfinyl, ethylsulfimyl, methylsulfonyl, ethylsulfonyl, trifluoromethyl, pentafluoroethyl, 2,2,2-trifluoroethyl, 2,2,3,3,3-pentafluoropropyl, tifluoromethoxy, 1,1,2,2,4-etrafluoroethoxy, fluoro, chloro, bromo, amidosulfonyl, N-methylamidosulfonyl, N,N-imethylamidosulfonyl, acetyl, propanoyl, trifluoroacetyl, pentafluoropropanoyl, hydroxymethyl, 1-hydroxyethyl, 2-hydroxyethyl, 2,2,2-bifluoro I-hydroxyethyl, 2,2,2-trifluoro-1-trifluoromethyl-1-hydroxyethyl, carboxymethyl, methoxycarbonyl, ethoxycarbonyl, amidocarbonyl, N-methylamidocarbonyl, N,N-dimethylamidocarbonyl, cyano, and Q^{b};

B can be selected from the group consisting of 1-propenyl, propyl, isopropyl, butyl, 2-butenyl, 3-butenyl, sec-butyl, isobutyl, 2-methylpropenyl, 1-pentyl, 2-pentenyl, 3-pentenyl, 4-pentenyl, 2-pentyl, 1-methyl-2-butenyl, 1-methyl-3-butenyl, 3-pentyl, 1-ethyl-2-propenyl, 2-methylbutyl, 2-methyl-2-butenyl, 2-methyl-3-butenyl, 3-methylbutyl, 3-methyl-2-butenyl, 3-methyl-3-butenyl, 2,2-difluoropropyl, 2-trifiuoromethyl-3,3,3-tiifluoropropyl, 1,1,1,2,2,2-hexafluoropropyl, 3,3,3-triuoroprop-1-yl, and 3,3,3-trifluoroprop-2-yl, wherein each member of group B may be optionally substituted at any carbon up to and including 5 atoms from the point of attachment of B to A with one or more of the group consisting of R₃₂, R₃₃, R₃₄, R₃₅, and R₃₆;

B can be selected from the group consisting of cyclopropyl, cyclobutyl, oxetan-2-yl, oxetan-3-yl, azetidin-1-yl, azetidin-2-yl, azetidin-3-yl, thiaetan-2-yl, thiaetan-3-yl, wherein each ring carbon may be optionally substituted with R₃₃, a ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, and a ring carbon or nitrogen atom adjacent to the R₁₃ position and two atoms from the point of attachment may be substituted with R₁₂;

R⁹, R¹⁰, R¹¹, R¹², and R¹³ are independently selected from the group consisting of amidino, guanidino, dimethylsulfonium, carboxy, methoxy, ethoxy, isopropoxy, propoxy, hydroxy, amino, methoxyamino, ethoxyamino, aminomethyl, 1-ammoethyl, 2-aminoethyl, N,N-methylamino, dimethylamiino, N-ethylamino, methylsulfinyl, ethylsulfinyl, methylsulfonyl, ethylsulfonyl, amidosulfonyl, N-methylamidosulfonyl, N,N-dimethylamidosulfonyl, acetyl, propanoyl, trifluoroacetyl, pentafluoropropanoyl, hydroxymethyl, 1-hydroxyethyl, 2-hydroxyethyl, 2,2,2-trifluorol-hydroxyethyl, 2,2,2-trifluoro-1-tifluoromethyl-1-hydroxyethyl, carboxymethyl, methoxycarbonyl, ethoxycarbonyl, amidocarbonyl, N-methylamidocarbonyl, N,N-dimethylamidocarbonyl, and cyano;

A is selected from the group consisting of single covalent bond, O, C(O), CH₂, CH₃CH, CF₃CH, CH₃CC(O), CF₃CC(O), C C(O)CCH₃, C(O)CCF₃, CH₂C(O), and (O)CCH₂;

R¹ is selected from the group consisting of hydrido, methyl, ethyl, and 2,2,2-trifluoroethyl;

R² is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, 5 isoxazolyl, 2-pyiidyl, 3-pyridyl, 4 pyridyl, 2-pyrazinyl, 2-pyrimidinyl, 4-pyrimidinyl, 5-pyrimidinyl, 3-pyridazinyl, 4-pyridazinyl, and 1,3,5-triazin-2-yl, wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;

K is CR^{4a}R^{4b} wherein R^{4a} and R^{4b} are independently selected from the group consisting of chloro, fluoro, and hydrido;

E⁰ is E¹, when K is CR^{4a}R^{4b} wherein El is selected from the group consisting of a covalent single bond, C(O)N(H), (H)NC(O), S(O)₂N(H), N(H)S(O)₂, S(O)₂N(H)C(O), and C(O)N(H)S(O)₂;

K can be N(H) and CH₂N(H);

E⁰ is E², when K is N(H) and CH₂N(H), wherein E² is selected from the group consisting of C(O)N(H), (H)NC(O), S(O)₂N(H), N(H)S(O)₂, S(O)₂N(H)C(O), and C(O)N(H)S(O)₂;

Y⁰ is selected from the group of formulas consisting of:

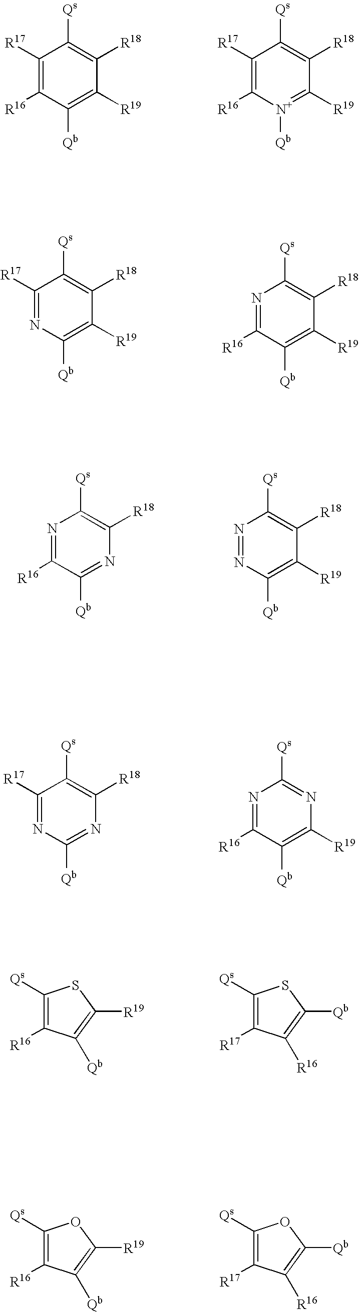

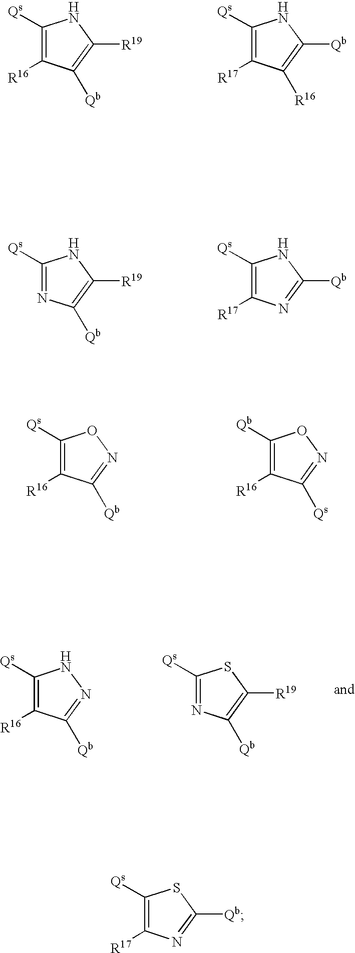

R¹⁶, R¹⁷, R¹⁸, and R¹⁹ are independently selected from the group consisting of hydrido, amidino, guanidino, methoxy, ethoxy, isopropoxy, methylthio, ethylthio, trifluoromethylthio, trifluoromethyl, pentafluoroethyl, 2,2,2-tifluoroethyl, 2,2,3,3,3-pentafluoropropyl, trifluoromethoxy, 1,1,2,2-tetratluoroethoxy, fluoro, chloro, bromo, acetyl, propanoyl, trifluoroacetyl, pentafluoropropanoyl, methoxycarbonyl, ethoxycarbonyl, and cyano;

Q^{b} is selected, when bonded to a carbon, from the group consisting of NR²⁰R²¹,NR²⁰ R²¹R²², dimethylsulfouium, methylethylsulfonium, diethylsulfonium, trimethylphosphonium, C(NR²⁵)NR²³R²⁴, N(R²⁶)C(NR²⁵)N(R²³)(R²⁴), C(O)N(R²⁶)C(NR²⁵)N(R²³)(R²⁴), N(R²⁶)N(R²⁶)C(NR²⁵)N(R²³)(R²⁴), N(R²⁶)N(R²⁶-)SO₂N(R²³)(R²⁴), C(O)NR²³R²⁴, and C(O)NR²³R²⁴ with the provisos that no more than one of R²⁰, R²¹, and R²² can be hydroxy, methoxy, ethoxy, N-methylamino, N,N-dimethylamino, N,N,N-trimethylamino, or amino and that no more than one of R²³, R²⁴, and R²⁶ can be hydroxy, methoxy, ethoxy, N-methylamino, N,N-dimethylamino, N,N,N-trimethylamino, or amino when two of the group consisting of R²³, R²⁴, and R²⁶ are bonded to the same atom and that said Qb group is bonded directly to a carbon atom;

R²⁰, R²¹, R²², R²³, R²⁴, R²⁵, and R²⁶ are independently selected from the group consisting of hydrido, methyl, ethyl, hydroxy, methoxy, ethoxy, 2-aminoethyl, 2-N-methylamino)ethyl, 2-(N,N-dimethylamino)ethyl, 2-(N,N,N-trimethylamino)ethyl, N-(2-hydroxyethyl)amino, N,N-bis-(2-hydroxyethyl)amino, N-(2-hydroxyethyl)-N-(2-aminoethyl)amino, N-methylamino, N,N-dimethylamino, and N,N,N-trimethylamino;

Q^{b} is selected, when bonded to a nitrogen, from the group consisting of oxy, methyl, ethyl, 2-aminoethyl, 2,(N-methylamino)ethyl, 2-(N,N-dimethylamino)ethyl, 2-(N,N,N-trimethylamino)ethyl, N-(2-hydroxyethyl)amino, N,N-bis-(2-hydroxyethyl)amino, amino, hydroxylamino, N-methoxyamino, N-methylamino, N,N-dimethylamino, and N,N,N-trimethylamino;

Q^{s} is selected from the group consisting of a single covalent bond, CH₂, CH₃CH, CF₂, CF₃CH, CH₂O, CH₃C(H)O, CF₃C(H)O, CH₂S, CH₃C(H)S, CF₃C(H)S, CH₂C(O), CH₃C(H)C(O), CF₃C(H)C(O), and CF₂C(O) with the proviso that Q^{s} is bonded to E⁰ through a carbon atom.

In an even more preferred specific embodiment of compounds of Formula I, compounds have the Formula I-EMPS:

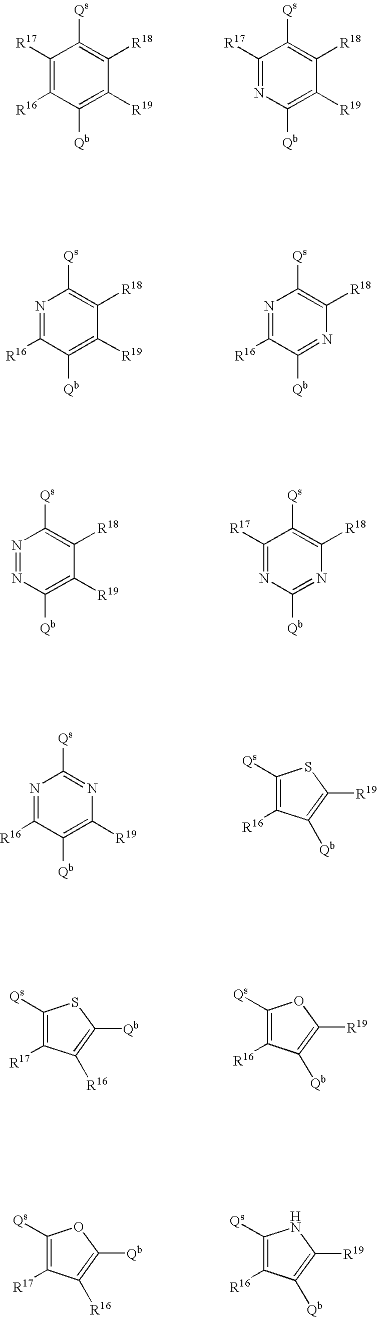

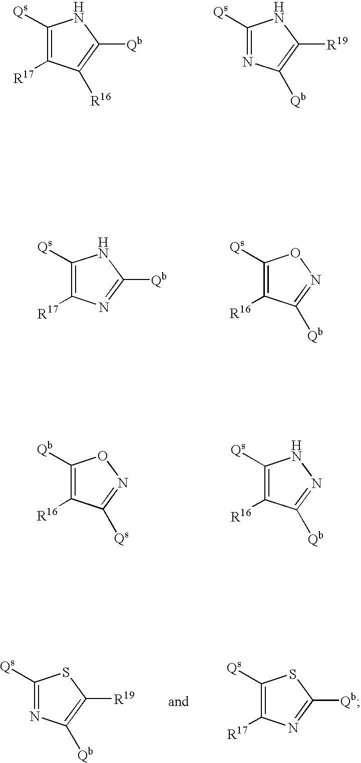

or a pharmaceutically acceptable salt thereof, wherein;

B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3 isoxazolyl, 5-isoxazolyl, 2-pyridyl, 3-pyridyl, and 4 pyridyl, wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;

R³², R³³, R³⁴, R³⁵, and R³⁶ are independently selected from the group consisting of hydrido, amidino, guanidino, methoxy, ethoxy, hydroxy, amino, methoxyamino, ethoxyamino, aminomethyl, 1-aminoethyl, 2-aminoethyl, N,N-methylamino, dimethylamino, N-ethylamino, trifluoromethyl, pentafluoroethyl, 2,2,2-trifluoroethyl, 2,2,3,3,3-pentafluoropropyl, fluoro, chloro, bromo, hydroxymethyl, 1-hydroxyethyl, 2-hydroxyethyl, 2,2,2-trifluoro-1-hydroxyethyl, 2,2,2-trifluoro-1-tifluoromethyl-1-hydroxyethyl, methoxycarbonyl, ethoxycarbonyl, cyano, and Q^{b};

B can be selected from the group consisting of propyl, isopropyl, butyl, sec-butyl, isobutyl, 1-pentyl, 2-pentyl, 3-pentyl, 2-methylbutyl, 3-methylbutyl, 2,2-difluoropropyl, 2-trifluoromethyl-3,3,3-trifluoropropyl, 1,1,1,2,2,2-hexafluoropropyl, 3,3,3-trifluoroprop-1-yl, and 3,3,3-trifluoroprop-2-yl, wherein each member of group B may be optionally substituted at any carbon up to and including 5 atoms from the point of attachment of B to A with one or more of the group consisting of R₃₂, R₃₃, R₃₄, R₃₅, and R₃₆;

B can be selected from the group consisting of cyclopropyl, cyclobutyl, oxetan-2-yl, oxetan-3-yl, azetidin-1-yl, azetidin-2-yl, azetidin-3-yl, thiaetan-2-yl, thiaetan-3-yl, wherein each ling carbon may be optionally substituted with R₃₃, a ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, and a ring carbon or nitrogen atom adjacent to the R₁₃ position and two atoms from the point of attachment may be substituted with R₁₂;

R⁹, R¹⁰, R¹¹, R¹², and R¹³ are independently selected from the group consisting of amidino, guanidino, carboxy, methoxy, ethoxy, hydroxy, amino, methoxyamino, ethoxyamino, aminomethyl, 1-aminoethyl, 2-aminoethyl, N,N-methylamino, dimethylamino, N-ethylamino, acetyl, propanoyl, trifluoroacetyl, pentafluoropropanoyl, hydroxymethyl, 1-hydroxyethyl, 2-hydroxyethyl, carboxymethyl, methoxycarbonyl, ethoxycarbonyl, amidocarbonyl, N-methylamidocarbonyl, N,N-dimethylamidocarbonyl, and cyano;

A is selected from the group consisting of single covalent bond, O, C(O), CH₂, CH₂C(O), and (O)CCH₂;

R¹ is selected from the group consisting of hydrido and methyl;

R² is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, pyrazolyl, 2-thiazolyl, 3-isoxazolyl, 5 isoxazolyl, 2-pyridyl, 3-pyridyl, 4 pyridyl, 2-pyrazinyl, 2-pyrimidinyl, 4-pyrimidinyl, 5-pyrimidnyl, 3-pyridazinyl, 4-pyridazinyl, and 1,3,5-triazin-2-yl, wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;

K is CR^{4a}R^{4b} wherein R^{4a} and R^{4b} are independently selected from the group consisting of chloro, fluoro, and hydrido;

E⁰ is E¹, when K is CR^{4a}R^{4b},wherein E is selected from the group consisting of a covalent single bond, C(O)N(H), (H)NC(O), S(O)₂N(H), N(H)S(O)₂, S(O)₂N(H)C(O), and C(O)N(H)S(O)₂;

K can be N(H) and CH₂N(H);

E⁰ is E², when K is N(H) and CH₂N(H), wherein E is selected from the group consisting of C(O)N(H), (H)NC(O), S(O)₂N(H), N(H)S(O)₂, S(O)₂N(H)C(O), and C(O)N(H)S(O)₂;

Y⁰ is selected from the group of formulas consisting of:

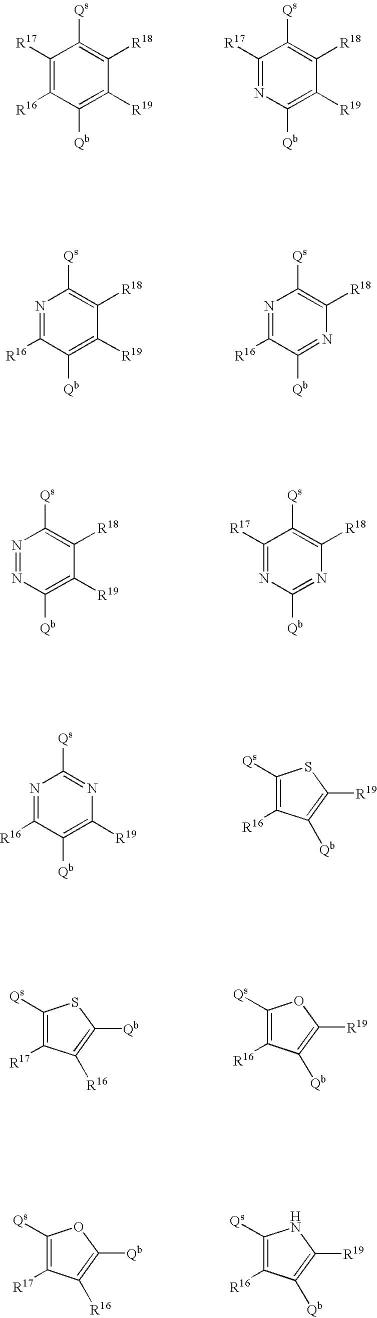

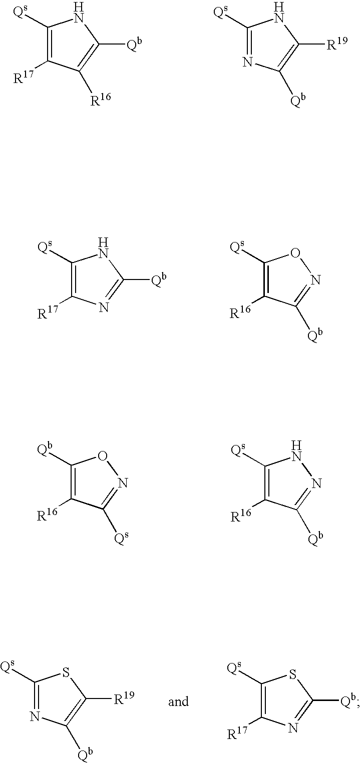

R¹⁶, R¹⁷, R¹⁸, and R¹⁹ are independently selected from the group consisting of hydrido, methoxy, ethoxy, trifluoromethyl, pentafluoroethyl, 2,2,2-trifluoroethyl, trfluoromethoxy, fluoro, chloro, bromo, acetyl, trifluoroacetyl, methoxycarbonyl, ethoxycarbonyl, and cyano;

Q^{b} is selected from the group consisting of NR²⁰R²¹, NR²⁰R²¹R²² dimethylsulfonium, methylethylsulfonium, diethylsulfonium, trimethylphosphonium, C(NR²⁵)NR²³R²⁴, N(R²⁶)C(NR²⁵)N(R²³)(R²⁴), C(O)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²⁰, R²¹, and R²² can be hydroxy, methoxy, ethoxy, N-methylamino, N,N-dimethylamino, N,N,N-trimethylamino, or amino and that no more than one of R²³, R²⁴, and R²⁶ can be hydroxy, methoxy, ethoxy, N-methylamino, N,N-dimethylamino, N,N,N-trimethylamino, or amino when two of the group consisting of R²³, R²⁴, and R²⁶ are bonded to the same atom and that said Q group is bonded directly to a carbon atom;

R²⁰, R²¹, R²², R²³, R²⁴, R²⁵ and R²⁶ are independently selected from the group consisting of hydrido, methyl, ethyl, hydroxy, methoxy, ethoxy, 2-aminoethyl, 2-N-methylamino)ethyl, 2-N,N-dimethylamino)ethyl, 2-(N,N,N-trimethylamino)ethyl, N-(2-hydroxyethyl)amino, N,N-bis-(2-hydroxyethyl)amino, N-(2-hydroxyethyl)-N-(2-aminoethyl)amino, N-methylamino, N,N-dimethylamino, and N,N,N-trimethylamino;

Q^{s} is selected from the group consisting of a single covalent bond, CH₂, CH₃CH, CF₂, CF₃CH, CH₂O, CH₃C(H)O, CF₃C(H)O, CH₂C(O), CH₃C(H)C(O), CF₃C(H)C(O), and CF₂C(O) with the proviso that Q^{s} is bonded to E⁰ through a carbon atom.

In a preferred embodiment of a compound of Formula I, said compound is the formula:

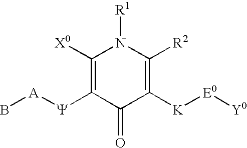

or a pharmaceutically acceptable salt thereof, wherein;

B is phenyl or a heteroaryl of 5 or 6 ring members, wherein a nitrogen with a removable hydrogen or a carbon adjacent to the carbon at the point of attachment of said phenyl or heteroaryl ring to A is optionally substituted by R³², a nitrogen with a removable hydrogen or a carbon at the other position adjacent to the point of attachment is optionally substituted by R³⁶, a nitrogen with a removable hydrogen or a carbon adjacent to R³² and two atoms from the point of attachment is optionally substituted by R³³, a nitrogen with a removable hydrogen or a carbon adjacent to R³⁶ and two atoms from the point of attachment is optionally substituted by R³⁵, and a nitrogen with a removable hydrogen or a carbon adjacent to both R³³ and R³⁵ is optionally substituted by R³⁴;

R⁹, R¹⁰, R¹¹, R¹², R¹³, R³², R³³, R³⁴, R³⁵, and R³⁶ are independently selected from the group consisting of hydrido, acetamido, haloacetamido, amidino, guanidino, alkylenedioxy, haloalkylthio, alkanoyloxy, alkoxy, cycloalkoxy, cycloalkylalkoxy, aralkoxy, aryloxy, heteroaryloxy, heteroaralkoxy, heterocyclyloxy, heterocyclylalkoxy, alkoxyalkyl, haloalkoxylalkyl, hydroxy, amino, alkoxyamino, nitro, alkylamino, N-alkyl-N-arylamino, arylamino, aralkylamino, heteroarylamino, heteroarlakylamino, heterocyclylamno, heterocyclylalkylammo, alkylthio, alkylthioalkyl, alkylsulfinyl, arylsulfinyl, aralkylsulfinyl, cycloalkylsutmnyl, heteroarylsulfinyl, alkylsulfonyl, arylsulfonyl, aralkylsulfonyl, cycloalkylsulfonyl, heteroarylsulfonyl, alkylsulfonylalkyl, aryl, aralkyl, cycloalkyl, cycloalkylalkyl, heteroaryl, heterocyclyl, alkylsulfonamido, amidosulfonyl, alkanoyl, haloalkanoyl, alkyl, alkenyl, halo, haloalkyl, haloalkenyl, haloalkoxy, hydroxyhaloalkyl, hydroxyalkyl, aminoalkyl, haloalkoxyalkyl, carboxyalkyl, carboalkoxy, carboxy, carboxamido, carboxamidoalkyl, and cyano;

R³², R³³, R³⁴, R³⁵, and R³⁶ are independently optionally Q^{b};

B is optionally selected from the group consisting of hydrido, trialkylsilyl, C2-C8 alkyl, C3-C8 alkylenyl, C3-C8 alkenyl, C3-C8 alynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶;

B is optionally a C3-C12 cycloalkyl or C4-C9 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen atom adjacent to the R⁹ position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;

A is selected from the group consisting of a bond, (W⁷)ᵣᵣ—C(CH(R¹⁵))ₚₐ, and (CH(R¹⁵))pₐ—(W⁷)ᵣᵣ wherein rr is 0 or 1, pa is an integer selected from 0 through 6, and W⁷ is selected from the group consisting of O, S, C(O), (R⁷)NC(O), (R⁷)NC(S), and N(R⁷) with the proviso that no more than one of the group consisting of rr and pa is O at the same time;

R⁷ is selected from the group consisting of hydrido, hydroxy, and alkyl;

R¹⁵ is selected from the group consisting of hydrido, hydroxy, halo, alkyl, and haloalkyl;

Ψ is NH or NOH;

X⁰ is selected from the group consisting of hydrido, alkyl, alkenyl, cyano, halo, haloalkyl, haloalkoxy, haloalkylthio, amino, aminoalkyl, alkylamino, amidino, hydroxy, hydroxyamino, alkoxy, hydroxyalkyl, alkoxyamino, thiol, and alkylthio;

R¹ is selected from the group consisting of hydrido, alkyl, alkenyl, haloalkyl, amino, aminoalkyl, alkylamino, hydroxy, and hydroxyalkyl;

X⁰ and R¹ or R¹ and R² is optionally bonded together to form a partially saturated C5-C8 heterocyclyl ring, wherein said heterocyclyl ring is optionally substituted with one or more of the group consisting of R⁹, R¹⁰, R¹¹, R¹², and R¹³;

R² is Z⁰-Q^{b};

Z⁰ is selected from the group consisting of a bond, (CR⁴¹R⁴²)_{q} wherein q is an integer selected from 1 through 3, and (CH(R⁴¹))_{g}—W⁰—(CH(R⁴²))ₚ wherein g and p are integers independently selected from 0 through 3 and W⁰ is selected from the group consisting of O, S, C(O), S(O), N(R⁴¹), and ON(R⁴¹);

Z⁰ is optionally (CH(R⁴¹))ₑ—W²²—(CH(R⁴²))ₕ wherein e and h are independently 0 or 1 and W²² is selected from the group consisting of CR⁴¹═CR⁴², 1,2-cyclopropyl, 1,2-cyclobutyl, 1,2-cyclohexyl, 1,3-cyclohexyl, 1,2-cyclopentyl, 1,3-cyclopentyl, 2,3-morpholinyl, 2,4-morpholinyl, 2,6-morpholinyl, 3,4-morpholinyl, 3 morpholinyl, 1,2-piperazinyl, 1,3-piperazinyl, 2,3-piperazinyl, 2,6-piperazinyl, 1,2-piperidinyl, 1,3-piperidinyl, 2,3-piperidinyl, 2,4-piperidinyl, 2,6-piperidinyl, 3,4-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2,3-tetrahydrofumanyl, 2,4-tetrahydrofuranyl, 2,5-tetrahydrofuranyl, and 3,4-tetrahydrofuranyl, wherein Z⁰ is directly bonded to the 4-pyridone ring and W²² is optionally substituted with one or more substituents selected from the group consisting of R⁹, R¹⁰, R¹¹, R¹², and R¹³;

R⁴¹ and R⁴² are independently selected from the group consisting of amidino, hydroxyamino, hydrido, hydroxy, amino, and alkyl;

Q is phenyl or a heteroaryl of 5 or 6 ring members, wherein a nitrogen with a removable hydrogen or a carbon adjacent to the carbon at the point of attachment of said phenyl or heteroaryl ring to Z⁰ is optionally substituted by R⁹ a nitrogen with a removable hydrogen or a carbon at the other position adjacent to the point of attachment is optionally substituted by R¹³, a nitrogen with a removable hydrogen or a carbon adjacent to R⁹ and two atoms from the point of attachment is optionally substituted by R¹⁰, a nitrogen with a removable hydrogen or a carbon adjacent to R¹³ and two atoms from the point of attachment is optionally substituted by R¹², and a nitrogen with a removable hydrogen or a carbon adjacent to both R¹⁰ and R¹² is optionally substituted by R¹¹;

Q is optionally hydrido with the proviso that Z⁰ is selected from other than a bond;

K is CR^{4a}R^{4b};

R^{4a} and R^{4b} are independently selected from the group consisting of halo, hydrido, hydroxyalkyl, alkyl, alkoxyalkyl, alkylthioalkyl, and haloalkyl;

E⁰, with the proviso that K is CR^{4a}R^{4b}, is E¹ wherein E¹ is selected from the group consisting of a covalent single bond, C(O)N(H), (H)NC(O), C(S)N(H), (H)NC(S), S(O)₂N(H), N(H)S(O)₂, S(O)₂N(H)C(O), and C(O)N(H)S(O)₂;

K is optionally (CH(R¹⁴))ⱼ-T wherein j is 0 or 1 and T is a bond or N(R⁷) with the proviso that (CH(R¹⁴))ⱼ is bonded to the phenyl ring;

R¹⁴ is selected from the group consisting of hydrido, halo, alkyl, and haloalkyl;

E⁰, with the proviso that K is (CH(R¹⁴))ⱼ-T, is E² wherein E² is selected from the group consisting of C(O)N(H), (H)NC(O), C(S)N(H), (H)NC(S), S(O)₂N(H), N(H)S(O)₂, S(O)₂N(H)C(O), and C(O)N(H)S(O)₂;

Y⁰ is phenyl or a heteroaryl of 5 or 6 ring members, wherein one carbon of said phenyl or said heteroaryl is substituted by Q^{s}, a carbon two or three contiguous atoms from the point of attachment of Q^{s} to said phenyl or said heteroaryl to said phenyl or said heteroaryl is substituted by Q^{b}, a carbon adjacent to the point of attachment of Q^{b} is optionally substituted by R another carbon adjacent to the point of attachment of Q^{b} is optionally substituted by R¹⁸, a carbon adjacent to Q^{b} is optionally substituted by R¹⁶, and another carbon adjacent to Q^{b}, optionally substituted by R¹⁹;

R¹⁶, R¹⁷, R¹⁸, and R¹⁹ are independently selected from the group consisting of hydrido, amidino, guanidino, carboxy, haloalkylthio, alkoxy, hydroxy, amino, nitro, alkoxyamino, alkylamino, alkylthio, alkylsulfinyl, alkylsulfonyl, alkanoyl, haloalkanoyl, alkyl, alkenyl, halo, haloalkyl, haloalkoxy, hydroxyalkyl, aminoalkyl, haloalkoxyalkyl, carboalkoxy, and cyano;

R¹⁶ or R¹⁹ is optionally selected from the group consisting of NR²⁰ R²¹, N(R²⁶)C(NR²⁵)N(R²³)(R²⁴), and C(NR²⁵)NR²³R²⁴, with the proviso that R¹⁶, R¹⁹, and Q are not simultaneously hydrido;

Q^{b} is selected from the group consisting of NR²⁰R²¹, aminoalkyl, hydrido, N(R²⁶)C(NR²⁵)N(R²³)(R²⁴), and C(NR²⁵)NR²³R²⁴, with the proviso that no more than one of R²⁰ and R²¹ is selected from the group consisting of hydroxy, amino, alkylamino, and dialkylamino at the same time, with the further proviso that no more than one of R²³ and R²⁴ is selected from the group consisting of hydroxy, amino, alkylamino, and dialkylamino at the same time;

R²⁰, R²¹, R²³, R²⁴, R²⁵, and R²⁶ are independently selected from the group consisting of hydrido, alkyl, hydroxy, aminoalkyl, amino, dialkylamino, alkylamino, and hydroxyalkyl;

Q^{b} is selected from the group consisting of a bond, (CR³⁷R³⁸)_{b} wherein b is an integer selected from 1 through 4, and (CH(R¹⁴))_{c}—W¹—(CH(R¹⁵))_{d} wherein c and d are integers independently selected from 1 through 3 and W¹ is selected from the group consisting of C(O)N(R¹⁴), (R¹⁴)NC(O), S(O), S(O)₂, S(O)₂N(R¹⁴), N(R¹⁴)S(O)₂, and N(R¹⁴), with the proviso that R¹⁴ is selected from other than halo when directly bonded to N, and with the additional proviso that (CR³⁷R³⁸)_{b} and (CH(R¹⁴))_{c} are bonded to E;

R³⁷ and R³⁸ are independently selected from the group consisting of hydrido, alkyl, and haloalkyl;

R³⁸ is optionally aroyl or heteroaroyl, wherein R³⁸ is optionally substituted with one or more substituents selected from the group consisting of R¹⁶, R¹⁷, R¹⁸, and R¹⁹;

Y⁰ is optionally Y^{AT} wherein Y^{AT} is Q^{b} Q^{s};

Y⁰ is optionally Q_{b}-Q^{ss} wherein Q^{ss} is (CH(R¹⁴))_{c}—W²—C(R¹⁵))ₕ, wherein e and h are independently 1 or 2 and W² is CR^{4a}CR^{4b}, with the proviso that (CH(R¹⁴))ₑ is bonded to E⁰;

Y⁰ is optionally Q^{b}-Qₛₛₛₛ or Q^{b}Q^{ssssr} wherein Q^{ssss} is (CH(R³⁸))ᵣ—W⁵, Q^{ssssr} is (CH(R³⁸))ᵣW⁶, r is 1 or 2, W⁵ and W are independently selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,7-imidazo(1,2-a)pyridinyl, 3,4-imidazo(1,2-a)pyridinyl, 3,5-imidazo(1,2-a)pyridinyl, 3,6-imidazo(1,2-a)pyridinyl, 3,7-imidazo(1,2-a)pyridinyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3 Squinolinyl, 3,6-quinoiinyl, 3,7-quinolinyl, 3,5-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,5-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoqninolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl, and each carbon and hyrido containing nitrogen member of the ring of the W⁵ and of the ring of the W⁶, other than the points of attachment of W⁵, and W⁶, is optionally substituted with one or more of the group consisting of R⁹, R¹⁰, R¹¹, and R¹², with the proviso that Q^{b} is bonded to lowest number substituent position of each W⁵, with the further proviso that Q^{b} is bonded to highest number substituent position of each W⁶, and with the additional proviso that (CH(R³⁸))ᵣ is bonded to E⁰.

In a more preferred embodiment of a compound of Formula I, said compound is the Formula:

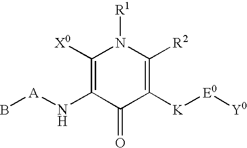

or a pharmaceutically acceptable salt thereof, wherein;

B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydrido, acetamido, haloacetamido, amidino, guanidino, alkylenedioxy, haloalkylthio, alkanoyloxy, alkoxy, hydroxy, amino, alkoxyamino, haloalkanoyl, nitro, alkylamino, alkylthio, aryl, aralkyl, cycloalkyl, cycloalkylalkyl, heteroaryl, heterocyclyl, alkylsulfonamido, amidosulfonyl, alkyl, alkenyl, halo, haloalkyl, haloalkenyl, haloalkoxy, hydroxyalkyl, hydroxyhaloalkyl, aminoalkyl, carboalkoxy, carboxy, carboxamido, cyano, and Q^{b};

B is optionally selected from the group consisting of hydrido, trialkylsilyl, C2-C8 alkyl, C3-C8 alkylenyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶;

B is optionally a C3-C12 cycloalkyl or a C4-C9 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen atom adjacent to the R⁹ position and two atoms from the point of attachment is optionally substituted with R¹⁰, a ring carbon or nitrogen atom adjacent to the R¹³ position and two atoms from the point of attachment is optionally substituted with R¹², a ring carbon or nitrogen atom three atoms from the point of attachment and adjacent to the R¹⁰ position is optionally substituted with R¹¹, a ring carbon or nitrogen atom three atoms from the point of attachment and adjacent to the R¹² position is optionally substituted with R³³, and a ring carbon or nitrogen atom four atoms from the point of attachment and adjacent to the R¹¹ and R³³ positions is optionally substituted with R³⁴;

R⁹, R¹⁰, R¹¹, R¹², and R¹³ are independently selected from the group consisting of hydrido, acetamido, haloacetamido, alkoxyamino, alkanoyl, haloalkanoyl, amidino, guanidino, alkylenedioxy, haloalkylthio, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkylamino, N-alkyl-N-arylamino, arylamino, aralkylamino, heteroarylamino, heteroaralkylamino, heterocyclylamino, heterocyclylalkylamino, alkylthio, alkylsulfinyl, arylsulfinyl, aralkylsulfinyl, cycloalkylsulfinyl, heteroarylsulfinyl, alkylsulfamido, alkylsulfonyl, arylsulfonyl, aralkylsulfonyl, cycloalkylsulfonyl, heteroarylsulfonyl, amidosulfonyl, alkyl, aryl, aralkyl, cycloalkyl, cycloalkylalkyl, heteroaryl, heterocyclyl, halo, haloalkyl, haloalkoxy, hydroxyalkyl, hydroxyhaloalkyl, aminoalkyl, carboalkoxy, carboxy, carboxyalkyl, carboxamido, and cyano;

A is bond or (CH(R¹⁵))ₚₐ—(W⁷)ᵣᵣ wherein rr is 0 or 1, pa is an integer selected from 0 through 3, and W⁷ is selected from the group consisting of 0, S, C(O), (R⁷)NC(O), (R⁷)NC(S), and N(R⁷), with the proviso that W⁷ is bonded to the N(H) on the 4-pyridone ring;

R⁷ is selected from the group consisting of hydrido, hydroxy and alkyl;

R¹⁵ is selected from the group consisting of hydrido, hydroxy, halo, alkyl, and haloalkyl;

X⁰ is selected from the group consisting of hydrido, alkyl, cyano, halo, haloalkyl, haloalkoxy, amino, aminoalkyl, alkylamino, amidino, hydroxy, hydroxyamino, alkoxy, hydroxyalkyl, alkoxyamino, thiol, and alkylthio;

R¹ is selected from the group consisting of hydrido, alkyl, haloalkyl, amino, aminoalkyl, alkylamino, hydroxy, and hydroxyalkyl;

X⁰ and R¹ or R¹ and R² is optionally bonded together to form a partially saturated C5-C8 heterocyclyl ring, wherein said heterocyclyl ring is optionally substituted with one or more of the group consisting of R⁹, R¹⁰, R¹¹, R¹², and R¹³;

R² is Z⁰-Q^{b};

Z⁰ is selected from the group consisting of a bond, (CR⁴¹R⁴²)_{q} wherein q is 1 or 2, and (CH(R⁴¹))_{g}—W⁰—(CH(R⁴²))ₚ wherein g and p are integers independently selected from 0 through 3 and W⁰ is selected from the group consisting of O, S, C(O), S(O), N(R⁴¹), and ON(R⁴¹);

Z⁰ is optionally (CH(R⁴¹))ₑ—W²—(CH(R⁴²))ₕ wherein e and h are independently 0 or 1 and W²² is selected from the group consisting of CR⁴¹═CR⁴², 1,2-cyclopropyl, 1,2-cyclobutyl, 1,2-cyclohexyl, 1,3-cyclohexyl, 1,2-cyclopentyl, 1,3-cyclopentyl, 2,3-morpholinyl, 2,4-morpholinyl, 2,6-morpholinyl, 3,4-morpholinyl, 3,5-morpholinyl, 1,2-piperazinyl, 1,3-piperazinyl, 2,3-piperazinyl, 2,6-piperazinyl, 1,2-piperidinyl, 1,3-piperidinyl, 2,3-piperidinyl, 2,4-piperidinyl, 2,6-piperidinyl, 3,4-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2,3-tetrahydrofuranyl, 2,4-tetrahydrofuranyl, 2,5-tetrahydrofuranyl, and 3,4-tetrahydrofuranyl, wherein Z⁰ is directly bonded to the 4-pyridone ring and W²² is optionally substituted with one or more substituents selected from the group consisting of R⁹, R¹⁰, R¹¹, R¹², and R¹³;

R⁴¹ and R⁴² are independently selected from the group consisting of hydrido, hydroxy, alkyl, and amino;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

Q is optionally hydrido with the proviso that Z⁰ is other than a bond;

K is CR^{4a}R^{4b};

R^{4a} and R^{4b} are independently selected from the group consisting of hydrido, hydroxyalkyl, alkyl, alkoxyallyl, alkylthioalkyl, and haloalkyl;

E⁰, with the proviso that K is CR^{4a}R^{4b}, is E¹ wherein E¹ is selected from the group consisting of a covalent single bond, C(O)N(H), (H)NC(O), S(O)₂N(H), and N(H)S(O)₂;

K is optionally (CH(R¹⁴))ⱼ-T wherein j is 0 or 1 and T is a bond or N(R⁷) with the proviso that (CH(R¹⁴))ⱼ is bonded to the phenyl ring;

R¹⁴ is hydrido or halo;

E⁰ with the proviso that K is (CH(R¹⁴))ⱼ.T, is E² wherein E² is selected from the group consisting of C(O)N(H), (H)NC(O), C(S)N(H), (H)NC(S), S(O)₂N(H), N(H)S(O)₂, S(O)₂N(H)C(O), and C(O)N(H)S(O)₂;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydrido, amidino, guanidino, carboxy, haloalkylthio, alkoxy, hydroxy, amino, alkoxyamino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally selected from the group consisting of NR²⁰R²¹,N(R²⁶)C(NR²⁵)N(R²³)(R²⁴),and C(NR²⁵)NR²³R²⁴, with the proviso that R¹⁶, R¹⁹, and Q are not simultaneously hydrido;

Q^{b} is selected from the group consisting of NR²⁰R²¹, hydrido, N(R²⁶)C(NR²⁵)N(R²³)(R²⁴), and C(NR²⁵)NR²³R²⁴, with the proviso that no more than one of R²³ and R²⁴ is selected from the group consisting of hydroxy, amino, alkylamino, and dialkylamino at the same time, with the further proviso that no more than one of R and R is selected from the group consisting of hydroxy, amino, alkylamino, and dialkylamino at the same time;

R²⁰, R²¹, R²³, R²⁴, R²⁵, and R²⁶ are independently selected from the group consisting of hydrido, alkyl, hydroxy, amino, alkylamino and dialkylamino;

Q^{s} is selected from the group consisting of a bond, (CR³⁷R³⁸)_{b} wherein b is an integer selected from 1 through 4, and (CH(R¹⁴))_{c}—W¹—(CH(R¹⁵))_{d} wherein c and d are integers independently selected from 1 through 3 and W¹ is selected from the group consisting of C(O)N(R¹⁴), (R¹⁴)NC(O), S(O), S(O)₂, S(O)₂N(R¹⁴), N(R¹⁴)S(O)₂, and N(R¹⁴), with the proviso that R¹⁴ is selected from other than halo when directly bonded to N, and with the additional proviso that (CR³⁷R³⁸)_{b} and (CR³⁷R³⁸)_{b}, and (CH(R¹⁴))_{c} are bonded to E;

R³⁷ and R³⁸ are independently selected from the group consisting of hydrido, alkyl, and haloalkyl;

R³⁸ is optionally aroyl or heteroaroyl, wherein R³⁸ is optionally substituted with one or more substituents selected from the group consisting of R¹⁶, R¹⁷, R¹⁸, and R¹⁹;

Y⁰ is optionally Y^{AT} wherein Y^{AT} is Q^{b}-Q^{s};

Y⁰ is optionally Q^{b}-Q^{ss} wherein Q^{ss} is (CH(R¹⁴))ₑ—W²—(CH(R¹⁵))ₕ, wherein e and h are independently 1 or 2 and W² is CR^{4a}=CR^{4b} with the proviso that (CH(R¹⁴))ₑ is bonded to E⁰.

In an even more preferred embodiment of a compound of Formula I, said compound is the formula:

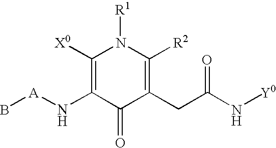

or a pharmaceutically acceptable salt thereof, wherein;

B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, cyano, and (b;

A is a bond or (CH(R¹⁵))ₚₐ—(W⁷)ᵣᵣ wherein rr is 0 or 1, pa is an integer selected from 0 through 3, and W⁷ is (R⁷)NC(O) or N(R⁷);

R⁷ is selected from the group consisting of hydrido, hydroxy and alkyl;

R¹⁵ is selected from the group consisting of hydrido, halo, alkyl, and haloalkyl;

X⁰ is selected from the group consisting of hydrido, amino, alkyl, halo, haloalkyl, haloalkoxy, aminoalkyl, hydroxy, alkoxy, hydroxyalkyl, and alkylthio;

R¹ is independently selected from the group consisting of hydrido, hydroxy, amino, hydroxyalkyl, alkyl, aminoalkyl, and haloalkyl;

R² is Z⁰-Q^{b};

Z⁰ is selected from the group consisting of a bond, CH₂, CH₂CH₂, W⁰—(CH(R⁴²))ₚ wherein p is 0 or 1 and W⁰ is selected from the group consisting of O, S, and N(R⁴¹);

R⁴¹ and R⁴² are independently hydrido or alkyl;

Q^{b} is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

R⁹, R¹¹, and R¹³ are independently selected from the group consisting of hydrido, hydroxy, amino, amidino, guanidino, alkylamino, alkylthio, alkylsulfonamido, alkylsulfinyl, allylsulfonyl, amidosulfonyl, alkyl, alkoxy, halo, haloalkyl, haloalkoxy, hydroxyalkyl, hydroxyhaloalkyl, carboxy, carboxamido, and cyano;

R¹⁰ and R¹² are independently selected from the group consisting of hydrido, acetamido, haloacetamido, amidino, guanidino, alkyl, aryl, arakyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkoxyamino, alkylamino, arylamino, aralkylamino, heteroarylamino, heteroaralkylamino, heterocyclylamino, heterocyclylalkyl amino, alkylsulfonamido, amidosulfonyl, arylsulfinyl, aralkylsulfinyl, cycloalkylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally NR²⁰R²¹ or C(NR²⁵)NR²³R²⁴ with the proviso that R¹⁶, R¹⁹, and Q^{b} are not simultaneously hydrido;

Q^{b} is selected from the group consisting of NR²⁰R²¹, hydrido, and C(NR²⁵)NR²³R²⁴, with the proviso that no more than one of R²⁰ and R²¹ is hydroxy at the same time and with the further proviso that no more than one of R²³ and R²⁴ is hydroxy at the same time;

R²⁰, R²¹, R²³, R²⁴, and R²⁵ are independently selected from the group consisting of hydrido, alkyl, and hydroxy;

Q^{s} is selected from the group consisting of a bond, CH₂, and CH₂CH₂.

In another even more preferred embodiment of a compound of Formula I, said compound is the Formula:

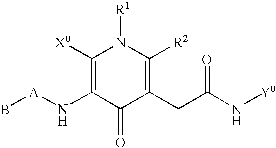

or a pharmaceutically acceptable salt thereof, wherein;

B is selected from the group consisting of hydfido, C2-C8 alkyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³ R³⁴ e35 d R³⁶;

R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, cyano, and Q^{b};

A is a bond or (CH(R¹⁵))^{pa}—(W⁷)ᵣᵣ wherein rr is 0 or 1, pa is an integer selected from 0 through 3, and W⁷ is (R⁷)NC(O) or N(R⁷);

R⁷ is selected from the group consisting of hydrid, hydroxy and alkyl;

R¹⁵ is selected from the group consisting of hydrido, halo, alkyl, and haloalkyl;

X⁰ is selected from the group consisting of hydrido, amino, alkyl, halo, haloalkyl, haloalkoxy, aminoalkyl, hydroxy, alkoxy, hydroxyalkyl, and alkylthio;

R¹ is independently selected from the group consisting of hydrido, hydroxy, amino, hydroxyalkyl, alkyl, aminoalkyl, and haloalkyl;

R² is Z⁰-Q^{b};

Z⁰ is selected from the group consisting of a bond, CH₂, CH₂CH₂, W⁰—(CH(R⁴²))ₚ wherein p is 0 or 1 and W⁰ is selected from the group consisting of O, S, and N(R⁴¹);

R⁴¹ and R⁴² are independently hydrido or alkyl;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

R⁹, R¹¹, and R¹³ are independently selected from the group consisting of hydrido, hydroxy, amino, amidino, guanidino, alkylamino, alkylthio, alkylsulfonamido, alkylsulfinyl, alkylsulfonyl, amidosulfonyl, alkyl, alkoxy, halo, haloalkyl, haloalkoxy, hydroxyalkyl, hydroxyhaloalkyl, carboxy, carboxamido, and cyano;

R¹⁰ and R¹² are independently selected from the group consisting of hydrido, acetamido, haloacetamido, amidino, guanidino, alkyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkoxyamino, alkylamino, arylamino, aralkylamino, heteroarylamino, heteroaralkylamino, heterocyclylamino, heterocyclylalkylamino, alkylsulfonamido, amidosulfonyl, arylsulfinyl, aralkylsulfinyl, cycloalcylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally selected from the group consisting of NR²⁰R²¹,N(R²⁶)C(NR²⁵)N(R²³)(R²⁴),and C(NR²⁵)NR²³R²⁴, with the proviso that R¹⁶, R¹⁹, and Q are not simultaneously hydrido;

Q^{b} is selected from the group consisting of NR²⁰R²¹, hydrido, C(NR²⁵)NR²³R²⁴, and N(R²⁶)C(NR²⁵)N(R²³)(R²⁴), with the proviso that no more than one of R²⁰ and R²¹ is hydroxy at the same time and with the further proviso that no more than one of R²³ and R²⁴ is hydroxy at the same time;

R²⁰, R²¹, R²³, R²⁴ R²⁵, and R²⁶ are independently selected from the group consisting of hydrido, alkyl, and hydroxy;

Q^{s} is selected from the group consisting of a bond, CH₂, and CH₂CH₂.

In still another even more preferred embodiment of a compound of Formula I, said compound is the Formula:

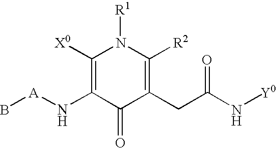

or a pharmaceutically acceptable salt thereof, wherein;

B is a C3-C7 cycloalkyl or a C4-C6 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;

R⁹ R¹¹, and R¹³ are independently selected from the group consisting of hydrido, hydroxy, amino, amidino, guanidino, alkylamino, alkylthio, alkylsulfonamido, alkylsulfinyl, alkylsulfonyl, amidosulfonyl, alkyl, alkoxy, halo, haloalkyl, haloalkoxy, hydroxyalkyl, hydroxyhaloalkyl, carboxy, carboxamido, and cyano;

R¹⁰ and R¹² are independently selected from the group consisting of hydrido, acetamido, haloacetamido, amidino, guanidino, alkyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkoxyamino, alkylamino, arylamino, aralkylamino, heteroarylamino, heteroaralkylamino, heterocyclylamino, heterocyclylalkylamino, alkylsulfonamido, amidosulfonyl, arylsulfinyl, aralkylsulfinyl, cycloalkylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;

R³³ and R³⁴ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, and cyano;

R³³ is optionally Q^{b};

A is a bond or (CH(R¹⁵))ₚₐ—(W⁷)ᵣᵣ wherein rr is 0 or 1, pa is an integer selected from 0 through 3, and W is (R⁷)NC(O) or N(R⁷);

R⁷ is selected from the group consisting of hydrido, hydroxy and alkyl;

R¹⁵ is selected from the group consisting of hydrido, halo, alkyl, and haloalkyl;

X⁰ is selected from the group consisting of hydrido, amino, alkyl, halo, haloalkyl, haloalkoxy, aminoalkyl, hydroxy, alkoxy, hydroxyalkyl, and alkylthio;

R¹ is independently selected from the group consisting of hydrido, hydroxy, amino, hydroxyalkyl, alkyl, aminoalkyl, and haloalkyl;

R² is Z⁰-Q^{b};

Z⁰ is selected from the group consisting of a bond, CH₂, CH₂CH₂, W⁰—(CH(R⁴²))ₚ wherein p is 0 or 1 and W is selected from the group consisting of O, S, and N(R⁴¹);

R⁴¹ and R⁴² are independently hydrido or alkyl;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally NR²⁰R²¹ or and C(NR²⁵)NR²³R²⁴, with the proviso that R¹⁶, R¹⁹, and Q are not simultaneously hydrido;

Q^{b} is selected from the group consisting of NR²⁰R²¹, hydrido, and C(NR²⁵)NR²³R²⁴, with the proviso that no more than one of R²⁰ and R²¹ is hydroxy at the same time and with the further proviso that no more than one of R²³ and R²⁴ is hydroxy at the same time;

R²⁰, R²¹, R²³, R²⁴, and R²⁵ are independently selected from the group consisting of hydrido, alkyl, and hydroxy;

Q^{s} is selected from the group consisting of a bond, CH₂, and CH₂CH₂.

In an additional even more preferred embodiment of a compound of Formula I, said compound is the Formula:

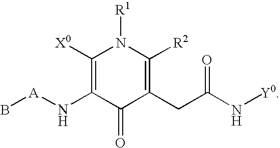

or a pharmaceutically acceptable salt thereof, wherein;

B is selected from the group consisting of hydrido, trialkylsilyl, C2-C4 alkyl, C3-C5 alkylenyl, C3-C4 alkenyl, C3-C4 alkynyl, and C2-C4 haloalkyl, wherein each member of group B is optionally substituted at any carbon up to and including 3 atoms from the point of attachment of B to A with one or more of the group consisting of R³², R³³, and R³⁴;

R³², R³³, and R³⁴ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, and cyano;

A is (CH(R¹⁵))ₚₐ—N(R⁷) wherein pa is an integer selected from 0 through 2 and R is hydrido or alkyl;

R¹⁵ is selected from the group consisting of hydrido, halo, alkyl, and haloalkyl;

X⁰ is selected from the group consisting of hydrido, amino, alkyl, halo, haloalkyl, haloalkoxy, aminoalkyl, hydroxy, alkoxy, hydroxyalkyl, and alkylthio;

R¹ is selected from the group consisting of hydrido, alkyl, haloalkyl, amino, aminoalkyl, hydroxy, and hydroxyalkyl;

R² is Z⁰-Q^{b};

Z⁰ is a bond or CH₂;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹¹ is optionally substituted by R¹¹;

R⁹, R¹¹, and R¹³ are independently selected from the group consisting of hydrido, hydroxy, amino, amidino, guanidino, alkylamino, alkylthio, alkylsulfonamido, alkylsulinyl, alkylsulfonyl, aridosulfonyl, alkyl, alkoxy, halo, haloalkyl, haloalkoxy, hydroxyalkyl, hydroxyhaloalkyl, carboxy, carboxamido, and cyano;

R¹⁰ and R¹² are independently selected from the group consisting of hydrido, acetamido, haloacetamido, amidino, guanidino, allyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy, heterocyclyloxy, heterocyclylalkoxy, hydroxy, amino, alkoxyamino, alkylamino, arylamino, aralkylamino, heteroarylamino, heteroarallamino, heterocyclylamino, heterocyclylalkylamino, alkylsulfonamido, amidosulfonyl, arylsulfinyl, aralkylsulfinyl, cycloalkylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;

Y⁰ is phenyl or a heteroaryl of 5 or 6 ring members, wherein one carbon of said phenyl or said heteroaryl is substituted by Q^{s},a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydnido, amidino, guanidino, carboxy, haloalkylthio, alkoxy, hydroxy, amino, alkylamino, alkylthio, alkylsulfinyl, alkylsuifonyl, alkanoyl, haloalkanoyl, alkyl, halo, haloalkyl, haloalkoxy, hydroxyalkyl, aminoalkyl, and cyano;

R¹⁶ or R¹⁹ is optionally selected from the group consisting of NR²⁰R²¹, N(R²⁶)C(NR²⁵)N(R²³)(R²⁴), and C(NR²⁵)NR²³R²⁴, with the proviso that R¹⁶, R¹⁹, and Q are not simultaneously hydrido;

Q^{b} is selected from the group consisting of NR²⁰R²¹, hydrido, C(NR²⁵)NR²³R²⁴, and N(R²⁶)C(NR²⁵)N(R²³)(R²⁴), with the proviso that no more than one of R²⁰ and R²¹ is hydroxy at the same time and with the further proviso that no more than one of R²³ and R²⁴ is hydroxy at the same time;

R²⁰, R²¹, R²³, R²⁴, R²⁵, and R²⁶ are independently selected from the group consisting of hydrido, alkyl, and hydroxy;

Q^{s} is selected from the group consisting of a bond, CH₂, and CH₂CH₂.

In a fifth even more preferred embodiment of a compound of Formula 1, said compound is Formula:

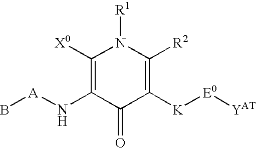

or a pharmaceutically acceptable salt thereof, wherein;

B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydrido, acetamido, haloacetamido, amidino, guanidino, alkylenedioxy, haloalkylthio, alkanoyloxy, alkoxy, hydroxy, amino, alkoxyamino, haloalkanoyl, nitro, alkylamino, allylthio, aryl, aralkyl, cycloalkyl, cycloalkylalkyl, heteroaryl, heterocyclyl, alkylsulfonamido, amidosulfonyl, alkyl, alkenyl, halo, haloalkyl, haloalkenyl, haloalkoxy, hydroxyalkyl, alkylamino, carboalkoxy, carboxy, carboxamido, cyano, and Q^{b};

B is optionally selected from the group consisting of hydrido, trialkylsilyl, C2-C8 alkyl, C3-C8 alkylenyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶;

B is optionally a C3-C12 cycloalkyl or a C4-C9 saturated heterocyclyl, wherein each ring carbon is optionally substituted with R³³, a ring carbon other than the ring carbon at the point of attachment of B to A is optionally substituted with oxo provided that-no more than one ring carbon is substituted by oxo at the same time, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;

R⁹, R¹⁰, R¹¹, R¹², and R¹³ are independently selected from the group consisting of hydrido, acetamido, haloacetamido, alkoxyamino, alkanoyl, haloalkanoyl, amidino, guanidino, alkylenedioxy, haloalkyltlio, alkoxy, cycloalkoxy, cycloalkylalkoxy, amlkoxy, aryloxy, heteroaryloxy, heteroaralkoxy,heterocyclyloxy, heterocyclylalkoxy, hydroxy, amino, alkylamino, N-alkyl-N-arylamino, arylamino, aralkylamiino, heteroarylamino, heteroaralkylamino, heterocyclylamino, heterocyclylalkylamino, alkylthio, alkylsulfinyl, arylsulfinyl, aralkylsulfinyl, cycloalkylsulfinyl, heteroarylsulfinyl, alkylsulfamido, alkylsulfonyl, arylsulfonyl, aralkylsulfonyl, cycloalkylsulfonyl, heteroarylsulfonyl, amidosulfonyl, alkyl, aryl, aralkyl, cycloalkyl, cycloalkylalkyl, heteroaryl, heterocyclyl, halo, haloalkyl, haloalkoxy, hydroxyalkyl, hydroxyhaloalkyl, aminoalkyl, carboalkoxy, carboxy, carboxyalkyl, carboxamido, and cyano;

A is a bond or (CH(R¹⁵))ₚₐ—(W⁷)ᵣᵣ wherein rr is 0 or 1, pa is an integer selected from 0 through 3, and W⁷ is selected from the group consisting of O, S, C(O), (R⁷)NC(O), (R⁷)NC(S), and N(R⁷);

R⁷ is selected from the group consisting of hydrido, hydroxy and alkyl;

R¹⁵ is selected from the group consisting of hydrido, hydroxy, halo, alkyl, and haloalkyl;

X⁰ is selected from the group consisting of hydrido, amino, alkyl, cyano, halo, haloalkyl, haloalkoxy, aminoalkyl, hydroxy, alkoxy, hydroxyalkyl, and alkylthio;

R¹ is selected from the group consisting of hydrido, alkyl, haloalkyl, amino, aminoalkyl, alkylamino, hydroxy, and hydroxyalkyl;

R² is Z⁰-Q^{b};

Z⁰ is selected from the group consisting of a bond, (CR⁴¹R⁴²)_{q} wherein q is 1 or 2, and (CH(R⁴¹))_{g}—W⁰—(CH(R⁴²))ₚ wherein g and p are integers independently selected from 0 through 3 and W⁰ is selected from the group consisting of O, S, C(O), S(O), N(R⁴¹), and ON(R⁴¹);

Z⁰ is optionally (CH(R⁴¹))ₑ—W²(CH(R⁴²))ₕ wherein e and b are independently 0 or 1 and W²² is selected from the group consisting of CR⁴¹═CR⁴², 1,2-cyclopropyl, 1,2,5-cyclobutyl, 1,2-cyclohexyl, 1,3-cyclohexyl, 1,2-cyclopentyl, 1,3-cyclopentyl, 2,3-morpholinyl, 2,4-mmpholinyl, 2,6-morpholinyl, 3,4-morpholinyl, 3,5-morpholinyl, 1,2-piperazinyl, 1,3-piperazinyl, 2,3-piperazinyl, 2,6-piperazinyl, 1,2-piperidinyl, 1,3-piperidinyl, 2,3-pipefidinyl, 2,4-piperidinyl, 2,6-piperidinyl, 3,4-piperidinyl, 1,2-pyrrolidinyl,1,3-pyrrolidinyl, 2,3-pyrrolidinyl, 2,4-pyrrolidinyl, 2,5-pyrrolidinyl, 3,4-pyrrolidinyl, 2,3-tetrahydrofuranyl, 2,4-tetrahydrofuranyl, 2,5-tetrahydrofuranyl, and 3,4-tetrahydrofuranyl, wherein Z⁰ is directly bonded to the 4-pyridone ring and W²² is optionally substituted with one or more substituents selected from the group consisting of R⁹, R¹⁰, R¹¹, R¹², and R¹³;

R⁴¹ and R⁴² are independently selected from the group consisting of hydrido, hydroxy, alkyl, and amino;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹, with the proviso that Q^{b} is other than a phenyl when Z⁰ is a bond;

Q is optionally hydrido with the proviso that Z⁰ is selected from other than a bond;

K is CHR^{4a} wherein R^{4a} is selected from the group consisting of hydrido, hydroxyalkyl, alkyl, alkoxyalkyl, alkylthioalkyl, and haloalkyl;

E⁰ is selected from the group consisting of a bond, C(O)N(H), (H)NC(O), (R⁷)NS(O)₂, and S(O)₂N(R⁷);

Y^{AT} is Q^{b}-Q^{s};

Q^{s} is (CR³⁷R³⁸)_{b} wherein b is an integer selected from 1 through 4, R³⁷ is selected from the group consisting of hydrido, alkyl and haloalkyl, and R³⁸ is selected from the group consisting of hydrido, alkyl,d haloalkyl, aroyl, and heteroaroyl with the proviso that there is at least one aroyl or heteroaroyl substituent, with the further proviso that no more than one aroyl or heteroaroyl is bonded to (CR³⁷R³⁸)_{b} at the same time, with the still further proviso that said aroyl and said heteroaroyl are optionally substituted with one or more substituents selected from the group consisting of R¹⁶, R¹⁷, R¹⁸, and R¹⁹, with another further proviso that said aroyl and said heteroaroyl are bonded to the CR³⁷R³⁸ that is directly bonded to E⁰, with still another further proviso that no more than one alkyl or one haloalkyl is bonded to a CR³⁷R³⁸ at the same time, and with the additional proviso that said alkyl and haloalkyl are bonded to a carbon other than the one bonding said aroyl or said heteroaroyl;

R¹⁶, R¹⁷, R¹⁸, and R¹⁹ are independently selected from the group consisting of hydrido, amidino, guanidino, carboxy, haloalkylthio, alkoxy, hydroxy, amino, alkoxyamino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally selected from the group consisting of NR²⁰R²¹, N(R²⁶)C(NR²⁵)N(R²³)(R²⁴), and C(NR²⁵)NR²³R²⁴, with the proviso that R¹⁶, R¹⁹, and Q^{b} are not simultaneously hydrido;

Q^{b} is selected from the group consisting of NR²⁰R²¹, hydrido, N(R²⁶)C(NR²⁵)N(R²³)(R²⁴) and C(NR²⁵)NR²³R²⁴, with the proviso that no more than one of R²⁰ and R²¹ is selected from the group consisting of hydroxy, amino, alkylamino, and dialkylamino at the same time and with the further proviso that no more than one of R²³ and R²⁴ is selected from the group consisting of hydroxy, amino, alkylamino, and dialkylamino at the same time;

R²⁰, R²¹, R²³, R²⁴, R²⁵, and R²⁶ are independently selected from the group consisting of hydrido, alkyl, hydroxy, amino, alkylamino and dialylamino.

In a most preferred embodiment of compounds of Formula I, said compound is the Formula:

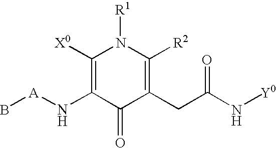

or a pharmaceutically acceptable salt thereof, wherein;

B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydzido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, carboalkoxy, carboxy, carboxamido, cyano, and Q^{b};

A is a bond or (CH(R¹⁵))ₚₐ—(W⁷)ᵣᵣ wherein rr is 0 or 1, pa is an integer selected from 0 through 3, and W⁷ is N(R⁷);

R⁷ is hydrido or alkyl;

R¹⁵ is selected from the group consisting of hydridd, halo, alkyl, and haloalkyl;

X⁰ is selected from the group consisting of hydrido, alkyl, halo, haloallyl, aminoalkyl, and hydroxyalkyl;

R¹ is selected from the group consisting of hydrido, alkyl, hydroxyalkyl, and haloalkyl;

R² is Z⁰-Q^{b};

Z⁰ is a bond;

Q is phenyl or a heteroaryl of 5 or 6 ring members, wherein a carbon adjacent to the carbon at the point of attachment of said phenyl or heteroaryl ring to Z⁰ is optionally substituted by R⁹ the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

R⁹, R¹¹, and R¹³ are independently selected from the group consisting of hydrido, hydroxy, amino, amidino, guanidino, alkylamino, allylthio, alkoxy, alkylsulfinyl, alkylsulfonyl, amidosulfonyl, alkyl, halo, haloalkyl, haloalkoxy, hydroxyalkyl, carboxy, carboxamido, and cyano;

R¹⁰ and R¹² are independently selected from the group consisting of hydrido, acetamido, haloacetamido, amidino, guanidino, alkyl, alkoxy, alkoxyamino, hydroxy, amino, alkylamino, alkylsulfonamido, amidosufonyl, hydzoxyalkyl, aminoalkyl, halo, haloalkyl, carboalkoxy, carboxy, carboxamido, carboxyalkyl, and cyano;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydrido, aridino, guanidino, carboxy, haloalkylthio, alkoxy, hydroxy, amino, alkylamino, alkylthio, alkylsulfinyl, alkylsulfonyl, alkanoyl, haloalkanoyl, alky, halo, haloalkyl, haloalkoxy, hydroxyalkyl, aminoalayl, and cyano;

R¹⁶ or R¹⁹ is optionally NR²⁰R²¹ or C(NR²⁵)NR²³R²⁴, with the proviso that R¹⁶, R¹⁹, and Q are not simultaneously hydrido;

Q^{b} is selected from the group consisting of NR²⁰R²¹, hydrido, and C(NR²⁵)NR²³R²⁴;

R²⁰, R²¹, R²³, R²⁴, and R²⁵ are independently hydrido or alkyl;

Q^{s} is CH₂.

In a further most preferred embodiment of compounds of Formula I, said compound is the Formula:

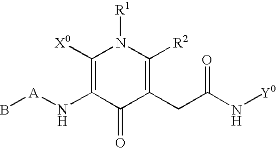

or a pharmaceutically acceptable salt thereof, wherein;

B is selected from the group consisting of hydrido, C2-C8 alkyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶;

R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, carboalkoxy, carboxy, carboxamido, cyano, and Q^{b};

A is a bond or (CH(R¹⁵))ₚₐ—(W⁷)ᵣᵣ wherein rr is 0 or 1, pa is an integer selected from 0 through 3, and W⁷ is N(R⁷);

R⁷ is hydrido or alkyl;

R¹⁵ is selected from the group consisting of hydrido, halo, alkyl, and haloalkyl;

X⁰ is selected from the group consisting of hydrido, alkyl, halo, haloalkyl, aminoalkyl, and hydroxyalkyl;

R¹ is selected from the group consisting of hydrido, alky, hydrcoyalkyl, and haloalkyl;

R² is Z⁰-Q^{b};

Z⁰ is a bond;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

R⁹, R¹¹, and R¹³ are independently selected from the group consisting of hydrido, hydroxy, amino, amidino, guanidino, alkylamino, alkylthio, alkoxy, alkylsulfinyl, alkylsulfonyl, amidosulfonyl, alkyl, halo, haloalkyl, haloalkoxy, hydroxyalkyl, carboxy, carboxamido, and cyano;

R¹⁰ and R¹² are independently selected from the group consisting of hydrido, acetamido, haloacetarido, amidino, guanidino, alkyl, alkoxy, alkoxyamino, hydroxy, amino, alkylamino, alkylsulfonamido, amidosulfonyl, hydroxyalkyl, aminoalkyl, halo, haloalkyl, carboalkoxy, carboxy, carboxamido, carboxyalkyl, and cyano;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydfido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally selected from the group consisting of NR²⁰R²¹,N(R²⁶)C(NR²⁵)N(R²³)(R²⁴), and C(NR²⁵)NR²³R²⁴, with the proviso that R¹⁶, R¹⁹, and Q^{b} are not simultaneously hydrido;

Q^{b} is selected from the group consisting of NR²⁰R²¹, hydrido, N(R²⁶)C(NR²⁵)N(R²³)(R²⁴), and C(NR²⁵)NR²³R²⁴;

R²⁰, R²¹, R²³, R²⁴, R²⁵, and R²⁶ are independently hydrido or alkyl;

Q^{s} is CH₂.

In a still further most preferred embodiment of compounds of Formula I, said compound is the Formula:

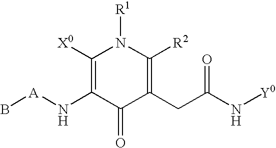

or a pharmaceutically acceptable salt thereof, wherein;

B is a C3-C7 cycloalkyl or a C4-C6 saturated heter6-cyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;

R⁹, R¹¹, and R¹³ are independently selected from the group consisting of hydtido, hydroxy, amino, amidino, guanidino, alkylamino, alkylthio, alkoxy, alkylsulfinyl, alkylsulfonyl, amidosulfonyl, alkyl, halo, haloalkyl, haloalkoxy, hydroxyalkyl, carboxy, carboxamido, and cyano;

R¹⁰ and R¹² are independently selected from the group consisting of hydrido, acetaindo, haloacetamido, amidino, guanidino, alkyl, alkoxy, alkoxyamino, hydroxy, amino, alkylamino, alkylsilfonamido, amidosulfonyl, hydroxyalkyl, aminoalkyl, halo, haloalkyl, carboalkoxy, carboxy, carboxamido, carboxyalkyl, and cyano;

R³³ and R³⁴ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, carboalkoxy, carboxy, carboxamido, and cyano;

R³³ is optionally Q^{b};

A is a bond or (CH(R¹⁵))ₚₐ—(W⁷)ᵣᵣ wherein rr is 0 or 1, pa is an integer selected from 0 through 3, and W⁷ is N(R⁷);

R⁷ is hydrido or alkyl;

R¹⁵ is selected from the group consistng of hydrido, halo, alkyl, and haloalkyl;

X⁰ is selected from the group consisting of hydrido, alkyl, halo, haloalkyl, aminoalkyl, and hydroxyalkyl;

R¹ is selected from the group consisting of hydrido, alkyl, hydroxyalkyl, and haloalkyl;

R² is Z⁰-Q^{b};

Z⁰ is a bond;

Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;

R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alIylsulfonyl, alkanoyl, haloalkanoyl, alkyl, halo, haloalkyl, haloalkoxy, hydroxyalkyl, aminoalkyl, and cyano;

R¹⁶ or R¹⁹ is optionally NR²⁰R²¹ or C(NR²⁵)NR²³ R²⁴ with the proviso that R¹⁶, R¹⁹, and Q^{b} are not simultaneously hydrido;

Q^{b} is selected from the group consisting of NR²⁰R²¹, hydrido, and C(NR²⁵)NR²³R²⁴;

R²⁰, R²¹, R²³, R²⁴, and R²⁵ are independently hydrido or alkyl;

Q^{s} is CH₂.

In a preferred specific embodiment of Formula I, compounds have the formula:

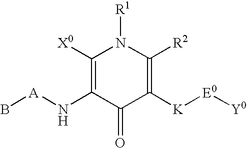

or a pharmaceutically acceptable salt thereof, wherein;

B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 1,2,4-triazol-3-yl, 1,2,4-triazol-5-yl, 1,2,4-oxadiazol-3-yl, 1,2,4-oxadiazol-5-yl, 1,3,4-oxadiazol-3-yl, 1,3,4-oxadiazol-5-yl, 3-isothiazolyl, 5-isothiazolyl, 2-oxazolyl, 2-thiazolyl, 3-isoxazolyl, 5-isoxazolyl, 2-pyridyl, 3-pyridyl, 4-pynidyl, pyrazinyl, 2-pyrnidinyl, 4-pyrimidinyl, 5-pyrirnidinyl, 3-pyridazinyl, 4-pyridazinyl, 1,3,5-triazin-2-yl, 1,2,4-triazin-3-yl, 1,2,4-triazin5-yl, 1,2,4-triazinyl, 1,2,3-triazin41-yl, and 1,2,3-triazin-5-yl, wherein a carbon adjacent to the carbon at the point of attachment is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydrido, amidino, guanidino, carboxy, methyl, ethyl, isopropyl, propyl, methoxy, ethoxy, isopropoxy, propoxy, hydroxy, amino, methoxyamino, ethoxyamino, acetamido, trifluoroacetamido, nitro, aminomethyl, 1-aminoethyl, 2-aminoethyl, N-methylamino, dimethylamino, N-ethylamino, methyltiio, ethylthio, isopropylthio, tifluoromethylthio, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, amidosulfonyl, N-methylamidosulfonyl, N,N-dimethylamidosulfonyl, acetyl, propanoyl, trifluoroacetyl, pentafluoropropanoyl, hydroxymethyl, 1-hydroxyethyl, 2-hydroxyethyl, 2,2,2-triuoro-1-hydroxyethyl, 2,2,2-trifluoro-1-triuoromethyl-1-hydroxyethyl, carboxymethyl, methoxycarbonyl, ethoxycarbonyl, amidocarbonyl, N-methylamidocarbonyl, N,N-dimethylamidocarbonyl, cyano, and Q^{b};

B is selected from the group consisting of hydrido, trimethylsilyl, ethyl, 2-propenyl, 2-propynyl, propyl, isopropyl, butyl, 2-butenyl, 3-butenyl, 2-butynyl, sec-butyl, tert-butyl, isobutyl, 2-methylpropenyl, 1-pentyl, 2-pentenyl, 3-pentenyl, 4-pentenyl, 2-pentynyl, 3-pentynyl, 2-pentyl, 1-methyl-2-butenyl, 1-methyl-3-butenyl, 1-methyl-2-butynyl, 3-pentyl, 1-ethyl-2-propenyl, 2-methylbutyl, 2-methyl-2-butenyl, 2-methyl-3-butenyl, 2-methyl-3-butynyl, 3-methylbutyl, 3-methyl-2-butenyl, 3-methyl-3-butenyl, 1-hexyl, 2-hexenyl, 3-hexenyl, 4-hexenyl, 5-hexenyl, 2-hexynyl, 3-hexynyl, 4-hexynyl, 2-hexyl, 1-methyl-2-pentenyl, 1-methyl-3-pentenyl, 1-methyl-4-pentenyl, 1-methyl-2-pentynyl, 1-methyl-3-pentynyl, 3-hexyl, 1-ethyl-2-butenyl, 1-ethyl-3-butenyl, 1-propyl-2-propenyl, 1-ethyl-2-butynyl, 1-heptyl, 2-heptenyl, 3-heptenyl, 4-heptenyl, sheptenyl, 6-heptenyl, 2-heptynyl, 3-heptynyl, 4-heptynyl, 5-heptynyl, 2-heptyl, 1-methyl-2-hexenyl, 1-methyl-3-hexenyl, 1-methylthexenyl, 1-methyl-5-hexenyl, 1-methyl-2-hexynyl, 1-methyl-3-hexynyl, 1-methylAhexynyl, 3-heptyl, 1-ethyl-2-pentenyl, 1-ethyl-3-pentenyl, 1-ethylpentenyl, 1-butyl-2-propenyl, 1-ethyl-2-pentynyl, 1-ethyl-3-pentynyl, 1-octyl, 2-octenyl, 3-octenyl, 4-octenyl, 5-octenyl, 6-octenyl, 7-octenyl, 2-octynyl, 3-octynyl, 4-octynyl, 5-octynyl, 6-octynyl, 2-octyl, 1-methyl-2-heptenyl, 1-methyl-3-heptenyl, 1-methylheptenyl, 1-methyl-Sheptenyl, 1-methyl-6-heptenyl, 1-methyl-2-heptynyl, 1-methyl-3-heptynyl, 1-methyl-4-heptenyl, 1-methyl-5-heptenyl, 1-methyl-6-heptenyl, 1-methyl-2-heptenyl, 1-methyl-3-heptynyl, 1-methylheptynyl, 1-methyl-5Sheptynyl, 3-octyl, 1-ethyl-2-hexenyl, 1-ethyl-3-hexenyl, 1-ethyl-4-hexenyl, 1-ethyl-2-hexynyl, 1-ethyl-3-hexynyl, 1-ethylAhexynyl, 1-ethyl-Shexenyl, 1-pentyl-2-propenyl, 4-octyl, 1-propyl-2-pentenyl, 1-propyl-3-pentenyl, 1-propyl pentenyl, 1-butyl-2-butenyl, 1-propyl-2-pentynyl, 1-propyl-3-pentynyl, 1-butyl-2,5-butynyl, 1-butyl-3-butenyl, 2,2,2-trifluoroethyl, 2,2-difluoropropyl, 4-trifluoromethyl-5,5,5-trifluoropentyl, 4-trifluoromethylpentyl, 5,5,6,6,6-pentafluorohexyl, and 3,3,3-tiifluoropropyl, wherein each member of group B is optionally substituted at any carbon up to and including 5 atoms from the point of attachment of B to A with one or more of the group consisting of R³², R³³, R³⁴, R³⁵, and R³⁶;

B is optionally selected from the group consisting of cyclopropyl, cyclobutyl, oxetan-2-yl, oxetan-3-yl, azetidin-1-yl, azetidin-2-yl, azetidin-3-yl, thiaetan-2-yl, thiaetan-3-yl, cyclopentyl, cyclohexyl, adamantyl, norbornyl, 3-trifluoromethylnorbornyl, 7-oxabicyclo[2.2.1]heptan-2-yl, bicyclo[3.1.0]hexan-6-yl, cycloheptyl, cyclooctyl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 4H-2-pyranyl, 4H-3-pyranyl,4H-4-pyranyl, 4H-pyran-4-one-2-yl, 4H-pyran4-one-3-yl, 2-tetrahydrofuranyl, 3-tetrahydrofuranyl, 2-tetrahydropyranyl, 3-tetrhydropyranyl, 4-tetrahydropyranyl, 2-tetrahydrothienyl, and 3-tetrahydrothienyl, wherein each ring carbon is optionally substituted with R³³, a ring carbon and nitrogen atoms adjacent to the carbon atom at the point of attachment is optionally substituted with R⁹ or R¹³, a ring carbon or nitrogen atom adjacent to the R position and two atoms from the point of attachment is optionally substituted with R¹⁰, and a ring carbon or nitrogen atom adjacent to the R¹³ position and two atoms from the point of attachment is optionally substituted with R¹²;

R⁹, R¹⁰, R¹¹, R¹², and R¹³ are independently selected from the group consisting of hydrido, amidino, guanidino, carboxy, carboxymethyl, methyl, ethyl, propyl, isopropyl, methoxy, ethoxy, isopropoxy, propoxy, hydroxy, amino, methoxyamino, ethoxyamino, acetamido, trifluoroacetamido, aminomethyl, 1-aminoethyl, 2-aminoethyl, N-methylamino, dimethylamino, N-ethylamino, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, N-benzylamidocarbonyl, N-(2-chlorobenzyl)amidocarbonyl, N-(3-fluorobenzyl)amidocarbonyl, N-(2-tfifluoromethylbenzyl)amidocarbonyl, N-(1-phenylethyl)amidocarbonyl, N-1-methyl-1-phenylethyl)amidocarbonyl, N-benzylamidosulfonyl, N-2-chlorobenzyl)anidosulfonyl, Nethylainidocarbonyl, N-isopropylamidocarbonyl, N-propylamidocarbonyl, N-isobutylamidocarbonyl, N-(2-butyl)amidocarbonyl, N-cyclobutylamidocarbonyl, N-cyclopentylamidocarbonyl, N-cycohexylamidocarbonyl, fluoro, chloro, bromo, cyano, cyclobutoxy, cyclohexoxy, cyclohexylmethoxy, 4-trifluoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, 5-bromopyrid-2-ylmethylamino, 4-butoxyphenamino, 3-chlorobenzyl, 4-chlorophenoxy, 4-chloro-3-ethylphenoxy, 4-chloro-3-ethylbenzylamino, 4-chloro-3-ethylphenylamino, 3-chlorobenzyloxy, 4-chlorobenzyloxy, 4-chlorobenzylsulfonyl, 4-chlorophenylamino, 4-chlorophenylsulfonyl, 5-chloropyrid-3-yloxy, 2-cyanopyrid-3-yloxy, 2,3-difluorobenzyloxy, 2,4-difluorobenzyloxy, 3,4-dfluorobenzyloxy, 2,5-difluorobenzyloxy, 3,5-difluorophenoxy, 3,5;difluorobenzyloxy, 4-difluoromethoxybenzyloxy, 2,3-difluorophenoxy, 2,4-difluorophenoxy, 2,5-difluorophenoxy, 3,5-dimethylphenoxy, 3,4-dimethylphenoxy, 3,4-dimethylbenzyloxy, 3,5-dimethylbenzyloxy, 4-ethoxyphenoxy, 4-ethylbenzyloxy, 3-ethylphenoxy, 4-ethylaminophenoxy, 3-ethyl-5-methylphenoxy, 4-fluorobenzyloxy, 2,5-fluoro-3-trifluoromethylbenzyloxy, 3-fluoro-5-trifluoromethylbenzyloxy, 4-fluoro-2-trifluoromethylbenzyloxy, 4-fluoro-3-trifluoromethylbenzyloxy, 2-fluorophenoxy, 4-fluorophenoxy, 2-fluoro-3-trifluoromethylphenoxy, 2-fluorobenzyloxy, 4-fluorophenylanno, 2-fluorotriuoromethylphenoxy, 4-isopropylbenzyloxy, 3-isopropylphenoxy, 4-isopropylphenoxy, 4-isopropyl-3-methylphenoxy, 4-isopropylbenzyloxy, 3-isopropylphenoxy, 4-isopropylphenoxy, 4-isopropyl-3-methylphenoxy, phenylamino, 1-phenylethoxy, 2-phenylethoxy, 2-phenylethyl, 2-phenylethylamino, phenylsulfonyl, 3-trifluoromethoxybenzyloxy, 4-trifluoromethoxybenzyloxy, 3-trifluoromethoxyphenoxy, 4-triuoromethoxyphenoxy, 3-trifluoromethylbenzyloxy, 4-trifluoromethylbenzyloxy, 2,4-bis-trifluoromethylbenzyloxy, 3-trifluoromethylbenzyl, 3,5-bis-trifluoromethylbenzyloxy, 4-t-iuoromethylphenoxy, 3-trifluoromethylphenoxy, 3-trifluoromethylthiobenzyloxy, 4-trifluoromethylthiobenzyloxy, 2,3,4-trifluorophenoxy, 2,3,5-trifluoropheaoxy, 3-pentafluoroethylphenoxy, 3,1,1,2,2-tetrfluoroethoxy)phenoxy, and 3-trifluoromethylthiophenoxy;

A is selected from the group consisting of a bond, O, S, NH, N(CH₃), N(OH), C(O), CH₂, CH₃CH, CF₃CH, NHC(O), N(CH₃)C(O), C(O)NH, C(O)N(CH₃), CFCC(O), C(O)CCH₃, C(O)CCF₃, CH₂C(O), (O)CCH₂, CH₂CH₂, CH₂CH₂CH₂, CH₃CHCH₂, CF₃CHCH₂, CH₃CC(O)CH₂, CF₃CC(O)CH₂, CH₂C(O)CCH₃, CH₂C(O)CCF₃, CH₂CH₂C(O), and CH₂(O)CCH₂;

A is optionally selected from the group consisting of CH₂N(CH₃), CH₂N(CH₂CH₃), CH₂CH₂N(CH₃), and CH₂CH₂N(CH₂CH₃) with the proviso that B is hydrido;

X^{o} is selected from the group consisting of hydrido, hydroxy, amino, thiol, amidino, hydroxyamino, aminomethyl, 1-aminoethyl, 2-aminoethyl, 3-aminopropyl, 2-amnopropyl, methylamino, dimethylamino, cyano, methyl, ethyl, isopropyl, propyl, butyl, trifluoromethyl, pentafluoroethyl, 2,2,2-trifluoroethyl, 3,3,3-trifluoropropyl, 2,2,3,3,3-pentafluoropropyl, methoxy, ethoxy, propoxy, hydroxymethyl, 1-hydroxyethyl, 2-hydroxyethyl, 3-hydroxypropyl, 2,3-dihydroxypropyl, methoxyamino, ethoxyamino, methylthio, ethylthio, trifluoromethoxy, 1, 1,2,2-tetrafluoroethoxy, fluoro, chloro, and bromo;

R² is Z⁰-Q^{b};

Z⁰ is selected from the group consisting of a bond, CH₂, CH₂CH₂, O, S, NH, N(CH₃), CH(OH), OCH₂, SCH₂, N(H)CH₂, CH₂O, CH₂S, CH₂N(H), CH(NH₂), CH₂CH(OH), CH₂CHNH₂, CH(OH)CH₂, and CH(NH₂)CH₂;

Q is selected from the group consisting of phenyl, 2-thienyl, 3-thienyl, 2,5-furyl, 3-furyl, 2-pyrrolyl, 3-pyrrolyl, 2-imidazolyl, 4-imidazolyl, 3-pyrazolyl, 4-pyrazolyl, 1,2,4-triazol-3-yl, 1,2,4-triazol-5-yl, 1,2,4-oxadiazol-3-yl, 1,2,4-oxadiazol-5-yl, 1,3,4-oxadiazol-3-yl, 1,3,4-oxadiazol-5-yl, 3-isothiazolyl, 5-isothiazolyl, 2-oxazolyl, 2-thiazolyl, 3-isoxazolyl, 5 isoxazolyl, 2-pyridyl, 3-pytidyl, 4-pyridyl, 2-pyrazinyl, 2-pyrimidinyl, 4-pyrimidinyl, 5-pyrimidinyl, 3-pyridazinyl, 4-pyridazinyl, 1,3,5-triazin-2-yl, 1,2,4-triazin-3-yl, 1,2,4-ttiazin-5-yl, 1,2,4-triazin-6-yl, 1,2,3-tiiazin-4-yl, and 1,2,3-triazin-5-yl, wherein a carbon adjacent to the carbon at the point of attachment is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

K is CR^{4a}R^{4b} wherein R^{4a} and R^{4b} are independently selected from the group consisting of methyl, ethyl, propyl, isopropyl, fluoro, chloro, hydroxy, hydroxymethyl, 1-hydroxyethyl, methoxymethyl, trifluoromethyl, pentafluoroethyl, 2,2,2-trifluoromethyl, methylthiomethyl, and hydrido;

E⁰ is a bond, C(O)N(H), (H)NC(O), and S(O)₂N(H);

Y⁰ is selected from the group of formulas consisting of:

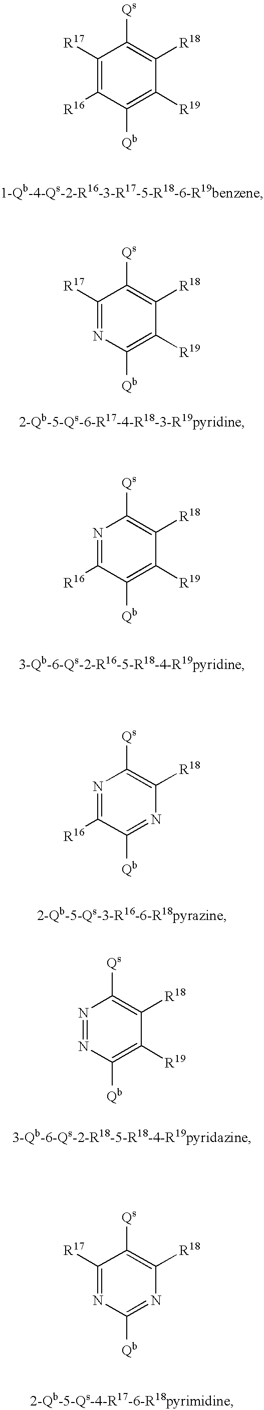

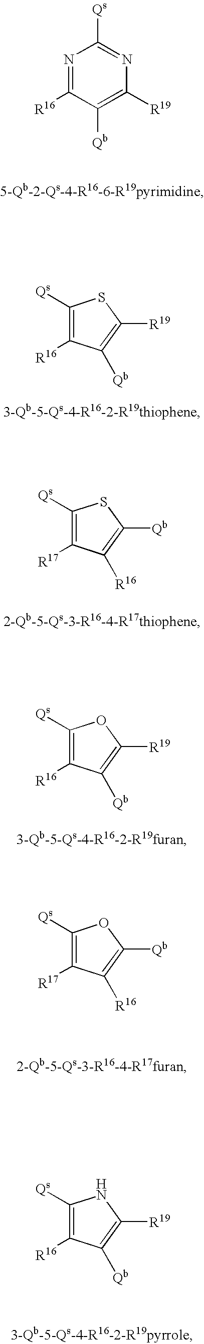

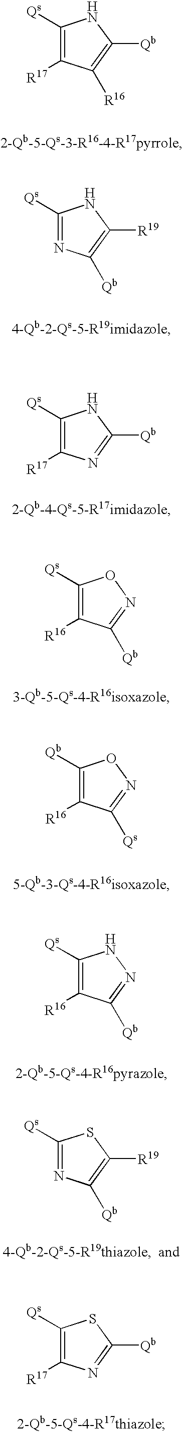

R¹⁶, R¹⁷, R¹⁸, and R¹⁹ are independenty selected from the group consisting of hydrido, methyl, ethyl, isopropyl, propyl, amidino, guanidino, carboxy, methoxy, ethoxy, isopropoxy, propoxy, hydroxy, amino, methoxyamino, ethoxyamino, aminomethyl, 1-aminoethyl, 2-aminoethyl, N-N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-tdfluoroethyl, 2,2,3,3,3-pentafluoropropyl, trifluoromethoxy, 1,1,2,2-tetrafluoroethoxy, fluoro, chloro, bromo, amidosulfonyl, N-methylamidosulfonyl, N,N-dimethylamidosulfonyl, acetyl, propanoyl, trifluoroacetyl, pentafluoropropanoyl, hydroxymethyl, 1-hydroxyethyl, 2-hydroxyethyl, and cyano;

Q^{b} is selected from the group consisting of NR²⁰R²¹, hydrido, C(NR²⁵)NR²³R²⁴ and N(R²⁶)C(NR²⁵)N(R²³)(R²⁴), with the proviso that no more than one of R²⁰ and R²¹ is hydroxy, N-methylamino, and N,N-dimethylamino at the same time and that no more than one of R²³ and R²⁴ is hydroxy, N-methylamino, and N,N-dimethylamino at the same time;

R²⁰, R²¹, R²³, R²⁴, R²⁵ and R²⁶ are independently selected from the group consisting of hydrido, methyl, ethyl, propyl, butyl, isopropyl, hydroxy, 2-aminoethyl, 2-N-methylamino)ethyl, and 2-N,N-dimethylamino)ethyl;

Q^{s} is selected from the group consisting of a bond, CH₂, CH₂CH₂, CH₃CH, CF₃CH, CH₃CHCH₂, CF₃CHCH₂, CH₂(CH₃)CH, CH═CH, CF═CH, C(CH₃)═CH, CH═CHCH₂, CF—CHCH₂, C(CH₃)═CHCH₂, CH₂CH═CH, CH₂CF═CH, CH₂C(CH₃)═CH, CH₂CH═CHCH₂, CH₂CF═CHCH₂, CH₂C(CH₃)═CHCH₂, CH₂CH═CHCH₂CH₂, CH₂CF═CHCH₂CH₂, and CH₂C(CH₃)═CHCH₂CH₂.

In a more preferred specific embodiment of Formula I, compounds have the formula:

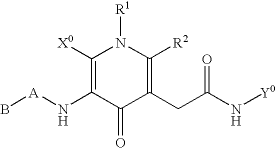

or a pharmaceutically acceptable salt thereof, wherein;

B is selected from the group consisting of phenyl, 2-thienyl, 3-thienyl, 24-wuy1, 3-furyl₁ 2-pyrrlyl, 3-pyffolyl, 2-imidaaolyl, Simidazolyl, 3-pyrazolyl, 4-pyrazoly1, 2-thiazolyl, 3-isoxazolyl, Sisoxazolyl, 2-pyridyl, 3-pyfldyl, 4-pyridyl, 2-pyrazinyl, 2-pyrimidinyl, 4-pyrimidinyl, 5-pyrimidinyl, 3-pyridazinyl, 4-pyridazinyl, and 1,3,5-triazin-2-yl, wherein a carbon adjacent to the carbon at the point of attachment of said phenyl or heteroyl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydrido, amidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ethylainino, methylthio, ethylthio, isopropylthio, trifluoromethyl, pentafluoroethyl, 2,2,2-trifluoroethyl, 2,2,3,3,3-pentafluoropropyl, trifluoromethoxy, 1,1,2,2-tetrfluoroethoxy, fluoro, chloro, bromo, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylanidocarbonyl, cyano, and Q^{b};

A is selected from the group consisting of a bond, NH, N(CH₃), N(OH), CH₂, CH₃CH, CF₃CH, NHC(O), N(CH₃)C(O), C(O)NH, C(O)N(CH₃), CH₂CH₂, CH₂CH₂CH₂, CH₃CHCH₂, and CF₃CHCH₂;

R¹⁶, R¹⁷, R¹⁸, and R¹⁹ are independently selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, hydroxymethyl, 1-hydroxyethyl, 2-hydroxyethyl, and cyano;

R¹⁶ or R¹⁹ is optionally C(NR²⁵)NR²³R²⁴ with the proviso that R¹⁶, R¹⁹, and Q^{b} are not simultaneously hydrido;

Q^{b} is C(NR²⁵)NR²³R²⁴ or hydrido, with the proviso that no more than one of R²³ and R²⁴ is hydroxy at the same time;

R²³, R²⁴, and R²⁵ are independently selected from the group consisting of hydrido, methyl, ethyl, and hydroxy.

In another more preferred specific embodiment of Formula I, compounds have the formula:

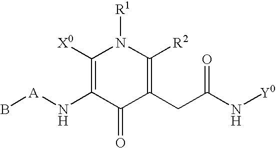

or a pharmaceutically acceptable salt thereof, wherein;

B is selected from the group consisting of hydrido, ethyl, 2-propynyl, 2-propenyl, propyl, isopropyl, butyl, 2-butenyl, 3-butenyl, 2-butynyl, sec-butyl, tert-butyl, isobutyl, 2-methylpropenyl, 1-pentyl, 2-pentenyl, 3-pentenyl, 4-pentenyl, 2-pentynyl, 3-pentynyl, 2-pentyl, 1-methyl-2-butenyl, 1-methyl-3-butenyl, 1-methyl-2-butynyl, 3-pentyl, 1-ethyl-2-propenyl, 2-methylbutyl, 2-methyl-2-butenyl, 2-methyl-3-butenyl, 2-methyl-3-butynyl, 3-methylbutyl, 3-methyl-2-butenyl, 3-methyl-3-butenyl, 1-hexyl, 2-hexenyl, 3-hexenyl, 4-hexenyl, 5-hexenyl, 2-hexynyl, 3-hexynyl, 4-hexynyl, 2-hexyl, 1-methyl-2-pentenyl, 1-methyl-3-pentenyl, 1-methyltpentenyl, 1-methyl-2-pentynyl, 1-methyl-3-pentynyl, 3-hexyl, 1-ethyl-2-butenyl, 1-ethyl-3-butenyl, 1-propyl-2-propenyl, 1-ethyl-2-butynyl, 1-heptyl, 2-heptenyl, 3-heptenyl, 4-heptenyl, Sheptenyl, 6-heptenyl, 2-heptynyl, 3-heptynyl, 4-heptynyl, 5-heptynyl, 2-heptyl, 1-methyl-2-hexenyl, 1-methyl-3-hexenyl, 1-methylhexenyl, 1-methyl-5-hexenyl, 1-methyl-2-hexynyl, 1-methyl-3-hexynyl, 1-methylhexynyl, 3-heptyl, Iethyl-2-pentenyl, 1-ethyl-3-pentenyl, 1-ethyl4-pentenyl, 1-butyl-2-propenyl, 1-ethyl-2-pentynyl, 1-ethyl-3-pentynyl, 2,2,2-trifluoroethyl, 2,2-difluoropropyl, 4-trifluoromethyl-5,5,5-trifluoropentyl, 4-tnfuoromethylpentyl, 5,5,6,6,6-pentafluorohexyl, and 3,3,3-trifluoropropyl, wherein each member of group B is optionally substituted at any carbon up to and including 5 atoms from the point of attachment of B to A with one or more of the group consisting of R³², R³³, R³⁴, R³⁵, and R³⁶;

R³², R³³, R³⁴, R³⁵, and R³⁶ are independently selected from the group consisting of hydrido, anidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ehylamino, methylthio, ethyltlhio, isopropylthio, trifluorxomethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, amidosulfonyl, N-methylamidosulfonyl, N,N-dimetliylaridosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, cyano, and Q^{b};

A is selected from the group consisting of bond, NH, N(CH₃), N(OH), CH₂, CH₃CH, CF₃CH, NHC(O), N(CH₃)C(O), C(O)NH, C(O)N(CH₃), CH₂CH₂, CH₂CH₂CH₂, CH₃CHCH₂, and CF₃CHCH₂;

R¹⁶, R¹⁷, R¹⁸, and R¹⁹ are independently selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-tuoroethyl, 2,2,3,3,3-pentafluoropropyl, trifluoromethoxy, 1,1,2,2-tetrafluoroethoxy, fluoro, chloro, bromo, hydroxymethyl, 1-hydroxyethyl, 2-hydroxyethyl, and cyano;

R¹⁶ or R¹⁹ is optionally selected from the group consisting of NR²⁰R²¹, C(NR²⁵)NR²³R²⁴, and N(R²⁶)C(NR²⁵)N(R²³)(R²⁴), with the proviso that R¹⁶, R¹⁹, and Q^{b} are not simultaneously hydrido;

Q^{b} is selected from the group consisting of NR²⁰R²¹, hydrido, C(NR²⁵)NR²³R²⁴, and N(R²⁶)C(NR²⁵)N(R²³)(R²⁴), with the proviso that no more than one of R²⁰ and R²¹ is hydroxy at the same time and with the further proviso that no more than one of R²³ and R²⁴ is hydroxy at the same time;

R²⁰, R²¹, R²³, R²⁴, R²⁵, and R²⁶ are independently selected from the group consisting of hydrido, methyl, ethyl, propyl, butyl, isopropyl, and hydroxy.

In still another more preferred specific embodifinent of Formula I, compounds have the formula:

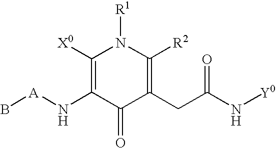

or a pharmaceutically acceptable salt thereof, wherein;

B is selected from the group consisting of cyclopropyl, cyclobutyl, oxetan-3-yl, azetidin-1-yl, azetidin-2-yl, azetidin-3-yl, thiaetan-3-yl, cyclopentyl, cyclohexyl, norbornyl, 7-oxabicyclo[2.2.1]heptan-2-yl, bicyclo[3.1.0]hexan-6-yl, cycloheptyl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 4H-2-pyranyl, 4H-3-pyranyl, 4Hpyranyl, 4H-pyranone-2-yl, 4H-pyran-4-one-3-yl, 2-tetrahydrofurauyl, 3-tetrahydrofuranyl, 2-tetrahydropyranyl, 3-tetrahydropyranyl, 4-tetrahydropyranyl, 2-tetrahydrothienyl, and 3-tetrahydrothienyl, wherein each ring carbon is optionally substituted with R³³, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, and a ring carbon or nitrogen adjacent to the R¹³ position and two atoms from the point of attachment is optionally substituted with R¹²;

R³³ is selected from the group consisting of hydrido, amidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ethylamino, methylthio, ethylthio, isopropylthio, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, amidosulfonyl, N-methylamidosulfonyl, N,N-dinethylamidosulfonyl, hydroxymethyl, 1-hydroxyethyl, 2-hydroxyethyl, 2,2,2-tifluoro-1-hydroxyethyl, methoxycarbonyl, ethoxycarbonyl, amidocarbonyl, N-methylamidocarbonyl, N,Ndimethylamidocarbonyl, cyano, and Q^{b};

A is selected from the group consisting of a bond, NH, N(CH₃), N(OH), CH₂, CH₃CH, CF₃CH, NHC(O), N(CH₃)C(O), C(O)NH, C(O)N(CH₃), CH₂CH₂, CH₂CH₂CH₂, CH₃CHCH₂, and CF₃CHCH₂;

R¹⁶, R¹⁷, R¹⁸, and R¹⁹ are independently selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, hydroxymethyl, 1-hydroxyethyl, 2-hydroxyethyl, and cyano;

R¹⁶ or R¹⁹ is optionally C(NR²⁵)NR²³R²⁴ with the proviso that R¹⁶, R¹⁹, and Q are not simultaneously hydrido;

Q^{b} is C(NR²⁵)NR²³R²⁴ or hydrido, with the proviso that no more than one of R²³ and R²⁴ is hydroxy at the same time;

R²³, R²⁴, and R²⁵ are independently selected from the group consisting of hydrido, methyl, ethyl, and hydroxy.

The more preferred specific embodiment compounds of Formula I having the formula:

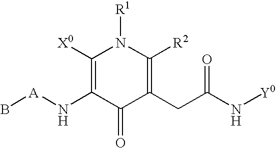

or a pharmaceutically acceptable salt thereof, have common structural units, wherein;

X^{o} is selected from the group consisting of hydrido, hydroxy, amino, aminomethyl, 1-aminoethyl, methyl, ethyl, trifluoromethyl, pentafluoroethyl, 2,2,2-trifluoroethyl, methoxy, hydroxymethyl, 1-hydroxyethyl, 2-hydroxyethyl, methylthio, ethylthio, trifluoromethoxy,1,1,2,2-tetrafluoroethoxy, fluoro, chloro, and bromo;

R¹ is selected from the group consisting of hydrido, hydroxy, amino, methyl, ethyl, propyl, n-butyl, 2,2,2-trifluoroethyl, 3,3,3-trifluoropropyl, 2,2,3,3,3-pentafluoropropyl, 2-aminoethyl, 3-aminopropyl, 2-hydroxyethyl, 3-hydroxypropyl, and 2,3-dihydroxypropyl;

R² is Z⁰-Q^{b};

Z⁰ is selected from the group consisting of a bond, CH₂, CH₂CH₂, O, S, NH, N(CH₃), OCH₂, SCH₂, N(H)CH₂, and N(CH₃)CH₂;

Q^{b} is selected from the group consisting of phenyl, 2-thienyl, 3-thienyl, 2-furyl, 3-furyl, 2-pyrrolyl, 3-pyn-olyl, 2-imidazolyl, 4-imidazolyl, 3-pyrazolyl, 4-pyrazolyl, 2-thiazolyl, 3-isoxazolyl, 5 isoxazolyl, 2-pyridyl, 3-pyridyl, 4-pyridyl, 2-pyrazinyl, 2-pyrimidinyl, 4-pyrimidinyl, 5-pyrimidinyl, 3-pyridazinyl, 4-pyridazinyl, and 1,3,5-triazin-2,5-yl, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

R⁹, R¹¹, and R¹³ are independently selected from the group consisting of hydrido, amidino, guanidino, carboxy, methyl, ethyl, propyl, isopropyl, methoxy, ethoxy, isopropoxy, propoxy, hydroxy, amino, N-methylamino, N,N-dimethylamino, N-ethylamino, methylthio, ethylthio, isopropylthio, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trnfluoro-1-hydroxyethyl, amidocarbonyl, N-methylamidocarbonyl, N,N-dimethylamidocarbonyl, and cyano;

R¹⁰ and R¹² are independently selected from the group consisting of hydrido, amidino, guanidino, carboxy, carboxymethyl, methyl, ethyl, propyl, isopropyl, methoxy, ethoxy, isopropoxy, propoxy, hydroxy, amino, methoxyamino, ethoxyamino, acetamido, trifluoroacetanido, aminomethyl, 1-aminoethyl, 2-aiwnoethyl, N-methylamino, dimethylamino, N-ehylamino, methanesulfonanido, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, N-benzylamidocarbonyl, N-(2-chlorobenzyl)amidocarbonyl, N-3-fluorobenzyl)awndocarbonyl, N-2-trifluoromethylbenzyl)amidocarbonyl, N-(1-phenylethyl)amidocarbonyl, N-(1-methyl-1-phenylethyl)amidocarbonyl, N-benzylamidosulfonyl, N-2-chlorobenzyl)amidosulfonyl, N-ethylamidocarbonyl, N-isopropylamidocarbonyl, N-propylamidocarbonyl, N-isobutylamidocarbonyl, N-(2-butyl)amidocarbonyl, N-cyclobutylamidocarbonyl, N-cyclopentylamidocarbonyl, N-cyclohexylamidocarbonyl, fluoro, chloro, bromo, cyano, cyclobutoxy, cyclohexoxy, cyclohexylmethoxy, 4-tiifiuoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, 5-bromopyrid-2-ylmethylamino, 4-butoxyphenamino, 3-chlorobenzyl, 4-chlorophenoxy, 4-chloro-3-ethylphenoxy, 4-chloro-3-ethylbenzylaniuno, 4-chloro-3-ethylphenylaniuno, 3-chlorobenzyloxy, 4-chlorobenzyloxy, 4-chlorobenzylsulfonyl, 4-chlorophenylamino, 4-chlorophenylsulfonyl, 5-chloropyrid-3-yloxy, 2-cyanopynd-3-yloxy, 2,3-difluorobenzyloxy, 2,4-difluorobenzyloxy, 3,4-difluorobenzyloxy, 2,5-difluorobenzyloxy, 3,5-difluorophenoxy, 3,5-difluorobenzyloxy, 4-difluoromethoxybenzyloxy, 2,3-difluorophenoxy, 2,4-difluorophenoxy, 2,5-difluorophenoxy, ,5 diethylphenoxy, 3,4-dimethylphenoxy, 3,4-dimethylbenzyloxy, 3,5-dimethylbenzyloxy, 4-ethoxyphenoxy, 4-ethylbenzy1-oxy, 3-ethylphenoxy, 4-ethylaminophenoxy, 3-ethyl-Imethylphenoxy, 4-fluorobenzyloxy, 2-fluoro-3-trifluoromethylbenzyloxy, 3-fluoro-5-trifluoromethylbenzyloxy, 4-fluom-2-trifluoromethylbenzyloxy, 4-fluoro-3-trifluoromethylbenzyloxy, 2-fluorophenoxy, 4-fluorophenoxy, 2-fluoro-3-triuoromethylphenoxy, 2-fluorobenzyloxy, 4-fluorophenylamino, 2-fluorotrifluorometiylphenoxy, 4-isopropylbenzyloxy, 3-isopropylphenoxy, 4-isopropylphenoxy, 4-isopropyl-3-methylphenoxy, 4-isopropylbenzyloxy, 3-isopropylphenoxy, 4-isopropylphenoxy, 4-isopropyl-3-methylphenoxy, phenylamino, 1-phenyletboxy, 2-phenylethoxy, 2-phenylethyl, 2-phenylethylamino, phenylsulfonyl, 3-trifuoromethoxybenzyloxy, 4-trifluoromethoxybenzyloxy, 3-trifluoromethoxyphenoxy, 4-trifluoromethoxyphenoxy, 3-trifluoroinethylbenzyloxy, 4-trifluoromethylbenzyloxy, 2,4-bis-trfluoromethylbenzyloxy, 3-triuoromethylbenzyl, 3,5-bis-trifluoromethylbenzyloxy, 4-trifiuoromethylphenoxy, 3-trifluoromethylphenoxy, 3-trifluoromethylthiobenzyloxy, 4-trifluoromethylthiobenzyloxy, 2,3,4-tr-luorophenoxy, 2,3,5-t-iiuorophenoxy, 3-pentafluoroethylphenoxy, 3-(1,1,2,2-tetrafluoroethoxy)phenoxy, and 3-trifluoromethylthiophenoxy;

Y⁰ is selected from the group of formulas consisting of:

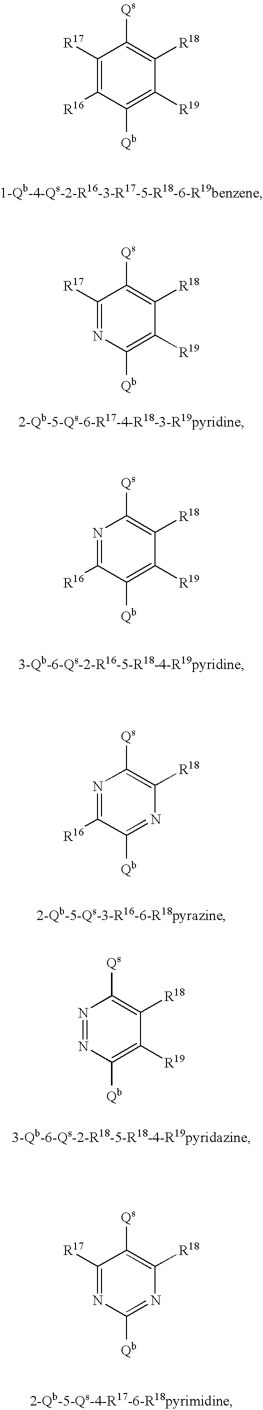

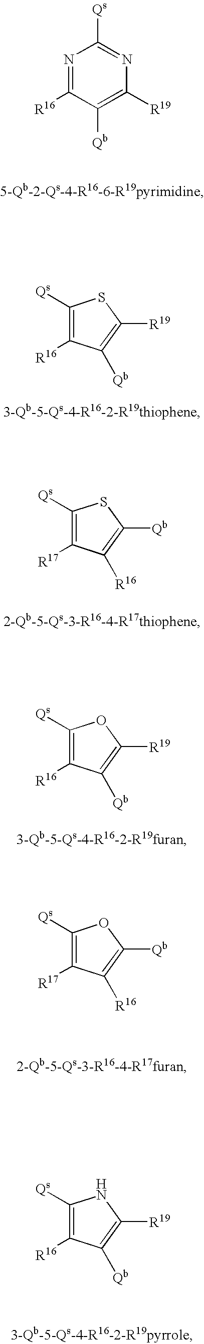

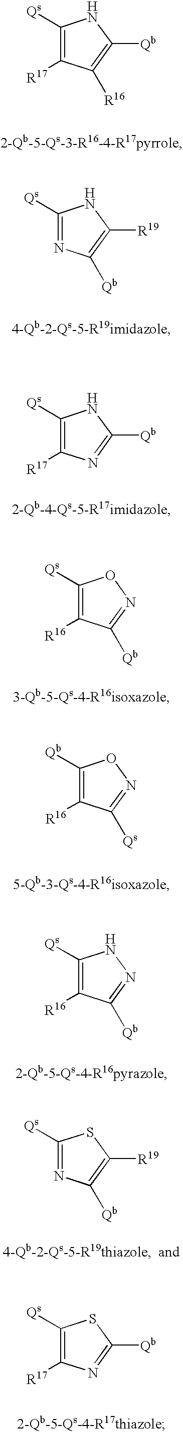

Q^{s} is selected from the group consisting of a bond, CH₂ and CH₂CH₂.

In a most preferned specific embodiment of Formula I, compounds have the formula:

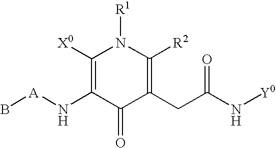

or a pharmaceutically acceptable salt thereof, wherein;

B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, and 5 isoxazolyl, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;

R³², R³³, R³⁴, R³⁵, and R³⁶ are independently selected from the group consisting of hydrido, amidino, guanidino, methyl, ethyl, methoxy, ethoxy, hydroxy, amino, N-methylamino, dimethylamino, methoxyamino, methylthio, ethylthio, trifluoromethyl, pentafluoroethyl, 2,2,2-trifluoroethyl, fluoro, chloro, bromo, amidosulfonyl, N-methylamidosulfonyl, hydroxymethyl, amidocarbonyl, carboxy, cyano, and Q^{b};

A is selected from the group consisting of a bond, NH, N(CH₃), CH₂, CH₃CH, and CH₂CH₂;

Q^{b} is NR²⁰R²¹ or C(NR²⁵)NR²³R²⁴;

R²⁰, R²¹, R²³, R²⁴, and R²⁵ are independently selected from the group consisting of hydrido, methyl, and ethyl.

In another most preferred specific embodiment of Formula I, compounds have the formula:

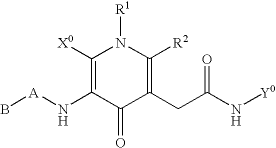

or a pharmaceutically acceptable salt thereof, wherein;

B is selected from the group consisting of hydrido, ethyl, 2-propenyl, 2-propynyl, propyl, isopropyl, butyl, 2-butenyl, 2-butynyl, secbutyl, tert-butyl, isobutyl, 2-methylpropenyl, 1-pentyl, 2-pentenyl, 3-pentenyl, 2-pentynyl, 3-pentynyl, 2-pentyl, 3-pentyl, 2-methylbutyl, 2-methyl-2-butenyl, 3-methylbutyl, 3-methyl-2-butenyl, 1-hexyl, 2-hexenyl, 3-hexenyl, 4-hexenyl, 2-hexynyl, 3-hexynyl, 4-hexynyl, 2-hexyl, 1-methyl-2-pentenyl, 1-methyl-3-pentenyl, 1-methyl-2-pentynyl, 1-methyl-3-pentynyl, 3-hexyl, 1-ethyl-2-butenyl, 1-heptyl, 2-heptenyl, 3-heptenyl, 4-heptenyl, 5-heptenyl, 2-heptynyl, 3-heptynyl, 4-heptynyl, 5-heptynyl, 2-heptyl, 1-methyl-2-hexenyl, 1-methyl-3-hexenyl, 1-methylthexenyl, 1-methyl-2-hexynyl, 1-methyl-3-hexynyl, 1-methylhexynyl, 3-heptyl, 1-ethyl-2-pentenyl, 1-ethyl-3-pentenyl, 1-ethyl-2-pentynyl, 1-ethyl-3-pentynyl, 2,2,2-trifluoroethyl, 2,2-difluoropropyl, 4-trifluoromethyl-5,5,5-tifluoropentyl, 4-trifuoromethylpentyl, 5,5,6,6,6-pentafluorohexyl, and 3,3,3-trifluoropropyl, wherein each member of group B is optionally substituted at any carbon up to and including 5 atoms from the point of attachment of B to A with one or more of the group consisting of R³² R³³, R³⁴, R³⁵, R³⁶;

R³², R³³, R³⁴, R³⁵, and R³⁶ are independently selected from the group consisting of hydrido, amnidino, guaidino, methyl, ethyl, methoxy, ethoxy, hydroxy, amino, N-methylamino, dimethylamino, methoxyamino, methylthio, ethylthio, tbfluoromethyl, pentafluoroethyl, 2,2,2-trifluoroethyl, fluoro, chloro, bromo, amidosulfonyl, N-methylamidosulfonyl, hydroxymethyl, amidocarbonyl, carboxy, cyano, and Q^{b};

A is selected from the group consisting of a bond, NH, N(CH₃), CH₂, CH₃CH, and CH₂CH₂;

A is optionally selected from the group consisting of CH₂N(CH₃), CH₂N(CH₂CH₃), CH₂CH₂N(CH₃), and CH₂CH₂N(CH₂CH₃) with the proviso that B is hydrido;

Q^{b} is selected from the group consisting of NR²⁰R²¹, C(NR²⁵)NR²³R²⁴, and N(R²⁶)C(NR²⁵)N(R²³)(R²⁴);

R²⁰, R²¹, R²³, R²⁴, R²⁵, and R²⁶ are independendy selected from the group consisting of hydrido, methyl, and ethyl.

In still another most preferred specific embodiment of Formula I, compounds have the formula:

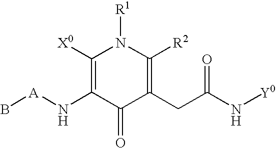

or a pharmaceutically acceptable salt thereof, wherein;

B is selected from the group consisting of cyclopropyl, cyclobutyl, cyclopentyl, cyclohexyl, cycloheptyl, oxalan-2-yl, 2-(2R)bicyclo[2.2.1]-heptyl, oxetan-3-yl, azetidin-1-yl, azetidin-2-yl, azetidin-3-yl, bicyclo[3.1.0]hexan-6-yl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 2-tetrahydrofuranyl, 3-etrahydrofuranyl, 2-tetrahydropyranyl, 3-tetrahydropyranyl, 4-tetrahydropyranyl, 2-tetrahydrothienyl, and 3-tetrahydrothienyl, wherein each ring carbon is optionally substituted with R³³, ring carbons and a nitrogen adjacent to the carbon atom at the point of attachment are optionally substituted with R⁹ or R¹³, a ring carbon or nitrogen adjacent to the R⁹ position and two atoms from the point of attachment are optionally substituted with R¹⁰, and a ring carbon or nitrogen atom adjacent to the R¹³ position and two atoms from the point of attachment is optionally substituted with R¹²;

R³³ is selected from the group consisting of hydrido, amidino, guanidino, methyl, ethyl, methoxy, ethoxy, hydroxy, carboxy, amino, N-methylamino, dimethylamino,-methoxyamino, methylthio, ethylthio, trifluoromethyl, pentafluoroethyl, 2,2,2-trifluoroethyl, fluoro, chloro, bromo, amidosulfonyl, N-methylamidosulfonyl, hydroxymethyl, amidocarbonyl, cyano, and Q^{b};

A is selected from the group consisting of a bond, NH, N(CH₃), CH₂, CH₃CH, CH₂CH₂, and CH₂CH₂CH₂;

Q_{b} is NR²⁰N²¹ or C(NR²⁵)NR²³R²⁴;

R²⁰, R²¹, R²³, R²⁴, and R²⁵ are independently selected from the group consisting of hydrido, methyl, and ethyl.

The most preferred specific embodiment compounds of Formula 1, said compounds having the formula:

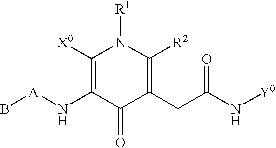

or a pharmaceutically acceptable salt thereof, have common structural units, wherein;

X^{o} is selected from the group consisting of hydxido, aminomethyl, methyl, ethyl, trifluoromethyl, 2,2,2-trifluoroethyl, hydroxymethyl, 2-hydroxyethyl, chloro, and fluoro;

R¹ is selected from the group consisting of hydrido, methyl, ethyl, propyl, 2,2,2-trifluoroethyl, 3,3,3-trifluoropropyl, 2,2,3,3,3-pentafluoropropyl, and 2-hydroxyethyl;

R² is selected from the group consisting of phenyl, 2-thienyl, 2-furyl, 2-pyrrolyl, 2-imidazolyl, 2-thiazolyl, 3-isoxazolyl, 2-pyridyl, and 3-pyridyl, wherein a carbon adjacent to the carbon at the point of attachment of said phenyl or heteroaryl ring to the 4-pyridonering is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;

R⁹, R¹¹, and R¹³ are independently selected from the group consisting of hydrido, methyl, ethyl, methoxy, ethoxy, hydroxy, amino, N-methylamino, N,N-dimethylamino, methylthio, trifluoromethyl, pentafluoroethyl, 2,2,2-trifluoroethyl, fluoro, chloro, bromo, umidosulfonyl, N-methylamidosulfonyl, N,N-dimethylamidosulfonyl, hydroxymethyl, 1-hydroxyethyl, amidocarbonyl, N-methylamidocarbonyl, carboxy, and cyano;

R¹⁰ and R¹² are independently selected from the group consisting of hydrido, amidino, amidocarbonyl, N-methylamidocarbonyl, N-benzylamidocarbonyl, N-(2-chlorobenzyl)amidocarbonyl, N-(3-fluorobenzyl)amidocarbonyl, N-(2-trifluoromethylbenzyl)amidocarbonyl, N-(1-phenylethyl)amidocarbonyl, N-(1-methyl-1-phenylethyl)amidocarbonyl, N-benzylamidosulfonyl, N-(2-chlorobenzyl)amidosulfonyl, N-ethylamidocarbonyl, N-isopropylamidocarbonyl, N-propylamidocarbonyl, N-isobutylamidocarbonyl, N-(2-butyl)amidocarbonyl, N-cyclobutylamidocarbonyl, N-cyclopentylamidocarbonyl, N-cyclohexylamidocarbonyl, guanidino, methyl, ethyl, methoxy, ethoxy, hydroxy, hydroxymethyl, 1-hydroxyethyl, 2-hydroxyethyl, carboxy, carboxymethyl, amino, acetamido, trifluoromethyl, pentafluoroethyl, 2,2,2-trifluoroethyl, trifluoroacetamido, aminomethyl, N-methylamino, dimethylamino, methoxyamino, amidosulfonyl, N-methylamidosulfonyl, N,N-dimethylamidosulfonyl, methanesulfonamido, methoxycarbonyl, fluoro, chloro, bromo, and cyano;

Y⁰ is selected from the group of formulas consisting of:

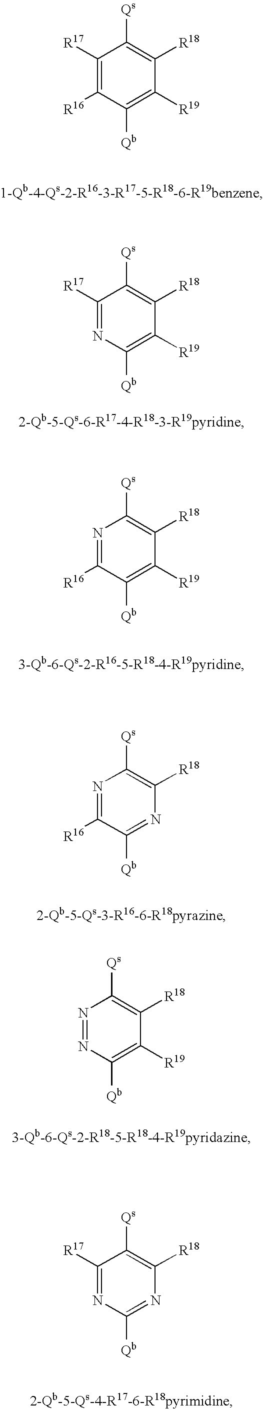

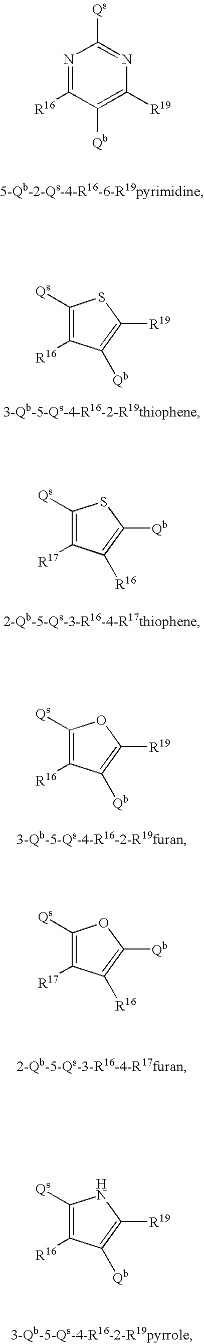

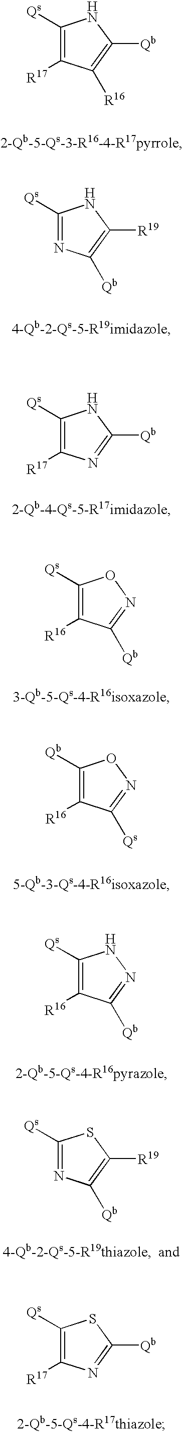

R¹⁶, R¹⁷, R¹⁸, and R¹⁹ are independently selected from the group consisting of hydrido, methyl, ethyl, amidino, guanidino, methoxy, hydroxy, amino, aminomethyl, 1-aminoethyl, 2-aminoethyl, N-methylamino, dimethylamino, methylthio, ethylthio, trifluoromethylthio, methylsulfinyl, methylsulfonyl, trifluoromethyl, pentafluoroethyl, 2,2,2-trifluoroethyl, tnfluoromethoxy, fluoro, chloro, hydroxymethyl, carbooxy, and cyano;

Q⁵ is CH₂.

The compounds of this invention can be used in anticoagulant therapy for the treatment and prevention of a variety of thrombotic conditions including coronary artery and cerebrovascular disease. The compounds of this invention can be used to inhibit serine protease associated with the coagulation cascade and factors II, VII, VIII, IX, X, XI, or XII. The compounds of the invention can inhibit the formation of blood platelet aggregates, inhibit the formation of fibrin, inhibit thrombus formation, and inhibiting embolus formation in a mammal, in blood, in blood products, and in mammalian organs. The compounds also can be used for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels in a mammal. The compounds can also be used in prophylactic treatment of subjects who are at risk of developing such disorders. The compounds can be used to lower the risk of atherosclerosis. The compounds of Formula (I) would also be useful in prevention of cerebral vascular accident (CVA) or stroke.

Besides being useful for human treatment, these compounds are also useful for veterinary treatment of companion animals, exotic animals and farm animals, including mammals, rodents, and the like. More preferred animals include horses, dogs, and cats.

The use of generic terms in the description of the compounds are herein defined for clarity. Standard single letter elemental symbols are used to represent specific types of atoms unless otherwise defined. The symbol “C” represents a carbon atom. The symbol “O” represents an oxygen atom. The symbol “N” represents a nitrogen atom. The symbol “P” represents a phosphorus atom. The symbol “S” represents a sulfur atom. The symbol “H” represents a hydrogen atom. Double letter elemental symbols are used as defined for the elements of the periodical table (i.e., Cl represents chlorine, Se represents selenium, etc.).

As utilized herein, the term “alkyl”, either alone or within other terms such as “haloalkyl” and “alkylthio”, means an acyclic alkyl radical containing from 1 to about 10, preferably from 3 to about 8 carbon atoms and more preferably 3 to about 6 carbon atoms. Said alkyl radicals may be optionally substituted with groups as defined below. Examples of such radicals include methyl, ethyl, chloroethyl, hydroxyethyl, n-propyl, oxopropyl, isopropyl, n-butyl, cyanobutyl, isobutyl, sec-butyl, tert-butyl, pentyl, aminopentyl, iso-amyl, hexyl, octyl and the like.

The term “alkenyl” refers to an unsaturated, acyclic hydrocarbon radical in so much as it contains at least one double bond. Such alkenyl radicals contain from about 2 to about 10 carbon atoms, preferably from about 3 to about 8 carbon atoms and more preferably 3 to about 6 carbon atoms. Said alkenyl radicals may be optionally substituted with groups as defined below. Examples of suitable alkenyl radicals include propenyl, 2-chloropropenyl, buten-1-yl, isobutenyl, penten-1-yl, 2-2-methylbuten-1-yl, 3-methylbuten-1-yl, hexen-1-yl, 3-hydroxyhexen-1-yl, hepten-1-yl, and octen-1-yl, and the like.

The term “alkynyl” refers to an unsatuaated, acyclic hydrocarbon radical in so much as it contains one or more triple bonds, such radicals containing about 2 to about 10 carbon atoms, preferably having from about 3 to about 8 carbon atoms and more preferably having 3 to about 6 carbon atoms. Said alkynyl radicals may be optionally substituted with groups as defined below. Examples of suitable alkynyl radicals include ethynyl, propynyl, hydroxypropynyl, butyn-1-yl, butyn-2-yl, pentyn-1-yl, pentyn-2-yl, 4 methoxypentyn-2-yl, 3-methylbutyn-1-yl, hexyn-1-yl, hexyn-2-yl, hexyn-3-yl, 3,3-dimethylbutyn-1-yl radicals and the like.

The term “hydrido” denotes a single hydrogen atom (H). This hydrido radical may be attached, for example, to an oxygen atom to form a “hydroxyl” radical, one hydrido radical may be attached to a carbon atom to form a “methine” radical —CH═, or two hydrido radicals may be attached to a carbon atom to form a “methylene” (—CH₂—) radical.

The term “carbon” radical denotes a carbon atom without any covalent bonds and capable of forming four covalent bonds.

The term “cyano” radical denotes a carbon radical having three of four covalent bonds shared by a nitrogen atonl

The term “hydroxyalkyl” embraces radicals wherein any one or more of the alkyl carbon atoms is substituted with a hydroxyl as defined above. Specifically embraced are monohydroxyalkyl, dihydroxyalkyl and polyhydroxyalkyl radicals.

The term “alkanoyl” embraces radicals wherein one or more of the terminal alkyl carbon atoms are substituted with one or more carbonyl radicals as defned below. Specifically embraced are monocarbonylalkyl and dicarbonylalkyl radicals. Examples of monocarbonylalkyl radicals include formyl, acetyl, and pentanoyl. Examples of dicarbonylalkyl radicals include oxalyl, malonyl, and succinyl.

The term “alkylene” radical denotes linear or branched radicals having from 1 to about 10 carbon atoms and having attachment points for two or more covalent bonds. Examples of such radicals are methylene, ethylene, methylethylene, and isopropylidene.

The term “alkenylene” radical denotes linear or branched radicals having from 2 to about 10 carbon atoms, at least one double bond, and having attachment points for two or more covalent bonds. Examples of such radicals are 1,1-vinylidene (CH₂═C), 1,2-vinylidene (—CH—CH—), and 1,4-butadienyl (—CH═CH—CH═CH—).

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either a bromo, chloro or a fluoro atom within the radical. Dihalo radicals may have two or more of the same halo atoms or a combination of different halo radicals and polyhaloalkyl radicals may have more than two of the same halo atoms or a combination of different halo radicals. More preferred haloalkyl radicals are “haloalkyl” radicals having one to about six carbon atoms. Examples of such haloalkyl radicals include fluoromethyl, difluoromethyl, trifluoromethyl, chloromethyl, dichloromethyl trichloromethyl, trifluoroethyl, pentafluoroethyl, heptafluoropropyl, difluorochloromethyl, dichlorofluoromethyl, difluoroethyl, difluoropropyl, dichloroethyl and dichloropropyl.

The term “hydroxyhaloalkyl” embraces radicals wherein any one or more of the haloalkyl carbon atoms is substituted with hydroxy as defined above. Examples of “hydroxyhaloalkyl” radicals include hexafluorohydroxypropyl.

The term “haloalkylene radical” denotes alkylene radicals wherein any one or more of the alkylene carbon atoms is substituted with halo as defined above. Dihalo alkylene radicals may have two or more of the same halo atoms or a combination of different halo radicals and polyhaloalkylene radicals may have more than two of the same halo atoms or a combination of different halo radicals. More preferred haloalkylene radicals are “haloalkylene” radicals having one to about six carbon atoms. Examples of “haloalkylene” radicals include difluoromethylene, tetrafluoroethylene, tetrachloroethylene, alkyl substituted monofluoromethylene, and amyl substituted ttifluoromethylene.

The term “haloalkenyl” denotes linear or branched radicals having from 1 to about 10 carbon atoms and having one or more double bonds wherein any one or more of the alkenyl carbon atoms is substituted with halo as defined above. Dihaloalkenyl radicals may have two or more of the same halo atoms or a combination of different halo radicals and polyhaloalkenyl radicals may have more than two of the same halo atoms or a combination of different halo radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having one or more alkoxy radicals attached to the alkyl radical, that is, to form monoalkoxyalkyl and dialkoxyalkyl radicals. More preferred alkoxy radicals are “alkoxy” radicals having one to six carbon atoms. Examples of such radicals include methoxy, ethoxy, propoxy, butoxy, isopropoxy and ten-butoxy alkyls. The “alkoxy” radicals may be further substituted with one or more halo atoms, such as fluoro, chloro or bromo, to provide “haloalkoxy” and “haloalkoxyalkyl” radicals. Examples of such haloalkoxy radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy. Examples of such haloalkoxyalkyl radicals include fluoromethoxymethyl, chloromethoxyethyl, trifluoromethoxymethyl, difluoromethoxyethyl, and trifluoroethbxymethyl.

The terms “alkenyloxy” and “alkenyloxyalkyl” embrace linear or branched oxycontaining radicals each having alkenyl portions of two to about ten carbon atoms, such as ethenyloxy or propenyloxy radical. The term “alkenyloxyalkyl” also embraces alkenyl radicals having one or more alkenyloxy radicals attached to the alkyl radical, that is, to form monoalkenyloxyalkyl and dialkenyloxyalkyl radicals. More preferred alkenyloxy radicals are “alkenyloxy” radicals having two to six carbon atoms. Examples of such radicals include ethenyloxy, propenyloxy, butenyloxy, and isopropenyloxy alkyls. The “alkenyloxy” radicals may be further substituted with one or more halo atoms, such as fluoro, chloro or bromo, to provide “haloalkenyloxy” radicals. Examples of such radicals include trifluoroethenyloxy, fluoroethenyloxy, difluoroethenyhloxy, and fluoropropenyloxy.

The term “haloalkoxyalkyl” also embraces alkyl radicals having one or more haloalkoxy radicals attached to the alkyl radical, that is, to form monohaloalkoxyalkyl and dihaloalkoxyalkyl radicals. The term “haloalkenyloxy” also embraces oxygen radicals having one or more haloalkenyloxy radicals attached to the oxygen radical, that is, to form monohaloalkenyloxy and dihaloalkenyloxy radicals. The term “haloalkenyloxyalkyl” also embraces alkyl radicals having one or more haloalkenyloxy radicals attached to the alkyl radical, that is, to form monohaloalkenyloxyalkyl and dihaloalkenyloxyalkyl radicals.

The term “alkylenedioxy” radicals denotes alkylene radicals having at least two oxygens bonded to a single alkylene group. Examples of “alkylenedioxy” radicals include methylenedioxy, ethylenedioxy, alkylsubstituted methylenedioxy, and arylsubsfituted methylenedioxy. The term “haloalkylenedioxy” radicals denotes haloalkylene radicals having at least two oxy groups bonded to a single haloalkyl group. Examples of “haloalkylenedioxy” radicals include difluoromethylenedioxy, tetrarluoroethylenedioxy, tetrachloroethylenedioxy, alkylsubstituted monofluoromethylenedioxy, and arylsubstituted monofluoromethylenedioxy.

The term “aryl”, alone or in combination, means a carbocyclic aromatic system containing one, two or three rings wherein such rings may be attached together in a pendant manner or may be fused. The term “fused” means that a second ring is present (ie, attached or formed) by having two adjacent atoms in common (ie, shared) with the first ring. The term “fused” is equivalent to the term “condensed”. The term “aryl” embraces aromatic radicals such as phenyl, naphthyl, tetrahydronaphthyl, indane and biphenyl.

The term “perhaloaryl” embraces aromatic radicals such as phenyl, naphthyl, tetrahydronaphthyl, indane and biphenyl wherein the aryl radical is substituted with 3 or more halo radicals as defined below.

The term “heterocyclyl” embraces saturated and partially saturated heteroatom-containing ring-shaped radicals having from 4 through 15 ring members, herein referred to as “C4-C15 heterocyclyl”, selected from carbon, nitrogen, sulfur and oxygen, wherein at least one ring atom is a heteroatom. Heterocyclyl radicals may contain one, two or three rings wherein such rings may be attached in a pendant manner or may be fused. Examples of saturated heterocyclic radicals include saturated 3 to 6-membered heteromonocylic group containing 1 to 4 nitrogen atoms[e.g. pyrrolidinyl, imidazolidinyl, piperidino, piperazinyl, etc.]; saturated 3 to 6-membered heteromonocyclic group containing 1 to 2 oxygen atoms and 1 to 3 nitrogen atoms [e.g. morpholinyl, etc.]; saturated 3 to 6-membered heteromonocyclic group containing 1 to 2 sulfur atoms and 1 to 3 nitrogen atoms [e.g., thiazolidinyl, etc.]. Examples of partially saturated heterocyclyl radicals include dihydrothiophene, dihydropyran, dihydrofuran and dihydrothiazole. Non-limiting examples of heterocyclic radicals include 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-ditbianyl, thiomorpholinyl, and the like. Said “heterocyclyl” group may be substituted as defined herein, Preferred heterocyclic radicals include five to twelve membered fused or unfused radicals.

The term “heteroaryl” embraces fully unsaturated heteroatom-containing ring-shaped aromatic radicals having from 4 through 15 zing members selected from carbon, nitrogen, sulfur and oxygen, wherein at least one ring atom is a heteroatom. Heteroaryl radicals may contain one, two or three rings wherein such rings may be attached in a pendant manner or may be fused. Examples of “heteroaryl” radicals, include the unsaturated heteromonocyclyl group of 5 to 6 contiguous members containing 1 to 4 nitrogen atoms, for example, pyrrolyl, pyrrolinyl, imidazolyl, pymwlyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl, etc.] tetraaolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl, etc.], etc.; unsaturated 3 to 6-membered heteromonocyclic group containing an oxygen atom, for example, pyranyl, 2-furyl, 3-furyl, etc.; unsaturated 5 to 6 membered heteromonocyclic group containing a sulfur atom, for example, 2-thienyl, 3-thienyl, etc.; unsaturated 5- to 6-imembered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.] etc.; unsaturated condensed heterocyclic group containing 1 to 2 oxygen atoms and 1 to 3 nitrogen atoms [e.g. benzoxazolyl, benzoxadiazolyl, etc.]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.] etc.; unsaturated condensed heterocyclic group containing 1 to 2 sulfur atoms and 1 to 3 nitrogen atoms [e.g., benzothiazolyl, benzothiadiazolyl, etc.] and the like. The term also embraces radicals where heterocyclic radicals are fused with aryl radicals. Examples of such fused bicyclic radicals include benzofuran, benzothiophene, and the like. Said “heteroaryl” group may be substituted as defined herein. Preferred heteroaryl radicals include five and six membered unfused radicals. Non4-imiting examples of heteroaryl radicals include 2-thienyl, 3-thienyl, 2-furyl, 3-furyl, 2-pyrrolyl, 3-pyrrolyl, 2-imidazolyl, 4 imidazolyl, 3-pyrazolyl, 4-pyrazolyl, 1,2,4-triazol-3-yl, 1,2,4-tiazol-5-yl, 1,2,4-oxadiazol-3-yl, 1,2,4-oxadiazol-5-yl, 1,3,4-xadiazol-3-yl, 1,3,4-oxadiazol-5-yl, 3-isothiazolyl, 5-isothiazolyl, 2-oxazolyl, 2,5-thiazolyl, 3-isoxazolyl, 5-isoxazolyl, 2-pyridyl, 3-pyridyl, 4-pyridyl, 2-pyrazinyl, 2-pyrimidinyl, 4-pyrimidinyl, 5-pyrimidinyl, 3-pyridazinyl, 4-pyridazinyl, 1,3,5-triazin-2-yl, 1,2,4-triazin-3-yl, 1,2,4-triazin-5-yl, 1,2,4-triazin-yl, 1,2,3-triazinAyl, and 1,2,3-triazin-5-yl, and the like.

The term “sulfonyl”, whether.used alone or linked to other terms such as alkylsulfonyl, denotes respectively divalent radicals —SO₂—. “Alkylsulfonyl”, embraces alkyl radicals attached to a sulfonyl radical, where alkyl is defined as above. “Alkylsulfonylalkyl”, embraces alkylsulfonyl radicals attached to an alkyl radical, where alkyl is defined as above. “Haloalkylsulfonyl”, embraces haloalkyl radicals attached to a sulfonyl radical, where haloalkyl is defined as above. “Haloalkylsulfonylalkyl”, embraces haloalkylsulfonyl radicals attached to an alkyl radical, where alkyl is defined as above.

The term “amidosulfonyl” embraces amino, monoalkylamino, dialkylamino, monocycloalkylamino, alkyl cycloalkylamino, dicycloalkylamino, N-alkyl-N-arylamino, arylamino, aralkylamino, nitrogen containing heterocyclyl, heterocyclylamino, N-alkyl-N-heterocyclylamino, heteroarylamino, and heteroarLacylamino radicals, attached to one of two unshared bonds in a sulfonyl radical.

The term “sulfinyl”, whether used alone or linked to other terms such as alkylsulfinyl, denotes respectively divalent radicals —S(O)—. “Alkylsulfinyl”, embraces alkyl radicals attached to a sulfinyl radical, where alkyl is defined as above. “Alkylsulfinylalkyl”, embraces alkylsulfinyl radicals attached to an alkyl radical, where alkyl is defined as above. Haloalkylsulfinylti, embraces haloalkyl radicals attached to a sulfinyl radical, where haloalkyl is defined as above. “Haloalkylsulfinylalkyl”, embraces haloalkylsulfinyl radicals attached to an alkyl radical, where alkyl is defined as above.

The term “aralkyl” embraces aryl-substit-ated alkyl radicals. Preferable aralkyl radicals are “aralkyl” radicals having aryl radicals attached to alkyl radicals having one to six carbon atoms. Examples of such radicals include benzyl, diphenylmethyl, triphenylmethyl, phenylethyl and diphenylethyl. The terms benzyl and phenylmethyl are interchangeable.

The term “heteroaralkyl” embraces heteroaryl-substituted alkyl radicals wherein the heteroaralkyl radical may be additionally substituted with three or more substituents as defined above for aralkyl radicals. The term “perhaloaralkyl” embraces aryl-substituted alkyl radicals wherein the analkyl radical is substituted with three or more halo radicals as defined above.

The term “aralkylsulfinyl”, embraces arwkyl radicals attached to a sulfinyl radical, where aralkyl is defined as above. “Aralkylsulfinylalkyl”, embraces aralkylsulfinyl radicals attached to an alkyl radical, where alkyl is defined as above.

The term “aralkylsulfonyl”, embraces aralkyl radicals attached to a sulfonyl radical, where aralkyl is defined as above. “Aralkylsulfonylalkyl”, embraces aralkylsulfonyl radicals attached to an alkyl radical, where alkyl is defined as above.

The term “cycloalkyl” embraces radicals having three to 15 carbon atoms. More preferred cycloalkyl radicals are “cycloalkyl” radicals having three to seven carbon atoms. Examples include radicals such as cyclopropyl, cyclobutyl, cyclopentyl, cyclohexyl and cycloheptyl. The term cycloalkyl embraces radicals having seven to 15 carbon atoms and having two to four rings. Exmaples incude radicals such as norbornyl (i.e., bicyclo[2.2.1]heptyl) and adamantyl. The term “cycloalkylalkyl” embraces cycloalkyl-substituted alkyl radicals. Preferable cycloalkylalkyl radicals are “cycloalkylalkyl” radicals having cycloalkyl radicals attached to alkyl radicals having one to six carbon atoms. Examples of such radicals include cyclohexylhexyl. The term “cycloalkenyl” embraces radicals having three to ten carbon atoms and one or more carbon-carbon double bonds. Preferred cycloalkenyl radicals are “cycloalkenyl” radicals having three to seven carbon atoms. Examples include radicals such as cyclobutenyl, cyclopentenyl, cyclohexenyl and cycloheptenyl. The term “halocycloalkyl” embraces radicals wherein any one or more of the cycloalkyl carbon atoms is substituted with halo as defined above. Specifically embraced are monohalocycloalkyl, dibalocycloalkyl and polyhalocycloalkyl radicals. A monobalocycloalkyl radical, for one example, may have either a bromo, chloro or a fluoro atom within the radical. Dihalo radicals may have two or more of the same halo atoms or a combination of different halo radicals and polyhalocycloalkyl radicals may have more than two of the same halo atoms or a combination of different halo radicals. More preferred halocycloalkyl radicals are “halocycloalkyl” radicals having three to about eight carbon atoms. Examples of such halocycloalkyl radicals include fluorocyclopropyl, difluorocyclobutyl, trifluorocyclopentyl, tetrafluorocyclohexyl, and dichlorocyclopropyl. The term “halocycloalkenyl” embraces radicals wherein any one or more of the cycloalkenyl carbon atoms is substituted with halo as defined above. Specifically embraced are monohalocycloalkenyl, dihalocycloalkenyl and polyhalocycloalkenyl radicals.

The term “cycloalkoxy” embraces cycloalkyl radicals attached to an oxy radical. Examples of such radicals includes cyclohexoxy and cyclopentoxy. The term “cycloalkoxyalkyl” also embraces alkyl radicals having one or more cycloalkoxy radicals attached to the alkyl radical, that is, to form monocycloalkoxyalkyl and dicycloalkoxyalkyl radicals. Examples of such radicals include cyclohexoxyethyl. The “cycloalkoxy” radicals may be further substituted with one or more halo atoms, such as fluoro, chloro or bromo, to provide “halocycloalkoxy” and “halocycloalkoxyalkyl” radicals.

The term “cycloalkylalkoxy” embraces cycloalkyl radicals attached to an alkoxy radical. Examples of such radicals includes cyclohexylmethoxy and cyclopentylmethoxy.

The term “cycloalkenyloxy” embraces cycloalkenyl radicals attached to an oxy radical. Examples of such radicals includes cyclohexenyloxy and cyclopentenyloxy. The term “cycloalkenyloxyalkyl” also embraces alkyl radicals having one or more cycloalkenyloxy radicals attached to the alkyl radical, that is, to form monocycloalkenyloxyalkyl and dicycloalkenyloxyalkyl radicals. Examples of such radicals include cyclohexenyloxyethyl. The “cycloalkenyloxy” radicals may be further substituted with one or more halo atoms, such as fluoro, chloro or bromo, to provide “halocycloalkenyloxy” and “halocycloalkenyloxyalkyl” radicals.

The term “cycloalkylenedioxy” radicals denotes cycloalkylene radicals having at least two oxygens bonded to a single cycloalkylene group. Examples of “alkylenedioxy” radicals include 1,2-dioxycyclohexylene.

The term “cycloalkylsulfinyl”, embraces cycloalkyl radicals attached to a sulfinyl radical, where cycloalkyl is defined as above. “Cycloalkylsulfinylalkyl”, embraces cycloalkylsulfinyl radicals attached to an alkyl radical, where alkyl is defined as above. The term “Cycloalkylsulfonyl”, embraces cycloalkyl radicals attached to a sulfonyl radical, where cycloalkyl is defined as above. “Cycloalkylsulfonylalkyl”, embraces cycloalkylsulfonyl radicals attached to an alkyl radical, where alkyl is defined as above.

The term “cycloalkylalkanoyl” embraces radicals wherein one or more of the cycloalkyl carbon atoms are substituted with one or more carbonyl radicals as defined below. Specifically embraced are monocarbonylcycloalkyl and dicarbonylcycloalkyl radicals. Examples of monocarbonylcycloalkyl radicals include cyclohexylcarbonyl, cyclohexylacetyl, and cyclopentylcarbonyl. Examples of dicarbonylcycloalkyl radicals include 1,2-dicarbonylcyclohexane.

The term “alkylthio” embraces radicals containing a linear or branched alkyl radical, of one to ten carbon atoms, attached to a divalent sulfur atom. More preferred alkylthio radicals are “alk-ylthio” radicals having one to six carbon atoms. An example of “alkylthio” is methylthio (CH₃—S—). The “alkylthio” radicals may be further substituted with one or more halo atoms, such as fluoro, chloro or bromo, to provide “haloalklylthio” radicals. Examples of such radicals include fluoromethylthio, chloromethylthio, trifluoromethylthio, difluoromethylthio, trifiuoroethylthio, fluoroethylthio, tetrluoroethylthio, pentafluoroethylthio, and fluoropropylthio.

The term “alkyl aryl amino” embraces radicals containing a linear or branched alkyl radical, of one to ten carbon atoms, and one aryl radical both attached to an amino radical. Examples include N-methylmethoxyaniline, N-ethyl-4-methoxyaniline, and N-methyltrifluoromethoxyaniline.

The term alkylamino denotes “monoalkylamino” and “dialkylamino” containing one or two alkyl radicals, respectively, attached to an amino radical. One or two alkyl radicals of the alkylamino may be optionally substituted with hydrogen bonding substitutents selected from the group consisting of hydroxy, amino, monoalkylamino, dialkylamino, amidino, guanidino, thiol, and alkoxy provided the alkyl radicals comprises two or more carbons.

The terms arylamino denotes “monoarylamino” and “diarylamiino” containing one or two aryl radicals, respectively, attached to an amino radical. Examples of such radicals include N-phenylamino and N-naphthylamino.

The term “aralkylamino”, embraces aralkyl radicals attached to an amino radical, where aralkyl is defined as above. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “arylsulfinyl” embraces radicals containing an aryl radical, as defined above, attached to a divalent S(O) atom. The term “arylsulfinylalkyl” denotes arylsulfinyl radicals attached to a linear or branched alkyl radical, of one to ten carbon atoms.

The term “arylsulfonyl”, embraces aryl radicals attached to a sulfonyl radical, where aryl is defined as above. “arylsulfonylalkyl”, embraces arylsulfonyl radicals attached to an alkyl radical, where alkyl is defined as above. The term “heteroarylsulfinyl” embraces radicals containing an heteroaryl radical, as defined above, attached to a divalent S(O) atom. The term “heteroarylsulfinylalkyl” denotes heteroarylsulfinyl radicals attached to a linear or branched alkyl radical, of one to ten carbon atoms. The term “Heteroarylsulfonyl”, embraces heteroaryl radicals attached to a sulfonyl radical, where heteroaryl is defined as above. “Heteroarylsulfonylalkyl”, embraces heteroarylsulfonyl radicals attached to an alkyl radical, where alkyl is defined as above.

The term “aryloxy” embraces aryl radicals, as defined above, attached to an oxygen atom. Examples of such radicals include phenoxy, 4-chloro-3-ethylphenoxy, 4-chloro-3-methylphenoxy, 3-chloroethylphenoxy, 3,4-dichlorophenoxy, 4-methylphenoxy, 3-trifluoromethoxyphenoxy, 3-triuoromethylphenoxy, 4-fluorophenoxy, 3,4-dimethylphenoxy, 5-bromo-2-fluorophenoxy, 4-bromo-3-fluorophenoxy, 4-fluoro-3-methylphenoxy, 5,6,7,8-tetrahydronaphthyloxy, 3-isopropylphenoxy, 3-cyclopropylphenoxy, 3-ethylphenoxy, 3-pentafuoroethylphenoxy, 3-(1,1,2,Ztetrafluoroethoxy)-phenoxy, and 4-tert-butylphenoxy.

The term “aroyl” embraces aryl radicals, as defined above, attached to an carbonyl radical as defined above. Examples of such radicals include benzoyl and toluoyl.

The term “aralkanoyl” embraces aralkyl radicals, as defined herein, attached to an carbonyl radical as defined above. Examples of such radicals include, for example, phenylacetyl.

The term “aralkoxy” embraces oxy-containing aralkyl radicals attached through an oxygen atom to other radicals. More preferred aralkoxy radicals are “aralkoxy” radicals having phenyl radicals attached to alkoxy radical as described above. Examples of such radicals include benzyloxy, 1-phenylethoxy, 3-trifluoromethoxybenzyloxy, 3-trifluoromethylbenzyloxy, 3,5-difluorobenyloxy, 3-bromobenzyloxy, 4-propylbenzyloxy, 2-fluoro-3-trifluoromethylbenzyloxy, and 2-phenylethoxy.

The term “aryloxyalkyl” embraces aryloxy radicals, as defined above, attached to an alkyl group. Examples of such radicals include phenoxymethyl.

The term “haloaryloxyalkyl” embraces aryloxyalkyl radicals, as defined above, wherein one to five halo radicals are attached to an aryloxy group.

The term “heteroaroyl” embraces heteroaryl radicals, as defined above, attached to an carbonyl radical as defined above. Examples of such radicals include furoyl and nicotinyl.

The term “heteroaralkanoyl” embraces heteroaralalkyl radicals, as defined herein, attached to an carbonyl radical as defined above. Examples of such radicals include, for example, pyridylacetyl and furylbutyryl.

The term “heteroaralkoxy” embraces oxy-containing heteroaralkyl radicals attached through an oxygen atom to other radicals. More preferred heteroaralkoxy radicals are “heteroaralkoxy” radicals having heteroaryl radicals attached to alkoxy radical as described above. The term “heterocyclylalkoxy” embraces oxy-containing heterocyclylalkyl radicals attached through an oxygen atom to other radicals.

The term “haloheteroaryloxyalkyl” embraces heteroaryloxyalkyl radicals, as defined above, wherein one to four halo radicals are attached to an heteroaryloxy group.

The term “heteroarylamino” embraces heteroaryl radicals, as defined above, attached to an amino group. Examples of such radicals include pyridylamino. The term “heterocyclylamino” embraces heterocyclyl radicals, as defined above, attached to an amino group.

The term “heteroaralkylamino” embraces heteroaralkyl radicals, as defined above, attached to an amino group. Examples of such radicals include pyridylmethylamino. The term “heterocyclylalkylamino” embraces heterocyclylalkyl radicals, as defined above, attached to an amino group.

The term “heteroaryloxy” embraces heteroaryl radicals, as defined above, attached to an oxy group. Examples of such radicals include 2-thiophenyloxy, 2-pyrirnidyloxy, 2-pyridyloxy, 3-pyridyloxy, and 4-pyridyloxy. The term “heterocyclyloxy” embraces heterocyclyl radicals, as defined above, attached to an oxy group.

The term “heteroaryloxyalkyl” embraces heteroaryloxy radicals, as defined above, attached to an alkyl group. Examples of such radicals include 2-pyridyloxymethyl, 3-pyridyloxyethyl, and 4-pyridyloxymethyl. The term “heterocyclyloxyalkyl” embraces heterocyclyloxy radicals, as defined above, attached to an alkyl group.

The term “arylthio” embraces aryl radicals, as defined above, attached to an sulfur atom. Examples of such radicals include phenylthio.

The term “arylthioalkyl” embraces arylthio radicals, as defined above, attached to an alkyl group. Examples of such radicals include phenylthiomethyl.

The term “alkylthioalkyl “embraces alkylthio radicals, as defined above, attached to an alkyl group. Examples of such radicals include methylthiomethyl. The term “alkoxyalkyl” embraces alkoxy radicals, as defined above, attached to an alkyl group. Examples of such radicals include methoxymethyl.

The term “carbonyl” denotes a carbon radical having two of the four covalent bonds shared with an oxygen atom. The term “carboxy” embraces a hydroxyl radical, as defined above, attached to one of two unshared bonds in a carbonyl group. The term “carboxamido” embraces amino, monoalkylamino, dialkylamino, monocycloalkylamino, alkylcycloalkylamino, dicycloalkylamino, N-alkyl-N-arylamino, arylamino, aralkylamino, nitrogen containing heterocyclyl, heterocyclylamino, N-alkyl-N-heterocyclylamino, heteroarylamino, and heteroaralkylamino radicals, attached to one of two unshared bonds in a carbonyl group. The term “carboxamidoalkyl” embraces carboxamido radicals, as defined above, attached to an alkyl group. The term “carboxyalkyl” embraces a carboxy radical, as defined above, attached to an alkyl group. The term “carboalkoxy” embraces alkoxy radicals, as defined above, attached to one of two unshared bonds in a carbonyl group. The term “carboaralkoxy” embraces aralkoxy radicals, as defined above, attached to one of two unshared bonds in a carbonyl group. The term “monocarboalkoxyalkyl” embraces one carboalkoxy radical, as defined above, attached to an alkyl group. The term “dicarboalkoxyalkyl” embraces two carboalkoxy radicals, as defined above, attached to an alkylene group. The term “monocyanoalkyl” embraces one cyano radical, as defined above, attached to an alkyl group. The term “dicyanoalkylene” embraces two cyano radicals, as defined above, attached to an alkyl group. The term “carboalkoxycyanoalkyl” embraces one cyano radical, as defined above, attached to an carboalkoxyalkyl group.

The term “acyl”, alone or in combination, means a carbonyl or thionocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like. The term “haloalkanoyl” embraces one or more halo radicals, as defined herein, attached to an alkanoyl radical as defined above. Examples of such radicals include, for example, chloroacetyl, trifluoroacetyl, bromopropanoyl, and heptafluorobutanoyl.

The term “phosphono” embraces a pentavalent phosphorus attached with two covalent bonds to an oxygen radical. The term “dialkoxyphosphono” denotes two alkoxy radicals, as defined above, attached to a phosphono radical with two covalent bonds. The term “diaralkoxyphosphono” denotes two aralkoxy radicals, as defined above, attached to a phosphono radical with two covalent bonds. The term “dialkoxyphosphonoalkyl” denotes dialkoxyphosphono radicals, as defined above, attached to an alkyl radical. The term “diaralkoxyphosphonoalkyl” denotes diamlkoxyphosphono radicals, as defined above, attached to an alkyl radical.

The term “amino” denotes a nitrogen atom containing two substituents such as hydrido, hydroxy or alkyl and having one covalent bond available for bonding to a single atom such as carbon. Examples of such amino radicals include, for example, —NH₂, —NHCH₃, —NHOH, and —NHOCH₃. The term “imino” denotes a nitrogen atom containing one substituent such as hydrido, hydroxy or alkyl and having two covalent bonds available for bonding to a single atom such as carbon. Examples of such imino radicals include, for example, ═NH, ═NCH₃, ═NOH, and —NOCH₃. The term “imino carbonyl” denotes a carbon radical having two of the four covalent bond sites shared with an imino group. Examples of such imino carbonyl radicals include, for example, C═NH, C═NCH₃, C═NOH, and C═NOCH₃. The term “amidino” embraces a substituted or unsubstituted amino group bonded to one of two available bonds of an iminocarbonyl radical. Examples of such anidino radicals include, for example, NH₂—C═NH, NH₂C═NCH₃, NH₂—C═NOCH₃ and CH₃NH—C═NOH. The term “guanidino” denotes an amidino group bonded to an amino group as defined above where said amino group can be bonded to a third group. Examples of such guanidino radicals include, for example, NH₂—C(NH)—NH—, NH₂—C(NCH₃)—NH—, NH₂—C(NOCH₃)—NH—, and CH₃NH—C(NOH)—NH—.

The term “sulfonium” denotes a positively charged trivalent sulfur atom where said sulfur is substituted with three carbon based groups such as alkyl, alkenyl, aralkyl, or aryl. The term “dialkyl sulfonium” denotes a sulfonium group where said sulfur is substituted with two alkyl groups. Examples of such dialkylsulfonium radicals include, for example, (CH₃)₂S⁺—. The term “dialkyl sulfonium alky” denotes a dialkyl sulfonium group where said group is bonded to one bond of an alkylene group as defined above. Examples of such dialkylsulfoniumalkyl radicals include (CH₃)₂S⁺—CH₂CH₂—.

The term “phosphonium” denotes a positively charged tetravalent phosphorus atom where said phosphorus is substituted with four carbon based groups such as alkyl, alkenyl, aralkyl, or aryl. The term “trialkyl phosphonium” denotes a phosphonium group where said phosphorus is substituted with three alkyl groups. Examples of such trialkylphosphonium radicals include, for example, (CH₃)₃P⁺—.

Said “alkyl”, “alkenyl”, “alkynyl”, “alkanoyl”, “alkylene”, “alkenylene”, “hydroxyalkyl”, “haloalkyl”, “haloalkylene”, “haloalkenyl”, “alkoxy”, “alkenyloxy”, “alkenyloxyalkyl”, “alkoxyalkyl”, “aryl”, “perhaloaryl”, “haloalkoxy”, “haloalkoxyalkyl”, “haloalkenyloxy”, “haloalkenyloxyalkyl”, “alkylenedioxy”, “haloalkylenedioxy”, “heterocyclyl”, “heteroaryl”, “hydroxyhaloalkyl”, “alkylsulfonyl”, “haloalkylsulfonyl”, “alkylsulfonylalkyl”, “haloalkylsulfonylalkyl”, “alkylsulfinyl”, “alkylsulfinylalkyl”, “haloalkylsulfinylalkyl”, “aralkyl”, “heteroaralkyl”, “perhaloaralkyl”, “aralkylsulfonyl”, “aralkylsulfonylalkyl”, “aralkylsulfinyl”, “aralkylsulfinylalkyl”, “cycloalkyl”, “cycloalkylalkanoyl”, “cycloalkylalkyl”, “cycloalkenyl”, “halocycloalkyl”, “halocycloalkenyl”, “cycloalkylsulfinyl”, “cycloalkylsulfinylalkyl”, “cycloalkylsulfonyl”, “cycloalkylsulfonylalkyl”, “cycloalkoxy”, ^{t}“cycloalkoxyalkyl”, “cycloalkylalkoxy”, “cycloalkenyloxy”, “cycloalkenyloxyalkyl”, “cycloalkylenedioxy”, “halocycloalkoxy”, “halocycloalkoxyalkyl”, “halocycloalkenyloxy”, “halocycloalkenyloxyalkyl”, “alkylthio”, “haloalkylthio”, “alkylsulfinyl”, “amino”, “oxy”, “thic”, “alkylamino”, “arylamino”, “aralkylamino”, “arylsulfinyl”, ““arylsulfinylalkyl”, “arylsulfonyl”, “arylsulfonylalkyl”, “heteroarylsulfinyl”, “heteroarylsulfinylalkyl”, “heteroarylsulfonyl”, “heteroarylsulfonylalkyl”, “heteroarylamino”, “heteroaralkylamino”, “heteroaryloxy”, “heteroaryloxylalkyl”, “aryloxy”, “aroyl”, “aralkanoyl”, “aralkoxy”, “aryloxyalkyl”, “haloaryloxyalkyl”, “heteroaroyl”, “heteroaralkanoyl”, “heteroaralkoxy”, “heteroaralkoxyalkyl”, “arylthio”, “arylthioalkyl”, “alkoxyalkyl”, “acyl”, “amidino”, “guanidino”, “dialkylsulfonium”, “trialkylphosphonium”, and “dialkylsulfoniumalkyl” groups defined above may optionally have 1 or more non-hydrido substituents such as amidino, guanidino, dialkylsulfonium, trialkylphosphonium, dialkylsulfoniumalkyl, perhaloamalkyl, aralkylsulfonyl, aralkylsulfonylalkyl, aralkylsulfinyl, amikylsulfinylalkyl, halocycloalkyl, halocycloalkenyl, cycloalkylsulfinyl, cycloalkylsufllnylalkyl, cycloalkylsulfonyl, cycloalkylsulfonylalkyl, heteroarylamino, N-heteroarylamino-N-alkylamino, heteroaralkylamino, heteroaryloxy, heteroaryloxylalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroarlkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, cycloalkylalkyl, cycloalkenylalkyl, halo, haloalkyl, haloalkenyl, haloalkoxy, hydroxyhaloalkyl, hydroxyaralkyl, hydroxyalkyl, aminoalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroaralkyl, arylalkenyl, heteroarylalkenyl, carboxyalkyl, carboalkoxy, alkoxycarbonyl, carboaralkoxy, carboxamido, carboxamidoalkyl, cyano, carbohaloalkoxy, phosphono, phosphonoalkyl, diaralkoxyphosphono, and diaralkoxyphosphonoalkyl.

The term “spacer” can include a covalent bond and a linear moiety having a backbone of 1 to 7 contiguous atoms. The spacer may have 1 to 7 atoms of a univalent or multi-valent chain. Univalent chains may be constituted by a radical selected from ═C(H)—, ═C(R^{2a})—, —O—, —S—, —S(O)—, —S(O)₂—, —NH—, —N(R^{2a}, —N═, —CH(OH)—, ═C(OH)—, —CH(OR^{2a})—, ═C(OR)—, and —C(O)— wherein R^{2a} is selected from alkyl, alkenyl, alkynyl, aryl, heteroaryl, aralkyl, aryloxyalkyl, alkoxyalkyl, alkylthioalkyl, arylthioalkyl, cycloalkyl, cycloalkylalkyl, haloalkyl, haloalkenyl, haloalkoxyalkyl, perhaloaralkyl, heteroarylalkyl, heteroaryloxyalkyl, heteroarylthioalkyl, and heteroarylalkenyl. Multi-valent chains may consist of a straight chain of 1 or 2 or 3 or 4 or 5 or 6 or 7 atoms or a straight chain of 1 or 2 or 3 or 4 or 5 or 6 atoms with a side chain. The chain may be constituted of one or more radicals selected from: alkylene, alkenyl, —O—, —CH₂—, —S—CH₂—, —CH₂CH₂—, ethenyl, —CH═CH(OH)—, —OCH₂O—, —O(CH₂)₂O—, —NHCH₂—, —OCH(O)O—, —O(CH₂CHR^{2a})O—, —OCF₂O—, —O(CF₂₂O—, —S—, —S(O)—, —S(O)₂-, —N(H)—, —N(H)O—, —N(R^{2a})O—, —N(R^{2a})—, —C(O)—, —C(O)NH—, —C(O)NR^{2a}—,N═, —OCH₂—, —SCH₂—, S(O)CH₂—, —CH₂C(O)—, —CH(OH)—, ═C(OH)—, —CH(OR^{2a}), ═C(OR^{2a})—, S(O)₂CH₂—, and —NR^{2a}CH₂— and many other radicals defined above or generally known or ascertained by one of skill-in-the art. Side chains may include substituents such as 1 or more non-hydrido substituents such as amidino, guanidino, dialkylsulfonium, trialkylphosphonium, dialkylsulfoniumalkyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroaralkylamino, heteroaryloxy, heteroaryloxylalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, thio, nitro, alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsulfinyl, heteroarylsulfonyl, alkanoyl, alkenoyl, aroyl, heteroaroyl, aralkanoyl, heteroaalkanoyl, haloalkanoyl, alkyl, alkenyl, alkynyl, alkenyloxy, alkenyloxyalky, alkylenedioxy, haloalkylenedioxy, cycloalkyl, cycloalkenyl, cycloalkylalkyl, cycloalkenylalkyl, halo, haloalkyl, haloalkenyl, haloalkoxy, hydroxyhaloalkyl, hydroxyaralkyl, hydroxyalkyl, aminoalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboxyalkyl, carboalkoxy, carboaralkoxy, carboxamido, carboxamidoalkyl, cyano, carbohaloalkoxy, phosphono, phosphonoalkyl, diaralkoxyphosphono, and diaralkoxyphosphonoalkyl.

Compounds of the present invention can exist in tautomeric, geometric or stereoisomeric forms. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof, as falling within the scope of the invention. Pharmaceutically acceptable sales of such tautomeric, geometric or stereoisomeric forms are also included within the invention.

The terms “cis” and “trans” denote a form of geometric isomerism in which two carbon atoms connected by a double bond will each have a hydrogen atom on the same side of the double bond (“cis”) or on opposite sides of the double bond (“trans”).

Some of the compounds described contain alkenyl groups, and are meant to include both cis and trans or “E” and “Z” geometric forms.

Some of the compounds described contain one or more stereocenters and are meant to include R, S, and mixtures of R and S forms for each stereocenter present.

Some of the compounds described herein may contain one or more ketonic or aldehydic carbonyl groups or combinations thereof alone or as part of a heterocyclic ring system. Such carbonyl groups may exist in part or principally in the “keto” form and in part or principally as one or more “enol” forms of each aldehyde and ketone group present. Compounds of the present invention having aldehydic or ketonic carbonyl groups are meant to include both “keto” and “enol” tautomeric forms.

Some of the compounds described herein may contain one or more amide carbonyl groups or combinations thereof alone or as part of a heterocyclic ring system. Such carbonyl groups may exist in part or principally in the “keto” form and in part or principally as one or more “enol” forms of each amide group present. Compounds of the present invention having amidic carbonyl groups are meant to include both “keto” and “enol” tautomeric forms. Said amide carbonyl groups may be both oxo (CEO) and thiono (C═S) in type.

Some of the compounds described herein may contain one or more imine or enamine groups or combinations thereof. Such groups may exist in part or principally in the “imine” form and in part or principally as one or more “enamine” forms of each group present. Compounds of the present invention having said imine or enamine groups are meant to include both “imine” and “enamine” tautomeric forms.

The present invention also comprises a treatment and prophylaxis in anticoagulant therapy for the treatment and prevention of a variety of thrombotic conditions including coronary artery and cerebrovascular disease in a subject, comprising administering to the subject having such disorder a therapeutically-effective amount of a compound of Formula (I):

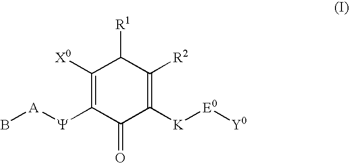

or a pharmaceutically-acceptable salt thereof.

As a further embodiment, compounds of the present invention of Formula (I) or a pharmaceutically-acceptable salt thereof as defined above, comprise a treatment and prophylaxis of coronary artery disease, cerebrovascular disease and other coagulation cascade related disorders in a subject, comprising administering to the subject having such disorder a therapeutically-effective amount of compounds of formula (I) of the present invention or a pharmaceutically-acceptable salt thereof.

Compounds of the present invention of Formula (I) or a pharmaceutically-acceptable salt thereof can also be used whenever inhibition of blood coagulation is required such as to prevent coagulation of stored whole blood and to prevent coagulation in other biological samples for testing or storage. Thus coagulation inhibitors of the present inhibition can be added to or contacted with stored whole blood and any medium containing or suspected of containing plasma coagulation factors and in which it is desired that blood coagulation be inhibited, e.g. when contacting the mammal's blood with material selected from the group consisting of vascular grafts, stents, orthopedic prothesis, cardiac prosthesis, and extracorporeal circulation systems.

Compounds of Formula (I) are capable of inhibiting activity of serine proteases related to the coagulation cascade, and thus could be used in the manufacture of a medicament, a method for the prophylactic or therapeutic treatment of diseases mediated by coagulation cascade serine proteases, such as inhibiting the formation of blood platelet aggregates, inhibiting the formation of fibrin, inhibiting thrombus formation, and inhibiting embolus formation in a mammal, in blood, in blood products, and in mammalian organs. The compounds also can be used for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels in a mammal. The compounds also can be used to study the mechanism of action of coagulation cascade serine proteases to enable the design of better inhibitors and development of better assay methods. The compounds of Formula (I) would be also useful in prevention of cerebral vascular accident (CVA) or stroke.

Also included in the family of compounds of Formula (I) are the pharmaceutically-acceptable salts thereof. The term “pharmaceutically-acceptable salt” embraces salts commonly used to form alkali metal salts and to form addition salts of free acids or free bases. The nature of the salt is not critical, provided that it is pharmaceutically acceptable. Suitable pharmaceutically-acceptable acid addition salts of compounds of Formula (I) may be prepared from inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, galacturonic acid. Suitable pharmaceutically-acceptable base addition salts of compounds of Formula (I) include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compound of Formula (I) by reacting, for example, the appropriate acid or base with the compound of Formula (I).

The present invention also comprises a pharmaceutical composition comprising a therapeutically-effective amount of a compound of Formulas (I) in association with at least one pharmaceutically-acceptable carrier, adjuvant or diluent. Pharmaceutical compositions of the present invention can comprise the active compounds of Formula (I) in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended.

The active compounds and composition may, for example, be administered orally, intravascularly, intraperitoneally, subcutaneously, intramuscularly, oculary, or topically. For treating ocular build up of fibrin, the compounds may be administered intraocularly or topically as well as orally or parenterally.

The compounds can be administered in the form of a depot injection or implant preparation which may be formulated in such a manner as to permit a sustained release of the active ingredient The active ingredient can be compressed into pellets or small cylinders and implanted subcutaneously or intramusculary as depot injections or implants. Implants may employ inert materials such as biodegradable polymers or synthetic silicones, for example, Silastic, silicone rubber or other silicon containing polymers.

The compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxy-propyl-methacrylamide-phenol, polyhydroxyethyl-aspartmide-phenol, or ployethyleneoxide-polylysine substituted with palritoyl residues.

Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphitpathic block copolymers of hydrogels.

For oral administration, the pharmaceutical composition may be in the form of, for example, tablets, capsules (each of which includes sustained release or timed release formulations), pills, powders, granules, elixers, tinctures, suspensions, liquids including syrups, and emulsions. The pharmaceutical composition is preferably made in the form of a dosage unit containing a particular amount of the active ingredient. Examples of such dosage units are tablets or capsules. The active ingredient may also be administered by injection as a composition wherein, for example, saline, dextrose or water may be used as a suitable carrier.

The amount of therapeutically active compounds which are administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely.

The pharmaceutical compositions may contain active ingredients in the range of about 0.1 to 2000 mg, and preferably in the range of about 0.5 to 500 mg. A daily dose of about 0.01 to 100 mg/kg body weight, and preferably between about 0.5 and about 20 mg/kg body weight, may be appropriate. The daily dose can be administered in one to four doses per day.

The compounds may be formulated in topical ointment or cream, or as a suppository, containing the active ingredients in a total amount of, for example, 0.075 to 30% w/w, preferably 0.2 to 20% w/w and most preferably 0A to 15% w/w. When formulated in an ointment, the active ingredients may be employed with either paraffimic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the socalled emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

For therapeutic purposes, the active compounds of the present invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

In practicing the methods of the present invention for the treatment and prevention of a variety of thrombotic conditions including coronary artery and cerebrovascular disease, the compounds and pharmaceutical compositions of the present invention are administered alone or in combination with one another, or in combination with other therapeutics or in vivo diagnostic agents. The coagulation cascade inhibitors of the present invention can also be co-administered with suitable anti-platelet agreggation agents, including, but not limited to ticlopidine or clopidrogel, fibrinogen receptor antagonists (e.g. to treat or prevent unstable angina or to prevent reocculsion after angioplasty and restenosis), anti-coagulants such as aspirin, warfarin or heparins, thrombolytic agents such as plasminogen activators or streptokinase to achieve synergistic effects in the treatment of various pathologies, lipid lowering agents including antihypercholesterolemics (e.g. HMG CoA reductase inhibitors such as mevastatin, lovastatin, simvastatin, pravastatin, and fluvastatin, HMG CoA synthatase inhibitors, etc.), anti-diabetic drugs, or other cardiovascular agents (loop diuretics, thiazide type diuretics, nitrates, aldosterone antagonistics (i.e., spironolactone and epoxymexlerenone), angiotensin converting enzyme (e.g. ACED inhibitors, angiotensin II receptor antagonists, beta-blockers, antiarrythmics, anti-hypertension agents, and calcium channel blockers) to treat or prevent atheriosclerosis. For example, patients suffering from coronary artery disease, and patients subjected to angioplasty procedures, would benefit from coadministration of fibrinogen receptor antagonists and coagulation cascade inhibitors of the present invention. Also, coagulation cascade inhibitors could enhance the efficiency of tissue plasminogen activator-mediated thrombolytic reperfusion.

Typical doses of coagulation cascade inhibitors of the present invention with other suitable anti-platelet agents, anticoagulation agents, cardiovascular therapeutic agents, or thrombolytic agents may be the same as those doses of coagulation cascade inhibitors administered without coadministration of additional anti-platelet agents, anticoagulation agents, cardiovascular therapeutic agents, or thrombolytic agents, or may be substantially less than those doses of coagulation cascade inhibitors administered without coadministration of additional anti-platelet agents, anticoagulation agents, cardiovascular therapeutic agents, or thrombolytic agents, depending on a patient's therapeutic needs.

The present novel methods preferably employ compounds which selectively inhibit human TF-VIIA over the inhibition of both human Thrombin II and human factor Xa. Preferably, the compounds have a human TF-VIIA IC₅₀ of less than 0.5 μM and also have a selectivity ratio of TF-VIIA inhibition over both human Thrombin II and human factor Xa inhibition of at least 10, and more preferably at least 100. Even more preferably, the compounds have a human TF-VIIA IC₅₀ of less than 0.1 μM and also have a selectivity ratio of TF-VIIA inhibition over both human Thrombin II and human factor Xa inhibition of at least 1000, and most preferably at least 10,000.

All mentioned references are incorporated by reference as if here written.

Although this invention has been described with respect to specific embodiments, the details of these embodiments are not to be construed as limitations. The following examples are provided to illustrate the present invention and are not intended to limit the scope thereof. Without further elaboration, it is believed that one skilled in the art can, using the preceding descriptions, utilize the present invention to its fullest extent. Therefore the following preferred specific embodiments are to be construed as merely illustrative and not limitative of the remainder of the disclosure in any way whatsoever. Compounds containing multiple variations of the structural modifications illustrated in the schemes or the following Examples are also contemplated. Those skilled in the art will readily understand that known variations of the conditions and processes of the following preparative procedures can be used to prepare these compounds.

One skilled in the art may use these generic methods to prepare the following specific examples, which have been or may be properly characterized by ¹H NMR, mass spectrometry, elemental composition, and similar procedures. These compounds also may be formed in vivo. The following examples contain detailed descriptions of the methods of preparation of compounds of Formula (a). These detailed descriptions fall within the scope and are presented for illustrative purposes only and are not intended as a restriction on the scope of the invention. All parts are by weight and temperatures are Degrees centigrade unless otherwise indicated. The following general synthetic sequences are useful in making the present invention. Abbreviations used in the schemes are as follows: “AA” represents amino acids, “AcCN” represents acetonitrile, “AcOH” represents acetic acid, “BINAP” represents 2,2′-bis(diphenylphosphino)-1,1′-binaphthyl, “BnOH” represents benzyl alcohol, “BnCHO” represents 2-phenylethanal, “BnSO₂Cl” represents benzylsulfonyl chloride, “Boc” represents tert-butyloxycarbonyl, “BOP” represents benzotriazol-1-yl-oxy-tris-(dimethylamino), “bu” represents butyl, “dba” represents dibenzylideneacetone, “DCC” represents 1,3-dicyclohexylcarbodiimide, “DCM” represents dichloromethane or methylene chloride, “DIBAH” or “DIBAL” represents diisobutylaluminum hydride, “DMF” represents dimethylformamide, “DMSO” represents dimethylsulfoxide, “DPPA” represents diphenylphosphoryl azide”, “EDC” represents 1-[3-(dimethylamino)propyl]-3-ethylcarbodiimide hydrochloride, “Fmoc” represents 9-fluorenylmethoxycarbonyl, “HOBT” represents hydroxybenzoltriazole”, “LDA” represents lithium diisopropylamide, “NMM” represents N-methylmorpholine, “Ph” represents phenyl or aryl, “PHTH” represents a phthaloyl group, “pnZ” represents 4 nitrobenzyloxycarbonyl, “PTC” represents a phase transfer catalyst, “py” represents pyridine, “RNH2” represents a primary organic amine, “p-TsOH” represents paratoluenesulfonic acid, “TBAF” represents tetrabutylamnuonium fluoride, “TBTU” represents 2(1H-benzotriozole-1-yl)-1,1,3,3-tetramethyl uronium tetmfluoroborate, “TEA” represents triethylamine, “TFA” represents trifluoroacetic acid, “ThF” represents tetrahydrofuran, “TMS” represents trimethylsilyl, ““TMSCN” represents trimethylsilyl cyanide, and “Cbz” or “Z” represents benzyloxycarbonyl.

### GENERAL SYNTHETIC PROCEDURES AND SPECIFIC EXAMPLES

The 4-pyridone compounds of the present invention can be synthesized, for example, according to the following procedures and Schemes given below.

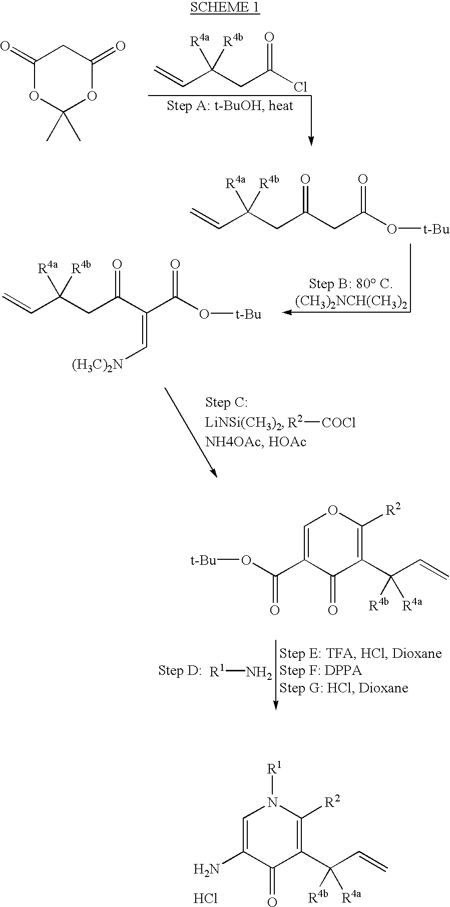

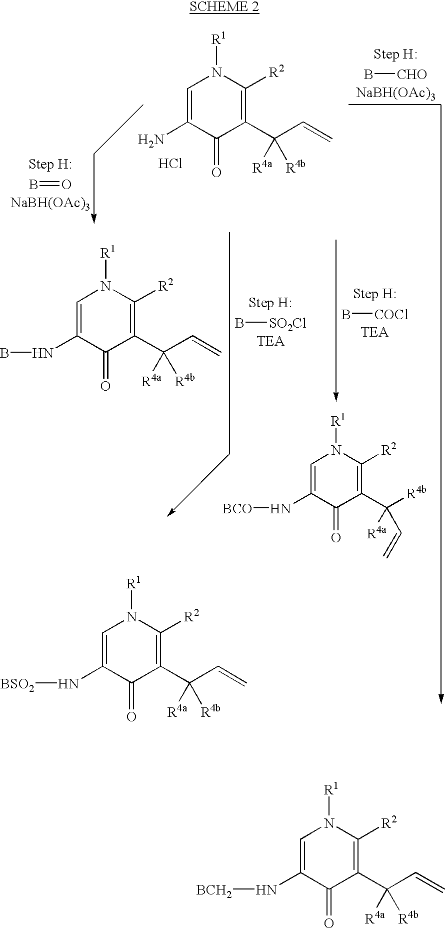

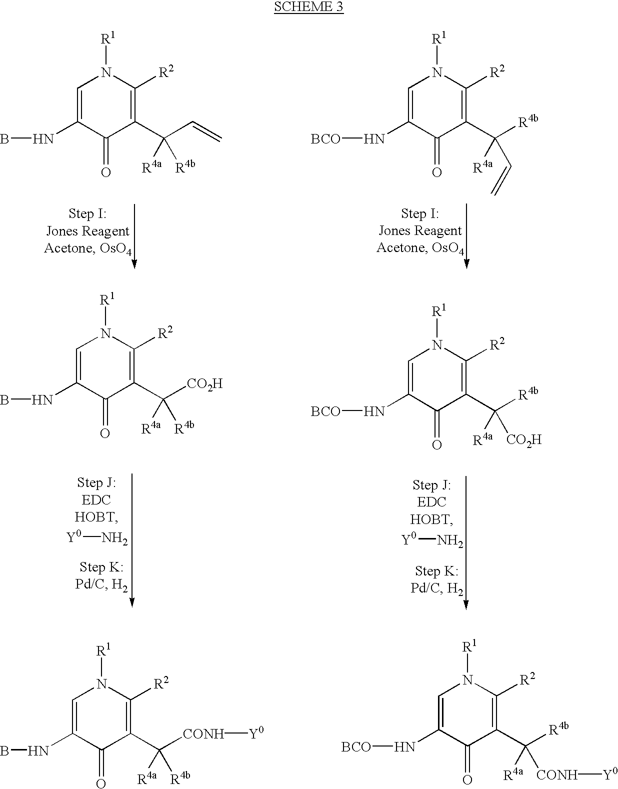

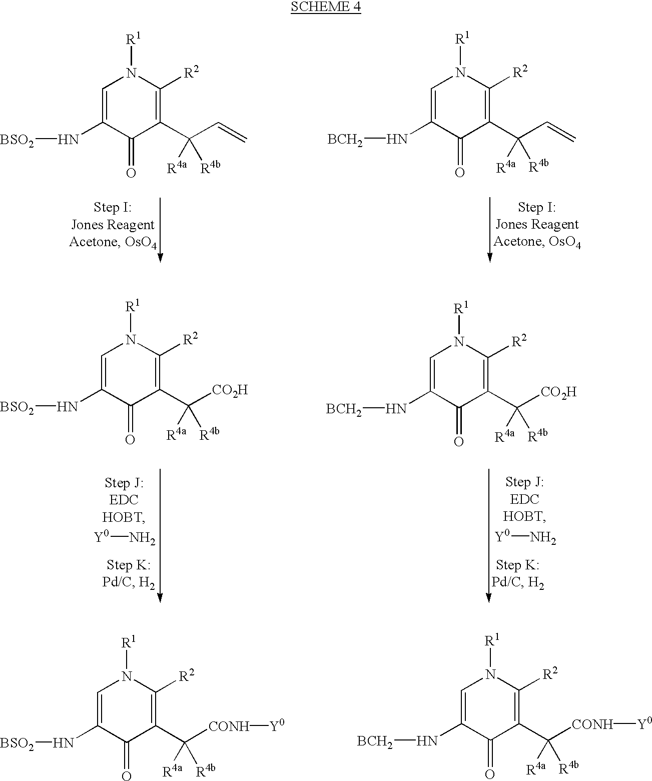

Schemes 1, 2, 3, and 4 above summarize generic procedures that permit the preparation of a wide variety of the compounds of the present invention through the ability to introduce numerous R² substituents represented by Z^{o}-Q, numerous R¹ substituents, a wide variety of amino substituting groups represented by B-, B-CH₂, B-CO, and B-SO₂, and a large number of amide forming Y^{o} groups at the carboxylic acid group in which E^{o} is C(O)NH. Examples 1 and 2 below describe the preparation of two representative compounds wherein X^{o} is hydrido and R¹ is methyl and propyl, respectively.

### Example 1

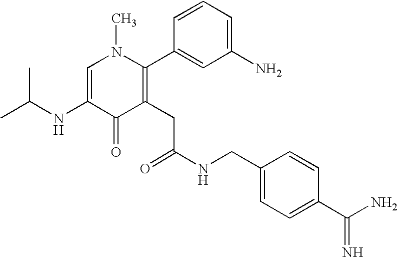

To a solution of 2,2-dimethyl-1,3-dioxane-“one (74.93 g, 0.52 mol) and pyridine (86.0 mL, 1.06 mol) in CH₂Cl₂ (1.0 L) at 0° C. was added 4-pentenoyl chloride (62.5 mL, 0.57 mol) over 15 hrs. After an additional 40 min, the reaction mixture was allowed to warm to room temperature. After an additional 1 hour, the reaction mixture was concentrated in vacuo, and the residue was dissolved in t-BuOH (750 mL). The mixture was heated to reflux for 4 hrs, and then concentrated in vacuo. The residue was triturated with ethyl acetate (500 mL) and filtered. The filtrate was concentrated in vacuo to give a dark red oil EX-1A (64.95 g, 63%). ¹H NMR (CDCl₃) δ 1.43 (s, 9H), 2.31 (m, 2H), 2.60 (t, J=7.2 Hz, 2H), 3.32 (s, 2H), 4.93-5.04 (m, 2H), 5.70-5.84 (m, 114). ¹³C NMR (CDCl₃) δ 27.3, 27.9, 41.8, 50.6, 115.4, 136.6, 166.3, 202.4. LRMS (ESI) [M+Li]⁺ 205.

A mixture of EX-1A (130.0 g, 0.6557 mol) and N,N-dimethylformamide dimethyl acetal (117.2 g, 0.9836 mol) was heated to 80° C. for 2 hrs. The reaction mixture was concentrated in vacuo and purified by flash chromatography on silica gel (EtOAc/hexane) to give a yellow oil EX-1B (140.9 g/85%). ¹H NMR (CDCl₃) δ 1.50 (s, 9H), 2.31-2.40 (m, 2H), 2.71 (t, J=7.8 Hz, 2H), 3.44 (s, 6H), 4.93 (dd, J=9.9, 1.8 Hz, 1H), 4.97-5.05 (m, 1H), 5.T7-5.90 (m, 1H), 7.53 (s, 1H). HRMS (ESI), calc'd for C₁₄H₂₃NO₃ 254.1756, found 254.1753.

A solution of EX-1B (2.50 g, 9.87 mmol) in THF (25.0 mL) was added to LiHMDS (25.0 mL, 25.0 mmol, IM in THF) at −70° C. After 6 min, a solution of 3-nitrobenzoyl chloride (2.19 g, 11.80 mmol) in THF (25 mL) was added dropwise over 9 min. After 2 min. more, the cooling bath was removed, and ethyl ether (20 mL) and 3N aq. HC (30 mL) was added. The mixture's pH=2. After 1 hr, the layers were separated, and the organic layer was washed with saturated aqueous NaHCO₃ (2×30 mL) and saturated aqueous NaCl (25 mL). The aqueous layer was extracted with ethyl ether (2×25 mL). Combined organic layers were dried (MgSO₄), filtered and concentrated in vacuo to give a dark reddish brown viscous liquid. Purification by flash chromatography on silica gel eluting with EtOAc/hexanes gave an off-white solid EX-IC (1.658 g/47%). ¹H NMR (CDCl₃) δ 1.59 (s, 9H), 3.24 (dd, J=4.0, 1.61 Hz, 2H), 5.05 (dd, J=17.2, 1.2 Hz, 1H), 5.15 (dd, J=10.0, 1.2 Hz, 1H), 5.97-6.07 (m, 1H), 7.70 (t, J=8.0 Hz, 1H), 7.94 (d, J=7.6 Hz, 1H), 8.39 (dd, J=8.0, 1.2 Hz, 1H), 8.50 (s, 2H). ¹³C NMR (CDCl₃) δ 28.2, 29.8, 82.7, 116.5, 120.1, 123.6, 125.3, 127.4, 129.8, 133.2, 134.1, 134.5, 148.2, 158.6, 160.0, 161.7, 174.2. HRMS (ESI) calc'd for C₁₉H₁₉NO₆ 358.1254, found 358.1290.

A solution of EX-1C (5.845 g, 1636 mmol), 40% aqueous methyl amine (35 mL, 45.1 mmol), and acetic acid (35.0 mL) in methanol (117.0 mL) was heated to 65° C. After 3.5 hrs, the mixture was cooled to room temperature and concentrated in vacuo to give a dark oil EX-ID (11.64 g/est. 100%). ¹H NMR (CDCl₃) δ 1.60 (s, 9H), 2.94 (d, J=6.0 Hz, 2H), 332 (s, 3H), 4.58 (dd, J=17.1, 1.5 Hz, 1H), 4.85 (dd, J=10.2, 0.9 Hz, 1H), 5.72-5.87 (m, 1H), 7.61 (d, J=7.50 Hz, 1H), 7.75 (t, J=7.8 Hz, 1H), 8.15 (s, 2H), 8.388.41 (m, 1H). HRMS (ES/M+H). Calc'd for C₂₀H₂₂N₂O₅: 371.1607. Found: 371.1608.

A mixture of crude EX-1D (7.147 g, 1930 mmol) and TFA (6.50 mL, 84.4 mmol) in CH₂Cl₂ (100.0 mL) was stirred at room temperature. After 2.5 hrs, the reaction mixture was concentrated in vacuo and CH₃CN (30 mL) and 4N HCl in dioxane (30.0 mL, 120 mmol) was added. After two hrs, the mixture was concentrated in vacuo to give a black oily solid. Purification by flash chromatography on silica gel (1% MeOH in CH₂Cl₂) gave a tan solid EX-1E (3.420 g/56%). ¹H NMR (CDCl₃) δ 2.99 (m, 2H), 3.46 (s, 3H), 4.69 (dd, J=17.2, 1.6 Hz, 1H), 4.89 (dd, J=10.0, 1.6 Hz, 1H), 5.65-5.75 (m, 1H), 7.72 (m, 1H), 7.79 (t, J=8.0 Hz, 1H), 8.21 (t, J=1.6 Hz, 1H), 8.38 (m, 1H), 8.68 (s, 1H). ¹³C NMR (CDCl₃) δ 313, 45.8, 114.8, 116.6, 1183, 125.0, 126.1, 130.3, 131.7, 133.8, 135.0, 136.0, 147.6, 177.6. HRMS (ESIM+H). Calc'd for C₁₆H₁₄N₂O₅: 315.0981. Found: 315.0975.

A mixture of EX-1E (0.267 g, 0.761 mmol) and Et₃N (0.210 mL, 1.51 mmol) in PhCH₃ (0.50 mL) and CH₃CN (0.50 mL) were stirred at room temperature in a 10 mL round-bottom flask under N₂. Note: It is important to have dry glassware, reagents and solvents for this reaction. After 10 min, diphenylphosphorylazide (0.280 mL, 1.30 mmol) was added, and the mixture was gradually heated to 80° C. After 5 hrs, the reaction mixture was cooled to room temperature. t-BuOH (1.00 mL, 10.4 mmol) was added, and the mixture was heated to 80° C. After 2.5 hrs, the mixture was cooled to room temperature and diluted with CH₂Cl₂ (50 mL). The solution was washed with saturated aqueous NaHCO₃ (2×15 mL), dried (MgSO4), filtered and concentrated in vacuo. Purification by flash chromatography on silica gel eluting with 20-50% EtOAc/hexanes gave EX-IF (0.084 g/29%). ¹H NMR (CDCl₃) δ 1.52 (s, 9H), 2.98 (dd, J=4.4, 1.2 Hz, 2H), 3.35 (s, 3H), 4.63 (dd, J=17.2, 2.0 Hz, 1H), 4.87 (dd, J=10.4, 1.6 Hz, 1H), 5.71-5.82 (m, 1H), 7.59-7.61 (m, 1H), 7.71-7.79 (m, 2H), 8.16 (t, J=1.6 Hz, 1H), 8.38 (m, 1H), 8.47 (s, 1H). ¹³C NMR (CDCl₃) δ 28.3, 31.2, 43.5, 80.7, 114.9, 123.5, 124.1, 124.4, 126.4, 128.8, 130.2, 134.8, 135.0, 135.3, 143.8, 148.3, 153.2, 168.5. HRMS (ES/M+H). Calc'd for C₂₀H₂₃N₃O₅: 386.1716. Found: 386.1730.

4Methylmorpholine N-oxide (0.098 g, 0.84 mmol), aq. OsO₄ (9.8 mL, 1.0 mg/mL, 0.038 mmol) and acetone (6.0 mL) were combined in a 50 mL round-bottom flask. The mixture was cooled to 2° C., and a solution of EX-1F (0.286 g, 0.742 mmol) in acetone (8.0 mL) was added over 2 minutes. The reaction mixture was allowed to warm to room temperature. After 3.5 hrs, NaHSO₃ (0.080 g, 0.77 mmol), water (60 mL) and brine (10 mL) was added. The mixture was extracted with EtOAc (4×30 mL), dried (MgSO₄), filtered and concentrated in vacuo to give a yellow oil EX-1G (0305 g/9⁸%). ¹H NMR (CDCl₃) a 1.49 (s, 9H), 236-2.57 (m, 2H), 3.22-332 (m, 2H),336 (s, 3), 3.47 (m, 1H),7.60 (m, if), 7.72-7.76 (m, 2H), 8.12-8.14 (m, 1H), 834-8.37 (m, 1H), 8.49 (s, 1H). H S (ES/M+H). Calc'd for C₂₀H₂₅N₃O₇: 420.1771. Found: 420.1767.

To a solution of EX-1G (0342 g, 0.815 mmol) in CHCl₃ (6.0 mL) was added a solution of NaIO₄ (0.176 g, 0.823 mmol) in water (3.0 mL). Acetonitrile (4.0 mL) was added, and the mixture was stirred at room temperature. After 2.5 hrs, water (50 mL) and brine (5 mL) was added. The mixture was extracted with CH₂Cl₂ (3×11 mL), dried (MgSO₄), filtered and concentrated in vacuo to give a dark oil EX-1H (0.257 g/81%). ¹H NMR (CDCl₃) δ 1.48 (s, 9), 3.30 (ABq, 2H), 3.36 (s, 3H), 7.55-7.59 (m, 1H), 7.67 (d, J=8A Hz, 1H), 7.72 (d, J=7.9 Hz, 1H), 8.10 (t, J=1.8 Hz, 1H), 832-836 (m, 1H), 8.49 (s, 1H), 9.57 (s, 1H). ¹³C NMR(CDCl₃) δ 28.1, 41.7, 43.5, 80.9, 118.2, 123.7, 124.7, 126.8, 128.8, 130.6, 134.4, 134.9, 144.8, 148.4, 153.0, 168.2, 198.4. HRMS (ES/M−CH₃)—Calc'd for C₁₉H₂₁N₃O₆: 374.1352. Found: 374.1363.

A solution of 2-methylbut-2-ene (25 mL), t-BuOH (20 mL) and THF (20 mL) was prepared. Compound EX-1H (0.240 g, 0.620 mmol) and the 2-methylbut-2-ene solution (1 mL) were combined in a 25 mL round-bottom flask. The mixture was cooled to 0° C., and a solution of NaClO₂ (0.441 g, 4.88 mmol) and NaH₂PO₄—H₂O (0.530 g, 3.84 mmol) in water (1.50 mL) was added. After 15 min, the mixture was allowed to warm to room temperature. After an additional 2.25 hrs, the mixture was concentrated in vacuo. Water (6.0 mL) was added, and aqueous 1N HCl was added until pH equaled 3. The mixture was extracted with 2 parts THF/1 part CH₂Cl₂ (4×7 mL). Combined organic layers were dried (MgSO₄), filtered and concentrated in vacuo to give a brown foam (0.287 g).

Trituration with ethyl ether (10 ml) and filtration gave a tan solid EX-1I (0.102 g/41%). ¹H NMR (CDCl₃) a 1.54 (s, 9H), 3.27 (ABq, 2), 3.51 (s, 3H), 7.65, 7.70 (m, 2H), 7.82 (d, J=8.1 Hz, 1H), 7.84 (d, J=7.8 Hz, 1H), 8.17 (t, J=1.8 Hz, 1H), 8.42-8.46 (m, 1H), 8.71 (s, 1H). ¹³C NMR (CDCl₃) δ 28.0, 33.9, 44.0, 81.2, 119.5, 123.8, 124.9, 127.6, 128.9, 130.8, 133.8, 135.0, 144.9, 148.4, 152.9, 168.5, 172.3. HRMS (ES/M+H). Calc'd for C₁₉H₂₁N₃O₇: 404.1458. Found: 404.1464.

Compound EX-1I (0.100 g, 0.248 mmol), N-cyclohexylcarbodiimide, N-methyl polystyrene resin (0.995 g, 1.51 mmol, 1.52 mmol/g), and HOBt (0.050 g, 0.37 mmol) were combined with CH₂Cl₂ (15.0 mL) and DMF (2.50 mL). After 40 min, a mixture of benzyl-[[(4-aminomethylphenyl)iminomethyl]amino]-carbamate hydrogen chloride salt (0.0835 g, 0.261 mmol) and EON (0.50 mL, 3.6 mmol) in DMF (0.75 mL) was added. After 21 hrs, the mixture was filtered, washing with CH₂Cl₂, and concentrated in vacuo to give a dark solid EX-1J (0.280 g/test. 100%). HRMS (ES/M+H). Calc'd for C₃₅H₃₆N₆O₈: 669.2673. Found: 669.2671.

Compound EX-1J (0.153 g, 0.229 mmol) and 4N HCl in dioxane (2.50 mL) were combined in a 10 mL round-bottom flask. The mixture was sonicated for 5 min. After standing at room temperature for 1.5 hers, a solution of NaBH(OAc)₃ (0.100 g, 0.472 mmol) and acetone (0.10 mL) in THF (0.90 mL) was added. After 2 hrs more, water (10 mL) was added, and the mixture was extracted with EtOAc (2×6 mL). Saturated aqueous NaHCO₃ was added to the aqueous layer until pH=7, and the aqueous layer was then extracted with EtOAc (3×6 mL). All organic extracts were combined, dried (MgSO₄) and concentrated in vacuo to give a dark oil (0.143 g). Purification by filtration through silica gel eluting with 5% MeOH/CH₂Cl₂ gave a tan solid EX-1K (0.083 g/59%). ¹H NMR (CDCl₃) δ 1.27 (d, J=6.3 Hz, 6H), 2.842.97 (m, 2H), 3.39-3.48 (m, 2H), 3.41 (s, 31), 3.60 (m, 1H), 4.39 (m, 2H), 4.80 (d, 1H), 7.287.46 (m, 7H), 7.64-7.80 (m, 41), 8.14 (s, 1H), 8.38 (m, 2H). HRMS (ES/M+H). Calc'd for C₃₃H₃₄N₆O₆: 611.2618. Found: 611.2629.

Compound EX-1K (0.070 g, 0.11 mmol), 4N HCl (2.0 mL), MEOH (9.0 mL) and 10% Pd on C (0.057 g) were combined in a Fischer-Porter bottle. The bottle was pressurized to 20 psig H₂. After 70 min, the bottle was vented, and its contents were filtered through celite washing with MeOH (100 mL). The filtrate was concentrated in vacuo to give a white solid (0.080 g). Purification by reverse phase HPLC (Gilson) eluting with CH₃CN/H₂O(0.1% TFA) gave the white solid product (0.029 g, 49%). ¹H NMR (D₂O) δ 1.25 (d, J=6.6 Hz, 6H), 3.21 (ABq, 2H), 3.38 (s, 3H), 3.69 (quin, J=630 Hz, 1H), 4.22 (ABq, 2H), 7.2&730 (m, 4H), 7.50-7.62 (m, 5H), 7.63-7.83 (m, 1H), 8.15 (s, 1H). HRMS (ES/M+H). Calc'd for C₂₅H₃₀N₆O₂: 447.2508. Found: 447.2526.

### Example 2

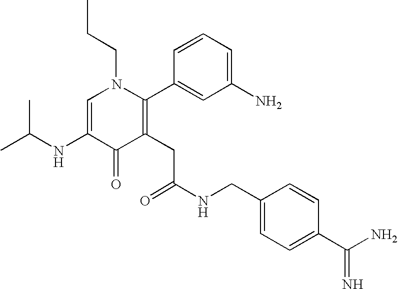

The compound was prepared as described for Example 1 with the exception that n-propylamine was substituted for methylamine in the process: ¹H NMR (D₂O) δ 1.20 (m, 12H), 3.08 (ABq, 2H), 3.25 (d, j=16.52 Hz, 1H), 3.68 (quin, J=6.24 Hz, 1H), 4.08 (quin, J=6.45 Hz, 1H), 4.24 (dd,J=16.12 Hz, J2=24.18 Hz, 2H), 7.26-7.40 (m, 4H),7507.66 (m, 4H), 8.20 (s, 1H). LC/MS (ESIM+H): 475.2.

Using the examples and methods described herein previously, the following examples having an amidinoaralkyl or amidinoheteroaralkyl type Y^{o} group could be prepared:

2-[3-[N-[[4-aminoiminomethylphenyl]methyl]-2-[3-aminophenyl]-5-[N,N-dimethylhydrazino]-4-pyridonyl]]acetamide;

2-[3-[N-[[4-aminoiminomethylphenyl]methyl]-2-[3-aminophenyl]-5 [N-ethyl-N-methylhydrazino]4-pyridonyl]]acetamide;

2-[3-[N-[[4-aminoininomethylphenyl]methyl]-2-[3-aminophenyl]-5 [N,N-dimethylhydrazino]-N-methylpyridonyl]]acetamide;

2-[3-[N-[4[aminoiminomethylphenyl]methyl]-2-[3-aminophenyl]-5-[N-ethyl-N-methylhydrazino]-N-methyl-pyridonyl]]acetamide;

2-[3-[N-[[4-aminoiminomethylphenyl]methyl]-2-[3,5-diaminophenyl]-5-[N,N-dimethylhydrazino[-4-pyridonyl]]acetamide;

2-[3-[N-[[4-aminoiminomethylphenyl]methyl]-2-[3,5-diaminophenyl]-5-[N-ethyl-N-methylhydrazino]-4-pyridonyl)]acetamide;

2-[3-[N-[[4-aminoiminomethylphenyl]methyl]-2-[3,5-diaminophenyl]-5[N,Ndimethylhydrazino]-N-methyl-4-pyridonyl]]acetamide;

2-[3-[N-[[4-aminoiminomethylphenyl]methyl]-2-[3,5-diamiophenyl]-5[N-ethyl-N-methylhydrazinol-N-methyl-4-pyridonyl]]acetamide;

2-[3-[N-[[4-amnoiminomethylphenyl]methyl]-2,[3-amino-5-carboxyphenyl]-S-[N,N-dimethylhydrazino]-4-pyridonyl]]acetamide;

2-[3-[N-[[4-aminoiminomethylphenyl]methyl]-2-[3-amino-5 carboxyphenyl]-5-N-ethyl-N-methylhydrazino]4-pyridonyl]]acetamide;

2-[3-[N-[[4-aminoiminomethylphenyl]methyl]-2-3-amino-5 carboxyphenyl]-5-[N,N-dimethylhydrazino]-N-methyl-4-pyridonyl]]acetamide;

2-[3-[N-[[4-aminoimnomethylphenyl]methyl]-2-[3-amino-5 carboxyphenyl]-5-[N-ethyl-N-methylhydrazino)-N-methyl-4 pyridonyl]]acetamide;

2-[3-[N-[[4-aminoiminomethylphenyl]methyl]-2-[3-amino-5-(N-benzylamidocarbonyl)phenyl]-5-[N,N-dimethylhydrazino]4 pyridonyl]]acetamide;

2-[3-[N-[[4-aminoiminomethylphenyl]methyl]-2-[3-amino-5-(N-benzylamidocarbonyl)phenyl]-5-[N-ethyl-N-methylhydrazino]4 pyridonyl]]acetamide;

2-[3-[N-[4[aminoiminomethylphenyl]methyl]-2-[3-amino-5-(N-benzylamidocarbonyl)phenyl]-5-[N,N-dimethylhydrazino]-N-methyl]pyridonyl]]acetamide; and

2-[3-[N-[[4-aminoiminomethylphenyl]methyl]-2-[3-amino-5-(N-benzylamidocarbonyl)phenyl]-5-[N-ethyl-N-methylhydrazino]-N-methyl-4-pyridonyl]]acetamide.

Using the examples and methods described herein previously, the following further examples having a amidinoaralkyl or amidinoheteroarallyl type Y⁰ group could be prepared of the formula:

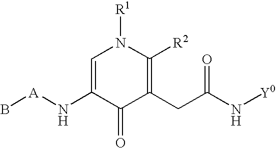

wherein B, A, R¹, R², X^{o}, and Y⁰ are selected to form the following compounds:

R² is 3-aminophenyl, B is phenyl, A is CH₂, Y⁰ is 4-amidinobenzyl, and R¹ is methyl;

R² is 3-aminophenyl, B is 3-chlorophenyl, A is CH₂CH₂, Y⁰ is 4-amidinobenzyl, and R¹ is methyl;

R² is 3-aminophenyl, B is phenyl, A is CH₂, Y⁰ is 4-amidinobenzyl, and R¹ is hydrido;

R² is 3-aminophenyl, B is 2-imidazoyl, A is CH₂CH₂CH₂, Y⁰ is 4 amidinobenzyl, and R¹ is methyl;

R² is 3-amidocarbonyl-5-aminophenyl, B is 3-chlorophenyl, A is CH₂CH₂, Y⁰ is 4-amidinobenzyl, and R¹ is methyl;

R² is 3-amino-5-(N-benzylamidocarbonyl)phenyl, B is 3-chlorophenyl, A is CH₂CH₂, Y⁰ is 4-amidinobenzyl, and R¹ is methyl;

R² is 3-amino-5-(N-(2-chlorobenzyl)amidocarbonyl)phenyl, B is 3-chlorophenyl, A is CH₂CH₂, Y⁰ is 4-amidinobenzyl, and R¹ is methyl;

R² is 3-amino-5-(N-(2-chlorobenzyl)amidosulfonyl)phenyl, B is 3-chlorophenyl, A is CH₂CH_{2,5}-e is amidinobenzyl, and R¹ is methyl;

R² is 3-amino-5-(N-(2-trifluoromethylbemyl)amidocarbonyl)-phenyl, B is 3-chlorophenyl, A is CH₂CH₂, Y⁰ is 4-amidinobenzyl, and R¹ is methyl;

R² is 3,5-aminophenyl, B is 3-chlorophenyl, A is CH₂CH₂, Y⁰ is 4 amidinobenzyl, and R¹ is methyl;

R² is 3-amino-5-carboxyphenyl, B is 3-chlorophenyl, A is CH₂CH₂, Y⁰ is 4-amidinobenzyl, and R¹ is methyl;

R² is 3-amidocarbonyl-5-aminophenyl, B is 3-chlorophenyl, A is CH₂CH₂, Y⁰ is 4-amidinobenzyl, and R¹ is hydrido;

R² is 3-amino-5-(N-benzylamidocarbonyl)phenyl, B is 3-chlorophenyl, A is CH₂CH₂, Y⁰ is 4-amidinobenzyl, and R¹ is hydrido;

R² is 3-amino-5-(N-(2-chlorobenzyl)awidocarbonyl)phenyl, B is 3-chlorophenyl, A is CH₂CH₂, Y⁰ is 4-amidinobenzyl, and R¹ is hydrido;

R² is 3-amino-5-(N-(2-chlorobenzyl)amidosulfonyl)phenyl, B is 3-chlorophenyl, A is CH₂CH₂, Y⁰ is 4-amidinobenzyl, and R¹ is hydrido;

R² is 3-amino-5-(N-2-trifluoromethylbenzyl)amidocarbonyl)-phenyl, B is 3-chlorophenyl, A is CH₂CH₂, Y⁰ is 4-amidinobenzyl, and R¹ is hydrido;

R² is 3,5-diaminophenyl, B is 3-chlorophenyl, A is CH₂CH₂, Y⁰ is 4-amidinobenzyl, and R¹ is hydrido;

R² is 3-amino-5-carboxyphenyl, B is 3-chlorophenyl, A is CH₂CH₂, Y⁰ is 4 amidinobenzyl, and R¹ is hydrido;

R² is 3-aminophenyl, B is 2,2,2-trifluoroethyl, A is a bond, Y⁰ is 4 amidinobenzyl, and R¹ is methyl;

R² is 3-aminophenyl, B is (S)-2-butyl, A is a bond, Y⁰ is 4-amidinobenzyl, and R¹ is methyl;

R² is 5-amino-2-fluorophenyl, B is isopropyl, A is a bond, Y⁰ is 4 amidinobenzyl, and R¹ is methyl;

R² is 2-methyl-3-aminophenyl, B is isopropyl, A is a bond, Y⁰ is 4 amidinobenzyl, and R¹ is methyl;

R² is 3-aminophenyl, B is ethyl, A is a bond, Y⁰ is 4-amidinobenzyl, and R¹ is methyl;

R² is 3-aminophenyl, B is ethyl, A is a bond, Y⁰ is 4-amidino-2-fluorobenzyl, and R¹ is methyl;

R² is 3-aminophenyl, B is 2-propenyl, A is a bond, Y⁰ is 4-amidinobenzyl, and R¹ is methyl;

R² is 3-aminophenyl, B is isopropyl, A is a bond, Y⁰ is 4-amidino-2-fluorobenzyl, and R¹ is methyl;

R² is 3-aminophenyl, B is isopropyl, A is a bond, Y⁰ is 4-amidinobenzyl, and R¹ is methyl;

R² is 3-aminophenyl, B is 2-butyl, A is a bond, Y⁰ is 4-amidinobenzyl, and R¹ is methyl;

R² is 3-aminophenyl, B is (R)-2-butyl, A is a bond, Y⁰ is 4-amidinobenzyl, and R¹ is methyl;

R² is 3-aminophenyl, B is 2-propynyl, A is a bond, Y⁰ is 4-amidinobenzyl, and R¹ is methyl;

R² is 3-aminophenyl, B is 3-pentyl, A is a bond, Y⁰ is 4-amidinobenzyl, and R¹ is hydrido;

R² is 3-aminophenyl, B is hydrido, A is CH₂, Y⁰ is 4-amidinobenzyl, and R¹ is methyl;

R² is 3-aminophenyl, B is ethyl, A is CH₂, Y⁰ is 4-aamidinobenzyl, and R¹ is methyl;

R² is 3-aminophenyl, B is 2-methypropyl, A is a bond, Y⁰ is 4-amidinobenzyl, and R¹ is methyl;

R² is 3-aminophenyl, B is 2-propyl, A is CH₃CH, Y⁰ is 4-amidinobenzyl, and R¹ is methyl;

R² is 3-aminophenyl, B is propyl, A is a bond, Y⁰ is 4-amidino-2-fluorobenzyl, and R¹ is methyl;

R² is 3-aminophenyl, B is 6-amidocarbonylhexyl, A is a bond, Y⁰ is 4 amidinobenzyl, and R¹ is methyl;

R² is 3-aminophenyl, B is tert-butyl, A is a bond, Y⁰ is 4-amidinobenzyl, and R¹ is hydrido;

R² is 3-aminophenyl, B is tert-butyl, A is a bond, Y⁰ is 4-amidinobenzyl, and R¹ is methyl;

R² is 3-aminophenyl, B is 3-hydroxypropyl, A is a bond, Y⁰ is 4-amidinobenzyl, and R¹ is methyl;

R² is 3-aminophenyl, B is 2-methylpropyl, A is a bond, Y⁰ is 4-amidino-2-fluorobenzyl, and R¹ is methyl;

R² is 3-aminophenyl, B is butyl, A is a bond, Y⁰ is 4-aamidinobenzyl, and R¹ is methyl;

R² is 3-aminophenyl, B is 1-methoxy-2-propyl, A is a bond, Y⁰ is 4 amidinobenzyl, and R¹ is methyl;

R² is 3-aminophenyl, B is 2-methoxyethyl, A is a bond, Y⁰ is 4 amidinobenzyl, and R¹ is methyl;

R² is 3-aminophenyl, B is 2-propyl, A is a bond, Y⁰ is 5-amidino-2-thienylmethyl, and R¹ is methyl;

R² is 5-amino-2-methylthiophenyl, B is 2-propyl, A is a bond, Y⁰ is 4 amidinobenzyl, and R¹ is methyl;

R² is 3-amino-5-carboxyphenyl, B is isopropyl, A is a bond, Y⁰ is 4 amidinobenzyl, and R¹ is methyl;

R² is 3-amino-5-carbomethoxyphenyl, B is isopropyl, A is a bond, Y⁰ is 4 amidinobenzyl, and R¹ is methyl;

R² is 3-aminophenyl, B is isopropyl, A is a bond, Y⁰ is 4-amidinobenzyl, and R¹ is ethyl;

R² is 3-amino-5-carboxamidophenyl, B is isopropyl, A is a bond, Y⁰ is 4 amidinobenzyl, and R¹ is methyl;

R² is 3-amino-5-(N-benzyl-N-methylamidocarbonyl)phenyl, B is isopropyl, A is a bond, Y⁰ is 4-amidinobenzyl, and R¹ is methyl;

R² is 3-amino-5-(N-(1-phenylethyl)amidocarbonyl)phenyl, B is isopropyl, A is a bond, Y⁰ is 4-amidinobenzyl, and R¹ is methyl;

R² is 3-amino-5-(N-(2-phenyl-2-propyl)amidocarbonyl)phenyl, B is isopropyl, A is a bond, Y⁰ is 4-amidinobenzyl, and R¹ is methyl;

R² is 3-amino-5-(N-(2,4-dichlorobenzyl)amidocarbonyl)phenyl, B is isopropyl, A is a bond, Y⁰ is 4-amidinobenzyl, and R¹ is methyl;

R² is 3-amino-5-N-(4-bromobenzyl)amidocarbonyl)phenyl, B is isopropyl, A is a bond, Y⁰ is 4-amidinobenzyl, and R¹ is methyl;

R² is 3-amino-5-(N-benzylamidocarbonyl)phenyl, B is isopropyl, A is a bond, Y⁰ is 4-amidinobenzyl, and R¹ is methyl;

R² is 3-amino-5-(N-(2-chlorobenzyl)amidocarbonyl)phenyl, B is isopropyl, A is a bond, Y⁰ is 4-amidinobenzyl, and R¹ is methyl;

R² is 3-amino-54N-(2-trifluoromethylbenzyl)amidocarbonyl)phenyl, B is isopropyl, A is a bond, Y⁰ is 4-amidinobenzyl, and R¹ is methyl;

R² is 3-amino-5-(N-(3-fluorobenzyl)amidocarbonyl)phenyl, B is isopropyl, A is a bond, Y⁰ is 4-amidinobenzyl, and R¹ is methyl;

R² is 3-amino-5-(N-(3-trifluoromethylbenzyl)amidocarbonyl)phenyl, B is isopropyl, A is a bond, Y⁰ is 4-amidinobenzyl, and R¹ is methyl;

R² is 3-amino-5-(N-isobutylamidocarbonyl)phenyl, B is isopropyl, A is a bond, Y⁰ is 4-amidinobenzyl, and R¹ is methyl;

R² is 3-amino-5-N-cylobutylamidocarbonyl)phenyl, B is isopropyl, A is a bond, Y⁰ is 4-amidinobenzyl, and R¹ is methyl;

R² is 3-amino-5-(N-cyclopentylamidocarbonyl)phenyl, B is isopropyl, A is a bond, Y⁰ is 4-amidinobenzyl, and R¹ is methyl;

R² is 3-amino-5-(N-cycloheptylamidocarbonyl)phenyl, B is isopropyl, A is a bond, Y⁰ is 4-amidinobenzyl, and R¹ is methyl;

R² is 3-amino-5-(N-(2-pyxidylmethyl)amidocarbonyl)phenyl, B is isopropyl, A is a bond, Y⁰ is 4-amidinobenzyl, and R¹ is methyl;

R² is 3-amino-5-(N-(3-pyridylmethyl)amidocarbonyl)phenyl, B is isopropyl, A is a bond, Y⁰ is 4-amidinobenzyl, and R¹ is methyl;

R² is 3-amino-5-(N-2-(4-methoxyphenyl)ethyl)amidocarbonyl)phenyl, B is isopropyl, A is a bond, Y⁰ is 4-amidinobenzyl, and R¹ is methyl;

R² is 3-amino-5-(N-(3-phenylpropyl)amidocarbonyl)phenyl, B is isopropyl, A is a bond, Y⁰ is 4-amidinobenzyl, and R¹ is methyl;

R² is 3-amino-5-N-(2,2-&phenylethyl)amidocarbonyl)phenyl, B is isopropyl, A is a bond, Y⁰ is 4-amidinobenzyl, and R¹ is methyl;

R² is 3-amino-5-(N-(2-naphthylmethyl)amidocarbonyl)phenyl, B is isopropyl, A is a bond, Y⁰ is 4-amidinobenzyl, and R¹ is methyl;

R² is 3-amino-5-(N-(1,2,3,4-tetrahydronaphth-2-ylmethyl)amidocarbonyl)phenyl, B is isopropyl, A is a bond, Y⁰ is 4-amidinobenzyl, and R¹ is methyl;

R² is 3-aminophenyl, B is 2-propyl, A is a bond, Y⁰ is 4-amidino-3-fluorobenzyl, and R¹ is hydrido;

R² is 3-carboxyphenyl, B is 2-propyl, A is a bond, Y⁰ is 4-aamidinobenzyl, and R¹ is hydrido;

R² is 3-aminophenyl, B is 2-propyl, A is a bond, Y⁰ is 4-amidino-3-fluorobenzyl, and R¹ is methyl;

R² is 3,5-diaminophenyl, B is 2,2,2-trifluoroethyl, A is a bond, Y⁰ is 4 amidinobenzyl, and R¹ is methyl;

R² is 3,5-diaminophenyl, B is (S)-2-butyl, A is a bond, Y⁰ is 4 amidinobenzyl, and R¹ is methyl;

R² is 3, diaminophenyl, B is isopropyl, A is a bond, Y⁰ is 4 amidinobenzyl, and R¹ is methyl;

R² is 3 Sdiaminophenyl, B is isopropyl, A is a bond, Y⁰ is 4-amidino-2-fluorobenzylbenzyl, and R¹ is methyl;

R² is 3,5-diaminophenyl, B is ethyl, A is a bond, Y⁰ is 4-amidinobenzyl, and R¹ is methyl;

R² is 3,5-diaminophenyl, B is ethyl, A is a bond, Y⁰ is 4-amidino-2-fluorobenzyl, and R¹ is methyl;

R² is 3-amino-5-carboxyphenyl, B is 2,2,2-trifluoroethyl, A is a bond, Y⁰ is 4-amidinobenzyl, and R¹ is methyl;

R² is 3-amino-5-carboxyphenyl, B is (S)-2-butyl, A is a bond, Y⁰ is 4 amidinobenzyl, and R¹ is methyl;

R² is 3-amino-5-carboxyphenyl, B is isopropyl, A is a bond, Y⁰ is 4 amidino-2-fluorobenzylbenzyl, and R¹ is methyl;

R² is 3-amino-5-carboxyphenyl, B is ethyl, A is a bond, Y⁰ is 4 amidinobenzyl, and R¹ is methyl;

R² is 3-amino-5-carboxyphenyl, B is ethyl, A is a bond, Y⁰ is 4-amidino-2-fluorobenzyl, and R¹ is methyl;

R² is 3-amino-5-(N-benzylamidocarbonyl)phenyl, B is 2,2,2-trifluoroethyl, A is a bond, Y⁰ is 4-amidinobenzyl, and R¹ is methyl;

R² is 3-amino-5-(N-benzylamidocarbonyl)phenyl, B is (S)₂-butyl, A is a bond, Y⁰ is 4-amidinobenzyl, and R¹ is methyl;

R² is 3-amino-(N-benzylamidocarbonyl)phenyl, B is isopropyl, A is a bond, Y⁰ is 4-amidino-2-fluorobenzylbenzyl, and R¹ is methyl;

R² is 3-amino-5-(N-benzylamidocarbonyl)phenyl, B is ethyl, A is a bond, is 4-amidinobenzyl, and R¹ is methyl;

R² is 3-amino-5-(N-benzylamidocarbonyl)phenyl, B is ethyl, A is a bond, Y⁰ is 4-amidino-2-fluorobenzyl, and R¹ is methyl;

R² is 3,5-diaminophenyl, B is isopropyl, A is a bond, Y⁰ is 4 amidinobenzylbenzyl, and R¹ is hydrido;

R² is 3-aminophenyl, B is cyclopropyl, A is a bond, Y⁰ is 4 amidinobenzyl, and R¹ is methyl;

R² is 3-aminophenyl, B is cyclobutyl, A is a bond, Y⁰ is 4-amidino-2-fluorobenzyl, and R¹ is methyl;

R² is 3-aminophenyl, B is cyclobutyl, A is a bond, Y⁰ is 4-amidinobenzyl, and R¹ is methyl;

R² is 3-aminophenyl, B is cyclopropyl, A is a bond, Y⁰ is 4-amidino-2-fluorobenzyl, and R¹ is methyl;

R² is 3-aminophenyl, B is cyclobutyl, A is a bond, Y⁰ is 4-amidinobenzyl, and R¹ is hydrido;

R² is 3-aminophenyl, B is cyclobutyl, A is a bond, Y⁰ is 4-amidino-3-fluorobenzyl, and R¹ is methyl;

R² is 3-aminophenyl, B is cyclopentyl, A is a bond, Y⁰ is 4-amidinobenzyl, and R¹ is methyl;

R² is 5-amino-2-thienyl, B is cyclobutyl, A is a bond, Y⁰ is 4 amidinobenzyl, and R¹ is methyl;

R² is 3-aminophenyl, B is cyclopropyl, A is CH₂, Y⁰ is 4-amidinobenzyl, and R¹ is methyl;

R² is 3-aminophenyl, B is 2-2R)-bicyclo[2.2.1]-heptyl, A is a bond, Y⁰ is 4-amidinobenzyl, and R¹ is methyl;

R² is 3-aminophenyl, B is cyclopentyl, A is a bond, Y⁰ is 4-amidino-2-fluorobenzyl, and R¹ is methyl;

R² is 3-aminophenyl, B is cyclohexyl, A is CH₂CH₂, Y⁰ is 4 amidinobenzyl, and R¹ is hydrido;

R² is 3-aminophenyl, B is oxalan-2-yl, A is CH₂, Ye is 4-amidinobenzyl, and R¹ is methyl;

R² is 3-aminophenyl, B is 1-piperidinyl, A is CH₂CH₂, Y⁰ is 4 amidinobenzyl, and R¹ is methyl;

R² is 3-aminophenyl, B is l-pyrrolidinyl, A is CH₂CH₂CH₂, Y⁰ is 4-amidinobenzyl, and R¹ is methyl;

R² is 3-amino-5-carbomethoxyphenyl, B is cyclobutyl, A is a bond, Y⁰ is 4-amidinobenzyl, and R¹ is hydrido;

R² is 3-amino-icarboxyphenyl, B is cyclobutyl, A is a bond, Y⁰ is 4 amidinobenzyl, and R¹ is hydrido;

R² is 3,5-diaminophenyl, B is cyclobutyl, A is a bond, Y⁰ is 4-amidinobenzyl, and R¹ is hydrido;

R² is 2-aminocarboxy-4-pyridyl, B is cyclobutyl, A is a bond, Y, is 4-amidinobenzyl, and R¹ is hydrido;

R² is 3-amino-5-carbomethoxyphenyl, B is cyclobutyl, A is a bond, Y⁰ is 4-amidinobenzyl, and R¹ is methyl;

R² is 3-amino-5-carboxyphenyl, B is cyclobutyl, A is a bond, Y⁰ is 4 amidinobenzyl, and R¹ is methyl;

R² is 3,5-diaminophenyl, B is cyclopropyl, A is a bond, Y⁰ is 4 amidinobenzyl, and R¹ is methyl;

R² is 3,5-diaminophenyl, B is cyclobutyl, A is a bond, Y⁰ is 4-amidino-2-fluorobenzyl, and R¹ is methyl;

R² is 3,5-diaminophenyl, B is cyclopropyl, A is a bond, Y⁰ is 4-amidino-2-fluorobenzyl, and R¹ is methyl;

R² is 3,5-iaininophenyl, B is cyclobutyl, A is a bond, Y⁰ is 4 amidinobenzyl, and R¹ is hydrido;

R² is 3,5-diaminophenyl, B is cyclobutyl, A is a bond, Y⁰ is 4-amidino-3-fluorobenzyl, and R¹ is methyl;

R² is 3,5-diaminophenyl, B is cyclopentyl, A is a bond, Y⁰ is 4 amidinobenzyl, and R¹ is methyl;

R² is 3-carboxy-5-aminophenyl, B is cyclopropyl, A is a bond, Y⁰ is 4 amidinobenzyl, and R¹ is methyl;

R² is 3-carboxy-5-ainiinophenyl, B is cyclobutyl, A is a bond, Y⁰ is 4 amidino-2-fluorobenzyl, and R¹ is methyl;

R² is 3-carboxy-5-aminophenyl, B is cyclopropyl, A is a bond, Y⁰ is 4 amidino-2-fluorobenzyl, and R¹ is methyl;

R² is 3-carboxy-5-aminophenyl, B is cyclobutyl, A is a bond, Y⁰ is 4-amidinobenzyl, and R¹ is hydrido;

R² is 3-carboxy-5-aminophenyl, B is cyclobutyl, A is a bond, Y⁰ is 4 amidino-3-fluorobenzyl, and R¹ is methyl;

R² is 3-carboxy-5-aminophenyl, B is cyclopentyl, A is a bond, Y⁰ is 4 amidinobenzyl, and R¹ is methyl;

R² is 3-amino-5-(N-benzylamidocarbonyl)phenyl, B is cyclopropyl, A is a bond, Y⁰ is 4-amidinobenzyl, and R¹ is methyl;

R² is 3-amino-5-(N-benzylamidocarbonyl)phenyl, B is cyclobutyl, A is a bond, Y⁰ is 4-amidino-2-fluorobenzyl, and R¹ is methyl;

R² is 3-amino-5-(N-benzylamidocarbonyl)phenyl, B is cyclobutyl, A is a bond, Y⁰ is 4-amidinobenzyl, and R¹ is methyl;

R² is 3-amino-5-(N-benzylamidocarbonyl)phenyl, B is cyclopropyl, A is a bond, Y⁰ is 4-amidino-2-fluorobenzyl, and R¹ is methyl;

R² is 3-amino-5-(N-benzylaridocarbonyl)phenyl, B is cyclobutyl, A is a bond, Y⁰ is 4-amidinobenzyl, and R¹ is hydnrdo;

R² is 3-amino-5-(N-benzylaridocarbonyl)phenyl, B is cyclobutyl, A is a bond, Y⁰ is 4-amidino-3-fluorobenzyl, and R¹ is methyl;

R² is 3-amino-5-(N-benzylamidocarbonyl)phenyl, B is cyclopentyl, A is a bond, Y⁰ is 4-amidinobenzyl, and R¹ is methyl;

R² is 3-amino-5-(N-(2-chlorobenzyl)aniidosulfonyl)phenyl, B is cyclopropyl, A is a bond, Y⁰ is 4-amidinobenzyl, and R¹ is methyl;

R² is 3-amino-5-(N-(2-chlorobenzyl)amidosulfonyl)phenyl, B is cyclobutyl, A is a bond, Y⁰ is 4-amidino-2-fluorobenzyl, and R¹ is methyl;

R² is 3-amino-5-(N-(2-chlorobenzyl)amidosulfonyl)phenyl, B is cyclobutyl, A is a bond, Y⁰ is 4-amidinobenzyl, and R¹ is methyl;

R² is 3-amino-5-(N-(2-chlorobenzyl)amidosulfonyl)phenyl, B is cyclopropyl, A is a bond, Y⁰ is 4-amidino-2-fluorobenzyl, and R¹ is methyl;

R² is 3-amino-5-(N-(2-chlorobenzyl)amidosulfonyl)phenyl, B is cyclobutyl, A is a bond, Y⁰ is 4-amidinobenzyl, and R¹ is hydrido;

R² is 3-amino-5-(N-(2-chlorobenzyl)amidosulfonyl)phenyl, B is cyclobutyl, A is a bond, Y⁰ is 4-amidino-3-fluorobenzyl, and R¹ is methyl;

R² is 3-amino-5-(N-(2-chlorobenzyl)amidosulfonyl)phenyl, B is cyclopentyl, A is a bond, Y⁰ is 4-amidinobenzyl, and R¹ is methyl;

R² is 3-amino-5-(N-(2-trifluoromethylbenzyl)amidocarbonyl)-phenyl, B is cyclopropyl, A is a bond, Y⁰ is 4-amidinobenzyl, and R¹ is methyl;

R² is 3-amino-5-(N-2-trifluoromethylbenzyl)amidocarbonyl)-phenyl, B is cyclobutyl, A is a bond, Y⁰ is 4-amidino-2-fluorobenzyl, and R¹ is methyl;

R² is 3-amino-5-(N-(2-trifluoromethylbenzyl)amidocarbonyl)-phenyl, B is cyclobutyl, A is a bond, Y⁰ is 4-amidinobenzyl, and R¹ is methyl;

R² is 3-amino-5-N-(2-trifluoromethylbenzyl)anndocarbonyl)-phenyl, B is cyclopropyl, A is a bond, Y⁰ is 4-amidino-2-fluorobenzyl, and R¹ is methyl;

R² is 3-amino-5-(N-(2-trifluoromethylbenzyl)miidocarbonyl)-phenyl, B is cyclobutyl, A is a bond, Y⁰ is 4-amidinobenzyl, and R¹ is hydrido;

R² is 3-amino-5-(N-2-tfluoromethylbenzyl)amidocaxbonyl)-phenyl, B is cyclobutyl, A is a bond, Y⁰ is 4-amidino-3-fluorobenzyl, and R¹ is methyl; and

R² is 3-amino-5-(N-(2-tnifluoromethylbenzyl)amidocarbonyl)-phenyl, B is cyclopentyl, A is a bond, Y⁰ is 4-amidinobenzyl, and R¹ is methyl.

Using the examples and methods described herein previously, the following additional examples having a guanidinoalkyl type Y^{AT} group could be prepared of the formula:

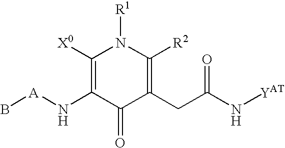

wherein B. A, R¹, R², X^{o}, and Y^{AT} are selected to form the following compounds:

R² is 3-aminophenyl, B is phenyl, A is CH₂CH₂, Y^{AT} is 5-guanidino-1-oxo-1-(2-thiazolyl)-2-pentyl, R¹ is hydrido, and X^{o} is hydrido;

R² is 3,5-diaminophenyl, B is phenyl, A is CH₂CH₂, Y^{AT} is 5-guanidino-1-oxo-1-(2-thiazolyl)-2-pentyl, R¹ is hydrido, and X⁰ is hydrido;

R² is 3-carboxy-5-aminophenyl, B is phenyl, A is CH₂CH₂, Y^{AT} is 5-guanidino-1-oxo-1-(2-thiazolyl)-2-pentyl, R¹ is hydrido, and X^{o} is hydrido;

R² is 3-amino-5-(N-benzylamidocarbonyl)phenyl, B is phenyl, A is CH₂CH₂, Y^{AT} is 5-guanidino-1-oxo-1-(2-thiazolyl)-2-pentyl, R¹ is hydrido, and X^{o} is hydrido;

R² is 3, diaminophenyl, B is isopropyl, A is single bond, Y^{AT} is 5-guanidino-1-oxo-112-thiazolyl)-2-pentyl, R¹ is hydrido, and X⁰ is hydrido;

R² is 3-carboxy-5-aminophenyl, B is isopropyl, A is single bond, Y^{AT} is 5-guanidino-1-oxo-1-(2-thiazolyl)-2-pentyl, R¹ is hydrido, and X⁰ is hydrido;

R² is 3-amino-5-(N-benzylamidocarbonyl)phenyl, B is isopropyl, A is single bond, Y^{AT} is 5-guanidino-1-oxo-1-(2-thiazolyl)₂-pentyl, R¹ is hydrido, and X^{o} is hydrido;

R² is 3,5-diaminophenyl, B is cyclobutyl, A is single bond, Y^{AT} is 5-guanidino-1-oxo-1-2-thiazolyl)-2-pentyl, R¹ is hydrido, and X⁰ is hydrido;

R² is 3-carboxy-5-aminophenyl, B is cyclobutyl, A is single bond, Y^{AT} is 5-guanidino-1-oxol-(2-thiazolyl)₂-pentyl, R¹ is hydrido, and X⁰ is hydrido;

R² is 3-amino-5-(N-benzylamidocarbonyl)phenyl, B is cyclobutyl, A is single bond, Y^{AT} is Sguanidino-1-oxo-1-(2-thiazolyl)₂-pentyl, R¹ is hydrido, and X⁰ is hydrido;

R² is 3-aminophenyl, B is phenyl, A is CH₂CH₂, YT is 5-guanidino-1-oxo-1-(2-thiazolyl)-2,5-pentyl, R¹ is methyl, and X⁰ is hydrido;

R² is 3,5-diaminophenyl, B is phenyl, A is CH₂CH₂, Y^{AT} is 5-guanidino-1-oxo-1-(2-thiazolyl)-2-pentyl, R¹ is methyl, and X⁰ is hydrido;

R² is 3-carboxy-5-aminophenyl, B is phenyl, A is CH₂CH₂, YT is 5_guanidino-1-oxo-1-(2-thiazolyl)-2-pentyl, R¹ is methyl, and X⁰ is hydrido;

R² is 3-amino-5-(N-benzylamidocarbonyl)phenyl, B is phenyl, A is CH₂CH₂, YA is 5-guanidino-1-oxo-1-(2-thiazolyl)-2-pentyl, R¹ is methyl, and X⁰ is hydrido;

R² is 3,5-diaminophenyl, B is isopropyl, A is single bond, Y^{AT} is 5 guanidino-1-oxo-1-(2-thiazolyl)₂-pentyl, R¹ is methyl, and X⁰ is hydrido;

R² is 3-carboxy-5-aminophenyl, B is isopropyl, A is single bond, Y^{AT} is 5 guanidino-1-oxo-1-(2-thiazolyl)-2,5-pentyl, R¹ is methyl, and X⁰ is hydrido;

R² is 3-amino-5-(N-benzylamidocarbonyl)phenyl, B is isopropyl, A is single bond, Y^{AT} is 5-guanidino-1-oxo-1-(2-thiazolyl)-2-pentyl, R¹ is methyl, and X⁰ is hydrido;

R² is 3,5-diaminophenyl, B is cyclobutyl, A is single bond, Y^{AT} is 5-guanidino-1-oxo-1-(2-thiazolyl)₂-pentyl, R¹ is methyl, and X⁰ is hydrido;

R² is 3-carboxy-5-aminophenyl, B is cyclobutyl, A is single bond, Y^{AT} is 5-guanidino-1-oxo-1-(2-thiazolyl)-2-pentyl, R¹ is methyl, and X⁰ is hydrido; and

R² is 3-amino-5-(N-benzylamidocarbonyl)phenyl, B is cyclobutyl, A is single bond, Y^{AT} is 5-guanidino-1-oxo-1-(2-thiazolyl)-2-pentyl, R¹ is methyl, and X⁰ is hydrido.

Formula (I) compounds of this invention possessing hydroxyl, thiol, and amine functional groups can be converted to a wide variety derivatives. A hydroxyl group in the form of an alcohol or phenol can be readily converted to esters of carboxylic, sulfonic, carbamic, phosphonic, and phosphoric acids. Acylation to form a carboxylic acid ester is readily effected using a suitable acylating reagent such as an aliphatic acid aahydxide or acid chloride. The corresponding aryl and heteroaryl acid anhydrides and acid chlorides can also be used. Such reactions are generally carried out using an amine catalyst such as pyridine in an inert solvent. Similarly, carbamic acid esters (urethanes) can be obtained by reacting a hydroxyl group with isocyanates and carbamoyl chlorides. Sulfonate, phosphonate, and phosphate esters can be prepared using the corresponding acid chloride and similar reagents. Compounds of Formula (I) that have at least one thiol group present can be converted to the corresponding thioesters derivatives analogous to those of alcohols and phenols using the same reagents and comparable reaction conditions. Compounds of Formula (I) that have at least one primary or secondary amine group present can be converted to the corresponding amide derivatives. Amides of carboxylic acids can be prepared using the appropriate acid chloride or anhydrides with reaction conditions analogous to those used with alcohols and phenols. Ureas of the corresponding primary or secondary amine can be prepared using isocyanates directly and carbamoyl chlorides in the presence of an acid scavenger such as triethylamine or pyridine. Sulfonamides can be prepared from the corresponding sulfonyl chloride in the presence of aqueous sodium hydroxide. Suitable procedures and methods for preparing these derivatives can be found in House's Modem Synthetic Reactions, W. A. Benjamin, Inc., Shriner, Fuson, and Curtin in The Systematic Identification of Organic Compounds, 5th Edition, John Wiley & Sons, and Fieser and Fieser in Reagents for Organic Synthesis, Volume 1, John Wiley & Sons. Reagents of a wide variety that can be used to derivatize hydroxyl, thiol, and amines of compounds of Formula (I) are available from commercial sources or the references cited above, which are incorporated herein by reference.

Formula (I) compounds of this invention possessing hydroxyl, thiol, and amine functional groups can be alkylated to a wide variety derivatives. A hydroxyl group of compounds of Formula (I) can be readily converted to ethers. Alkylation to form an ether is readily effected using a suitable alkylating reagent such as an alkyl bromide, alkyl iodide or alkyl sulfonate. The corresponding aralkyl, heteroaralkyl, alkoxyalkyl, aralkyloxyalkyl, and heteroaralkyloxyalkyl bromides, iodides, and sulfonates can also be used. Such reactions are generally carried out using an alkoxide forming reagent such as sodium hydride, potassium t-butoxide, sodium amide, lithium amide, and n-butyl lithium using an inert polar solvent such as DMF, DMSO, THF, and similar, comparable solvents. amine catalyst such as pyridine in an inert solvent. Compounds of Formula (I) that have at least one thiol group present can be converted to the corresponding thioether derivatives analogous to those of alcohols and phenols using the same reagents and comparable reaction conditions. Compounds of Formula (I) that have at least one primary, secondary or tertiary amine group present can be converted to the corresponding quaternary ammonium derivatives. Quaternary ammonium derivatives can be prepared using the appropriate bromides, iodides, and sulfonates analogous to those used with alcohols and phenols. Conditions involve reaction of the amine by warming it with the alkylating reagent with a stoichiometric amount of the amine (i.e., one equivalent with a tertiary amine, two with a secondary, and three with a primary). With pnmary and secondary amines, two and one equivalents, respectively, of an acid scavenger are used concurrently. Tertiary amines can be prepared from the corresponding primary or secondary amine by reductive alkylation with aldehydes and ketones using reduction methods. Suitable procedures and methods for preparing these derivatives can be found in House's Modern Synthetic Reactions, W. A. Benjamin, Inc., Shriner, Fuson, and Curtin in The Systematic Identification of Organic Compounds, 5th Edition, John Wiley & Sons, and Fieser and Fieser in Reagents for Organic Synthesis, Volume 1, John Wiley & Sons. Perfluoroalkyl derivatives can be prepared as described by DesMarteau in J. Chem. Soc. Chem. Commun. 2241 (1998). Reagents of a wide variety that can be used to derivatize hydroxyl, thiol, and amines of compounds of Formula (I) are available from commercial sources or the references cited above, which are incorporated herein by reference.

The biological activity of the compounds of Example 1 and Example 2 were evaluated using bioassay procedures below. The biological activity of the compounds of Schemes 1, 2, 3, and 4 could also be evaluated using the same bioassay procedures below.

### Assays for Biological Activity TF-VIIa Assay

In this assay 100 nM recombinant soluble tissue factor and 2 nM recombinant human factor VIIa are added to a 96-well assay plate containing 0.4 mM of the substrate, N-Methylsulfonyl-D-phe-gly-arg-p-nitroaniline and either inhibitor or buffer (5 mM CaCl₂, 50 mM Tris-HCl, pH 8.0, 100 mM NaCl, 0.1% BSA). The reaction, in a final volume of 100 ul is measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of TF-VIIa activity is calculated from OD₄₀₅ₙₘ value from the experimental and control sample.

### Xa Assay

0.3 nM human factor Xa and 0.15 mM N-a-Benzyloxycarbonyl-D-arginyl-L-glycyl-L-arginne-nitroaniline-dihydrochloride (S-2765) are added to a 96-well assay plate containing either inhibitor or buffer (50 mM Tris-HCl, pH 8.0, 100 mM NaCl, 0.1% BSA). The reaction, in a final volume of 100 ul is measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of Xa activity is calculated from OD₄₀₅ₙₘ value from the experimental and control sample.

### Thrombin Assay

0.28 nM human thrombin and 0.06 mM H-D-Phenylalanyl-L-pipecolyl-L, arginine-nitroaniline, dihydrochloride are added to a 96-well assay plate containing either inhibitor or buffer (50 mM Tris-HCl, pH 8.0, 100 mM NaCl, 0.1% BSA). The reaction, in a final volume of 100 ul is measured immediately at 405 nm to determine background absorbance. The plate is incubated at room temperature for 60 min, at which time the rate of hydrolysis of the substrate is measured by monitoring the reaction at 405 nm for the release of pnitroaniline. Percent inhibition of thrombin activity is calculated from OD₄₀₅ₙₘ value from the experimental and control sample.

### Trypsin Assay

5 ug/ml trypsin, type IX from porcine pancreas and 0.375 mM N-α-Benzoyl-L-arginine-p-nitroanilide (LBAPNA) are added to a 96-well assay plate containing either inhibitor or buffer (50 mM Tris-HCl, pH 8.0, 100 mM NaCl, 0.1% BSA). The reactions, in a final volume of 100 ul are measured immediately at 405 nm to determine background absorbance. The plate is incubated at room temperature for 60 min, at which time the rate of hydrolysis of the substrate is measured by monitoring the reaction at 405 nm for the release of p-nitroaniline. Percent inhibition of trypsin activity is calculated from OD₄₀₅ₙₘ value from the experimental and control sample.

Recombinant soluble TF, consisting of amino acids 1-219 of the mature protein sequence was expressed in E. coli and purified using a Mono Q Sepharose FPLC. Recombinant human VIa was purchased from American Diagnostica, Greenwich Cr and chromogenic substrate N-Methylsulfonyl-D-phe-gly-arg-pnitroaniline was prepared by American Peptide Company, Inc., Sunnyvale, Calif. Factor Xa was obtained from Enzyme Research Laboratories, South Bend IN, thrombin from Calbiochem, La Jolla, Calif., and trypsin and L-BAPNA from Sigma, St. Louis Mo. The chromogenic substrates S-2765 and S-2238 were purchased from Chromogenix, Sweden.

TABLE 1
Inhibitory Activity of 4-Pyridones toward TF-VIIA, Thrombin II,
Factor Xa, and Trypsin II.

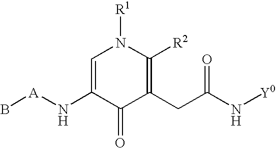

| | IC50 or % | IC50 or % | IC50 or % | IC50 or % |
|---|---|---|---|---|
| | Inhibition | Inhibition | Inhibition | Inhibition |
| Ex. | TF-VIIa | Thrombin | Factor Xa | Trpysin II |
| No. | (30 υM) | (30 υM) | (30 υM) | (30 υM) |
| 1 | 0.28 | 100 μM | 32% at 100 μM | 0.033 |
| 2 | 0.56 | 45% at 100 μM | 34% at 100 μM | 0.151 |

What we claim is:

1. A compound having the Formula (I): 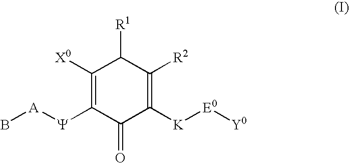
or a pharmaceutically acceptable salt thereof, wherein;
B is formula (V): 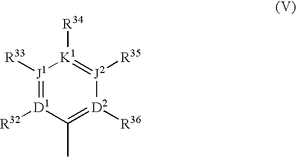
wherein D¹ D², J¹, J² and K¹ are independently selected from the group consisting of C, N, O, S and a covalent bond with the provisos that no more than one is a covalent bond, no more than one of D¹ D², J¹, J² and K¹ is O, no more than one of D¹ D², J¹, J² and K¹ is S, one of D¹ D², J¹, J² and K¹ must be a covalent bond when two of D¹ D², J¹, J² and K¹ are O and S, and no more than four of D¹ D², J¹, J² and K¹ are N with the proviso that R³², R³³, R³⁴, R³⁵, and R³⁶ are each independently selected to maintain the tetravalent nature of carbon, trivalent nature of nitrogen, the divalent nature of sulfur, and the divalent nature of oxygen;
R³², R³³, R³⁴, R³⁵, and R³⁶ are independently selected to be Q^{b};
R⁹, R¹⁰, R¹¹, R¹², R¹³, R¹⁶, R¹⁷, R¹⁸, R¹⁹, R³², R³³, R³⁴, R³⁵, and R³⁶ are independently selected from the group consisting of hydrido, amidino, guanidino, dialkylsulfonium, trialkylphosphonium, dialkylsulfoniumalkyl, carboxy, heteroaralkylthio, heteroaralkoxy, cycloalkylamino, acylalkyl, acylalkoxy, aryloylalkoxy, heterocyclyloxy, aralkylaryl, arakyl, aralkenyl, aralkynyl, heterocyclyl, perhaloaralkyl, aralkylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, alkoxyamino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylanidosulfonyl, arylsulfonamido, diarylamidosulfonyl, monoalkyl monoaryl amidosulfonyl, arylsufinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyarlkyl, hydroxyalkyl, aminoalkyl, hydoxyheteroaialkyl, haloalkoxyalkyl, aryl, aalkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl;
R³² and R³³, R³³ and R³⁴, R³⁴ and R³⁵ and R³⁵ and R³⁶ substituent pairs are independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R³² and R³³, R³³ and R³⁴, R³⁴ and R³⁵, and R³⁵ and R³⁶ are used at the same time;
B is formula (VI): 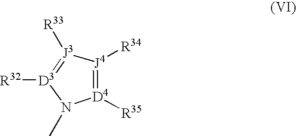
wherein D³, D⁴, J³, and J⁴ are independently selected from the group consisting of C, N, O, and S, no more than one of D³, D⁴, J³, and J4 is O, no more than one of D³, D⁴, J³, and J⁴ is S, and no more than three of D¹, D², J¹, and J² are N with the proviso that R³², R³³, R³⁴, and R³⁵ are each independently selected to maintain the tetravalent nature of carbon, trivalent nature of nitrogen, the divalent nature of sulfur, and the divalent nature of oxygen;
B is selected from the group consisting of C3-C alkyl, C3-C8 alkenyl, C3S alkynyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;
B is selected from the group consisting of C3-C10 cycloalkyl, C5-C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R¹⁰ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;
R⁹ and R¹⁰, R¹⁰ and R¹¹, R¹¹ and R¹², and R¹² and R¹³ spacer pairs are independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R⁹ and R¹⁰, R¹⁰ and R¹¹, R¹¹ and R¹², and R¹² and R¹³ is used at the same time;
A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, Se(O), Se(O)₂, Se(O)₂N(R⁷), (R⁷)NSe(O)₂, P(O)(R⁸), N(R⁷)P(O)(R⁸), P(O)(R⁸)N(R⁷), C(NR⁷)N(R⁷), (R⁷)NC(NR⁷), and N(R⁷);
R⁷ and R⁸ are independently selected from the group consisting of hydrido, hydroxy, alkyl, alkenyl, aryl, aralkyl, aryloxy, alkoxy, alkenyloxy, alkylthio, alkylamino, arylthio, arylamino, acyl, aroyl, heteroaroyl, aralkoxyalkyl, heteroaralkoxyalkyl, aryloxyalkyl, alkoxyalkyl, alkenyloxyalkyl, alkylthioalkyl, arylthioalkyl, aralkoxyalkyl, heteroaralkoxyalkyl, alkylsulfinylalkyl, alkylsulfonylalkyl, heteroaryl, heteroaryloxy, heteroarylamino, heteroaralkyl, heteroaralkyloxy, heteroaralkylamino, and heteroaryloxyalkyl;
R¹⁴, R¹⁵, R³⁷, R³⁸, R³⁹ R⁴⁰, R⁴¹ and R⁴² are independently selected from the group consisting of hydrido, hydroxy, halo, cyano, aryloxy, amino, alkylamino, dialkylamino, hydroxyalkyl, aminoalkyl, acyl, aroyl, heteroaroyl, heteroaryloxyalkyl, sulfhydryl, acylamido, alkoxy, alkylthio, arylthio, alkyl, alkenyl, alkynyl, aryl, aralkyl, aryloxyalkyl, aralkoxyalkylalkoxy, alkylsulfinylalkyl, alkylsulfonylalkyl, aralkylthioalkyl, heteroaralkoxythioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, saturated heterocyclyl, partially saturated heterocyclyl, heteroaryl, heteroarylalkyl, heteroarylthioalkyl, heteroaralkylthioalkyl, monocarboalkoxyalkyl, dicarboalkoxyalkyl, monocyanoalkyl, dicyanoalkyl, carboalkoxycyanoalkyl, alkylsulfinyl, alkylsulfonyl, haloalkylsulfinyl, haloalkylsulfonyl, arylsulfinyl, arylsulfinylalkyl, arylsulfonyl, arylsulfonylalkyl, aralkylsulfinyl, alkylsulfonyl, cycloalkylsulfinyl, cycloalkylsulfonyl, cycloalkylsulfinylalkyl, cycloalkylsufonylalkyl, heteroarylsulfonylalkyl, heteroarylsulfinyl, heteroarylsulfonyl, heteroarylsulfinylalkyl, aralkylsulfinylalkyl, aralkylsulfonylalkyl, carboxy, carboxyalkyl, carboalkoxy, carboxamide, carboxamidoalkyl, carboaralkoxy, trialkylsilyl, dialkoxyphosphono, diaralkoxyphosphono, dialkoxyphosphonoalkyl, and diaralkoxyphosphonoalkyl;
R¹⁴ and R¹⁴, when bonded to different carbons, are taken together to form a group selected from the group consisting of covalent bond, alkylene, haloalkylene, and a linear moiety spacer selected to form a ring selected from the group consisting of cycloalkyl ring having from 5 through 8 contiguous members, cycloalkenyl ring having from 5 through 8 contiguous members, and a heterocyclyl having from 5 through 8 contiguous members;
R¹⁴ and R¹⁵, when bonded to different carbons, are taken together to form a group selected from the group consisting of covalent bond, alkylene, haloalkylene, and a linear moiety spacer selected to form a ring selected from the group consisting of a cycloalkyl ring having from 5 through 8 contiguous members, a cycloalkenyl ring having from 5 through 8 contiguous members, and a heterocyclyl having from S through 8 contiguous members;
R¹⁵ and R¹⁵, when bonded to different carbons, are taken together to form a group selected from the group consisting of covalent bond, alkylene, haloalkylene, and a linear moiety spacer selected to form a ring selected from the group consisting of cycloalkyl ring having from 5 through 8 contiguous members, cycloalkenyl ring having from 5 through 8 contiguous members, and a heterocyclyl having from 5 through 8 contiguous members;
R¹⁴ and R¹⁵, when bonded to the same carbon, are taken together to form a group selected from a group consisting of oxo, thiono, alkylene, haloalkylene, and a linear moiety spacer selected to form a ring selected from the group consisting of cycloalkyl zing having from 5 through 8 contiguous members, cycloalkenyl ring having from 5 through 8 contiguous members, and a heterocyclyl having from 5 through 8 contiguous members;
Ψ is selected from the group consisting of NR⁵, O, C(O), C(S), S, S(O), S(O)₂, ON(R⁵), P(O)(R⁸), and CR³⁹R⁴⁰;
R⁵ is selected from the group consisting of hydrido, alkyl, alkenyl, alkynyl, aryl, aralkyl, aryloxy, alkoxy, alkenyloxy, alkylthio, arylthio, aralkoxyalkyl, heteroaralkoxyalkyl, aryloxyalkyl, alkoxyalkyl, alkenyloxyalkyl, alkylthioalkyl, arylthioalkyl, aralkoxyalkyl, heteroaralkoxyalkyl, alkylsulfinylalkyl, alkylsulfonylalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxyalkyl, halocycloalkenyloxyalkyl, heteroaryl, heteroarylalkyl, monocarboalkoxyalkyl, monocarboalkoxy, dicarboalkoxyalkyl, monocarboxamido, monocyanoalkyl, dicyanoalkyl, carboalkoxycyanoalkyl, acyl, aroyl, heteroaroyl, heteroaryloxyalkyl, and dialkoxyphosphonoalkyl;
R¹ is selected from the group consisting of hydrido, alkyl, alkenyl, haloalkyl, and alkoxyalkyl;
X⁰ and R² are independently selected from the group consisting of Z⁰-Q, hydrido, alkyl, alkenyl, and halo;
X⁰ and R² are independently selected from the group consisting of amidino, guanidino, dialkylsulfonium, trialkylphosphonium, dialkylsulfoniumalkyl, heteroarylamino, amino, nitro, alkylamino, arylamino, aralkylamino, alkanoyl, alkenoyl, aroyl, heteroaroyl, aralkanoyl, heteroaralkanoyl, haloalkanoyl, hydroxyhaloalkyl, cyano, and phosphono;
X⁰ and R¹ are taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X⁰ and R¹ and spacer pair R² and R¹ is used at the same time;
R² and R¹ are taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X⁰ and R¹ and spacer pair R² and R¹ are used at the same time;
X⁰ and R⁵ are taken together to form a spacer pair wherein the spacer pair forms a linear spacer moiety having from 2 through 5 contiguous atoms connecting the points of bonding of said spacer pair members to form a heterocyclyl ring having from 5 through 8 contiguous members;
X⁰ and R³⁹ are taken together to form a spacer pair wherein the spacer pair forms a linear spacer moiety having from 2 through 5 contiguous atoms connecting the points of bonding of said spacer pair members to form a heterocyclyl ring having from 5 through 8 contiguous members;
X⁰ and R⁴⁰ are taken together to form a spacer pair wherein the spacer pair forms a linear spacer moiety having from 2 through 5 contiguous atoms connecting the points of bonding of said spacer pair members to form a heterocyclyl ring having from 5 through 8 contiguous members;
X⁰ is selected to form a linear moiety having from 2 through 5 contiguous atoms linked to the points of bonding of both R³⁹ and R⁴⁰ to form a heterocyclyl ring having from 5 through 8 contiguous members;
R² and R^{4a}, R² and R^{4b}, R² and R¹⁴, and R² and R¹⁵ spacer pairs are independently selected to form spacer pairs wherein a spacer pair is taken together to form a linear moiety having from 2 through 5 contiguous atoms connecting the points of bonding of said spacer pair members to form a heterocyclyl ring having from 5 through 8 contiguous members with the proviso that no more than one of the group of spacer pairs consisting of R² and R^{4a}, R² and R^{4b}, R² and R¹⁴, and R² and R¹⁵ is used at the same time;
R² is independently selected to form a linear moiety having from 2 through 5 contiguous atoms linked to the points of bonding of both R and R^{4b} to form a heterocyclyl ring having from 5 through 8 contiguous members;
Z⁰ is selected from the group consisting of covalent single bond, (CR⁴¹R⁴²)_{q} wherein q is an integer selected from 1 through 6, (CH(R⁴¹))_{g}—W⁰—(CH(R⁴²))ₚ wherein g and p are integers independently selected from O through 3 and W⁰ is selected from the group consisting of O, S, C(O), C(S), C(O)O, C(S)O, C(O)S, C(S)S, C(O)N(R⁴¹), (R⁴¹)NC(O), C(S)N(R⁴¹), (R⁴¹)NC(S), OC(O))N(R⁴¹), (R⁴¹)NC(O)O, SC(S)N(R⁴¹), (R⁴¹)NC(S)S, SC(O)N(R⁴¹), (R⁴¹)NC(O)S, OC(S)N(R⁴¹), (R⁴¹)NC(S)O, N(R⁴²)C(O)N(R⁴¹), (R⁴¹)NC(O)N(R⁴²), N(R⁴²)C(S)N(R⁴¹), (R⁴¹)NC(S)N(R⁴²), S(O), S(O)₂, S(O)₂N(R⁴¹), N(R⁴¹)S(O)₂, Se, Se(O), Se(O)₂, Se(O)₂N(R⁴¹), N(R⁴¹)Se(O)₂, P(O)(R⁸), N(R⁷)P(O)(R⁸), P(O)(R⁸)N(R⁷), N(R⁴¹), ON(R⁴¹), and SiR²⁸R²⁹, and (CH(R⁴¹))ₑ—W²—(CH(R⁴²))ₕ wherein e and h are integers independently selected from 0 through 2 and W² is selected from the group consisting of CR⁴¹═CR⁴², CR⁴¹R⁴²═C; vinylidene), and ethynylidene (C—C; 1,2-ethynyl), with the provisos that R⁴¹ and R⁴² are selected from other than halo and cyano when directly bonded to N and Z⁰ is directly bonded to the pyridone ring;
Q^{b} is formula (II): 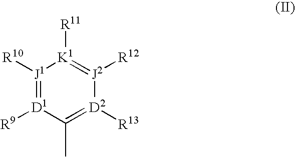
wherein D¹ D², J¹, J² and K¹ are independently selected from the group consisting of C, N, O, S and a covalent bond with the provisos that no more than one is a covalent bond, no more than one of D¹ D², J¹, J² and K¹ is O, no more than one of D¹ D², J¹, J² and K¹ is S, one of D¹ D², J¹, J² and K¹ must be a covalent bond when two of D¹ D², J¹, J² and K¹ are O and S, and no more than four of D¹ D², J¹, J² and K¹ are N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;
Q^{b} is formula (III): 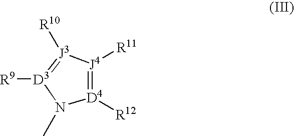
wherein D³, D⁴, J³, and J⁴ are independently selected from the group consisting of C, N, O, and S, no more than one of D³, D⁴, J³, and J⁴ is O, no more than one of D³,D⁴, J³, and J⁴ is S, and no more than three of D¹,D²,J¹, and J² are N with the proviso that R⁹, R¹⁰, R¹¹, and R¹² are each independently selected to maintain the tetravalent nature of carbon, trivalent nature of nitrogen, the divalent nature of sulfur, and the divalent nature of oxygen;
Q is selected from the group consisting of alkyl, alkoxy, alkylamino, alkylthio, haloalkylthio, alkenyl, alkynyl, saturated heterocyclyl, partially saturated heterocyclyl, acyl, aroyl, heteroaroyl, cycloalkyl, cycloalkylalkyl, cycloalkenyl, cycloalkenylalkyl, cycloalkylalkenyl, haloalkyl, haloalkoxy, haloalkenyl, halocycloalkyl, halocycloalkenyl, haloalkoxyalkyl, haloalkenyloxyalkyl, halocycloalkoxyalkyl, and halocycloalkenyloxyalkyl;
K is (CR^{4a}R^{4b})ₙ wherein n is an integer selected from 1 through 4;
R^{4a} and R^{4b} are independently selected from the group consisting of halo, hydrido, hydroxy, cyano, hydroxyalkyl, alkyl, alkenyl, aryl, aralkyl, aralkoxyalkyl, aryloxyalkyl, alkoxyalkyl, heteroaryloxyalkyl, alkenyloxyalkyl, alkylthioalkyl, aralkylthioalkyl, arylthioalkyl, cycloalkyl, cycloalkylalkyl, haloalkyl, haloalkenyl, heteroaryl, heteroarylalkyl, heteroarylthioalkyl, heteroaralkylthioalkyl, cyanoalkyl, alkylsulfinylalkyl, alkylsulfonylalkyl, haloalkylsulfinyl, arylsulfinylalkyl, arylsulfonylalkyl, heteroarylsulfonylalkyl, heteroarylsulfinylalkyl, aralkylsulfinylalkyl, and aralklsulfonylalkyl;
R^{4a} and R^{4b}, when bonded to the same carbon, are taken together to form a group selected from the group consisting of oxo, thiono, and a linear spacer moiety having from 2 through 7 contiguous atoms connected to form a ring selected from the group consisting of a cycloalkyl ring having 3 through 8 contiguous members, a cycloalkenyl ring having 5 through 8 contiguous members, and a heterocyclyl ring having 5 through 8 contiguous members;
E⁰ is E¹, when K is (CR^{4a}R^{4b})ₙ, wherein E⁰ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, S(O)₂N(R⁷)C(O), C(O)N(R⁷)S(O)₂, Se, Se(O), Se(O)₂, Se(O)₂N(R⁷), N(R⁷)Se(O)₂, P(O)(R⁸), N(R⁷)P(O)(R⁸), P(O)(R⁸)N(R⁷), N(R⁷), ON(R⁷), SiR²⁸R²⁹, CR^{4a}CR^{4b}, ethynylidene (C—C; 1,2-ethynyl), and C—CR^{4a}R^{4b};
K is (CH(R¹⁴))ⱼ-T wherein j is selected from a integer from 0 through 3 and T is selected from the group consisting of single covalent bond, O, S, and N(R⁷) with the proviso that (CH(R¹⁴))ⱼ is bonded to the pyridone ring;
E⁰ is E², when K is (CH(R¹⁴))ⱼ-T, wherein B is selected from the group consisting of a covalent single bond, C(O), C(S), C(O)O, C(S)O, C(O)S, C(S)S, C(O)N(R⁷), (R⁷)NC(O), C(S)N(R⁷), (R⁷)NC(S), (R⁷)NC(O)O, (R⁷)NC(S)S, (R⁷)NC(O)S, (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O), S(O)₂N(R⁷), N(R⁷)S(O)₂, S(O)₂N(H)C(O), C(O)N(H)S(O)₂, Se(O), Se(O)₂, Se(O)₂N(R⁷), N(R⁷)Se(O)₂, P(O)(R⁸), N(R⁷)P(O)(R⁸), P(O)(R⁸)N(R⁷), and N(R⁷);
K is G-(CH(R¹⁵))ₖ wherein k is selected from an integer from 1 through 3 and G is selected from the group consisting of O, S, and N(R⁷) with the proviso that R¹⁵ is other than hydroxy, cyano, halo, amino, alkylamino, dialkylamino, and sulfhydryl when k is 1;
E⁰ is E³, when K is G-(CH(R¹⁵))ₖ, wherein E³ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, Se, Se(O), Se(O), Se(O)₂N(R⁷), N(R⁷)Se(O)₂, P(O)(R⁸), N(R⁷)P(O)(R⁸), P(O)(R⁸)N(R⁷), N(R⁷), ON(R⁷), SiR²⁸R²⁹, CR^{4a}═CR^{4b}, ethynylidene (C≡C; 1,2-ethynyl), and C═CR^{4a}R^{4b};
Y⁰ is formula (IV): 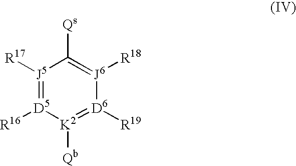
wherein D⁵, D⁶, J⁵, and J⁶ are independently selected from the group consisting of C, N, O, S and a covalent bond with the provisos that no more than one is a covalent bond, K² is independently selected from the group consisting of C, and N⁺, no more than one of D⁵, D⁶, J⁵, and J⁶ is O, no more than one of D⁵, D⁶, J⁵, and J⁶ is S, one of D⁵, D⁶, J⁵, and J⁶ must be a covalent bond when two of D⁵, D⁶, J⁵, and J⁶ are O and S, no more than three of D⁵, D⁶, J⁵, and J⁶ are N when K is N⁺, and no more than four of D⁵, D⁶, J⁵, and J⁶ are N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;
R¹⁶ and R¹⁷ are independently taken together to form a linear moiety spacer having from 3 through 6 contiguous atoms connected to form a ring selected from the group consisting of a cycloalkenyl ring having from 5 through 8 contiguous members, a partially saturated heterocyclyl ring having from 5 through 8 contiguous members, a heteroaryl having from 5 through 6 contiguous members, and an aryl;
R¹⁸ and R¹⁹ are independently taken together to form a linear moiety spacer having from 3 through 6 contiguous atoms connected to form a ring selected from the group consisting of a cycloalkenyl ring having from 5 through 8 contiguous members, a partially saturated heterocyclyl ring having from 5 through 8 contiguous members, a heteroaryl having from 5 through 6 contiguous members, and an aryl;
Q^{b} is selected from the group consisting of NR²⁰R²¹, ⁺NR²⁰R²¹R²², oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² is hydroxy, alkoxy, alkylamino, amino, and dialkylambno and that R²⁰, R²¹, and R²² must be other than be hydroxy, alkoxy, alkylamino, amino, and dialkylamino when K is N⁺;
R²⁰ and R²¹, R²⁰ and R²², and R²¹ and R²² pairs are independently selected to form a spacer pair wherein a spacer pair is taken together to form a linear moiety having from 4 through 7 contiguous atoms connecting the points of bonding of said spacer pair members to form a heterocyclyl ring having 5 through 8 contiguous members with the proviso that no more than one of the group consisting of spacer pairs R²⁰ and R²¹, R²⁰ and R²², and R²¹ and R²² is used at the same time;
Q^{b} is selected from the group consisting of N(R²⁶)SO₂N(R²³)(R²⁴), N(R²⁶)C(O)OR⁵, N(R²⁶)C(O)SR⁵, N(R²⁶)C(S)OR⁵ and N(R²⁶)C(S)SR⁵ with the proviso that no more than one of R²³, R²⁴, and R²⁶ are hydroxy, alkoxy, alkylamino, amino, or dialkylamino when two of the group consisting of R²³, R²⁴, and R²⁶ are bonded to the same atom;
Q^{b} is selected from the group consisting of dialkylsulfonium, trialkyphosphonium, C(NR²⁵)NR²³R²⁴, N(R²⁶)C(NR²⁵)N(R²³)(R²⁴), N(R²⁶)C(O)N(R²³)(R²⁴), N(R²⁶)C(S)N(R²³)(R²⁴), CNR²⁵)OR⁵, C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(NR²⁵)SR⁵, C(O)NR²³R²⁴, and C(O)NR²³R²⁴ with the provisos that no more than one of R²³, R²⁴, and R²⁶ is hydroxy, alkoxy, alkylamino, amino, or dialkylamino when two of the group consisting of R²³, R²⁴, and R²⁶ are bonded to the same atom and that said Q^{b} group is bonded directly to a carbon atom;
R²³, R²⁴, R²⁵, and R²⁶ are independently selected from the group consisting of hydrido, alkyl, hydroxy, alkoxy, alkylamino, dialkylamino, aminoalkyl, and hydroxyalkyl;
R²³ and R²⁴ are taken together to form a linear spacer moiety having from 4 through 7 contiguous atoms connecting the points of bonding to form a heterocyclyl ring having 5 through 8 contiguous members;
R²³ and R²⁵, R²⁴ and R²⁵, R²⁵ and R²⁶, R²⁴ and R²⁶ and R²³ and R²⁶ pairs are independently selected to form a spacer pair wherein a spacer pair is taken together from the points of bonding of selected spacer pair members to form the group L-U-V wherein L, U, and V are independently selected from the group of 1,2-disubstituted radicals consisting of a cycloalkyl radical, a cycloalkenyl radical wherein cycloalkyl and cycloalkenyl radicals are substituted with one or more groups selected from R³⁰ and R³¹, an aryl radical, an heteroaryl radical, a saturated heterocyclic radical and a partially saturated heterocyclic radical wherein said 1,2-substitutents are independently selected from C═O, C═S, C(R²⁸)R³², S(O), S(O)₂, OP(OR³¹)R₃₀ P(O)R³⁰, P(S)R³⁰ and Si(R²⁸)R²⁹;
R²³ and R²⁵, R²⁴ and R²⁵, R²⁵ and R²⁶, R²⁴ and R²⁶ and 23 and R²⁶ pairs are independently selected to form a spacer pair wherein a spacer pair is taken together from the points of bonding of selected spacer pair members to form the group L-U-V wherein L, U, and V are independently selected from the group of radicals consisting of 1,2-disubstituted alkylene radicals and 1,2-disubstituted alkenylene radical wherein said 1,2-substitutents are independently selected from C═o, C═S, C(R²⁸)R²⁹, S(O), S(O)₂” OP(OR³¹)R³⁰, P(O)R³⁰, P(S)R³⁰, and Si(R²⁸)R²⁹ and said alkylene and alkenylene radical are substituted with one or more R³⁰ or R³¹ substituents;
R²³ and R²⁵, R²⁴ and R²⁵, R²⁵ and R²⁶, R²⁴ and R²⁶, and R²³ and R²⁶ pairs are independently selected to form a spacer pair wherein a spacer pair is taken together from the points of bonding of selected spacer pair members to form the group L-U-V wherein L, U, and V are independently selected from the group consisting of O, S, C(O), C(S), C(JH)₂ S(O), SO₂, OP(OR³¹)R³⁰, P(O)R³⁰, P(S)R³⁰, C(R³⁰)R³¹, C—C(R³⁰)R³¹, (O)₂POP(O)₂, R³⁰(O)POP(O)R³⁰, Si(R²⁹)R²⁸, Si(R²⁹)R²⁸Si(R²⁹)R²⁸, Si(R²⁹)R²⁸OSi(R²⁹)R²⁸, (R²⁸)R²⁹COC(R²⁸)R²⁹(R²⁸)R²⁹CSC(R²⁸)R²⁹, C(O)C(R³⁰)═C(R³¹), C(S)C(R³⁰)=C(R³¹), S(O)C(R³⁰)═C(R³¹), SO₂C(R³⁰)=C(R³¹), PR³⁰C(R³⁰)C(R³¹), P(O)R³⁰C(R³⁰)═C(R³¹), P(S)R³⁰C(R³⁰)═C(R³¹), DC(R³⁰)(R³¹)D, OP(OR³¹)R³⁰, P(O)R³⁰, P(S)R³⁰, Si(R²⁸)R²⁹ and N(R³⁰), and a covalent bond with the proviso that no more than any two of L, U and V are simultaneously covalent bonds and the heterocyclyl comprised of by L, U, and V has from 5 through 10 contiguous member;
D is selected from the group consisting of oxygen, C═O, C═S, and S(O)ₘ wherein m is an integer selected from 0 through 2;
J_{H} is independently selected from the group consisting of OR²⁷, SR²⁷ and N(R²⁰)R²¹;
R²⁷ is selected from the group consisting of hydrido, alkyl, alkenyl, alkynyl, aralkyl, aryloxyalkyl, aralkoxyalkyl, alkylsulfinylalkyl, alkylsulfonylalkyl, aralkylthioalkyl, heteroaralkylthioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxyalkyl, halocycloalkenyloxyalkyl, perhaloaryloxyalkyl, heteroaryl, heteroarylalkyl, heteroarylthioalkyl, heteroaralkylthioalkyl, arylsulfinylalkyl, arylsulfonylalkyl, cycloalkylsulfinylalkyl, cycloalkylsufonylalkyl, heteroarylsulfonylalkyl, heteroarylsulfinylalkyl, aralkylsulfinylalkyl and aralkylsulfonylalkyl;
R²⁸ and R²⁹ are independently selected from the group consisting of hydrido, hydroxyalkyl, alkyl, alkenyl, alkynyl, aryl, aralkyl, aryloxyalkyl, acyl, aroyl, aralkanoyl, heteroaroyl, aralkoxyalkyl, alkylsulfinylalkyl, alkylsulfonylalkyl, aralkylthioalkyl, heteroaralkylthioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxyalkyl, haloalkenyloxyalkyl, halocycloalkoxy, halocycloalkoxyalkyl, halocycloalkenyloxyalkyl, perhaloaryl, perhaloaralkyl, perhaloaryloxyalkyl, heteroaryl, heteroarylalkyl, heteroarylthioalkyl, heteroaralkylthioalkyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, arylsulfinylalkyl, arylsulfonylalkyl, aralkylsulfinyl, cycloalkylsulfinylalkyl, cycloalkylsufonylalkyl, heteroarylsulfonylalkyl, heteroarylsulfinylalkyl, aralkylsulfinylalkyl, aralkylsulfonylalkyl, carboxy, dialkoxyphosphono, diaralkoxyphosphono, dialkoxyphosphonoalkyl and diaralkoxyphosphonoalkyl;
R²⁸ and R²⁹ are taken together to form a linear moiety spacer having from 2 through 7 contiguous atoms and forming a ring selected from the group consisting of a cycloalkyl ring having from 3 through 8 contiguous members, a cycloalkenyl ring having from 3 through 8 contiguous members, and a heterocyclyl ring having from 3 through 8 contiguous members;
R³⁰ and R³¹ are independently selected from hydrido, hydroxy, thiol, aryloxy, amino, alkylamino, dialkylamino, hydroxyalkyl, heteroaryloxyalkyl, alkoxy, alkylthio, arylthio, alkyl, alkenyl, alkynyl, aryl, aralkyl, aryloxyalkyl, aralkoxyalkyl, alkylsulfinylalkyl, alkylsulfonylalkyl, aralkylthioalkyl, heteroaralkoxythioalkyl, alkoxyalkyl, heteroaryloxyalkyl, alkenyloxyalkyl, alkylthioalkyl, arylthioalkyl, cycloalkyl, cycloalkylalkyl, cycloalkylalkenyl, ycyoalkenyl, cycloalkenylalkyl, haloalkyl, haloalkenyl, haloaralkylsulfinylalkyl, aralkylsulfonylalkyl, cyanoalkyl, dicyanoalkyl, carboxamidoalkyl, dicarboxamidoalkyl, cyanocarboalkoxyalkyl, carboalkoxyalkyl, dicarboalkoxyalkyl, cyanocycloalkyl, dicyanocycloalkyl, carboxamidocycloalkyl, dicarboxamidocycloalkyl, carboalkoxycyanocycloalkyl, carboalkoxycycloalkyl, dicarboalkoxycycloalkyl, formylalkyl, acylalkyl, dialkoxyphosphonoalkyl, diaralkoxyphosphonoalkyl, phosphonoalkyl, dialkoxyphosphonoalkoxy, diaralkoxyphosphonoalkoxy, phosphonoalkoxy, dialkoxyphosphonoalkylamino, diaralkoxyphosphonoalkylamino, phosphonoalkylamino, dialkoxyphosphonoalkyl, diaralkoxyphosphonoalkyl, sulfonylalkyl, alkoxysulfonylalkyl, aralkoxysulfonylalkyl, alkoxysulfonylalkoxy, aralkoxysulfonylalkoxy, sulfonylalkoxy, alkoxysulfonylalkylamino, aralkoxysulfonylalkylamino, and suifonylalkylamino;
R³⁰ and R³¹ are taken to form a linear moiety spacer group having from 2 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;
Q^{s} is selected from the group consisfing of a single covalent bond, (CR³⁷R³⁸)_{b}—(W⁷)_{az} wherein az is an integer selected from 0 through 1, b is an integer selected from 1 through 4, and W⁰ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), SC(S)N(R¹⁴), SC(O)N(R¹⁴), OC(S)N(R¹⁴), N(R¹⁵)C(O)N(R¹⁴) (R¹⁴)NC(O)N(R¹⁵), N(R¹⁵)C(S)N(R¹⁴), (R¹⁴)NC(S)N(R¹⁴), S(O), S(O)₂, S(O)₂N(R¹⁴), N(R¹⁴)S(O)₂, Se, Se(O), Se(O)₂, Se(O)₂N(R¹⁷), N(R¹⁴)Se(O)₂, P(O)(R⁸), N(R⁷)P(O)(R⁸), P(O)(R⁸)N(R⁷), N(R¹⁴), N(R¹⁴), and SiR²⁸R²⁹, (CH(R¹⁴))_{c}—W¹—(CH(R¹⁵))_{d} wherein c and d are integers independently selected from 1 through 4, and W is selected from the group consisting of 0, S, C(O), C(S), C(O)O, C(S)O, C(O)S, C(S)S, C(O)N(R¹⁴), (R¹⁴)NC(O),
C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁴), N(R¹⁴)C(S)N(R¹⁴), (R¹⁴)NC(S)N(R¹⁵), S(O), S(O)₂, S(O)₂N(R¹⁴), N(R¹⁴)S(O)₂, Se, Se(O), Se(O)₂, Se(O)₂N(R¹⁴), N(R¹⁴)Se(O)₂, P(O)(R⁸), N(R⁷)P(O)(R⁸), P(O)(R⁸)N(R⁷), N(R¹⁴), ON(R¹⁴), SiR²⁸R²⁹, and (CH(R¹⁴))ₑ—W²—(CH(R¹⁵))ₕ wherein e and h are integers independently selected from 0 through 2 and W² is selected from the group consisting of CR^{4a}CR^{4b}, ethynylidene (IC; 1,2-ethynyl), and C═CR^{4a}CR^{4b} with the provisos that R¹⁴ and R¹⁵ are selected from other than halo and cyano when directly bonded to N and that (CR³⁷R³⁸)_{b}, (CH(R¹⁴))^{c}, (CH(R¹⁴))ₑ and are bonded to E
Y⁰ is Q^{b}-Q^{ss} wherein Q^{b} is selected from the group consisting of (CR³⁷R³⁸)_{f} wherein f is an integer selected from 1 through 6, (CH(R¹⁴))_{c}—W¹—(CH(R¹⁵))_{d} wherein c and d are integers independently selected from 1 through 4, and W is selected from the group consisting of W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁵), (R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, Se, Se(O), Se(O)₂, Se(O)₂N(R¹⁴), N(R¹⁴)Se(O)₂, P(O)(R⁸), N(R⁷)P(O)(R⁸), P(O)(R⁸)N(R⁷), N(R¹⁴), ON(R¹⁴), SiR²⁸R²⁹, and (CH(R¹⁴))ₑ—W²—(CH(R¹⁵))ₕ wherein e and h are integers independently selected from 0 through 2 and W² is selected from the group consisting of CR^{4a}═CR^{4b}, ethynylidene (CaC; 1,2-ethynyl), and C═CR^{4a}R^{4b} with the provisos that R¹⁴ and R¹⁵ are selected from other than halo and cyano when directly bonded to N and that (CR³⁷R³⁸)_{f}, (CH(R¹⁵))_{c}, and (CH(R¹⁵))ₑ are bonded to E⁰;
Y⁰ is Q^{b}-Q^{sss} wherein Q^{sss} is (CH(R³⁸))ᵣ—W³, r is an integer selected from 1 through 3, and W³ is selected from the group consisting of 1,1-cyclopropyl, 1,2-cyclopropyl, 1,1-cyclobutyl, 1,2-yclobutyl, 1,2-cyclohexyl, 1,3-yclohexyl, 1,4-cyclohexyl, 1,2-cyclopentyl, 1,3-cyclopentyl, 2,3-morpholinyl, 2,4-morpholinyl, 2,5-morpholinyl, 2,6-morpholinyl, 3,4-morpholinyl, 3,5-morpholinyl, 1,2-piperazinyl, 1,3-piperazinyl, 1,4-piperazinyl, 2,3-piperazinyl, 2,5-piperazinyl, 2,6-piperaziyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,%pyranyl, 2H-pyran-2-one-3,4-yl, 2H-pyran-2-one-4;yl, 4H-pyran-one-2,3-yl, 2,3-tetrahydrofuranyl, 2,4-tetrahydrofuranyl, 2,5-tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the proviso that (CH(R³⁸))ᵣ is bonded to E⁰ and Q^{b} is bonded to lowest numbered substituent position of each W³;
Y⁰ is Q^{b}-Q^{sssr} wherein Q^{sssr} is (CH(R³⁸))ᵣ—W⁴, r is an integer selected from 1 through 3, and W⁴ is selected from the group consisting of 1,2-cyclobutyl, 1,2-cyclohexyl, 1,3-cyclohexyl, 1,4-cyclohexyl, 1,2-cyclopentyl, 1,3-cyclopentyl, 2,3-morpholinyl, 2,4-morpholinyl, 2,5-morpholinyl, 2,6-morpholinyl, 3,4-morpholinyl, 3,5-morpholinyl, 1,2-piperazinyl, 1,3-piperazinyl, 1,4-piperazinyl, 2,3-piperazinyl, 2,5-piperidinyl, 2,6-piperazinyl, 1,2-piperidinyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrrolidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4,5-yl, 4H-pyran-one-2,3-yl, 2,3-tetrahydrofuranyl, 2,4-tetrahydrofuranyl, 2, tetrahydrofuranyl, 3,4-tetrahydrofuranyl, 2,3-tetrahydropyranyl, 2,4-tetrahydropyranyl, 2,5-tetrahydropyranyl, 2,6-tet;ahydropyranyl, 3,4-tetrahydropyranyl, and 3,5-tetrahydropyranyl with the provisos that (CH(R³⁸))ᵣ is bonded to E⁰ and Q^{b} is bonded to highest number substituent position of each W⁴;
Y⁰ is Q^{b}-Q^{ssss} wherein Q^{ssss} is (CH(R³⁸))ᵣ—W⁵, r is an integer selected from 1 through 3, and W⁵ is selected from the group consisting of 1,4-indenyl, 1,5-indenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3, indolyl, 3,6-indolyl-3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl-3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1.4 isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to lowest number substituent position of each W and that (CH(R³⁸))ᵣ is bonded to E⁰;
Y⁰ is Q^{b}-Q^{sssr} wherein Q^{sssr} is (CH(R³⁸))ᵣ—W⁶, r is an integer selected from 1 through 3, and W is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4, quinolinyl, 4,6-quinolinyl, 4,7-quinolinyl, 4,8-quinolinyl, 1,4-isoquinolinyl, 1, isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-nnolinyl, 3,8-cinnolinyl, 4,5-cinnolinyl, 4,6-cinnolinyl, 4,7-cinnolinyl, and 4,8-cinnolinyl with the proviso that Q^{b} is bonded to highest number substituent position of each W⁶ and that (CH(R³⁸))ᵣ is bonded to E⁰;
R³⁷ and R³⁷, when bonded to different carbons, are taken together to form a linear moiety spacer having from 1 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;
R³⁷ and R³⁸, when bonded to different carbons, are taken together to form a linear moiety spacer having from 1 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;
R³⁸ and R³⁸, when bonded to different carbons, are taken together to form a linear moiety spacer having from 1 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;
R³⁷ and R³⁸, when bonded to the same carbon, are taken together to form a group selected from a group consisting of oxo, thiono, alkylene, haloalkylene, and a linear moiety spacer having from 2 through 7 contiguous atoms to form a ring selected from the group consisting of a cycloalkyl ring having from 3 through 8 contiguous members, a cycloalkenyl ring having from 3 through 8 contiguous members, and a heterocyclyl ring having from 3 through 8 contiguous members;
R³⁹ and R⁴⁰, when bonded to the same carbon, are taken together to form a group selected from a group consisting of oxo, thiono, R¹-N, alkylene, haloalkylene, and a linear moiety spacer having from 2 through 7 contiguous atoms to form 8 ring selected from the group consisting of a cycloalkyl ring having from 3 through 8 contiguous members, a cycloalkenyl ring having from 3 through 8 contiguous members, and a heterocyclyl ring having from 3 through 8 contiguous members.

2. The compound as recited in claim 1 or a pharmaceutically acceptable salt thereof, wherein;
B is formula (V): 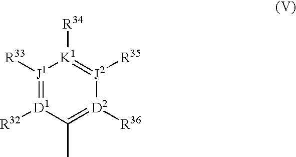
wherein D¹, D², J¹, J² and K¹ are independently selected from the group consisting of C, N, O, S and a covalent bond with the provisos that no more than one is a covalent bond, no more than one of D¹, D², J¹, J² and K¹ is O, no more than one of D¹, D², J¹, J² and K¹ is S, one of D¹, D², J¹, J² and K¹ must be a covalent bond when two of D¹, D², J¹, J² and K¹ are O and S, and no more than four of D¹, D², J¹, J² and K¹ are N with the proviso that 2 R³³, R³⁴, R³⁵, and R³⁶ are each independently selected to maintain the tetravalent nature of carbon, trivalent nature of nitrogen, the divalent nature of sulfur, and the divalent nature of oxygen;
R³², R³³, R³⁴, R³⁵, and R³⁶ are independently selected to be Q^{b};
R⁹, R¹⁰, R¹¹, R¹², R¹³, R¹⁶, R¹⁷, R¹⁸, R¹⁹, R³²R³³, R³⁴, R³⁵, and R³⁶ are independently selected from the group consisting of hydrido, amidino, guanidino, dialkylsulfonium, trialkylphosphonium, dialkylsulfoniumalkyl, carboxy, heteroaralkylthio, heteroaralkoxy, cycloalkylamino, acylalkyl, acylalkoxy, aryloylalkoxy, heterocyclyloxy, aralkylaryl, aralkyl, aralkenyl, aralynyl, heterocyclyl, perhaloaralkyl, aralcylsulfonyl, aralkylsulfonylalkyl, aralkylsulfinyl, aralkylsulfinylalkyl, halocycloalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxyalkyl, cycloalkylenedioxy, halocycloalkoxy, halocycloalkoxyalkyl, halocycloalkenyloxy, halocycloalkenyloxyalkyl, hydroxy, amino, alkoxyamino, thio, nitro, lower alkylamino, alkylthio, alkylthioalkyl, arylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamidosulfonyl, arylsulfonamido, diarylamidosulfonyl, monoalkyl monoaryl amidosulfonyl, arylsulfinyl, arylsulfonyl, heteroarylthio, heteroarylsuifinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylakanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, aminoalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoalkyl, cyano, carbohaloalkoxy, phosphono, phosphonoalkyl, diaralkoxyphosphono, and diaralkoxyphosphonoalkyl;
R³² and R³³, R³³ and R³⁴, R³⁴ and R³⁵, and R³⁵ and R³⁶ pairs are independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 9 contiguous members, a heteroaryl ring having 5 through 6 contiguous members, and an aryl with the proviso that no more than one of the group consisting of spacer pairs R³² and R³³, R³³ and R³⁴, R³⁴ and R³⁵, and R³⁵ and R³⁶ is used at the same time;
B is selected from the group consisting of C3-C8 alkyl, C3-C8 alkenyl, C3 alkynyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;
B is selected from the group consisting of C3-C10 cycloalkyl, C5-C1O cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₁, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;
R⁹ and R¹⁰, R¹⁰ and R¹¹, R¹¹ and R¹², and R¹² and R¹³ pairs are independently selected to form a spacer pair wherein a spacer pair is taken together to form a linear moiety having from 3 through 6 contiguous atoms connecting the points of bonding of said spacer pair members to form a ring selected from the group consisting of a cycloalkenyl ring having 5 through 8 contiguous members, a partially saturated heterocyclyl ring having 5 through 8 contiguous members, a heteroaryl ring having 5 through 6 contiguous members, and an aryl with the proviso that no more than one of the group consisting of spacer pairs R⁹ and R¹⁰, R¹⁰ and R¹¹, R¹¹ and R¹², and R¹² and R¹³ is used at the same time;
A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, P(O)(R⁸), N(R⁷)P(O)(R⁸), P(O)(R⁸)N(R⁷), C(NR⁷)N(R⁷), (R⁷)NC(NR⁷), and N(R⁷);
R⁷ and R⁸ are independently selected from the group consisting of hydrido, hydroxy, alkyl, acyl, aroyl, heteroaroyl, and alkoxyalkyl;
R¹⁴, R¹⁵, R³⁷, and R³⁸ are independently selected from the group consisting of hydrido, hydroxy, halo, cyano, hydroxyalkyl, alkoxy, alkyl, alkoxyalkyl, cycloalkyl, cycloalkylalkyl, cycloalkenyl, cycloalkenylalkyl, haloalkyl, haloalkenyl, haloalkoxy, haloalkoxyalkyl, haloalkenyloxyalkyl, halocycloalkoxy, halocycloalkoxyalkyl, halocycloalkenyloxyalkyl, carboxy, carboxyalkyl, carboalkoxy, carboxamide, and carboxamidoalkyl;
Ψ is selected from the group consisting of NR⁵, O, C(O), C(S), S, S(O), S(O)₂, ON(R⁵), P(O)(R⁸), and CR³⁹R⁴⁰;
R⁵ is selected from the group consisting of hydrido, allyl, alkoxy, alkoxyalkyl, haloalkyl, acyl, aroyl, and heteroaroyl;
R³⁹ and R⁴⁰ are independently selected from the group consisting of hydrido, hydroxy, halo, cyano, hydroxyalkyl, acyl, aroyl, heteroaroyl, acylamido, alkoxy, alkyl, alkoxyalkyl, haloalkyl, haloalkoxy, haloalkoxyalkyl, alkylsulfonyl, haloalkylsulfonyl, carboxy, carboxyalkyl, carboalkoxy, carboxamide, and carboxamidoalkyl;
R¹ is selected from the group consisting of hydrido, alkyl, alkenyl, haloalkyl, and alkoxyalkyl;
X⁰ and R² are independently selected from the group consisting of Z⁰-Q, hydrido, alkyl, alkenyl, and halo;
X⁰ and R² are independently selected from the group consisting of amidino, guanidino, dialkylsulfonium, trialkylphosphonium, dialkylsulfoniumalkyl, heteroarylamino, amino, nitro, alkylamino, arylamino, aralkylamino, alkanoyl, alkenoyl, aroyl, heteroaroyl, aralkanoyl, heteroaralkanoyl, haloalkanoyl, hydroxyhaloalkyl, cyano, and phosphono;
X⁰ and R¹ are taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair x⁰ and R¹ and spacer pair R² and R¹ is used at the same time;
R² and R¹ are taken together to form a spacer pair wherein the spacer pair forms a linear moiety having from 3 through 6 contiguous atoms connecting the points of bonding of said spacer pair members to form a heterocyclyl ring having from 5 through 8 contiguous members with the proviso that no more than one of the group consisting of spacer pair X and R¹ and spacer pair R² and R¹ is used at the same time;
Z⁰ is selected from the group consisting of covalent single bond, (CR⁴¹R⁴²)_{q} wherein q is an integer selected from 1 through 6, (CH(R⁴¹))—W⁰—(CH(R⁴²))ₚ wherein g and p are integers independently selected from 0 through 3 and W⁰ is selected from the group consisting of O, S, C(O), C(S), C(O)O, C(S)O, C(O)S, C(S)S, C(O)N(R⁴¹), (R⁴¹)NC(O), C(S)N(R⁴¹), (R⁴¹)NC(S), OC(O)N(R⁴¹), (R⁴¹)NC(O)O, SC(S)N(R⁴¹), (R⁴¹)NC(S)S, SC(O)N(R⁴¹),(R⁴¹)NC(O)S, OC(S)N(R⁴¹)(R⁴¹)NC(S)O, N(R⁴¹)C(O)N(R⁴²), (R⁴¹)NC(O)N(R⁴²),N(R⁴²)C(S)N(R⁴¹), (R⁴¹)NC(S)N(R⁴²), S(O), S(O)₂, S(O)₂N(R⁴¹),N(R⁴¹)S(O)₂, P(O)(R⁸),
N(R⁷)ₚ(O)(R⁸), P(O)(R⁸)N(R⁷), N(R⁴¹), ON(R⁴¹), and (CH(R⁴¹))ₑ—W²—(CH(R⁴²))ₕ wherein e and h are integers independently selected from 0 through 2 and W² is selected from the group consisting of CR⁴¹—CR⁴², CR⁴¹R⁴²═C; vinylidene), and ethynylidene (C≡C; 1,2-ethynyl), with the provisos that R⁴¹ and R⁴² are selected from other than halo and cyano when directly bonded to N and Z⁰ is directly bonded to the pyridone ring;
R⁴¹ and R⁴² are independently selected from the group consisting of hydrido, hydroxy, halo, cyano, aryloxy, hydroxyatkyl, acyl, aroyl, heteroaroyl, heteroaryloxyalkyl, alkoxy, alkyl, aryl, aralkyl, aryloxyalkyl, amikoxyalkylalkoxy, alkoxyalkyl, heteroaryloxyalkyl, eycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyallyl, halocycloalkoxy, balocycloalkoxyalkyl, halocycloalkenyloxyalkyl, saturated heterocyclyl, partially saturated heterocyclyl, heteroaryl, heteroaralkyl, heteroarylthioalkyl, heteroaralkylthioalkyl, alkylsulfonyl, haloalkylsulfonyl, arylsulfonyl, arylsulfonylalkyl, arlkylsulfonyl, cycloalkylsulfonyl, cycloalkylsufonylalkyl, heteroaryisulfonylalkyl, heteroarylsulfonyl, aold aralkylsulfonylalkyl;
Q^{b} is formula (II): 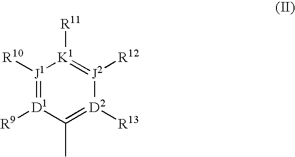
wherein D¹, D², J¹, J² and K¹ are independently selected from the group consisting of C, N, O, S and a covalent bond with the provisos that no more than one is a covalent bond, no more than one of D¹, D², J¹, J² and K¹ is O, no more than one of D¹, D², J¹, J² and K¹ is S, one of D¹, D², J¹, J² and K¹ must be a covalent bond when two of D¹, D², J¹, J² and K¹ are O and S, and no more than four of D¹, D², J¹, J² and K¹ are N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;
Q^{b} is formula (III): 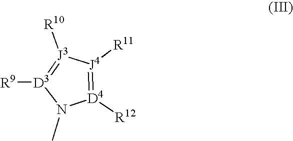
wherein D³, D⁴, J³, and J⁴ are independently selected from the group consisting of C, N, O, and S, no more than one of D³, D⁴, J³, and J⁴ is O, no more than one of D³, D⁴, J³, and J⁴ is S, and no more than three of D¹, D², J¹, and J² are N with the proviso that R⁹, R¹⁰, R¹¹, and R¹² are each independently selected to maintain the tetravalent nature of carbon, trivalent nature of nitrogen, the divalent nature of sulfur, and the divalent nature of oxygen;
Q is selected from the group consisting of alkyl, alkoxy, alkylamino, alkylthio, haloalkylthio, alkenyl, alkynyl, saturated heterocyclyl, partially saturated heterocyclyl, acyl, aroyl, heteroaroyl, cycloalkyl, cycloalkylalkyl, cycloalkenyl, cycloalkenylalkyl, cycloalkylalkenyl, haloalkyl, haloalkoxy, haloalkenyl, halocycloaklyl, halocycloalkenyl, haloalkoxyalkyl, haloalkenyloxyalkyl, halocycloalkoxyalkyl, and balocycloalkenyloxyalkyl;
K is (CR^{4a}R^{4b})ₙ wherein n is an integer selected from 1 through 2;
R^{4a} and R^{4b} are independently selected from the group consisting of halo, hydrido, hydroxy, cyano, hydroxyalkyl, alkyl, alkenyl, alkoxyalkyl, haloalkyl, haloalkenyl, and cyanoalkyl;
R^{4a} and R^{4b}, when bonded to the same carbon, are taken together to form a group selected from the group consisting of oxo, and a linear spacer moiety having from 2 through 7 contiguous atoms connected to form a ring selected from the group consisting of a cycloalkyl ring having 3 through 8 contiguous members, a cycloalkenyl ring having 5 through 8 contiguous members, and a heterocyclyl ring having 5 through 8 contiguous members;
E⁰ is E¹, when K is (CR^{4a}R^{4b})ₙ, wherein E¹ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, S(O)₂N(R⁷)C(O), C(O)N(R⁷)S(O)₂, P(O)(R⁸), N(R⁷)P(O)(R⁸), P(O)(R⁸)N(R⁷), N(R⁷), ON(R⁷), CR^{4a}═CR^{4b}, ethynylidene (C≡C; 1,2-ethynyl), and ═CR^{4a}R^{4b};
K is (CH(R¹⁴))ⱼ-T wherein j is selected from a integer from 0 through 2 and T is selected from the group consisting of single covalent bond, O, S, and N(R⁷) with the proviso that (CH(R¹⁴))ⱼ is bonded to the pyridone ring;
E⁰ is E ², when K is (CH(R¹⁴))ⱼ-T, wherein 2 is selected from the group consisting of a covalent single bond, C(O), C(S), C(O)O, C(S)O, C(O)S, C(S)S, C(O)N(R⁷), (R⁷)NC(O), C(S)N(R⁷), (R⁷)NC(S), (R⁷)NC(O)O, (R⁷)NC(S)S, (R⁷)NC(O)S, (R⁷)NC(S)O, N(R⁸)C(O)N(R⁷), (R⁷)NC(O)N(R⁸), N(R⁸)C(S)N(R⁷), (R⁷)NC(S)N(R⁷), S(O), S(O)₂, S(O)₂N(R⁷), N(R⁷)S(O)₂, S(O)₂N(H)C(O), C(O)N(H)S(O)₂, P(O)(R⁸), N(R⁷)P(O)(R⁸), P(O)(R⁸)N(R⁷), and N(R⁷);
K is G-(CH(R¹⁵))ₖ wherein k is selected from an integer from 1 through 2 and G is selected from the group consisting of O, S, and N(R⁷) with the proviso that R is other than hydroxy, cyano, halo, amino, alklyamino, dialkylamino, and sulfhydryl when k is 1;
E⁰ is E³, when K is G-(CH(R¹⁵))ₖ, wherein B is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R), S(O), S(O)₂, S(O)₂N(R⁷), N(R⁸)S(O)₂, P(O)(R⁸), N(R⁷)P(O)(R⁸), P(O)(R⁸)N(R⁷),
N(R⁷), ON(R⁷), CR^{4a}-CR^{4b}, ethynylidene (CiC; 1,2-ethynyl), and C═CR^{4a}R^{4b};
Y⁰ is formula (IV): 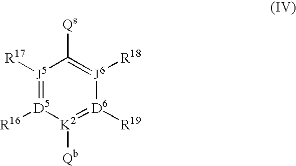
wherein D⁵, D⁶, J⁵, and J⁶ are independently selected from the group consisting of C, N, O, S and a covalent bond with the provisos that no more than one is a covalent bond, K2 is independently selected from the group consisting of C, and N⁺, no more than one of D⁵, D⁶, J⁵, and J⁶ is O, no more than one of D⁵, D⁶, J⁵, and J⁶ is S, one of D⁵, D⁶, J⁵, and J⁶ must be a covalent bond when two of D⁵,D⁶, J⁵, and J⁶ are O and S, no more than three of D⁵, D⁶, J⁵, J⁶ are N when K is N⁺, and no more than four of D⁵, D⁶, J⁵, and J⁶ are N when K² is carbon with the provisos that R¹⁶, R¹⁷, R¹⁸, and R¹⁹ are each independently selected to maintain the tetravalent natur of carbon, trivalent nature of nitrogen, the divalent nature of sulfur, and the divalent nature of oxygen;
R¹⁶ and R¹⁷ are taken together to form a linear moiety spacer having from 3 through 6 contiguous atoms connected to form a ring selected from the group consisting of a cycloalkenyl ring having from 5 through 8 contiguous members, a partially saturated heterocyclyl ring having from 5 through 8 contiguous members, a heteroaryl having from 5 through 6 contiguous members, and an aryl;
Q^{b} is selected from the group consisting of NR²⁰R²¹, NR²⁰R ²¹R²², oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² is hydroxy, alkoxy, alkylamino, amino, and dialkylano and that R²⁰, R²¹ and R²² must be other than be hydroxy, alkoxy, alkylamino, amino, and dialkylano when K2 is N;
R²⁰ and R²¹, R²⁰ and R²², and R²¹ and R²² pairs are independently selected to form a spacer pair wherein a spacer pair is taken together to form a linear moiety having from 4 through 7 contiguous atoms connecting the points of bonding of said spacer pair members to form a heterocyclyl nng having 5 through 8 contiguous members with the proviso that no more than one of the group consisting of spacer pairs R²⁰ and R²¹, R²⁰-and R²², and R²¹ and R²² is used at the same time;
Q^{b} is selected from the group consisting of N(R²⁶)SO₂N(R²³)(R²⁴), N(R²⁶)C(O)OR⁵, N(R²⁶)C(O)SR⁵, N(R²⁶)C(S)OR⁵ and N(R²⁶)C(S)SR⁵ with the proviso that no more than one of R²³, R²⁴, and R²⁶ is hydroxy, alkoxy, alkylamino, amino, or dialkylamino when two of the group consisting of R²³, R²⁴, and R²⁶ are bonded to the same atom;
Q^{b} is selected from the group consisting of dialkylsulfonium, trialkylphosphonium, C(NR²⁵)NR²³R²⁴, N(R²⁶)C(R²⁵)N(R²³)(R²⁴), N(R²⁶)C(O)N(R²³)(R²⁴), N(R²⁶)C(S)N(R²³)(R²⁴), C(NR²⁵)OR⁵, C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁴),N(R²⁶)N(R²⁶)SO₂N(R²³)(R²⁴), C(NR²⁵)SR⁵, C(O)NR²³R²⁴, and C(O)NR²³R²⁴ with the provisos that no more than one of R²³, R²⁴, and R²⁶ is hydroxy, alkoxy, alkylamino, amino, or dialkylamino when two of the group consisting of R²³, R²⁴, and R²⁶ are bonded to the same atom and that said Q group is bonded directly to a carbon atom;
R²³, R²⁴, R²⁵, and R²⁶ are independently selected from the group consisting of hydrido, alkyl, hydroxy, alkoxy, alkylamino, dialkylamino, aminoalkyl, and hydroxyalkyl;
R²³ and R²⁴ are taken together to form a linear spacer moiety having from 4 through 7 contiguous atoms connecting the points of bonding to form a heterocyclyl ring having 5 through 8 contiguous members;
Q^{s} is selected from the group consisting of a single covalent bond, (CR³⁷R³⁸)_{b}—(W⁷)_{az} wherein az is an integer selected from 0 through 1, b is an integer selected from 1 through 4, and W⁰ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), SC(S)N(R¹⁴), SC(O)N(R¹⁴), OC(S)N(R⁴), N(R¹⁵)C(O)N(R¹⁴), (R¹⁴)NC(O)N(R¹⁴), N(R¹⁵)C(S)N(R¹⁴), (R¹⁴)NC(S)N(R¹⁵), S(O), S(O)₂, S(O)₂N(R¹⁴), N(R¹⁴)S(O)₂, P(O)(R⁸), N(R⁷)P(O)(R⁷), P(O)(R⁸)N(R⁷), N(R¹⁴), ON(R¹⁴), (CH(R¹⁴))c-w —(CH(R¹⁵))_{d} wherein c and d are integers independently selected from 1 through 4, and W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴) (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁴), N(R¹⁵)C(S)N(R⁴) (R¹⁴)NC(S)N(R¹⁵), S(O), S(O)₂, S(O)₂N(R¹⁴), N(R¹⁴)S(O)₂, P(O)(R⁸), N(R⁷)P(O)(R⁸), P(O)(R⁸)N(R⁷), N(R¹⁴), ON(R¹⁴), and (CH(R¹⁴))ₑ—W—(CH(R¹⁵))ₕ wherein e and h are integers independently selected from 0 through 2 and W² is selected from the group consisting of CR^{4a}═CR^{4b}, ethynylidene (CGC; 1,2-ethynyl), and C═CR^{4a}R^{4b} with the provisos that R¹⁴ and R¹⁵ are selected from other than halo and cyano when directly bonded to N and that (CR³⁷R³⁸)_{b}, (CH(R¹⁴))_{c} (CH(R¹⁴))e and are bonded to E°;
Y⁰ is Q^{b}-Q^{ss} wherein Q^{ss} is selected from tihe group consisting of (CR^{4a}R^{4b})_{f} wherein f is an integer selected from 1 through 6, (CH(R¹⁴))_{c}—W¹—(CH(R¹⁵))_{d} wherein c and d are integers independently selected from 1 through 4, and W is selected from the group consisting of W is selected from the group consisting of O, S, C(O), C(S), C(O)O, C(S)O, C(O)S, C(S)S, C(O)N(R¹⁴) (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, P(O)(R⁸), N(R⁷)P(O)(R⁸), P(O)(R⁸)N(R⁷), N(R¹⁴), ON(R¹⁴), and (CH(R¹⁴))e, W²(CH(R¹⁵))ₕ wherein e and h are integers independently selected from 0 through 2 and W² is selected from the group consisting of CR^{4a} CR^{4b}, ethynylidene (C≡C; 1,2-ethynyl), and C═CR^{4a}R^{4b} with the provisos that R and R are selected from other than halo and cyano when direcdy bonded to N and that (CR³⁷R³⁸)f, (CH(R¹⁵))c, and (CH(R¹⁵))ₑ are bonded to E;
Y⁰ is Q^{b}-Q^{sss}, wherein Q^{sss} is (CH(R³⁸))ᵣ—W³, r is an integer selected from 1 through 3, and W³ is selected from the group consisting of 1,1-cycloproyl, 1,2-cyclopropyl, 1,2-cyclobutyl, 1,2-cyclobutyl, 1,2-cydohexyl, 1-3-cyclohexyl, 1,4-cyclohexyl, 1,2-cyclopentyl, 1,3-cyclopentyl, 2, morpholinyl, 2,4-rorpholinyl, 2,5-morpholinyl, 2,5-morpholinyl, 3,4-morpholinyl, 3,5-imorpholinyl, 1,2-piperazinyl, 1,3-pipezinyl, 1,4-piperzinyl, 2,3-pipemzinyl, 2,5-piperazhyl, 2,6-piperaziyl, 1,2-pipendinyl, 1,3-pipendinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrolidinyl, 1,3-pyrrolidinyl, 2,3-pyrrolidinyl, 2,4-pyrrolidinyl, 2,5-pyrrolidinyl, 3,4-pynrolidinyl, 2H-2,3-pyranyl, ²H-2,4-pyranyl, 2H-2,5-pyranyl, 4H-2,3-pyranyl, 4H-2,4-pyranyl, 4H-2,%pyranyl, ²H-pyran-2-one-3,4-yl, ²H-pyran-2-one-4,5-yl, 4H-pyran4-one-2,3-yl, 2,3-tetrahydrofuranyl, 2,4-tetrahydrofuianyl, 2, tetrahydrofuranyl, 3,4-tetrahydrofuranyl, 2,3-tetrahydropyranyl, 2,4-tetrahydropyranyl, 2,5-tetrahydropyaanyl, 2,6-tetmhydropyranyl, 3,4-tetrahydropyranyl, and 3,5-tetrahydropyranyl with the proviso that (CH(R³⁸))ᵣ is bonded to E⁰ and Q^{b} is bonded to lowest numbered substituent position of each W³;
Y⁰ is Q_{b}-Q^{sssr}, wherein Q^{sssr} is (CH(R³⁸))ᵣ—W⁴, r is an integer selected from 1 through 3, and W is selected from the group consisting of 1,2-cyclobutyl, 1,2-yclohexyl, 1,3-cyclohexyl, 1,4-yclohexyl, 1,2-cyclopentyl, 1,3-cyclopentyl, 2,3-morpholinyl, 2,4-morpholinyl, 2,5-morpholinyl, 2,6-morpholinyl, 3,4-morpholinyl, 3,5-iorpholinyl, 1,2-piperazinyl, 1,3-piperazinyl, 1,4-pipemzinyl, 2,3-piperazinyl, 2,5-piperazinyl, 2,6-piperaziyl, 1,2-piperidinyl, 1,3-piperidinyl, 1,4-piperidinyl, 2,3-piperidinyl, 2,4-piperidinyl, 2,5-piperidiuyl, 2,6-pipeuidinyl, 3,4-piperidinyl, 3,5-piperidinyl, 3,6-piperidinyl, 1,2-pyn-olidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyrauyl, 2H-2,5-pyranyl, 4H-2,3-pyranyl, 4H-2,4-pyranyl, 4H-2,5-pyranyl, 2H-pyran-2-one-3,4-yl, 2H-pyran-2-one4,5-yl, 4H-pyrantone-2,3-yl, 2,3-tetmhydrofuranyl, 2,4-tetrhydrofuranyl, 2, tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the provisos that (CH(R³⁸))ᵣ is bonded to E⁰ and Q^{b} is bonded to highest number substituent position of each W⁴;
Y⁰ is Q^{b}-Q^{ssss}, wherein Q^{ssss} is (CH(R³⁸))ᵣ—W⁵, r is an integer selected from 1 through 3, and W⁵ is selected from the group consisting of 1,4-indenyl, 1, indenyl, 1,6-indenyl, 1,7-indenyl, 2,7-indenyl, 2,6-indenyl, 2, indenyl, 2,4-indenyl, 3,4-indenyl, 3 Sindenyl, 3,6-indenyl, 3,7-indenyl, 2,4-benzofuranyl, 2,5-benzofuranyl, 2,6-benzofuranyl, 2,7-benzofuranyl, 3,4-benzofurmnyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-sbenzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6 isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyi, 2,1-benzoxazolyl, 2,7-benzoxazolyl, 3,4-benzisoxazolyl, 3,5-benzsoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,9-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnoliyl, 3,6-cinnoiinyl, 3,7-cinolinyl, 3,8-cinnolinyl, 4,5-cinnoliyl, 4,6-cinnolinyl, 4,7-cinnolinyl, and 4,8-cinnolinyl with the proviso that Q^{b} is bonded to lowest number substituent position of each W⁵ and that (CH(1))ᵣ is bonded to E⁰;
Y⁰ is Q^{b}-Q^{ssssr}, wherein Q^{ssssr} is (CH(R³⁸))ᵣ—W⁶, r is an integer selected from 1 through 3, and W is selected from the group consisting of 1,4-indenyl, 1,5-indenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothophenyl, 3,4-benzothiopheiiyl, 3, benzothiophenyl, 3,& benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyi, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-uaphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4S quinolinyl, 4,6-quinoliiiyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnoliny), 3,7-cinnolinyl, 3,8-cinmolinyl, 4,5-cinnolinyl, 4,6-cinnolinyl, 4,7-cinnolinyl, and 4,8-cinnoliyl with the proviso that Q^{b} is bonded to highest nunber substituent position of each W⁶ and that (CH(R³⁸))ᵣ is bonded to E⁰.

3. The compound as recited in claim 1 or a pharmaceutically acceptable salt thereof, wherein;
B is formula (V): 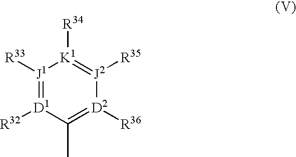
wherein D¹, D², J¹, J² and K¹ are independently selected from the group consisting of C, N, O, S and a covalent bond with the provisos that no more than one is a covalent bond, no more than one of D¹, D², J¹, J² and K¹ is O, no more than one of D¹, D², J¹, J² and K¹ is S, one of D¹, D², J¹, J² and K¹ must be a covalent bond when two of D¹, D², J¹, J² and K¹ are O and S, and no more than four of D¹, D², J¹, J² and K¹ are N;
R³², R³³, R³⁴, R³⁵, and R³⁶ are independently selected to be Q^{b};
R⁹, R¹⁰, R¹¹, R¹², R¹³, R¹⁶, R¹⁷, R¹⁸, R¹⁹, R³², R³⁴, R³⁵, and R³⁶ are independently selected from the group consisting of hydrido, amidino, guanidino, dialkylsulfonium, trialkylphosphonium, dialkylsulfoniumalkyl, carboxy, heteroamlkylthio, beteraralkoxy, cycloalkylamino, acylalkyl, acylalkoxy, aryloylalkoxy, heterocyclyloxy, aralkylaryl, arlll, arlkenyl, aralkynyl, heterocyclyl, perhaloaralkyl, Mamkylsulfonyl, arlkylsulfonylalkyl, arllkylsulfinyl, aralkylsulfinylkyl, halocycoalkyl, halocycloalkenyl, cycloalkylsulfinyl, cycloalkylsulfinylalkyl, cycloalkylsulfonyl, cycloalkylsulfonylalkyl, heteroarylamino, N-heteroarylamino-N-alkylamino, heteroarylaminoalkyl, haloalkylthio, alkanoyloxy, alkoxy, alkoxyalkyl, haloalkoxylalkyl, heteroaralkoxy, cycloalkoxy, cycloalkenyloxy, cycloalkoxyalkyl, cycloalkylalkoxy, cycloalkenyloxylyl, cycloalkylenedioxy, halocycloalkoxy, halocycloalkoxyalkyl, balocycloalkenyloxy, halocycloalkenyloxyalkyl, hydroxy, amino, alkoxyamino, thio, nitro, lower alkylamino, alkylthio, allylthioalkyl, alylamino, aralkylamino, arylthio, arylthioalkyl, heteroaralkoxyalkyl, alkylsulfinyl, alkylsulfinylalkyl, arylsulfinylalkyl, arylsulfonylalkyl, heteroarylsulfinylalkyl, heteroarylsulfonylalkyl, alkylsulfonyl, alkylsulfonylalkyl, haloalkylsulfinylalkyl, haloalkylsulfonylalkyl, alkylsulfonamido, alkylaminosulfonyl, amidosulfonyl, monoalkyl amidosulfonyl, dialkyl amidosulfonyl, monoarylamiidosulfonyl, arylsulfonamido, diarylamidosulfonyl, monoalkyl monoaryl amidosulfonyl, arylsufinyl, arylsulfonyl, heteroarylthio, heteroarylsulfinyl, heteroarylsulfonyl, heterocyclylsulfonyl, heterocyclylthio, alkanoyl, alkenoyl, aroyl, heteroaroyl, aralkanoyl, heteroaralkanoyl, haloalkanoyl, alkyl, alkenyl, alkynyl, alkenyloxy, alkenyloxyalky, alkylenedioxy, haloalkylenedioxy, cycloalkyl, cycloalkylalkanoyl, cycloalkenyl, lower cycloalkylalkyl, lower cycloalkenylalkyl, halo, haloalkyl, haloalkenyl, haloalkoxy, hydroxyhaloalkyl, hydroxyaralkyl, hydroxyalkyl, aminoalkyl, hydoxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, heteroarylalkenyl, carboxyalkyl, carboalkoxy, alkoxycarboxamido, alkylamidocarbonylamido, arylamidocarbonylamido, carboalkoxyalkyl, carboalkoxyalkenyl, carboaralkoxy, carboxamido, carboxamidoaltyl, cyano, carbohaloalkoxy, phosphono, phosphonoalkyl, diaralkoxyphosphono, and diaralkoxyphosphonoalkyl;
B is selected from the group consisting of C3-C8 alkyl, C3-C8 alkenyl, C3-C8 alkynyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R and R₃₆;
B is selected from the group consisting of C3-C10 cycloalkyl, C5-C10 cycloalkenyl, C4-C9 saturated heterocyclyl, and C4-C9 partially saturated heterocyclyl, wherein each Ting carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₀, a ring carbon or nitrogen atom three atoms from the point of attachment and adjacent to the R₁₂ position may be substituted with R₃₃, and a ring carbon or nitrogen atom four atoms from the point of attachment and adjacent to the R₁₁ and R₃₃ positions may be substituted with R₃₄;
A is selected from the group consisting of single covalent bond, (W⁷)ᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, C(NR⁷)N(R⁷), (R⁷)NC(NR⁷), and N(R⁷);
R⁷ and R⁸ are independently selected from the group consisting of hydrido, hydroxy, alkyl, and alkoxyalkyl;
R¹⁴, R¹⁵, R³⁷, and R³⁸ independently selected from the group consisting of hydrido, hydroxy, halo, alkyl, alkoxyalkyl, haloalkyl, haloalkoxy, and haloalkoxyalkyl;
Ψ is selected from the group consisting of NR⁵, O, C(O), C(S), S, S(O), S(O)₂, and CR³⁹R⁴⁰;
R⁵ is selected from the group consisting of hydrido, alkyl, and alkoxy;
R³⁹ and R⁴⁰ are independently selected from the group consisting of hydrido, hydroxy, halo, hydroxyalkyl, alkyl, alkoxyalkyl, haloalkyl, haloalkoxy, and haloalkoxyalkyl;
R¹ is selected from the group consisting of hydrido, alkyl, alkenyl, haloalkyl, and alkoxyalkyl:
X⁰ and R² are independently selected from the group consisting of Z°-Q, hydrido, alkyl, alkenyl, and halo;
X⁰ and R² are independently selected from the group consisting of amidino, guanidino, dialkylsulfonium, trialkylphosphonium, dialkylsulfoniumalkyl, heteroarylamino, amino, nitro, alkylamino, arylamino, aralkylamino, alkanoyl, alkenoyl, aroyl, heteroaroyl, aralkanoyl, heteroaralkanoyl, haloalkanoyl, hydroxyhaloalkyl, cyano, and phosphono;
Z⁰ is selected from the group consisting of covalent single bond, (CR⁴¹R⁴²)_{q} wherein q is an integer selected from 1 through 2, (CH(R⁴¹))_{g}—W⁰—(CH(R⁴²))ₚ wherein g and p are integers independently selected from 0 through 2 and W⁰ is selected from the group consisting of O, S, C(O), C(S), C(O)O, C(S)O, C(O)S, C(S)S, C(O)N(R⁴¹), (R⁴¹)NC(O), C(S)N(R⁴¹), (R⁴¹)NC(S), OC(O)N(R⁴¹), (R⁴¹)NC(O)O, SC(S)N(R⁴¹), (R⁴¹)NC(S)S, SC(O)N(R⁴¹), (R⁴¹)NC(O)S, OC(S)N(R⁴¹),(R⁴¹)NC(S)O, N(R⁴²)C(O)N(R⁴¹), (R⁴¹)NC(O)N(R⁴²), N(R⁴²)C(S)N(R⁴¹), (R⁴¹)NC(S)N(R⁴²), S(O), S(O)₂, S(O)₂N(R⁴¹), N(R⁴¹)S(O)₂, N(R⁴¹), ON(R⁴¹), and (CH(R⁴¹))ₑ—W²(CH(R⁷))ₕ wherein e and h are integers independently selected from 0 through 2 and W² is selected from the group consisting of CR⁴¹═CR⁴² CR⁴¹R⁴²=C; vinylidene), and ethynylidene (C≡C; 1,2-ethynyl), with the provisos that R⁴¹ and R⁴² are selected from other than halo and cyano when directly bonded to N and Z⁰ is directly bonded to the pyridone ring,
R⁴¹ and R⁴² are independently selected from the group consisting of hydrido, hydroxy, halo, cyano, aryloxy, hydroxyallyl, acyl, aroyl, heteroaroyl, heteroaryloxyalkyl, alkoxy, alkyl, aryl, aralkyl, aryloxyalkyl, wmlkoxyalkylalkoxy, alkoxyalkyl, heteroaryloxyalkyl, cycloalkyl, cycloalkylalkyl, cycloalkylalkenyl, cycloalkenyl, cycloalkenylalkyl, haloalkyl, haloalkenyl, halocycloalkyl, halocycloalkenyl, haloalkoxy, haloalkoxyalkyl, haloalkenyloxyalkyl, halocycloalkoxy, halocycloalkoxyalkyl, halocycloalkenyloxyalkyl, saturated heterocycdyl, partially saturated heterocyclyl, heteroaryl, and heteroaralkyl;
Q^{b} is formula (II): 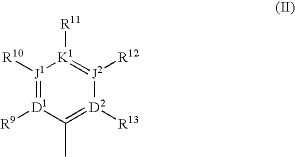
wherein D¹, D², J¹, J² and K¹ are independently selected from the group consisting of C, N, O, S and a covalent bond with the provisos that no more than one is a covalent bond, no more than one of D¹, D², J¹, J² and K¹ is O, no more than one of D¹, D², J¹, J² and K¹ is S, one of D¹, D², J¹, J² and K¹ must be a covalent bond when two of D¹, D², J¹, J² and K¹ are O and S, and no more than four of D¹, D², J¹, J² and K¹ are N, with the proviso that R⁹, R¹⁰, R¹¹, R¹², and R¹³ are each independently selected to maintain the tetravalent nature of carbon, trivalent nature of nitrogen, the divalent nature of sulfur, and the divalent nature of oxygen;
Q^{b} is selected from the group consisting of alkyl, alkoxy, alkylamino, alkylthio, haloalkylthio, saturated heterocyclyl, alkyl, partially saturated heterocyclyl, acyl, aroyl, heteroaroyl, cycloalkyl, cycloalkylalkyl, cycloallenyl, cycloalkenylalkyl, cycloalkylalkenyl, haloalayl, haloalkoxy, haloalkenyl, halocycloalkyl, halocycloalkenyl, haloalkoxyalkyl, haloalkenyloxyalkyl, halocycloalkoxyalkyl, and halocycloalkenyloxyalkyl;
K is (CR^{4a}R^{4b})ₙ wherein n is the integer 1;
R^{4a} and R⁴ are independently selected from the group consisting of halo, hydrido, hydroxy, hydroxyalkyl, alkyl, alkoxyalkyl, and haloalkyl;
E⁰ is E¹, when K is (CR^{4a}R^{4b})ₙ, wherein E¹ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷),(R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂, S(O)₂N(R⁷), N(R⁷)S(O)₂, S(O)₂N(R⁷)C(O), C(O)N(R⁷)S(O)₂, N(R⁷), ON(R⁷), CR^{4a}═CR^{4b}, ethynylidene (C≡C; 1,2-ethynyl), and C═CR^{4a}R^{4b};
K is (CH(R¹⁴))ⱼ-T wherein j is selected from a integer from 0 through 1 and T is selected from the group consisting of single covalent bond, O, S, and N(R⁷) with the proviso that (CH(R¹⁴))ⱼ is bonded to the pyridone ring;
E⁰ is E², when K is (CH(R¹⁴))ⱼ-T, wherein E² is selected from the group consisting of a covalent single bond, C(O), C(S), C(O)O, C(S)O, C(O)S, C(S)S, C(O)N(R⁷), (R⁷)NC(O), C(S)N(R⁷), (R⁷)NC(S), (R⁷)NC(O)O, (R⁷)NC(S)S, (R⁷)NC(O)S, (R⁷)NC(S)O, N(R⁸)C(O)N(R⁷), (R⁷)NC(O)N(R⁸), N(O)C(S)N(R⁷), (R⁷)NC(S)N(R⁸), S(O), S(O)₂,
S(O)₂N(R⁷), N(R⁷)S(O)₂, S(O)₂N(H)C(O), C(O)N(H)S(O)₂, and N(R⁷);
K is G-(CH(R¹⁵))ₖ wberein k is the integer I and G is selected from the group consisting of O, S, and N(R⁷);
E⁰ is E³, when K is G-(CH(R¹⁵))ₖ, wherein E³ is selected from the group consisting of a covalent single bond, O, S, C(O), C(S), C(O)O, C(S)O, C(O)S, C(S)S, C(O)N(R⁷), (R⁷)NC(O), C(S)N(R⁷), (R⁷)NC(S), OC(O)N(R⁷), (R⁷)NC(O)O, SC(S)N(R⁷), (R⁷)NC(S)S, SC(O)N(R⁷), (R⁷)NC(O)S, OC(S)N(R⁷), (R⁷)NC(S)O, N(R⁸)C(O)N(R⁷), (R⁷)NC(O)N(R⁸), N(R⁸)C(S)N(R⁷), (R⁷)NC(S)N(R⁸), S(O), S(O)₂,
S(O)₂N(R⁷), N(R⁷)S(O)₂, N(R⁷), ON(R⁷), CR^{4a}═CR^{4b}, ethynylidene (C≡C; 1,2-ethynyl), and C═CR^{4a}R^{4b};
Y⁰ is formula (IV): 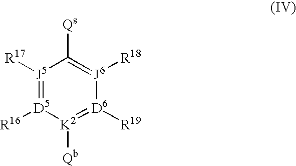
wherein D⁵, D⁶, J⁵, and J⁶ are independently selected from the group consisting of C, N, O, S and a covalent bond with the provisos that no more than one is a covalent bond, K² is independently selected from the group consisting of C and N⁺, no more than one of D⁵, D⁶, J⁵, and J⁶ is O, no more than one of D⁵, D⁶, J⁵, and J⁶ is S, one of D⁵, D⁶, J⁵, and J⁶ must be a covalent bond when two of D, D, J, and J are O and S, no more than three of D⁵, D⁶, J⁵, and J⁶ are N when K² is N⁺, and no more than four of D⁵, D⁶, J⁵ and J⁶ are N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;
Q^{b} is selected from the group consisting of NR²⁰R²¹, ⁺NR²⁰R²¹R²², oxy, alkyl, alkylaminoalkyl, aminoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² is hydroxy, alkoxy, alkylamino, amino, or dialkylamino and that R²⁰, R²¹, and R²² must be other than be hydroxy, alkoxy, alkylamino, amino, and dialkylamino when K2 is N;
Q^{b} is selected from the group consisting of N(R²⁶)SO₂N(R²³)(R²⁴), N(R²⁶)C(O)OR⁵, N(R²⁶)C(O)SR⁵, N(R²⁶)C(S)OR⁵ and N(R²⁶)C(S)SR⁵ with the proviso that no more than one of R²³, R²⁴, and R²⁶ is hydroxy, alkoxy, alkylamino, amino, or dialkylamino when two of the group consisting of R²³, R²⁴, and R²⁶ are bonded to the same atom;
Q^{b} is selected from the group consisting of dialkylsulfonium, trialkylphosphonium, C(NR²⁵)NR²³R²⁴, N(R²⁶)C(NR²⁵)N(R²³)(R²⁴), N(R²⁶)C(O)N(R²³)(R²⁴), N(R²⁶)C(S)N(R²³)(R²⁴), C(NR²⁵)OR⁵, C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁴), N(R²⁶)N(R²⁶)SO,N(R²³)(R²⁴), C(NR²⁵)SR⁵, C(O)NR²³R²⁴, and C(O)NR²³R²⁴ with the provisos that no more than one of R²³, R²⁴, and R²⁶ is hydroxy, alkoxy, alkylamino, amino, or dialkylamino when two of the group consisting of R²³, R²⁴, and R²⁶ are bonded to the same atom and that said Q^{b} group is bonded directly to a carbon atom;
R²³, R²⁴, R²⁵, and R²⁶ are independently selected from the group consisting of hydrido, alkyl, hydroxy, alkoxy, alkylamino, dialkylamino, aminoalkyl, and hydroxyalkyl;
Q^{b} is selected from the group consisting of a single covalent bond, (CR³⁷R³⁸)_{b}—(W⁰)_{az} wherein az is an integer selected from 0 through 1, b is an integer selected from 1 through 2, and W⁰ is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), SC(S)N(R¹⁴), SC(O)N(R¹⁴), OC(S)N(R¹⁴), N(R¹⁵)C(O)N(R¹⁴), (R¹⁴)NC(O)N(R¹⁵), N(R¹⁵)C(S)N(R¹⁴), (R¹⁴)NC(S)N(R¹⁵), S(O), S(O), S(O)₂N(R¹⁴), N(R¹⁴)S(O)₂, N(R¹⁴), ON(R¹⁴), (CH(R¹⁴))_{c}—W¹—(CH(R¹⁵))_{d} wherein c and d are integers independently selected from 1 through 2, and W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁵)C(O)N(R¹⁴), (R¹⁴)NC(O)N(R¹⁵), N(R¹⁵)C(S)N(R¹⁴), (R¹⁴)NC(S)N(R¹⁵), S(O), S(O)₂ S(O)₂N(R¹⁵), N(R¹⁴)S(O)₂, N(R¹⁴), ON(R¹⁴), and (CH(R¹⁴))eCW—(CH(R¹⁵))ₕ wherein e and h are integers independently selected from 0 through 2 and W² is selected from the group consisting of CR^{4a}═CR^{4b}, ethynylidene (C—C; 1,2-ethynyl), and C—CR^{4a}R^{4b} with the provisos that R¹⁴ and R¹⁵ are selected from other than halo and cyano when directly bonded to N and that (CR³⁷R³⁸)_{b}, (CH(R¹⁴))_{c}, and (CH(R¹⁴))ₑ are bonded to B;
Y⁰ is Q^{b}-Q^{ss} wherein Q^{ss} is selected from the group consisting of (CR³⁷R³⁸)_{f} wherein f is an integer selected from 1 through 4, (CH(R¹⁴))_{c}—W¹—(CH(R¹⁵))_{d} wherein c and d are integers independently selected from 1 through 2, and W is selected from the group consisting of W is selected from the group consisting of O, S, C(O), C(S), C(O)O, C(S)O, C(O)S, C(S)S, C(O)N(R¹⁴), (R¹⁴)NC(O), C(S)N(R¹⁴), (R¹⁴)NC(S), OC(O)N(R¹⁴), (R¹⁴)NC(O)O, SC(S)N(R¹⁴), (R¹⁴)NC(S)S, SC(O)N(R¹⁴), (R¹⁴)NC(O)S, OC(S)N(R¹⁴), (R¹⁴)NC(S)O, N(R¹⁴)C(O)N(R¹⁴) (R¹⁴)NC(O)N(R¹⁵), N(R¹⁵)C(S)N(R¹⁴), (R¹⁴)NC(S)N(R¹⁵), S(O), S(O)₂, S(O)₂N(R¹⁴), N(R¹⁴)S(O)₂, N(R¹⁴), ON(R¹⁴), and (CH(R¹⁴))ₑ—W²_(CH(R”)), wherein e and h are integers independently selected from 0 through 2 and W² is selected from the group consisting of CR^{4a}═CR^{4b}, ethynylidene (C≡C; 1,2-ethynyl), and C═CR^{4a}R^{4b} with the provisos that R¹⁴ and R¹⁵ are selected from other than halo when directly bonded to N and that (CR³⁷R³⁸)_{f}, (CH(R¹⁵)), and (CH(R¹⁵))ₑ are bonded to E;
Y⁰ is Q^{b}-Q^{sss} wherein Q^{sss} is (CH(R³⁸))ᵣ—W³, r is an integer selected ftom 1 through 2, and W is selected from the group consisting of 1,1-cyclopropyl, 1,2-cyclopropyl, 1,1-cyclobutyl, 1,2-cyclobuty1,2-cyclohexyl, 1,3-cyclohexyl, 1,4-cyclohexyl, 1,2-cyclopentyl, 1,3-cyclopentyl, 2,3-morpholinyl, 2,4-morpholinyl, 2,5-morpholinyl, 2,6-morpholinyl, 3,4-moipholinyl, 3,5-morpholinyl, 1,2-piperazinyl, 1,3-piperazinyl, 1,4-piperazinyl, 2,3-piperazinyl, 2,5-piperazinyl, 2,6-piperazinyl, 1,2-piperidinyl, 1,3-piperidinyl, 1,4-piperidinyl, 2,3-piperidinyl, 2,4-pipendinyl, 2,5-piperidinyl, 2,6-piperidinyl, 3,4-piperidinyl, 3,5-pipenidinyl, 3,6-piperidinyl, 1,2-pyrrolidinyl, 1,3-pyrrolidinyl, 2,3-pyffolidinyl, 2,4-pyrrolidinyl, 2-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2-one-3,4-yl, 2H-pyran-2-one-4,5-yl, 4H-pyranone-2,3-yl, 2,3-tetrahydrofuranyl, 2,4-tetrahydrofuranyl, 2,5-tetrahydrofuranyl, 3,4-tetrahydrofuranyl, 2,3-tetrahydropyranyl, 2,4-tetahydropyranyl, 2,5-tetrahydropyranyl, 2,6-tetrahydropyranyl, 3,4-tetrahydropyranyl, and 3,5-tetrahydropyranyl with the proviso that (CH( ))ᵣ is bonded to E⁰ and Q^{b} is bonded to lowest numbered substituent position of each W³;
Y⁰ is Q^{b}-Q^{sssr} wherein Q^{sssr} is (CH(R³⁸))ᵣ—W⁴, r is an integer selected from 1 through 2, and W is selected from the group consisting of 1,2-cyclobutyl, 1,2-cyclohexyl, 1,3-cyclohexyl, 1,4-cyclohexyl, 1,2-cyclopentyl, 1, cyclopentyl, 2,3-morpholinyl, 2,4-morpholinyl, 2,5-morpholinyl, 2,6-morpholinyl, 3,4-morphohnyl, 3,5-morpholinyl, 1,2-piperazinyl, 1,3-piperazinyl, 1,4-pipeiazinyl, 2,3-piperinyl, 2,5-piperazinyl, 2,6-pipemzuryl, 1,2-piperidmnyl, 1,3-piperidinyl, 1,4-piperidinyl, 2,3-piperidinyl, 2,4-piperidinyl, 2,5-piperidinyl, 2,6-piperidinyl, 3,4-piperidinyl, 3,5-piperidinyl, 3,6-piperidinyl, 1,2-pyrltidinyl, 1,3-pyrrolidinyl, 2,3-pyrrolidinyl, 2,4-pyrrolidinyl, 2,5-pyrrolidinyl, 3,4-pyrrolidinyl, 2H-2,3-pyranyl, 2H-2,4-pyranyl, 2H-2,5-pyranyl, 4H-2,3-pyranyl, 4H-2,4-pyranyl, 4H-2,5-pyranyl, 2H-pyran-2,5-one,3,4-yl, 2H-pyran-2-one4,5-yl, 4H-pyran-4-one-2,3-yl, 2,3-tetrahydrofuranyl, 2,4-tetrahydrofuranyl, 2, tetrahydrofuranyl, 3,4-tetrahydrofuranyl, 2,3-tetrahydropyranyl, 2,4-tetrahydropyranyl, 2,5-tetrahydropyranyl, 2,6-tetrahydropyranyl, 3,4-tetrahydropyranyl, and 3,5-tetrahydropyranyl with the provisos that (CH(R³⁸))ᵣ is bonded to E⁰ and Q^{b} is bonded to highest number substituent position of each W⁴;
Y⁰ is Q^{b}-Q^{ssss} wherein Q^{ssss} is (CH(R⁸))ᵣ—W⁵, r is an integer selected from 1 through 2, and W⁵ is selected from the group consisting of 1,4-indenyl, lSindenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1-l isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazelyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,;naphthyl, 2,6-naphthyl, 2,7-naphthyl, 2,8-naphthyl, 2,4-quinolinyl, 2,5-uinolinyl, 2,6-quinolinyl, 2,7-quinohinyl, 2,8-quinolinyl, 3,4-quinolinyl, 3,5-quinolinyl, 3,6-quinolinyl, 3,7-quinolinyl, 3,9-quinolinyl, 4,5-quinolinyl, 4,6-quinolinyl, 4,7-quinolinyl, 4,9-quinolinyl, 1,4-isoquinolinyl, 1, isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,9-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinolinyl, 3,7-cinnolinyl, 3,& cinnolinyl, 4,5-cinnolinyl, 4,6-cinnoiinyl, 4,7-cinnolinyl, and 4,8-cinnolinyl with the proviso that Q^{b} is bonded to lowest number substituent position of each W⁵ and that (CH(R³⁸))ᵣ is bonded to E⁰;
Y⁰ is Q^{b}-Q^{ssssr} wherein Q^{ssssr} is (CH(R³⁸))ᵣ—W⁶, r is an integer selected from 1 through 2, and W⁶ is selected from the group consisting of 1,4-indenyl, 1,5-indenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofumnyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,& benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, l, isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-uinolinyl, 3,6-quinolinyl, 3,7-quinolinyl, 3,8-quinolinyl, 4,5-quinolinyl, 4,6-quinolinyl, 4,7-quinolinyl, 4,9-quinolinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to highest number substituent position of each W⁶ and that (CH(R³⁸))ᵣ is bonded to E⁰.

4. The compound as recited in claim 3 or a pharmaceutically acceptable salt thereof, wherein;
B is formula (V): 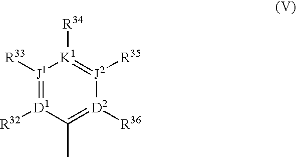
wherein D¹, D², J¹, J² and K¹ are independently selected from the group consisting of C, N, O, S and a covalent bond with the provisos that no more than one is a covalent bond, no more than one of D¹, D², J¹, J² and K¹ is O, no more than one of D¹, D², J¹, J² and K¹ is S, one of D¹, D², J¹, J² and K¹ must be a covalent bond when two of D¹, D², J¹, J² and K¹ are O and S, and no more than four of D¹, D², J¹, J² and K¹ are N;
R³², R³³, R³⁴, R³⁵, and R³⁶ are independently selected to be Q^{b};
R⁹, R¹⁰, R¹¹, R¹², R¹³, R³², R³³, R³⁴, R³⁵, and R³⁶ are independently selected from the group consisting of hydrido, amidiuo, guanidino, diallylsulfonium, carboxy, haloalkylthio, alkanoyloxy, alkoxy, alkoxyalkyl, haloalkoxylalkyl, hydroxy, amino, alkoxyamnuo, thio, nitro, lower alkylamino, alkylthio, alkylthioalkyl, alkylsulfinyl, alkylsulmylalkyl, alkylsulfonyl, alkylsulfonylalkyl, haloalkylsulfinylalkyl, haloalkylsulfonylalkyl, alkylsulfonamido, alkylanunosulfonyl, amidosulfonyl, monoalkyl amidosulfonyl, dialkyl amidosulfonyl, monoarylamidosulfonyl, alkanoyl, alkenoyl, haloalkanoyl, alkyl, alkenyl, alkenyloxy, alkenyloxyalky, halo, haloalkyl, haloalkenyl, haloalkoxy, hydroxyhaloalkyl, hydroxyalkyl, aminoalkyl, haloalkoxyalkyl, carboxyalkyl, carboalkoxy, alkoxycarboxamido, alkylamidocarbonylamido, cazboalkoxyalkyl, carboalkoxyakenyl, carboxamido, carboxamidoalkyl, and cyano;
B is selected from the group consisting of C3-C8 alkyl, C3-C alkenyl, C3-C8 alkynyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;
B is selected from the group consisting of C3-CO1 cycloalkyl, C5-C10 cycloalkenyl, C4-C9 saturated heterocyclyl, and C4-C9 partially saturated heterocyclyl, wherein each ring carbon may be optionally substituted with R₃₃, a rng carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, a ring carbon or nitrogen atom adjacent to the R₁₃ position and two atoms from the point of attachment may be substituted with R₁₂, a ring carbon or nitrogen atom three atoms from the point of attachment and adjacent to the R₁₀ position may be substituted with R₁₁, a ring carbon or nitrogen atom thre atoms from the point of attachment and adjacent to the R₁₂ position may be substituted with R₃₃, and a ring carbon or nitrogen atom four atoms from the point of attachunent and adjacent to the R₁₁ and R₃₃ positions may be substituted with R₃₄;
A is selected from the group consisting of single covalent bond, (W⁷)ᵣᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W is selected from the group consisting of O, S, C(O), C(S), C(O)S, C(S)O, C(O)N(R⁷), C(S)N(R⁷), (R⁷)NC(O), (R⁷)NC(S), S(O), S(O)₂, S(O)₂N(R⁷), (R⁷)NS(O)₂, C(NR⁷)N(R⁷), (R⁷)NC(NR⁷), and N(R⁷) with the proviso that no more than one of the group consisting of rr and pa is O at the same time;
R⁷ and R⁸ are independently selected from the group consisting of hydrido, hydroxy, alkyl, and alkoxyalkyl;
R¹⁵ is selected from the group consisting of hydrido, hydroxy, halo, alkyl, and haloalkyl;
Ψ is NH;
X⁰ is hydrido;
R¹ is selected from the group consisting of hydrido, alkyl, and haloalkyl;
R² is selected from the group consisting of Z⁰-Q, hydrido, alkyl, alkenyl, and halo;
Z⁰ is a covalent single bond;
Q is formula (II): 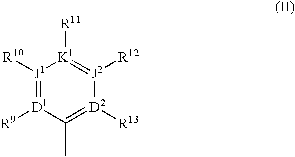
wherein D¹, D², J¹, J² and K¹ are independently selected from the group consisting of C, N, O, S and a covalent bond with the provisos that no more than one is a covalent bond, no more than one of D¹, D², J¹, J² and K¹ is O, no more than one of D¹, D², J¹, J² and K¹ is S, one of D¹, D², J¹, J² and K¹ must be a covalent bond when two of D¹, D², J¹, J² and K¹ are O and S, and no more than four of D¹, D², J¹, J² and K¹ are N, with the proviso that R⁹, R¹⁰, R¹¹, R¹², and R¹³ are each independently selected to rnrintain the tetravalent nature of carbon, trivalent nature of nitrogen, the divalent nature of sulfur, and the divalent nature of oxygen;
K is CR^{4a}R^{4b};
R^{4a} and R⁴ are independently selected from the group consisting of halo, hydrido, hydroxy, alkyl, and haloalkyl;
E⁰ is E¹, when K is CR^{4a}R^{4b}, wherein E is selected from the group consisting of a covalent single bond, (O)N(H), (H)NC(O), C(S)N(H), (H)NC(S), S(O)₂N(H), N(H)S(O)₂, S(O)₂N(H)C(O), and C(O)N(H)S(O)₂;
K is (CH(R¹⁴))ⱼ-T wherein j is selected from an integer from 0 through 1 and T is selected from the group consisting of single covalent bond and N(R⁷) with the proviso that (CH(R¹⁴))ⱼ is bonded to the pyridone ring;
R¹⁴ is selected from the group consisting of hydrido, halo, alkyl, and haloalkyl;
E⁰ is E², when K is (CH(R¹⁴))ⱼ_T, wherein E is selected from the group consisting of C(O)N(H), (H)NC(O), C(S)N(H), (H)NC(S), S(O)N(H), N(H)S(O)₂, S(O)₂N(H)C(O), and C(O)N(H)S(O)₂;
Y⁰ is formula (IV): 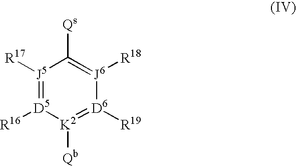
wherein D⁵, D⁶, J⁵, and J⁶ are independently selected from the group consisting of C, N, O, S and a covalent bond with the provisos that no more than one is a covalent bond,K² is independently selected from the group consisting of C and N⁺, no more than one of D⁵, D⁶, J⁵, and J⁶ is O, no more than one of D⁵, D⁶, J⁵, and J⁶ is S, one of D⁵, D⁶, J⁵, and J⁶ must be a covalent bond when two of D⁵, D⁶, J⁵ and J⁶ are O and S, no more than three of D⁵, D⁶, J⁵, and J⁶ are N when K is N⁺, and no more than four of D⁵,D⁶, J⁵, and J⁶ are N when K is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;
R¹⁶, R¹⁷, R¹⁸, and R¹⁹ are independently selected from the group consisting of hydrido, amidino, guanidino, dialkylsulfonium, carboxy, haloalkylthio, alkoxy, hydroxy, amino, thio, nitro, lower alkylaino, alkylthio, allylsulfrnyl, alkylsulfonyl, alkanoyl, alkenoyl, haloalkanoyl, alkyl alkenyl, halo, haloalkyl, haloalkenyl, haloalkoxy, hydroxyalkyl, aminoalkyl, haloalkoxyalkyl, carboalkoxy, carboalkoxyalkyl, and cyano;
Q^{b} is selected from the group consisting of NR²⁰R²¹, ⁺NR²⁰R²¹R²², oxy, alkyl, alkylaminoalkyl, ammoalkyl, dialkylsulfoniumalkyl, and acylamino wherein R²⁰, R²¹, and R²² are independently selected from the group consisting of hydrido, alkyl, hydroxy, alkoxy, alkylamino, dialkylamino, aminoalkyl, and hydroxyalkyl with the provisos that no more than one of R²⁰, R²¹, and R²² is hydroxy, alkoxy, alkylamino, amino, or dialkylamino and that R²⁰, R²¹, and R²² must be other than be hydroxy, alkoxy, alkylamino, amino, or dialkylamino when K² is N⁺;
Q^{b} is N(R²⁶)SO₂N(R²³)(R²⁴) with the proviso that no more than one of R²³, R²⁴, and R²⁶ is hydroxy, alkoxy, alkylamino, amino, or dialkylainino when two of the group consisting of R²³, R²⁴, and R²⁶ are bonded to the same atom;
R²³, R²⁴, R²⁵, and R²⁶ are independently selected from the group consisting of hydrido, alkyl, hydroxy, alkoxy, alkylamino, dialkylammo, aminoalkyl, and hydroxyalkyl;
Q^{b} is selected from the group consisting of dialkylsulfonium, trialkylphosphonium, C(NR²⁵)NR²³R²⁴, N(R²⁶)C(NR²⁵)N(R²³)(R²⁴), N(R²⁶)C(O)N(R²³)(R²⁴),N(R²⁴)C(S)N(R²³)(R²⁴), C(O)N(R²⁶)C(NR²⁵)N(R²³)(R²⁴), C(S)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²³, R²⁴, and R²⁶ is hydroxy, alkoxy, alkylainino, amino, or dialkylamino when two of the group consisting of R²³, R²⁴, and R²⁶ are bonded to the same atom and that said Q^{b} group is bonded directly to a carbon atom;
Q^{s} is selected from the group consisting of a single covalent bond and (CR³⁷R³⁸)_{b}—(WO)_{az} wherein az is an integer selected from 0 through 1, b is an integer selected from 1 through 2, and W⁰ is selected from the group consisting of O, S, C(O), S(O)₂, N(R¹⁴), and ON(R¹⁴) with the proviso thatR is selected from other than halo when directly bonded to N and that (CR³⁷R³⁸)_{b} is bonded to E⁰;
R³⁷ and R³⁸ are independently selected from the group consisting of hydrido, halo, alkyl, and haloalkyl;
Y⁰ is Q^{b}-Q^{ssss} wherein Q^{ssss} is (CH(R³⁸))ᵣ—W⁵, r is an integer selected from 1 through 2, and W⁵ is selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-soindolyl, 3,4-indazolyl, 3,5-indazolyl, 3,6-indazolyl, 3,7-indazolyl, 2,4-benzo xazolyl, 2,5-benzoxazoily, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,8-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinolinyl, 3,4-quinolinyl, 3,5-quinolinyl, 3,6-quinolinyl, 3,7-quinolinyl, 3,8-quinolinyl, 4,5-quinolinyl, 4,6-quinolinyl, 4,7-quinolinyl, 4,8-quinotinyl, 1,4-isoquinolinyl, 1,5-isoquinolinyl, 1,6-isoquinolinyl, 1,7-isoquinolinyl, 1,8-isoquinolinyl, 3,4-isoquinolinyl, 3,5-isoquinolinyl, 3,6 isoquinoliny), 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinolinyl, and 4,8-cinnolinyl with the proviso that Q^{b} is bonded to lowest number substituent position of each W⁵ and that (CH(R³⁸))ᵣ is bonded to E⁰;
Y⁰ is Q^{b}-Q^{ssssr} wherein Q^{ssssr} is (CH(R³⁸))ᵣ—W⁶, r is an integer selected from 1 through 2, and W⁶ is selected from the group consisting of 1,4-indenyl, 1,5-indenyl, 1,6-indenyl, 1,7-indenyl, 2,7-indenyl, 2,6-indenyl, 2, indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofuranyl, 3,6-benzofuranyl, 3,7-benwzofuranyl, 2,4-benzothiophenyl, 2,5-benzothiophenyl, 2,6-benzothiophenyl, 2,7-benzothiophenyl, 3,4-benzothiophenyl, 3,5-sbenzothiophenyl, 3,6-benzothiophenyl, 3,7-benzothiophenyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxazolyl, 1,4-naphthyl, 1,5-naphthyl, 1,6-naphthyl, 1,7-naphthyl, 1,5-naphthyl, 2,4-naphthyl, 2,5-naphthyl, 2,6-naphthyl, 2,7-naphthyl, 2,8-naphthyl, 2,4-quinolinyl, 2,5-quinolinyl, 2,6-quinolinyl, 2,7-quinolinyl, 2,8-quinotinyl, 3,4-quinolinyl, 3,5-quiolinyl, 3,6-quinolinyl, 3,7-quinolinyl, 3,8-quinolinyl, 4,5-quinolinyl, 4,6-quinolinyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinolinyl, 3,4-isoqunolinyl, 3,5-isoquinolinyl, 3,6-isoquinolinyl, 3,7-isoquinolinyl, 3,8-isoquinolinyl, 4,5-isoquinolinyl, 4,6-isoquinolinyl, 4,7-isoquinolinyl, 4,8-isoquinolinyl, 3,4-cinnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl with the proviso that Q^{b} is bonded to highest number substituent position of each W⁶ and that (CH(R₈))ᵣ is bonded to E⁰.

5. The compound as recited in claim 4 having the Formula I-S: 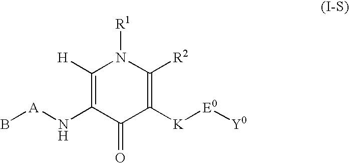
or a pharmaceutically acceptable salt thereof, wherein;
B is selected from the group consisting of phenyl, 2-thienyl, 3-thienyl, 2-furyl, 3-furyl, 2-pyrrolyl, 3-pyrrolyl, 2-imidazolyl, 4-iridazolyl, 3-pyrazolyl, 4-pyrazolyl, 1,2,4-triazol-3-yl, 1,2,4-triazol-5-yl, 1,2,4-oxadiazol-3-yl, 1,2,4-oxadiazol-5-yl, 1,3,4-oxadiazol-3-yl, 1,3,4-oxadiazol-5-yl, 3-isothiazolyl, 5-isothiazolyl, 2-oxazolyl, 2-thiazolyl, 3-isoxazolyl, 5-isoxazolyl, 2-pyridyl, 3-pyridyl, 4-pyridyl, 2-pyrazinyl, 2-pyrinidinyl, 4-pynmidinyl, 5-pyrimidinyl, 3-pynidazinyl,4-pyridazinyl, 1,3,5-triazin-2-yl, 1,2,4-triazin-3-yl, 1,2,4-tnazin-5-yl, 1,2,4-triazin6-yl, and 1,2,3-triazintyl, wherein a carbon adjacent to tIe carbon at the point of attachment may be substituted by R³², the other carbon adjacent to the carbon at the point of attachment may be substituted by R³⁶, a carbon adjacent to R³³ and two atoms from the carbon at the point of attachment may be substituted by R³⁵, a carbon adjacent to R³⁵ and two atoms from the carbon at the point of attachment may be substituted by R³⁵, and any carbon adjacent to both R³³ and R³⁵ may be substituted by R³⁴;
R³², R³³, R³⁴, R³⁵, and R³⁶ are independently selected from the group consisting of hydrido, amidino, guanidino, dimethylsulfoniumn, carboxy, methoxy, ethoxy, isopropoxy, propoxy, hydroxy, amino, methoxyamino, ethoxyamino, thio, ritro, aminomethyl, 1-aminoethyl, 2-aminoethyl, N,N-methylamino, dimethylamino, N-ethylamino, methylthio, ethylthio, isopropyltio, trifluoromethylthio, methylsulfinyl, ethylsilfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-eteafluoroethoxy, fluoro, chloro, bromo, amitdosulfonyl, N-methylamidosulfonyl, N,N dimethylamidosulfonyl, acetyl, propanoyl, trifluoroacetyl, pentafluoropropanoyl, hydroxymethyl, 1-hydroxyethyl, 2-hydroxyethyl, 2,2,2-trifluoro-1-hydroxyethyl, 2,2,2-trnluoro-1-trifiuoromethyl-1-hydroxyethyl, carboxymethyl, methoxycarbonyl, ethoxycarbonyl, amidocarbonyl, N-methylamidocarbonyl, N,N-dimethylamidocarbonyl, cyano, and Q^{b};
B is selected from the group consisting of 1-propenyl, propyl, isopropyl, butyl, 2-butenyl, 3-butenyl, 2-butynyl, sec-butyl, isobutyl, 2-methylpropenyl, 1-pentyl, 2-pentenyl, 3-pentenyl, 4-pentenyl, 2-pentynyl, 3-pentynyl, 2-pentyl, 1-methyl-2-butenyl, 1-methyl-3-butenyl, 1-methyl-2-butynyl, 3-pentyl, 1-ethyl-2-propenyl, 2-methylbutyl, 2-methyl-2-butenyl, 2-methyl-3-butenyl, 2-methyl-3-butynyl, 3-methylbutyl, 3-mnethyl-2-butenyl, 3-methyl-3-butenyl, 1-hexyl, 2-hexenyl, 3-hexenyl, 4-hexenyl, Shexenyl, 2-hexynyl, 3-hexynyl, 4-hexynyl, 2-hexyl, 1-methyl-2-pentenyl, 1-methyl-3-pentenyl, 1-methylpentenyl, 1-methyl-2-pentynyl, 1-methyl-3-pentynyl, 3-hexyl, 1-ethyl-2-butenyl, 1-ethyl-3-butenyl, 1-propyl-2-propenyl, 1-ethyl-2, butynyl, 1-heptyl, 2-heptenyl, 3-heptenyl, 4-heptenyl, 5-heptenyl, 6-heptenyl, 2-heptynyl, 3-heptynyl, 4-heptynyl, 5-heptynyl, 2-heptyl, 1-methyl-2-hexenyl, 1-methyl-3-hexenyl, 1-methylhexenyl, 1-methyl-5-hexenyl, 1-methyl-2-hexynyl, 1-methyl-3-hexynyl, 1-methylhexynyl, 3-heptyl, 1-ethyl-2-pentenyl, 1-ethyl-3-pentenyl, 1-ethyltpentenyl, 1-butyl-2-propenyl, 1-ethyl-2-pentynyl, 1-ethyl-3-pentynyl, 1-octyl, 2-octenyl, 3-octenyl, 4-octenyl, 5-octenyl, 6-octenyl, 7-octenyl, 2-octynyl, 3-octynyl, 4-octynyl, 5-octynyl, 6-octynyl, 2-octyl, 1-methyl-2-heptenyl, 1-methyl-3-heptenyl, 1-methylheptenyl, 1-methyl-5 heptenyl, 1-methyl-beptenyl, 1-methyl-2-heptynyl, 1-methyl-3-heptynyl, 1-methyl4-heptenyl, 1-methyl-5-heptenyl, 1-methylheptenyl, 1-methyl-2-heptenyl, 1-methyl-3-heptynyl, 1-methyl4-heptynyl, 1-methyl-Sheptynyl, 3-octyl, 1-ethyl-2-hexenyl, 1-ethyl-3-hexenyl, 1-ethyl4-hexenyl, 1-ethyl-2-hexynyl, 1-ethyl-3-hexynyl, 1-ethyl4-hexynyl, 1-ethyl-5-hexenyl, 1-pentyl-2-propenyl, 4-octyl, 1-propyl-2-pentenyl, 1-propyl-3-pentenyl, 1-propylX pentenyl, 1-butyl-2-butenyl, 1-propyl-2-pentynyl, 1-propyl-3-pentynyl, 1-butyl-2-butynyl, 1-butyl-3-butenyl, 2,2-difluoropropyl, 4-trifluoromethyl-5,5S trifluoropentyl, 4-trifluoromethylpentyl, 5,5,6,6,6-pentatluorohexyl, and 3,3,3-trifluoropropyl, wherein each member of group B may be optionally substituted at any carbon up to and including 5 atoms from the point of attachment of B to A with one or more of the group consisting of R₃₂, R₃₃, R₃₄, R₃₅, and R₃₆;
B is selected from the group consisting of cyclopropyl, cyctobutyl, oxetan-2-yl, oxetan-3-yl, azetidin-1-yl, azetidin-2-yl, azetidin-3-yl, thiaetan-2-yl, thiaetan-3-yl, cyclopentyl, cyclopent-2-enyl, cyclopent-3-enyl, cyclohexyl, 4 methylcyclohexyl, 4-chloro3-ethylphenoxycyclohexyl, 3-trifiuoromethoxyphenoxycyclohexyl, 3-tuifluoromethylcyclohexyl, 4-trilluoromethylcyclohexyl, 3,5-bis-trifuoromethylcyclohexyl, adamantyl, 3-triuoromethyladamantyl, norbornyl, 3-trifluoromethylnorbornyl, norbornenyl, 7-oxabicyclo[2.2.1]heptan-2-yl, bicyclo[3.1.0]hexanyl, cycdohex-2-enyl, cyclohex-3-enyl, cycloheptyl, cyclohept-2-enyl, cyclohept-3-enyl, cyclooctyl, cyclooct-2,5-enyl, cyclooct-3-enyl, cyclooct4-enyl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 2H-2-pyranyl, 2H-3-pyranyl, 2H-4-pyranyl, 4H-2-pyranyl, 4H-3-pyranyl, 4HApyranyl, 2H-pyran-2-one-3-yl, 2H-pyran-2-one-4-yl, 2H-pyran-2-one-5-yl, 4H-pyran-4-one-2-yl, 4H-pyran-4-one-3-yl, 2-tetrahydrufuranyl, 3-tetrahydrofuimnyl, 2-tttrydropyranyl, 3-tetraiydropyranyl, 4-tetmhydropyranyl, 2-tetrahydrotbienyl, and 3-tetrahydrothienyl, wherein each ring carbon may be optionally substituted with R₃₃, a ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R¹⁰, and a ring carbon or nitrogen atom adjacent to the R₁₃ position and two atoms from the point of attachment may be substituted with R₁₂;
R⁹, R¹⁰, R¹¹, R¹², and R¹³ are independently selected from the group consisting of nmidino, guanidino, dimethylsulfonium, methylethylsulfonium, carboxy, methoxy, ethoxy, isopropoxy, propoxy, butoxy, hydroxy, amino, methoxyamino, ethoxyamino, aminomethyl, 1-aminoethyl, 2-aminoethyl, N-N-dimethylamino, N-methylamino, N-ethylaminno, methylsulfinyl, ethylsulmyl, methylsulfonyl, ethylsulfonyl, amidosulfonyl, N-methylamidosulfonyl, N,N-dimethylamidosulfonyl, acetyl, propanoyl, butanoyl, trifluoroacetyl, pentafluoropropanoyl, hydroxymethyl, 1-hydroxyethyl, 2-hydroxyethyl, 2,2,2-trifluoro-1-hydroxyethyl, 2,2,2-triuoro-1-tdfluoroinethyl-1-hydroxyethyl, carboxymethyl, 2-carboxyethyl, methoxycarbonyl, ethoxycarbonyl, amidocarbonyl, N-methylamidocarbonyl, N,N-dimethylyaidocarbonyl, and cyano;
A is selected from the group consisting of single covalent bond, O, C(O), CH₂, CH₃CH, CF3CH, CH₃CC(O), CF₃CC(O), C(O)CCH₃, C(O)CCF₃, CH₂C(O), (O)CCH₂, CH₂CH₂, CH₂CH₂CH₂, CH₃CCH₂, CF₃CCH₂, CH₃CC(O)CH₂, CF₃CC(O)CH₂, CH₂C(O)CCH₃, CH₂C(O)CCF₃, CH₂CH₂C(O), and CH₂(O)CCH₂;
R¹ is selected from the group consisting of hydrido, methyl, ethyl, propyl, butyl, 2,2,2-trifluoroethyl, and 2,2,3,3,3-pentafluoropropyl;
R² is selected from the group consisting of phenyl, 2-thienyl, 3-thienyl, 2-furyl, 3-fury, 2-pyrrolyl, 3-pyrrolyl, 2-imidazolyl, 4-imidazolyl, 3-pyrazolyl, 4-pyrazolyl, 1,2,4-triazol-3-yl, 1,2,4-triazol-5-yl, 1,2,4-oxadiazol-3-yl, 1,2,4-oxadiazol-5-yl, 1,3,4-oxadiazol-3-yl, 1,3,4-oxadiazol-5-yl, 3-isothiazolyl, 5-isothiazolyl, 2,5-oxazolyl, 2-thiazolyl, 3-isoxazolyl, 5 isoxazolyl, 2-pyridyl, 3-pynidyl, 4-pyridyl, 2-pyrazinyl, 2-pyrimidinyl, 4-pyrnudinyl, 5-pyrinidinyl, 3-pyridazinyl, 4-pytidazinyl, 1,3,5-trian-2-yl, 1,2,4-tr-nn-3-yl, 12,4-triazm-5-yl, 1,2,4-triazin-6-yl, and 1,2,3,4-riazinyl, wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;
K is CR^{4a}R^{4b} wherein R^{4a} and R^{4b} are independently selected from the group consisting of chloro, fluoro, and hydrido;
E⁰ is E¹, when K is CR^{4a}R^{4b}, wherein E¹ is selected from the group consisting of a covalent single bond, C(O)N(H), (H)NC(O), S(O)₂N(H), N(H)S(O)₂, S(O)₂N(H)C(O), and C(O)N(H)S(O)₂;
K is selected from the group consisting of N(H) and CH₂N(H);
E⁰ is E², when K is N(H) and CH₂N(H), wherein E² is selected from the group consisting of C(O)N(H), (li)NC(O), S(O)₂N(H), N(H)S(O)₂, S(O)₂N(H)C(O), and C(O)N(H)S(O)₂;
Y⁰ is selected from the group of formulas consisting of: 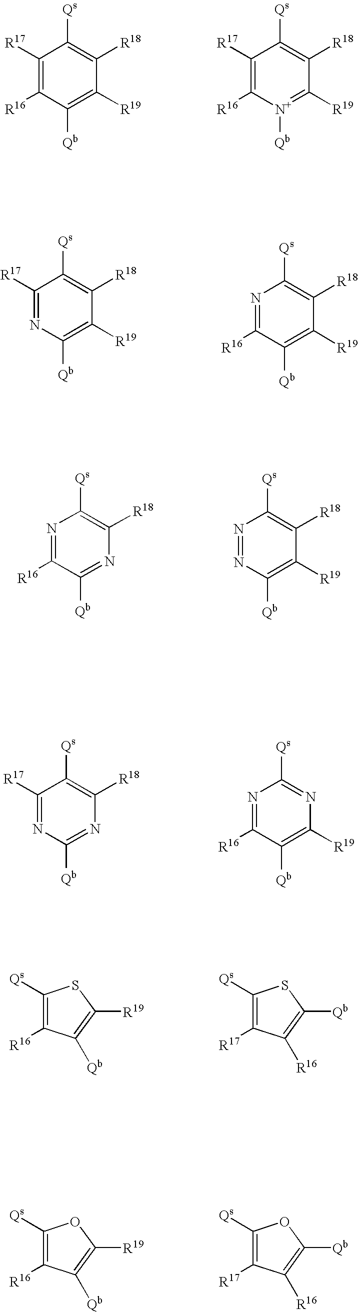 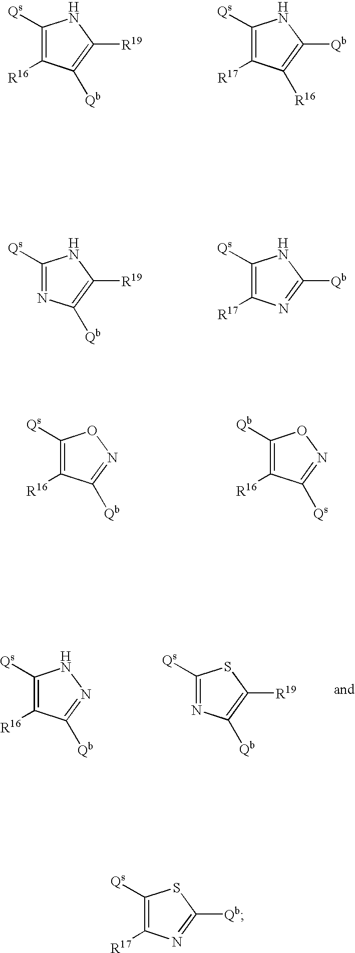
R¹⁶, R¹⁷, R¹⁸, and R¹⁹ are independently selected from the group consisting of hydrido, amidino, guanidino, dimethylsufonium, carboxy, methoxy, etboxy, isopropoxy, propoxy, hydroxy, amino, methoxyamino, ethoxyamino, thio, nitro, aminomethyl, 1-aminoethyl, 2-aminoethyl, N,N-methylamino, dimethylamino, N-ethylamino, methylthio, ethylthio, isopropylthio, triuoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, amidosulfonyl, N-methylamidosulfonyl, N,N-ditnethylaraidosulfonyl, acetyl, propanoyl, trifluoroacetyl, pentafluoropropanoyl, hydroxymethyl, 1-hydroxyethyl, 2-hydroxyethyl, 2,2,2-trifluoro l-hydroxyethyl, 2,2,-trifluoro-1-trifluoromethyl-1-hydroxyethyl, carboxymethyl, methoxycarbonyl, ethoxycarbonyl, amidocarbonyl, N-methylamiidocarbonyl, N,N-dimethylamidocarbonyl, and cyano;
Q^{b} is selected, when bonded to a carbon, from the group consisting of NR²⁰R²¹,NR²⁰R²¹R²², dimethylsulfonium, methylethylsulfonium, diethylsulfonium, trimethylphosphonium, C(NR²⁵)NR²³R²⁴, N(R²⁶)C(NR²⁵)N(R²³)(R²⁴), C(O)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²⁰, R²¹, and R²² is hydroxy, methoxy, ethoxy, N-methylamino, N,N-dimethylamino, N,N,N-trimethylamino, or amino and that no more than one of R²³, R²⁴ and R²⁶ is hydroxy, methoxy, ethoxy, N-methylamino, N,N-dimethylamino, N,N,N-trimethylamino, or amino when two of the group consisting of R²³, R²⁴, and R²⁶ are bonded to the same atom and that said Q group is bonded directly to a carbon atom;
R²⁰, R²¹, R²², R²³, R²⁴, R²⁵ and R²⁶ are independentlyselected from the group consisting of hydrido, methyl, ethyl, propyl, butyl, isopropyl, hydroxy, methoxy, ethoxy, isopropoxy, propoxy, 2-aminoethyl, 2-(N-methylamino)ethyl, 2-(N,N-dimethylamino)ethyl, 2-(N,N,N-trimethylamino)ethyl, N-(2-hydroxyethyl)amino, N,N-bis-(2-hydroxyethyl)amino, N-(2-hydroxyethyl)-N-(2-aminoethyl)amino, N-methylamino, N-ethylamino, N,N-dimethylamino, N,N-diethylamino, and N,N,N-trimethylamino;
Q^{b} is selected, when bonded to a nitrogen, from the group consisting of oxy, methyl, ethyl, 2-aminoethyl, 2-N-methylamino)ethyl, 2-N,N-dimethylamino)ethyl, 2-(N,N,N-trimethylamino)ethyl, N-(2-hydroxyethyl)amino, N,N-bis-(2-hydroxyethyl)amino, amino, hydroxylamino, N-methoxyamino, N-methylamino, N,N-methylamino, and N,N,N-trimethylamino;
Q^{s} is selected from the group consisting of a single covalent bond, CH₂, CH₃CH, CF₂, CF₃CH, CH₂O, CH₃C(H)O, CF₃C(H)O, CH₂S, CH₃C(H)S, CF₃C(H)S, CH₂C(O), CH₃C(H)C(O), CF₃C(H)C(O), and CF₂C(O) with the proviso that Q^{s} is bonded to E⁰ through a carbon atom.

6. The compound as recited in claim 1 or a pharmaceutically acceptable salt thereof, wherein;
B is selected from the group consisting of aryl and heteroaryl wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;
R³², R³³, R³⁴, R³⁵, and R³⁶ are independently selected from the group consisting of hydrido, amidino, guanidino, dialkylsulfonium, carboxy, haloalkylthio, alkoxy, hydroxy, amino, alkoxyamino, thio, nitro, lower alkylamino, alkylthio, alkylsulfinyl, alkylsulfonyl, amidosulfonyl, alkanoyl, haloalkanoyl, allftl, halo, haloalkyl, haloalkenyl, haloalkoxy, hydroxyhaloalkyl, hydroxyalkyl, aminoalkyl, carboxyalkyl, carboalkoxy, carboxamido, cyano, and Q^{b};
B is selected from the group consisting of C3-C8 alkyl, C3-C8 alkenyl, C3-C8 haloalkyl, and C3-C8 haloalkenyl wherein each member of group B may be optionally substituted at any carbon up to and including 6 atoms from the point of attachment of B to A with one or more of the group consisting of R₃₂, R₃₃, R₃₄, R₃₅, and R₃₆;
B is selected from the goup consisting of C3-C10 cycloalkyl, C5-C10 cycloalkenyl, C4-C9 saturated heterocyclyl, and C4-C9 partially saturated heterocyclyl, wherein each ring carbon may be optionally substituted with R₃₃, a ring carbon other than the ring carbon at the point of attachment of B to A may be optionally substituted with oxo provided that no more than one ring carbon is substituted by oxo at the same time, ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R¹⁰, a ring carbon or nitrogen atom adjacent to the R₁₃ position and two atoms from the point of attachment may be substituted with R₁₂, a ring carbon or nitrogen atom three atoms from the point of attachment and adjacent to the R¹⁰ position may be substituted with R₁₁, a ring carbon or nitrogen atom tffie atoms from the point of attachment and adjacent to the R₁₂ position may be substituted with R₃₃, and a ring carbon or nitrogen atom four atoms fron the point of attachment and adjacent to the R₁₁ and R₃₃ positions may be substituted with R₃₄;
R⁹, R¹⁰, R¹¹, R¹², and R¹³ are independently selected from the group consisting of hydrido, amidino, guanidino, dialkylsulfonium, carboxy, haloalkylthio, alkoxy, hydroxy, amino, alkoxyamino, thio, nitro, lower alkylamino, alkylthio, alkylsulfinyl, alkylsulfonyl, amidosulfonyl, alkanoyl, haloalkanoyl, alkyl, halo, haloalkyl, haloalkenyl, haloalkoxy, hydroxyhaloalkyl, hydroxyalkyl, aminoalkyl, carboxyalkyl, carboalkoxy, carboxamido, and cyano;
A is selected from the group consisting of single covalent bond, (W⁷)ᵣ—(CH(R¹⁵))ₚₐ and (CH(R¹⁵))ₚₐ—(W⁷)ᵣᵣ wherein rr is an integer selected from 0 through 1, pa is an integer selected from 0 through 6, and W⁷ is selected from the group consisting of O, S, and C(O) with the proviso that no more than one of the group consisting of rr and pa is the integer 0 at the same time;
R¹⁵ is selected from the group consisting of hydrido, hydroxy, halo, alkyl, and haloalkyl;
Ψ is NH;
X⁰ is hydrido;
R¹ is selected from the group consisting of hydrido, alkyl, and haloalkyl;
R² is Q, wherein Q^{b} is selected from the group consisting of aryl and heteroaryl wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;
K is CR^{4a}R^{4b} wherein R^{4a} and R^{4b} are independendy selected from the group consisting of halo and hydrido;
E⁰ is E¹, when K is CR^{4a}R^{4b}, wherein E is selected from the group consisting of a covalent single bond, C(O)N(H), (H)NC(O), S(O)₂N(H), N(H)S(O)₂, S(O)₂N(H)C(O), and C(O)N(H)S(O)₂;
K is (CH(R¹⁴))ⱼ-T wherein j is selected from an integer from 0 through 1 and T is selected from the group consisting of single covalent bond and N(R⁷) with the proviso that (CH(R¹⁴))ⱼ is bonded to the pyridone ring;
R⁷ is selected from the group consisting of hydrido, hydroxy, alkyl, and alkoxyalkyl;
R¹⁴ is selected from the group consisting of hydrido and halo;
E⁰ is E², when K is (CH(R¹⁴))ⱼ-T, wherein E² is selected from the group consisting of C(O)N(H), (H)NC(O), S(O)₂N(H), N(H)S(O)₂, S(O)₂N(H)C(O), and C(O)N(H)S(O)₂;
Y⁰ is formula (IV): 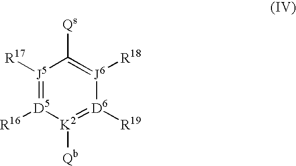
wherein D⁵, D⁶, J5, and J⁶ are independently selected from the group consisting of C, N, O, S and a covalent bond with the provisos that no more than one is a covalent bond, K is independently selected from the group consisting of C and N⁺, no more than one of D⁵, D⁶, J⁵, and J⁶ is O, no more than one of D⁵,D⁶, J⁵, and J⁶ is S, one of D⁵,D⁶, J⁵, and J⁶ must be a covalent bond when two of D⁵,D⁶, J⁵, and J⁶ are O and S, no more than three of D¹, D⁶, J⁵, and J⁶ are N when K2 is N⁺, and no more than four of D⁵,D⁶, J⁵, and J⁶ are N when K² is carbon with the provisos that R¹⁶, R¹⁷, R¹⁸, and R¹⁹ are each independently selected to maintain the tetravalent nature of carbon, trivalent nature of nitrogen, the divalent nature of sulfur, and the divalent nature of oxygen;
R¹⁶, R¹⁷, R¹⁸, and R¹⁹ are independently selected from the group consisting of hydrido, amidino, guanidino, dialkylsulfonium, carboxy, haloalkylthio, alkoxy, hydroxy, amino, thio, nitro, lower alkylaino, alkylthio, alkylsulfinyl, alkylsulfonyl, alkanoyl, alkenoyl, haloalkanoyl, alkyl, alkenyl, halo, haloalkyl, haloalkenyl, haloalkoxy, hydroxyallyl, aminoalkyl, haloalkoxyalkyl, carboalkoxy, carboalkoxyalkyl, and cyano;
Q^{b} is selected from the group consisting of NR²⁰R²¹, ⁺NR²⁰R²¹R²² oxy, alkyl, alkylaminoalkyl, aminoalkyl, dialkylsulfoniumalkyl, and acylamino wherein R²⁰, R²¹, and R²² are independently selected from the group consisting of hydrdo, alkyl, hydroxy, alkoxy, alkylmino, dialkylamino, aminoalkyl, and hydroxyalkyl with the provisos that no more than one of R²⁰, R²¹, and R²² is hydroxy, alkoxy, alkylamino, amino, or dialkylamino and that R²⁰, R²¹, and R²² must be other than be hydroxy, alkoxy, alkylamino, amino, or dialkylamino when K² is N;
Q^{b} is selected from the group consisting of dialkylsulfonium, trialkylphosphonium, C(NR²⁵)NR²³R²⁴, N(R²⁶)C(NR²⁵)N(R²³)(R²⁴), C(O)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²³, R²⁴, and R²⁶ is hydroxy, alkoxy, alkylamino, amino, or dialkylamino when two of the group consisting of R²³, R²⁴, and R²⁶ are bonded to the same atom and that said Q group is bonded directly to a carbon atom;
R²³, R²⁴, R²⁵, and R²⁶ are independently selected from the group consisting of hydrido, alkyl, hydroxy, alkoxy, alkylamino, dialkylamino, aminoalkyl, and hydroxyalkyl;
Q^{s} is selected from the group consisting of a single covalent bond and (CR³⁷R³⁸)_{b}—(W⁷)_{az} wherein az is an integer selected from 0 through 1, b is the integer 1, and W⁰ is selected from the group consisting of O, S, and C(O) with the proviso that (CR³⁷R³⁸)_{b} is bonded to E;
R³⁷ and R³⁸ are independently selected from the group consisting of hydrido, halo, alkyl, and haloalkyl.

7. The compound as recited in claim 6 having the Formula I-MPS: 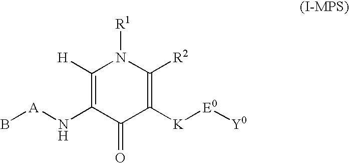
or a pharmaceutically acceptable salt thereof, wherein;
B is selected from the group consisting of phenyl, 2-thienyl, 3-thienyl, 2-furyl, 3-furyl, 2-pyrrolyl, 3-pyrrolyl, 2-imidazolyl, 4-ixndazolyl, 3-pyrazolyl, 4-pymzolyl, 2-thiazolyl, 3-isoxazolyl, 5-isoxazolyl, 2-pyridyl, 3-pyridyl, 4 pyridyl, 2-pyrazinyl, 2-pyrmidinyl, 4-pynmidinyl, 5-pyrimidinyl, 3-pyridazinyl, 4-pyridazinyl, and 1,3,5-triazin-2-yl, wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;
R³², R³³, R³⁵, and R³⁶ are independently selected from the group consisting of hydrido, amidino, guanidino, dimethylsulfonium, carboxy, methoxy, ethoxy, isopropoxy, propoxy, hydroxy, amino, methoxyamino, ethoxyamino, thio, nitro, aminomethyl, 1-aminoethyl, 2-aminoethyl, N,N-methylamino, dimethylamino, N-ethylamino, methylthio, ethylthio, trifluoromethylthio, methylsulfinyl, ethylsulfinyl, methylsulfonyl, ethylsulfonyl, bifluoromethyl, pentafluoroethyl, 2,2,2-ftrfluoroethyl, 2,2,3,3,3-pentafluoropropyl, tifluoromethoxy, 1,1,2,2-tetrafluoroethoxy, fluoro, chloro, bromo, amidosulfonyl, N-methylamidosulfonyl, N,N-dimethylamidosulfonyl, acetyl, propanoyl, trifluoroacetyl, pentafluoropropanoyl, hydroxymethyl, 1-hydroxyethyl, 2-hydroxyethyl, 2,2,2-trifluoro-1-hydroxyethyl, 2,2,2-trifluoro-1-trifluoromethyl-1-hydroxyethyl, carboxymethyl, methoxycarbonyl, ethoxycarbonyl, amidocarbonyl, N-methylamidocarbonyl, N,N-dimethylamidocarbonyl, cyano, and Q^{b};
B is selected from the group consisting of 1-propenyl, propyl, isopropyl, butyl, 2-butenyl, 3-butenyl, sec-butyl, isobutyl, 2-methylpropenyl, 1-pentyl, 2-pentenyl, 3-pentenyl, 4-pentenyl, 2-pentyl, 1-methyl-2-butenyl, 1-methyl-3-butenyl, 3-pentyl, 1-ethyl-2-propenyl, 2-methylbutyl, 2-methyl-2-butenyl, 2-methyl-3-butenyl, 3-methylbutyl, 3-methyl-2-butenyl, 3-methyl-3-butenyl, 2,2-difluoropropyl, 2-trifluoromethyl-3,3,3-trifluoropropyl, 1,1,1,2,2,2-hexafluoropropyl, 3,3,3-tnfluoroprop 1-yl, and 3,3,3-trifluoroprop-2-yl, wherein each member of group B may be optionally substituted at any carbon up to and including 5 atoms from the point of attachment of B to A with one or more of the group consisting of R₃₂, R₃₃, R₃₄, R₃₅, and R₃₆;
B is selected from the group consisting of cyclopropyl, cyclobutyl, oxetan-2-yl, oxetan-3-yl, azetidin-1-yl, azetidin-2-yl, azetidin-3-yl, tbiaetan-2-yl, thiaetan-3-yl, wherein each ring carbon may be optionally substituted with R₃₃, a ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R₁₀, and a ring carbon or nitrogen atom adjacent to the R₁₃ position and two atoms from the point of attachment may be substituted with R₁₂;
R⁹, R¹⁰, R¹¹, R¹², and R¹³ are independently selected from the group consisting of amidino, guanidino, dimethylsulfonium, carboxy, methoxy, ethoxy, isopropoxy, propoxy, hydroxy, amino, methoxyamino, ethoxyamino, aminomethyl, 1-aminoethyl, 2-aminoethyl, N—N-methylamino, dimethylamino, N-ethylamino, methylsulfinyl, ethylsulfinyl, methylsulfonyl, ethylsulfonyl, amidosulfonyl, N-methylamidosulfonyl, N,N-dimethylamidosulfonyl, acetyl, propanoyl, trifluoroacetyl, pentafluoropropanoyl, hydroxymethyl, 1-hydroxyethyl, 2-hydroxyethyl, 2,2,2-tnfluoro-1-hydroxyethyl, 2,2,2-trifluoro-1-trifluoromethyl-1-hydroxyethyl, carboxymethyl, methoxycarbonyl, ethoxycarbonyl, amidocarbonyl, N-methylamidocarbonyl, N,N-dimethylamidocarbonyl, and cyano;
A is selected from the group consisting of single covalent bond, O, C(O), CH₂, CH₃CH, CF3CH, CH₃CC(O), CF3CC(O), CC(O)CCH₃,
C(O)CCF₃, CH₂C(O), and (O)CCH₂;
R¹ is selected from the group consisting of hydrido, methyl, ethyl, and 2,2,2-trifluoroethyl;
R² is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, 5 isoxazolyl, 2-pyridyl, 3-pyridyl, 4 pyridyl, 2-pyrazinyl, 2-pyriridinyl, 4-pyrirnidinyl, 5-pyrimidinyl, 3-pyridazinyl, 4-pyridazinyl, and 1,3,5-triazin-2-yl, wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;
K is CR^{4a}R^{4b} wherein R^{4a} and R^{4b} are independently selected from the group consisting of chloro, fluoro, and hydrido;
E⁰ is E², when K is CR^{4a}R^{4b}, wherein E is selected from the group consisting of a covalent single bond, C(O)N(H), (H)NC(O), S(O)₂N(H), N(H)S(O)₂, S(O)₂N(H)C(O), and C(O)N(H)S(O)₂;
K is selected from die group consisting of N(H) and CH₂N(H);
E⁰ is E², when K is selected from the group consisting of N(H) and CH₂N(H), wherein E² is selected from the group consisting of C(O)N(H), (H)NC(O), S(O)₂N(H), N(H)S(O)₂, S(O)₂N(H)C(O), and C(O)N(H)S(O)₂;
Y⁰ is selected from the group of formulas consisting of: 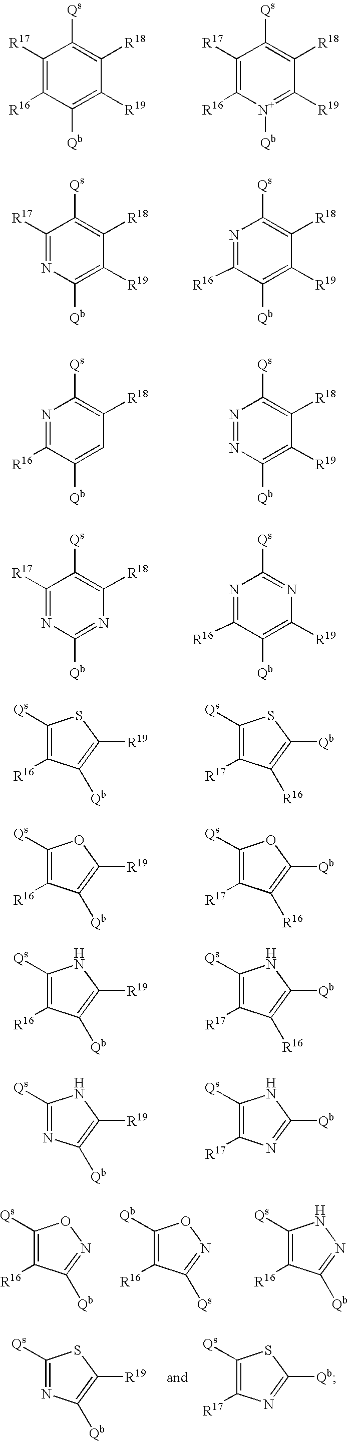
R¹⁶, R¹⁷, R¹⁸, and R¹⁹ are independendy selected from the group consisting of hydrido, amidino, guanidino, methoxy, ethoxy, isopropoxy, methylthio, ethyltdio, trifluoromethylthio, trifluoromethyl, pentafluoroethyl, 2,2,2-trifluoroethyl, 2,2,3,3,3-pentafluoropropyl, trifluoromethoxy, 1,1,2,2-tetrfluoroethoxy, fluoro, chloro, bromo, acetyl, propanoyl, trifluoroacetyl, pentafluoropropanoyl, methoxycarbonyl, ethoxycarbonyl, and cyano;
Q^{b} is selected, when bonded to a carbon, from the group consisting of NR²⁰R²¹, ⁺NR²⁰R²¹, R²², dimethylsulfonium, methylethylsulfonium, diethylsulfonium, trimethylphosphonium, C(NR²⁵)NR²³R²⁴ N(R²⁶)C(NR²⁵)N(R²³)(R²⁴), C(O)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴) C(O)NR²³R²⁴, and C(O)NR²³R²⁴ with the provisos that no more than one of R²⁰, R²¹, and R²² is hydroxy, methoxy, ethoxy, N-methylamino, N,N-dimethylamino, N,N,N-trimethylamino, or amino and that no more than one of R²³, R²⁴, and R²⁶ is hydroxy, methoxy, ethoxy, N-methylamino, N,N-dimethylamino, N,N,N-trimethylamino, or amino when two of the group consisting of R²³, R²⁴, and R²⁶ are bonded to the same atom and that said Q^{b} group is bonded direcdy to a carbon atom;
R²⁰, R²¹, R²², R²³, R²⁴, R²⁵, and R²⁶ are independently selected from the group consisting of hydrido, methyl, ethyl, hydroxy, methoxy, ethoxy, 2-aminoethyl, 2-N-methylamino)ethyl, 2-,N-dimethylamino)ethyl, 2-(,N,N-trimethylamino)ethyl, N-(2-hydroxyethyl)amino, N,N-bis-2-hydroxyethyl)amino, N-(2-hydroxyethyl)-N-(2-aminoethyl)amino, N-methylamino, N,N-dimethylamino, and N,N,N-trimethylamino;
Q^{b} is selected, when bonded to a nitrogen, from the group consisting of oxy, methyl, ethyl, 2-aminoethyl, 2-(N-methylamino)ethyl, 2-(N,N-dimethylamino)ethyl, 2-N,N,N-trimethylamino)ethyl, N-2-hydroxyethyl)amino, N,N-bis-(2-hydroxyethyl)amino, amino, hydroxylamino, N-methoxyamino, N-methylarniino, N,N-dimethylamino, and N,N,N-trimethylamino;
Q^{s} is selected from the group consisting of a single covalent bond, CH₂, CH₃CH, CF₂, CF3CH, CH₂O, CH₃C(H)O, CF₃C(H)O, CH₂₈, CH₃C(H)S,
CF₃C(H)S, CH₂C(O), CH₃C(H)C(O), CF₃C(H)C(O), and CF₂C(O) with the proviso that Q^{s} is bonded to E⁰ through a carbon atom.

8. The compound as recited in claim 7 having the Formula I-EMPS: 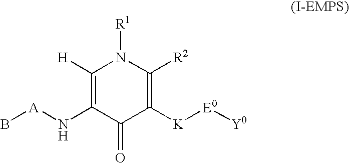
or a pharmaceutically acceptable salt thereof, wherein;
B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2,5-thiazolyl, 3-isoxazolyl, 5 isoxazolyl, 2,5-pyridyl, 3-pyridyl, and 4 pyridyl, wherein a carbon adjacent to the carbon at the point of attachment may be substituted by R³², the other carbon adjacent to the carbon at the point of attachment may be substituted by R³⁶, a carbon adjacent to R³² and two atoms from the carbon at the point of attachment may be substituted by R³³, a carbon adjacent to R³⁶ and two atoms from the carbon at the point of attachment may be substituted by R³⁵, and any carbon adjacent to both R³³ and R³⁵ may be substituted by R³⁴;
R³², R³³, R³⁴, R³⁵, and R³⁶ are independently selected from the group consisting of hydrido, amidino, guanidino, methoxy, ethoxy, hydroxy, amino, methoxyamino, ethoxyamino, aminomethyl, 1-aminoethyl, 2-aminoethyl, N-N-methylamino, dimethylamino, N-thylamino, trifluoromethyl, pentafluoroethyl, 2,2,2-triluoroethyl, 2,2,3,3,3-pentafluoropropyl, fluoro, chloro, bromo, hydroxymethyl, 1-hydroxyethyl, 2-hydroxyethyl, 2,2,2-trifluoro-1-hydroxyethyl, 2,2,2-trifluoro-1-trifluoromethyl-1-hydroxyethyl, methoxycarbonyl, ethoxycarbonyl, cyano, and Q^{b};
B is selected from the group consisting of propyl, isopropyl, butyl, sec-butyl, isobutyl, 1-pentyl, 2-pentyl, 3-pentyl, 2-methylbutyl, 3-methylbutyl, 2,2-difluoropropyl, 2-trifluoromethyl-3,3,3-trifluoropropyl, 1,1,1,2,2,2-hexafluoropropyl, 3,3,3-trifluoroprop-1-yl, and 3,3,3-trifluoroprop-2-yl, wherein each member of group B may be optionally substituted at any carbon up to and including 5 atoms from the point of attachment of B to A with one or more of the group consisting of R₃₂, R₃₃, R₃₄, R₃₅, and R₃₆;
B is selected from the group consisting of cyclopropyl, cyclobutyl, oxetan-2-yl, oxetan-3-yl, azetidin-1-yl, azetidin-2-yl, azetidin-3-yl, thiaetan-2-yl, thiaetan-3-yl, wherein each ring carbon may be optionally substituted with R₃₃, a ring carbon and nitrogen atoms adjacent to the carbon atom at the point of attachment may be optionally substituted with R₉ or R₁₃, a ring carbon or nitrogen atom adjacent to the R₉ position and two atoms from the point of attachment may be substituted with R¹⁰, and a ring carbon or nitrogen atom adjacent to the R₁₃ position and two atoms from the point of attachment may be substituted with R₁₂;
R⁹, R¹⁰, R¹¹, R¹², and R¹³ are independentdy selected from the group consisting of amidino, guanidino, carboxy, methoxy, ethoxy, hydroxy, amino, methoxyamino, ethoxyamino, aminomethyl, 1-aminoethyl, 2-aminoethyl, N,N-methylamino, dimethylamino, N-ethylamino, acetyl, propanoyl, trifluoroacetyl, pentafluoropropanoyl, hydroxymethyl, 1-hydroxyethyl, 2-hydroxyethyl, carboxymethyl, methoxycarbonyl, ethoxycarbonyl, amidocarbonyl, N-methylamidocarbonyl, N,N-dimethylamidocarbonyl, and cyano;
A is selected from the group consisting of single covalent bond, O, C(O), CH₂, CH₂C(O), and (O)CCH₂;
R¹ is selected from the group consisting of hydrido and methyl;
R² is selected from the group consisting of phenyl, 2-thienyl, 3-thienyl, 2-furyl, 3-furyl, 2-pyrrolyl, 3-pyrrolyl, 2-;midazolyl, 4-imidazolyl, 3-pyrazoIyl, 4-pyrazolyl, 2-thiazolyl, 3-isoxazolyl, 5 isoxazolyl, 2-pyridyl, 3-pyridyl, 4 pyridyl, 2-pyrazinyl, 2-pyrimidinyl, 4-pyrinidinyl, Spyrimidinyl, 3-pyridazinyl, 4-pyridazinyl, and 1,3,5-triazin-2-yl, wherein a carbon adjacent to the carbon at the point of attachment may be substituted by R⁹, the other carbon adjacent to the carbon at the point of attachment may be substituted by R¹³, a carbon adjacent to R⁹ and two atoms from the carbon at the point of attachment may be substituted by R¹⁰, a carbon adjacent to R¹³ and two atoms from the carbon at the point of attachment may be substituted by R¹², and any carbon adjacent to both R¹⁰ and R¹² may be substituted by R¹¹;
K is CR^{4a}R^{4b} wherein R^{4a} and R^{4b} are independently selected from the group consisting of chloro, fluoro, and hydrido;
E⁰ is E¹, when K is CR^{4a}R^{4b}, wherein E is selected from the group consisting of a covalent single bond, C(O)N(H), (H)NC(O), S(O)₂N(H), N(H)S(O)₂, S(O)₂N(H)C(O), and C(O)N(H)S(O)₂;
K is selected from the group consisting of N(H) and CH₂N(H);
E⁰ is E², when K is selected from the group consisting of N(H) and CH₂N(H), wherein E² is selected from the group consisting of C(O)N(H), (H)NC(O), S(O)₂N(H), N(H)S(O)₂, S(O)₂N(H)C(O), and C(O)N(H)S(O)₂;
Y⁰ is selected from the group of formulas consisting of: 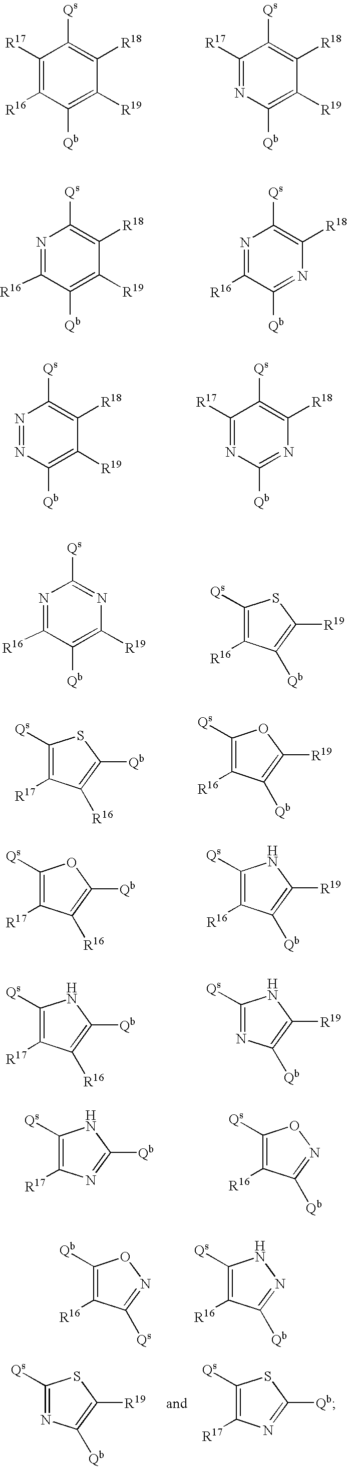
R¹⁶, R¹⁷, R¹⁸, and R¹⁹ are independently selected from the group consisting of hydrido, methoxy, ethoxy, trifluoromethyl, pentafluoroethyl, 2,2,2-trifluoroethyl, trifluoromethoxy, fluoro, chloro, bromo, acetyl, trifluoroacetyl, methoxycarbonyl, ethoxycarbonyl, and cyano;
Q^{b} is selected from the group consisting of NR²⁰R²¹, ⁺NR²⁰R²¹R²², dimethylsulfonium, methylethylsulfonium, diethylsulfonium, trimethylphosphonium, C(NR²⁵)NR²³R²⁴, N(R²⁶)C(NR²⁵)N(R²³)(R²⁴), C(O)N(R²⁶)C(NR²⁵)N(R²³)(R²⁴), N(R²⁶)N(R²⁶)C(NR²⁵)N(R²³)(R²⁴), N(R²⁶)N(R²⁶)SO₂N(R²³)(R²⁴), C(O)NR²³R²⁴, and C(O)NR²³R²⁴ with the provisos that no more than one of R²⁰, R²¹, and R²² is hydroxy, methoxy, ethoxy, N-methylamino, N,N-dimethylamino, and N,N,N-trimethylamino, or amino and that no more than one of R²³, R²⁴, and R²⁶ is hydroxy, methoxy, ethoxy, N-methylamino, N,N-dimethylamino, N,N,N-trinethylamino, or amiino when two of the group consisting of R²³, R²⁴, and R²⁶ are bonded to the same atom;
R²⁰, R²¹, R²², R²³, R²⁴, R²⁵, and R²⁶ are independently selected from the group consisting of hydrido, methyl, ethyl, hydroxy, methoxy, ethoxy, 2-aminoethyl, 2(N-methylamino)ethyl, 2-(N,N-dimethylamiko)ethyl, 2-(N,N,N-trimethylamino)ethyl, N-(2-hydroxyethyl)amino, N,N-bis42-hydroxyethyl)amino, N-(2-hydroxyethyl)-N-2,5-aminoethyl)amino, N-methylamino, N,N-dimethylamino, and N,N,N-trimethylamino;
Q^{s} is selected from the group consisting of a single covalent bond, CH₂, CH₃CH, CF₂, CF3CH, CH₂O, CH₃C(H)O, CF3C(H)O, CH₂C(O), CH₃C(H)C(O), CF₃C(H)C(O), and CF₂C(O) with the proviso that Q^{s} is bonded to EI through a carbon atom.

9. A composition for inhibiting thrombotic conditions in blood comprising a compound of any one of claims 1 through 8 and a pharmaceutically acceptable carrier.

10. A method for inhibiting thrombotic conditions in blood comprising adding to blood a therapeutically effective amount of a composition of claim 9.

11. A method for inhibiting formation of blood platelet aggregates in blood comprising adding to blood a therapeutically effective amount of a composition of claim 9.

12. A method for inhibiting thrombus formation in blood comprising adding to blood a therapeutically effective amount of a composition of claim 9.

13. A method for treating or preventing venuous thromboembolism and pulmonary embolism in a mammal comprising administering to the mammal a therapeutically effective amount of a composition of claim 9.

14. A method for treating or preventing deep vein thrombosis in a mammal comprising administering to the mammal a therapeutically effective amount of a composition of claim 9.

15. A method for treating or preventing cardiogenic thromboembolism in a marmnal comprising administering to the mammal a therapeutically effective amount of a composition of claim 9.

16. A method for treating or preventing thromboembolic stroke in humans and other mammals comprising administering to the mammal a therapeutically effective amount of a composition of claim 9.

17. A method for treating or preventing thrombosis associated with cancer and cancer chemotherapy in humans and other mammals comprising administering to the mammal a therapeutically effective amount of a composition of claim 9.

18. A method for treating or preventing unstable angina in humans and other mammals comprising administering to the mammal a therapeutically effective amount of a composition of claim 9.

19. A method for inhibiting thrombus formation in blood comprising adding to blood a therapeutically effective amount of a compound of any one of claims 1 through 8 with a therapeutically effective amount of fibrinogen receptor antagonist.

20. The use of a compound of any one of claims 1 through X, or a pharmaceutically acceptable salt thereof, in the manufacture of medicament for inhibiting thrombus formation, treating thrombus formation, or preventing thrombus formation in a mammal.

21. A compound of the Formula: 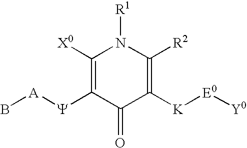
or a pharmaceutically acceptable salt thereof, wherein;
B is phenyl or a heteroaryl of 5 or 6 ring members, wherein a nitrogen with a removable hydrogen or a carbon adjacent to the carbon at the point of attachment of said phenyl or heteroaryl ring to A is optionally substituted by R³² a nitrogen with a removable hydrogen or a carbon at the other position adjacent to the point of attachment is optionally substituted by R³⁶, a nitrogen with a removable hydrogen or a carbon adjacent to R³² and two atoms from the point of attachment is optionally substituted by R³³, a nitrogen with a removable hydrogen or a carbon adjacent to R³⁶ and two atoms from the point of attachment is optionally substituted by R³⁵, and a nitrogen with a removable hydrogen or a carbon adjacent to both R³³ and R³⁵ is optionally substituted by R³⁴;
R⁹, R¹⁰, R¹¹, R¹², R¹³, R³², R³³, R³⁴, R³⁵, and R³⁶ are independently selected from the group consisting of hydrido, acetamido, haloacetamido, amidino, guanidino, alkylenedioxy, haloalkyltlio, alkanoyloxy, alkoxy, cycloalkoxy, cycloalkylalkoxy, antikoxy, aryloxy, heteroaryloxy, heteroaralkoxy,heterocyclyloxy, heterocyclylalkoxy, alkoxyalkyl, haloalkoxylalkyl, hydroxy, amino, alkoxyamino, nitro, alkylamino, N-alkyl-N-arylamino, arylamino, aralkylamino, heteroarylamino, heteroaralkylamino, heterocyclylamino, heterocyclylalkylamino, alkylthio, alkylthioalkyl, alkylsulfinyl, arylsulfrnyl, aralkylsulfinyl, cycloalkylsulfinyl, heteroarysulinyl, alkylsulfonyl, atylsulfonyl, aralkylsulfonyl, cycloalkylsulfonyl, heteroarylsulfonyl, alkylsulfonylalkyl, aryl, aralkyl, cycloalkyl, cycloalkylalkyl, heteroaryl, heterocyclyl, alkylsulfonanudo, amidosulfonyl, alkanoyl, haloalumoyl, alkyl, alkenyl, halo, haloalkyl, haloalkenyl, haloalkoxy, hydroxyhaloalkyl, hydroxyalkyl, aminoalkyl, haloalkoxyalkyl, carboxyalkyl, carboalkoxy, carboxy, carboxartido, carboxamidoalkyl, and cyano;
R³² R³³ R³⁴, R³⁵, and R³⁶ areindependently optionally Q
B is optionally selected from the group consisting of hydrido, trialkylsilyl, C2-C8 alkyl, C3-C8 alkylenyl, C3-Cs alkenyl, C3-s alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶;
B is optionally a C3-C12 cycloalkyl or C4-C9 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen atom adjacent to the R⁹ position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;
A is selected from the group consisting of a bond, (W⁷)ᵣ—(CH(R¹⁵))ₚₐ, and (CH(R¹⁵))ₚₐ, (W⁷)ᵣᵣ wherein rr is 0 or 1, pa is an integer selected from 0 through 6, and W is selected from the group consisting of O, S, C(O), (R⁷)NC(O), (R⁷)NC(S), and N(R⁷) with the proviso that no more than one of the group consisting of rr and pa is O at the same time;
R⁷ is selected from the group consisting of hydrido, hydroxy, and alkyl;
R¹⁵ is selected from the group consisting of hydrido, hydroxy, halo, alkyl, and haloalkyl;
Ψ is NH or NOH;
X⁰ is selected from the group consisting of hydrido, alkyl, alkenyl, cyano, halo, haloalkyl, haloalkoxy, haloalkylthio, amino, aminoalkyl, alkylanuno, amidino, hydroxy, hydroxyamino, alkoxy, hydroxyalkyl, alkoxyamino, thiol, and alkylthio;
R¹ is selected from the group consisting of hydrido, alkyl, alkenyl, haloalkyl, amino, aminoalkyl, alkylamino, hydroxy, and hydroxyalkyl;
X⁰ and R¹ or R¹ and R² is optionally bonded together to form a partially saturated C5-C8 heterocyclyl ring, wherein said heterocyclyl ring is optionally substituted with one or more of the group consisting of R⁹, R¹⁰, R¹¹, R¹², and R¹³;
R² is Z⁰-Q^{b};
Z⁰ is selected from the group consisting of a bond, (CR⁴¹R⁴²)_{q} wherein q is an integer selected from 1 through 3, and (CH(R⁴¹))_{g}—W⁰—(CH(R⁴²))ₚ wherein g and p are integers independently selected from 0 through 3 and W⁰ is selected from the group consisting of O, S, C(O), S(O), N(R⁴¹), and ON(R⁴¹);
Z⁰ is optionally (CH(R⁴¹))ₑ—W²²—(CH(R⁴²))ₕ wherein e and h are independently 0 or 1 and W²² is selected from the group consisting of CR⁴¹═CR⁴², 1,2-cyclopropyl, 1,2-cyclobutyl, 1,2-cyclohexyl, 1,3-cyclohexyl, 1,2-cyclopentyl, 1,3-cyclopentyl, 2,3-morpholinyl, 2,4-morpholinyl, 2,6-morpholinyl, 3,4-morpholinyl, 3,5-morpholinyl, 1,2-piperazinyl, 1,3-piperainyl, 2,3-piperazinyl, 2,6-piperazinyl, 1,2-piperidinyl, 1,3-piperidinyl, 2,3-piperidinyl, 2,4-piperidinyl, 2,6-piperidinyl, 3,4-piperidinyl, 1,2-pyrrolidityl, 1,3-pynolidinyl, 2,3-pyrrolidinyl, 2,4-pyrrolidinyl, 2,5-pyrrolidinyl, 3,4-pyrrolidinyl, 2,3-tetrahydrofuranyl, 2,4-tetmhydrofuranyl, 2,5-tetrahydrofuranyl, and 3,4-tetrahydrofuranyl, wherein Z⁰ is directly bonded to the 4-pyridone ring and W²² is optionally substituted with one or more substituents selected from the group consisting of R⁹, R¹⁰, R¹¹, R¹², and R¹³;
R⁴¹ and R⁴² are independently selected from the group consisting of amnidino, hydroxyamino, hydrido, hydroxy, amino, and alkyl;
Q^{b} is phenyl or a heteroaryl of 5 or 6 ring members, wherein a nitrogen with a removable hydrogen or a carbon adjacent to the carbon at the point of attachment of said phenyl or heteroaryl ring to Z⁰ is optionally substituted by R⁹, a nitrogen with a removable hydrogen or a carbon at the other position adjacent to the point of attachment is optionally substituted by R¹³, a nitrogen with a removable hydrogen or a carbon adjacent to R⁹ and two atoms from the point of attachment is optionally substituted by R¹⁰, a nitrogen with a removable hydrogen or a carbon adjacent to R¹³ and two atoms from the point of attachment is optionally substituted by R¹², and a nitrogen with a removable hydrogen or a carbon adjacent to both R¹⁰ and R¹² is optionally substituted by R¹¹;
Q is optionally hydrido with the proviso that Z⁰ is selected from other than a bond;
K is CR^{4a}R^{4b};
R^{4a} and R^{4b} are independently selected from the group consisting of halo, hydrido, hydroxyalkl, alkyl, alkoxyalkyl, alkylthioalkyl, and haloalkyl;
E⁰, with the proviso that K is CR^{4a}R^{4b}, is E¹ wherein E¹ is selected from the group consisting of a covalent single bond, C(O)N(H), (H)NC(O), C(S)N(H), (H)NC(S), S(O)₂N(H), N(H)S(O), S(O)₂N(H)C(O), and C(O)N(H)S(O)₂;
K is optionally (CH(R¹⁴))ⱼ-T wherein j is 0 or 1 and T is a bond or N(R⁷) with the proviso that (CH(R¹⁴))ⱼ is bonded to the phenyl ring;
R¹⁴ is selected from the group consisting of hydrido, halo, alkl, and haloalkyl;
E⁰, with the proviso that K is (CH(R¹⁴))ⱼ-T, is E² wherein 2 is selected from the group consisting of C(O)N(H), (H)NC(O), C(S)N(H), (H)NC(S), S(O)₂N(H), N(H)S(O)₂, S(O)₂N(H)C(O), and C(O)N(H)S(O)₂;
Y⁰ is phenyl or a heteroaryl of 5 or 6 ring members, wherein one carbon of said phenyl or said heteroaryl is substituted by Q^{s}, a carbon two or three contiguous atoms from the point of attachment of Q^{s} to said phenyl or said heteroaryl to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon ad jacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, nitro, alkoxyamino, alkylamino, alkylthio, alkylsulfinyl, alkylsulfonyl, allanoyl, haloalkanoyl, alkyl, alkenyl, halo, haloalkyl, haloalkoxy, hydroxyalkyl, aminoalkyl, haloalkoxyalkyl, carboalkoxy, and cyano;
R¹⁶ or R¹⁹ is optionally selected from the group consisting of NR²⁰R²¹, N(R²⁶)C(NR²⁵)N(R²³)(R²⁴), and C(NR²⁵)NR²³R²⁴, with the proviso that R¹⁶, R¹⁹, and Q^{b} are not simultaneously hydrido;
Q^{b} is selected from the group consisting of NR²⁰R²¹, aminoalkyl, hydrido, N(R²⁶)C(NR²⁵)N(R²³)(R²⁴), and C(NR²⁵)NR²³R²⁴, with the proviso that no more than one of R²⁰ and R²¹ is selected from the group consisting of hydroxy, amino, alkylamino, and dialkylamino at the same time, with the further proviso that no more than one of R²³ and R²⁴ is selected from the group consisting of hydroxy, amino, alkylamino, and dialkylamino at the same time;
R²⁰, R²¹, R²³, R²⁴, R²⁵, and R²⁶ are independendy selected from the group consisting of hydrido, alkyl, hydroxy, aminoalkyl, amino, dialkylamino, alkylamino, and hydroxyalkyl;
Q^{s} is selected from the group consisting of a bond, (CR³⁷R³⁸)_{b} wherein b is an integer selected from 1 through 4, and (CH(R¹⁴))_{c}—W¹—(CH(R¹⁵))_{d} wherein c and d are integers independently selected from 1 through 3 and W is selected from the group consisting of C(O)N(R¹⁴), (R¹⁴)NC(O), S(O), S(O)₂, S(O)₂N(R¹⁴), N(R¹⁴)S(O)₂, and N(R¹⁴), with the proviso that R¹⁴ is selected from other than halo when directly bonded to N, and with the additional proviso that (CR³⁷R³⁸)_{b} and (CH(R¹⁴))_{c} are bonded to E⁰;
R³⁷ and R³⁸ are independently selected from the group consisting of hydrido, alkyl, and haloalkyl;
R³⁸ is optionally aroyl or heteroaroyl, wherein R³⁸ is optionally substituted with one or more substituents selected from the group consisting of R¹⁶, R¹⁷, R¹⁸, and R¹⁹;
Y⁰ is optionally Y wherein Y^{AT} iS Q^{b}-Q^{s};
Y⁰ is optionally Q^{b}-Q^{ss} wherein Q^{ss} is (CH(R¹⁴))ₑ—W²—(CH(R¹⁵))ₕ, wherein e and h are independently 1 or 2 and W² is CR^{4a}═CR^{4b}, with the proviso that (CH(R¹⁴))ₑ is bonded to E⁰;
Y⁰ is optionally Q^{b}-Q^{ssss} or Q_{b}-Q^{ssssr} wherein Q^{ssss} is (CH(R³⁸))ᵣ—W⁵, Q^{ssssr} is (CH(R³⁸))ᵣ—W⁶, r is 1 or 2, W and W⁶ are independently selected from the group consisting of 1,4-indenyl, 1,5-indenyl, 1,6-indenyl, 1,7-indenyl, 2,7-indenyl, 2,6-indenyl, 2,5-indenyl, 2,4-indenyl, 3,4-indenyl, 3,5-indenyl, 3,6-indenyl, 3,7-indenyl, 2,4-benzofuranyl, 2,5-benzofuranyl, 2,6-benzofuranyl, 2,7-benzofuranyl, 3,4-benzofuranyl, 3,5-benzofumnyl, 3,6-benzofuranyl, 3,7-benzofuranyl, 2,4-benzothiophenyl, 2,5-benzothiophenyl, 2,6-benzothiophenyl, 2,7-benzothiophenyl, 3,4-benzothiophenyl, 3,5-benzothiophenyl, 3,6-benzothiophenyl, 3,7-benzothiophenyl, 2,7-imidazo(1,2-a)pyndinyl, 3,4-imidazo(1,2-a)pyjidinyl, 3,5-imidazo(1,2-a)pynidinyl, 3,6-imidazo(1,2-a)pyridinyl, 3,7-imidazo(1,2-a)pyridinyl, 2,4-indolyl, 2,5-indolyl, 2,6-indolyl, 2,7-indolyl, 3,4-indolyl, 3,5-indolyl, 3,6-indolyl, 3,7-indolyl, 1,4-isoindolyl, 1,5-isoindolyl, 1,6-isoindolyl, 2,4-isoindolyl, 2,5-isoindolyl, 2,6-isoindolyl, 2,7-isoindolyl, 1,3-isoindolyl, 3,4-indazolyl, 3,5-indazolyl, 3,6-indazolyl, 3,7-indazolyl, 2,4-benzoxazolyl, 2,5-benzoxazolyl, 2,6-benzoxazolyl, 2,7-benzoxazolyl, 3,4-benzisoxazolyl, 3,5-benzisoxazolyl, 3,6-benzisoxazolyl, 3,7-benzisoxzlyl, 1,4-naphthyl, 1,5-naphthyl, 1,6-naphthyl, 1,7-naphthyl, 1,9-naphthyl, 2,4-naphthyl, 2,5-naphthyl, 2,6-naphthyl, 2,7-naphthyl, 2,8-naphthyl, 2,4-quiolinyl, 2,5-quinolinyl, 2,6-quinolinyl, 2,7-quinolinyl, 2,8-inolinyl, 3,4-quinolinyl, 3,5-quinolinyl, 3,6-quinolinyl, 3,7-quinolinyl, 3,5-quinolinyl, 4,5-quinolinyl, 4,6-suinolyl, 4,7-quinolinyl, 4,8-quinolinyl, 1,4-isoquinolinyl, 1,5-isoquinolinyl, 1,6-isoquinolinyl, 1,7-isoquinolinyl, 1,8-isoquinoiinyl, 3,4-isoquinolinyl, 3,5-isoquinolinyl, 3,6-isoquinolinyl, 3,7-isoquinolinyl, 3,8-isoquinolinyl, 4,5-isoquinotinyl, 4,6-isoquinolinyl, 4,7-isoquinolinyl, 4,8-isoquinolinyl, 3,4-nnolinyl, 3,5-cinnolinyl, 3,6-cinnolinyl, 3,7-cinnolinyl, 3,8-cinnolinyl, 4,5-cinnolinyl, 4,6-cinnolinyl, 4,7-cinnolinyl, and 4,8-cinnolinyl, and each carbon and hyfido containing nitrogen member of the ring of the W⁵ and of the ring of the W⁶, other than the points of attachment of W⁵ and W⁶, is optionally substituted with one or more of the group consisting of R⁹, R¹⁰, R¹¹, and R¹², with the proviso that Q^{b} is bonded to lowest number substituent position of each W⁵, with the further proviso that Q^{b} is bonded to highest number substituent position of each W⁶, and with the additional proviso that (CH(R³⁸))ᵣ is bonded to E⁰.

22. Compound of claim 21 of the Formula: 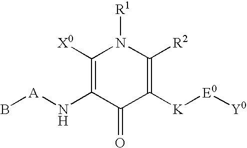
or a pharmaceutically acceptable salt thereof, wherein;
B is pbenyl or a heteroaryl of 5 or 6 ring menibers, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independently selected from the group consisting of hydrido, acetamido, haloacetamido, amidino, guanidino, alkylenedioxy, haloalkylthio, alkanoyloxy, alkoxy, hydroxy, amino, alkoxyamino, haloalkanoyl, nitro, alkylamino, alkylthio, aryl, aralkyl, cycloalkyl, cycloalkylalkyl, heteroaryl, heterocyclyl, alkylsulfonamido, anaidosulfonyl, alkyl, alkenyl, halo, haloalkyl, aloalkenyl, haloalkoxy, hydroxyalkyl, hydroxyhaloalkyl, aminoalkyl, carboalkoxy, catboxy, carboxamido, cyano, and Q^{b};
B is optionally selected from the group consisting of hydrido, trialkylsilyl, C2-C8 alkyl, C3-C8 alkylenyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶
B is optionally a C3-C12 cycloalkyl or a C4-C9 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen atom adjacent to the R⁹ position and two atoms from the point of attachment is optionally substituted with R¹⁰, a ring carbon or nitrogen atom adjacent to the R¹³ position and two atoms from the point of attachment is optionally substituted with R¹², a ring carbon or nitrogen atom three atoms from the point of attachment and adjacent to the R¹⁰ position is optionally substituted with R¹¹, a ring carbon or nitrogen atom three atoms from the point of attachment and adjacent to the R¹² position is optionally substituted with R³³, and a ring carbon or nitrogen atom four atoms from the point of attachment and adjacent to the R¹¹ and R³³ positions is optionally substituted with R³⁴;
R⁹, R¹⁰, R¹¹, R¹², and R¹³ are independently selected from the group consisting of hydrido, acetamido, haloacetamido, alkoxyamino, akanoyl, haloalkanoyl, amidino, guanidino, alkylenedioxy, haloalkylthio, alkoxy, cycloalkoxy, cycloalkylalkoxy, aalkoxy, aryloxy, heteroaryloxy, heteroaralkoxy,heterocyclyloxy, heterocyclylalkoxy, hydroxy, amino, alkylamino, N-alkyl-N-arylamino, arylamino, aralkylamino, heteroarylamino, heteroaralkylamino, heterocyclylamino, heterocyclylalkylamino, alkylthio, alkylsulfinyl, arylsulfinyl, aralkylsulfinyl, cycloalkylsulfinyl, heteroarylsulfinyl, alkylsulfamido, alkylsulfonyl, arylsulfonyl, aralkylsulfonyl, cycloalkylsulfonyl, heteroarylsulfonyl, amidosulfonyl, alkyl, aryl, aralkyl, cycloalkyl, cycloalkylalkyl, heteroaryl, heterocyclyl, halo, haloalkyl, haloalkoxy, hydroxyalkyl, hydroxyhaloalkyl, aminoalkyl, carboalkoxy, carboxy, carboxyalkyl, carboxamido, and cyano;
A is bond or (CH(R¹⁵))ₚₐ—(W⁷)ᵣᵣ wherein rr is 0 or 1, pa is an integer selected from 0 through 3, and W is selected from the group consisting of O, S, C(O), (R⁷)NC(O), (R⁷)NC(S), and N(R⁷), with the proviso that W is bonded to the N(H) on the 4-pyridone ring;
R⁷ i selected from the group consisting of hydrido, hydroxy and alkyl;
R¹⁵ is selected from the group consisting of hydrido, hydroxy, halo, akyl, and haloalyl;
X⁰ is selected from the group consisting of hydrido, alkyl, cyano, halo, haloalkyl, haloalkoxy, amino, aminoalkyl, alkylamino, amidino, hydroxy, hydroxyamino, alkoxy, hydroxyalkyl, alkoxyamino, thiol, and alkylthio;
R¹ is selected from the group consisting of hydrido, alkyl, haloalkyl, waino, aminoalkyl, alkylamino, hydroxy, and hydroxyalkyl;
X⁰ and R¹ or R and R² is optionally bonded t6-gether to form a partially saturated C5-C8 heterocyclyl ring, wherein said heterocyclyl ring is optionally substituted with one or more of the group consisting of R⁹, R¹⁰, R¹¹, R¹², and R¹³;
R² is Z⁰-Q^{b};
Z⁰ is selected from the group consisting of a bond, (CR⁴¹R⁴²)_{q} wherein q is 1 or 2, and (CH(R⁴¹))_{g}—W⁰—(CH(R⁴²))ₚ wherein g and p are integers independently selected from 0 through 3 and W⁰ is selected from the group consisting of O, S, C(O), S(O), N(R⁴¹), and QN(R⁴¹);
Z⁰ is optionally (CH(R⁴¹))ₑ—W²²(CH(R⁴²))ₕ wherein e and h are independently 0 or 1 and W²² is selected from the group consisting of CR⁴¹═CR⁴², 1,2-cyclopropyl, 1,2-cyclobutyl, 1,2-yclohexyl, 1,3-cyclohexyl, 1,2-cyclopentyl, 1,3-cyclopentyl, 2,3-morpholinyl, 2,4-morpholinyl, 2,6-morpholinyl, 3,4-morpholinyl, 3,5-moipholinyl, 1,2-piperazinyl, 1,3-piperazinyl, 2,3-piperazinyl, 2,6-piperazinyl, 1,2-piperidinyl, 1,3-piperidinyl, 2,3-piperidinyl, 2,4-piperidinyl, 2,6-piperidinyl, 3,4-piperidinyl, 1,2-pyrrolidinyl,1,3-pyrrolidinyl, 2,3-pyrrolidinyl, 2,4-pyrrolidinyl, 2,5-pyrrolidinyl, 3,4-pyrrolidinyl, 2,3-tetrahydrofuranyl, 2,4-tetrahydrofuranyl, 2,5-tetrahydrofuranyl, and 3,4-tetrahydrofuranyl, wherein Z⁰ is directly bonded to the 4-pyridone ring and W²² is optionally substituted with one or more substituents selected from the group consisting of R⁹, R¹⁰, R¹¹, R¹², and R¹³;
R⁴¹ and R⁴² are independently selected from the group consisting of hydrido, hydroxy, alkyl, and amino;
Q is phenyl or a heteroaryl of 5 or 6 ring members, wherein a carbon adjacent to the carbon at the point of attachment of said phenyl or heteroaryl ring to Z⁰ is optionally substituted by R⁹ the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
Q is optionally hydrido with the proviso that Z⁰ is other than a bond;
K is CR^{4a}R^{4b};
R^{4a} and R^{4b} are independently selected from the group consisting of hydrido, hydroxyalkyl, alkyl, alkoxyalkyl, alkylthioalkyl, and haloalkyl;
E⁰, with the proviso that K is CR^{4a}R^{4b}, is E¹ wherein E¹ is selected from the group consisting of a covalent single bond, C(O)N(H), (H)NC(O), S(O)₂N(H), and N(H)S(O)₂;
K is optionally (CH(R¹⁴))ⱼ-T wherein j is 0 or 1 and T is a bond or N(R⁷) wit the proviso that (CH(R¹⁴))ⱼ is bonded to the phenyl ring;
R¹⁴ is hydrido or halo;
E⁰ with the proviso that K is (CH(R¹⁴))ⱼ-T, is E² wherein E¹ is selected from the group consisting of C(O)N(H), (H)NC(O), C(S)N(H), (H)NC(S), S(O)₂N(H), N(H)S(O)₂, S(O)₂N(H)C(O), and C(O)N(H)S(O)₂;
Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{b} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, alkoxyamino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally selected from the group consisting of NR²⁰R²¹,N(R²⁶)C(NR²⁵)N(R²³)(R²⁴) and C(NR²⁵)NR²³R²⁴ with the proviso that R¹⁶, R¹⁹, and Q are not simultaneously hydrido;
Q^{b} is selected from the group consisting of NR²⁰R²¹, hydrido, N(R²⁶)C(NR²⁵)N(R²³)(R²⁴), and C(NR²⁵)NR²³R²⁴, with the proviso that no more than one of R²⁰ and R²¹ is selected from the group consisting of hydroxy, amino, alkylamino, and dialkylamino at the same time, with the further proviso that no more than one of R²³ and R²⁴ is selected from the group consisting of hydroxy, amino, alkylamino, and dialkylamino at the same time;
R²⁰, R²¹, R²³, R²⁴, R²⁵, and R²⁶ are independently selected from the group consisting of hydrido, alkyl, hydroxy, amino, alkylamino and dialkylamino;
Q^{s} is selected from the group consisting of a bond, (CR³⁷R³⁸)_{b} wherein b is an integer selected from 1 through 4, and (CH(R¹⁴))_{c}—W¹—(CH(R¹⁵))_{d} wherein c and d are integers independently selected from 1 through 3 and W¹ is selected from the group consisting of C(O)N(R¹⁴), (R¹⁴)NC(O), S(O), S(O)₂, S(O)₂N(R¹⁴), N(R¹⁴)S(O)₂, and N(R¹⁴), with the proviso that R is selected from other than halo when directly bonded to N, and with the additional proviso that (CR³⁷R³⁸) and (CR³⁷R³⁸)_{b}, and (CH(R¹⁴))_{c} are bonded to E⁰;
R³⁷ and R³⁸ are independently selected from the group consisting of hydrido, alkyl, and haloalkyl;
R³⁸ is optionally aroyl or heteroaroyl, wherein R³⁸ is optionally substituted with one or more substituents selected from the group consisting of R¹⁶, R¹⁷, R¹⁸, and R¹⁹;
Y⁰ is optionally Y^{AT} wherein Y^{AT} is Q^{b}-Q^{s};
Y⁰ is optionally Q^{b}-Q^{ss} wherein Q^{ss} is (CH(R¹⁴))ₑ—W²—(CH(R¹⁵))ₕ, wherein e and h are independently 1 or 2 and W² is CR^{4a}═CR^{4b} with the proviso that (CH(R¹⁴))ₑ is bonded to E⁰.

23. Compound of claim 22 or a pharmaceutically acceptable salt thereof, wherein;
B is selected from the group consisting of hydtido, trialkylsilyl, C2-C8 alkyl, C3-C8 alkylenyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³² R³³, R³⁴-, R³⁵, R³⁶;
R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alyl, halo, haloallyl, haloalkoxy, hydroxyalkyl, hydroxyhaloalkyl, carboalkoxy, carboxy, carboxamido, cyano, and Q^{b};
A is (CH(R¹⁵))ₚₐ—W wherein pa is an integer selected from 0 through 3 and W⁷ is selected from the group consisting of O, S, and N(R⁷) wherein R is hydrido or alkyl;
R¹⁵ is selected from the group consisting of hydrido, hydroxy, halo, alkyl, and haloalkyl;
X⁰ is selected from the group consisting of hydndo, amino, alkyl, cyano, halo, haloalkyl, haloalkoxy, aminoalkyl, hydroxy, alkoxy, hydroxyalkyl, and alkylthio;
R¹ is selected from the group consisting of hydrido, alkyl, haloalkyl, amino, aminoalkyl, alkylamino, hydroxy, and hydroxyalkyl;
R² is Z⁰-Q^{b};
Z⁰ is a bond or (CR⁴¹R⁴²)_{q} wherein q is 1 or 2;
R⁴¹ and R⁴² are independently selected from the group consisting of hydrido, hydroxy, and amino;
Q is phenyl or a heteroaryl of 5 or 6 ring members, wherein a carbon adjacent to the carbon at the point of attachment of said phenyl or heteroaryl ring to Z 0 is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹, with the proviso that Q^{b} is other than a phenyl when Z⁰ is a bond;
R⁹, R¹⁰, R¹¹, R¹², and R¹³ are independently selected from the group consisting of hydrido, acetamido, haloacetamido, alkoxyamino, alkanoyl, haloalkanoyl, amidino, guanidino, alkylenedioxy, haloalkylthio, alkoxy, cycloalkoxy, cycloalkylalkoxy, aralkoxy, aryloxy, heteroaryloxy, heteroaralkoxyheterocyclyloxy, heterocyclylalkoxy, hydroxy, amino, alkylamino, N-alkyl-N-arylamino, arylamino, aralkylamino, heteroarylamino, heteroaralkylamino, heterocyclylamino, heterocyclylalkylammno, alkyltdio, alkylswfinyl, arylsilfmyl, aralkylsulfinyl, cycloalkylsulfinyl, heteroarylsulfinyl, alkylsulfamido, alkylsulfonyl, arylsulfonyl, aralkylsulfonyl, cycloalkylsulfonyl, heteroarylsulfonyl, amidosulfonyl, alkyl, aryl, aralkyl, cycloalkyl, cycloalkylallyl, heteroaryl, heterocyclyl, halo, haloalkyl, haloalkoxy, hydroxyalkyl, hydroxyhaloalkyl, aminoalkyl, carboalkoxy, carboxy, carboxyalkyl, carboxamido, and cyano;
K is CHR^{4a} wherein R^{4a} is selected from the group consisting of hydrido, hydroxyalkyl, alkyl, alkoxyalkyl, alkylthioalkyl, and haloalkyl;
E⁰ is selected from the group consisting of a bond, C(O)N(H), (H)NC(O), (R⁷)NS(O)₂, and S(O)₂N(R⁷);
Y⁰ is phenyl or a heteroaryl of 5 or 6 ring members, wherein one carbon of said phenyl or said heteroaryl is substituted by Q^{s}, a carbon two or three contiguous atoms from the point of attachment of Q^{s} to the phenyl or heteroaryl ring is substituted by Q^{b}, a carbon adjacent to the point of attachment of Q^{b} is optionally substituted by R¹⁷, another carbon adjacent to the point of attachment of Q⁵ is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydnido, amidino, guanidino, carboxy, haloalkylthio, alkoxy, hydroxy, amino, alkoxyamino, lower alkylamino, alkylthio, alkylsulfinyl, alkylsulfonyl, alkanoyl, haloalkanoyl, alky, halo, haloalkyl, haloalkoxy, hydroxyalkyl, aminoalkyl, and cyano;
R¹⁶ or R¹⁹ is optionally selected from the group consisting of NR²⁰R²¹,N(R²⁶)C(NR²⁵)N(R²³)(R²⁴),and C(NR²⁵)NR²³R²⁴, with the proviso that R¹⁶, R¹⁹, and Q are not simultaneously hydrido;
Q^{b} is selected from the group consisting of NR²⁰R²¹, hydrido, N(R²⁶)C(NR²⁵)N(R²³)(R²⁴), and C(NR²⁵)NR²³R²⁴, with the proviso that no more than one of R²⁰ and R²¹ is selected from the group consisting of hydroxy, amino, alkylamino, and dialkylamino at the same time and with the further proviso that no more than one of R²³ and R²⁴ is selected from the group consisting of hydroxy, amino, alkylamino, and dialkylamino at the same time;
R²⁰, R²¹, R²³, R²⁴, R²⁵, and R²⁶ are independently selected from the group consisting of hydrido, alkyl, hydroxy, amino, alkylamino and dialkylamino;
Q^{b} is selected from the group consisting of a bond, (CR³⁷R³⁸)_{b} wherein b is an integer selected from 1 through 3, and (CH(R¹⁴))_{c}—W¹—(CH(R¹⁵))_{d} wherein c and d are independently 1 or 2 and W¹ is selected from the group consisting of C(O)N(R¹⁴), (R¹⁴)NC(O), S(O), S(O)₂, S(O)₂N(R¹⁴), N(R¹⁴)S(O)₂, and N(R¹⁴), with the proviso that R¹⁴ is selected from other than halo when directly bonded to N and with the further proviso that (CR³⁷R³⁸)_{b}, and (CH(R¹⁴))_{c} are bonded to E°;
R¹⁴ is selected from the group consisting of hydrido, halo, alky, and haloalkyl;
R³⁷ and R³⁸ are independently selected from the group consisting of hydrido, alkyl, and haloalkyl;
R³⁸ is optionally aroyl and heteroaroyl;
Y⁰ is optionally QQ QSs wherein Q(is (CH(R¹⁴))ₑ—W²-(CH(R¹⁵))h, wherein e and h are integers independently selected from 1 through 2 and W² is CR^{4a}═CH with the proviso that (CH(R¹⁴))ₑ is bonded to E⁰.

24. Compound of claim 23 of the Formula: 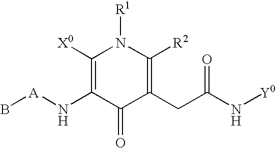
or a pharmaceutically acceptable salt thereof, wherein;
B is selected from the group consisting of hydrido, trialkylsilyl, C2-C4 alkyl, C3-C5 alkylenyl, C3-C4 alkenyl, C3-C4 alkynyl, and C2-C4 haloalkyl, wherein each member of group B is optionally substituted at any carbon up to and including 3 atoms from the point of attachment of B to A with one or more of the group consisting of R³², R³³, and R³⁴;
R³², R³³, and R³⁴ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, and cyano;
A is (CH(R¹⁵))ₚN(R⁷) wherein pa is an integer selected from 0 through 2 and R is hydrido or alkyl;
R¹⁵ is selected from the group consisting of hydrido, halo, alkyl, and haloalkyl;
X⁰ is selected from the group consisting of hydrido, amino, alkyl, halo, haloalkyl, haloalkoxy, amwnoalkyl, hydroxy, alkoxy, hydroxyalkyl, and alkylthio;
R¹ is selected from the group consisting of hydrido, alky, haloalkyl, amino, aminoalkyl, hydroxy, and hydroxyalkyl;
R² is Z⁰-Q^{b};
Z⁰ is a bond or CH₂;
Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, hydroxy, amino, amidino, guanidino, alkylamino, alkylthio, alkylsulfonamido, alkylsufinyl, alkylsulfonyl, anidosulfonyl, alkyl, alkoxy, halo, haloalkyl, haloalkoxy, hydroxyalkyl, hydroxyhaloalkyl, carboxy, carboxamido, and cyano;
R¹⁰ and R¹² are independently selected from the group consisting of hydrido, acetamido, haloacetamido, aridino, guanidino, alkyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy, heterocyclyloxy, heterocyclylalkoxy, hydroxy, amino, alkoxyamino, alkylamino, arylamino, aralkylamino, heteroarylafino, heteroaralkylamino, heterocyclylamino, heterocyclylalkylamino, alkylsulfonamido, amidosulfonyl, arylsulfinyl, aralkylsulfinyl, cycloalkylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;
Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfiyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally selected from the group consisting of NR²⁰R²¹,N(R²⁶)C(NR²⁵)N(R²³)(R²⁴),and C(NR²⁵)NR²³R²⁴, with the proviso that R¹⁶, R¹⁹, and Q are not simultaneously hydrido;
Q^{b} is selected from the group consisting of NR²⁰R²¹ hydrido, C(NR²⁵)NR²³R²⁴, and N(R²⁶)C(NR²⁵)N(R²³)(R²⁴), with the proviso that no more than one of R²⁰ and R²¹ is hydroxy at the same time and with the further proviso that no more than one of R²³ and R²⁴ is hydroxy at the same time;
R²⁰, R²¹, R²³, R²⁴, R²⁵, and R²⁶ are independently selected from the group consisting of hydrido, alkyl, and hydroxy;
Q^{s} is selected from the group consisting of a bond, CH₂, and CH₂CH₂.

25. Compound of claim 24 or a pharmaceutically acceptable salt thereof, wherein;
B is selected from the group consisting of ethyl, 2-propenyl, 2-propynyl, propyl, isopropyl, —CH₂CH₂CH₂—, —CH₂CH₂CH₂CH₂—, butyl, 2-butenyl, 3-butenyl, 2-butynyl, sec-butyl, tert-butyl, isobutyl, 2-methylpropenyl, 2,2,2-trifluoroethyl, 3,3,3-trifluoropropyl, and 2,2-difluoropropyl, wherein each member of group B is optionally substituted at any carbon up to and including 3 atoms from the point of attachment of B to A with one or more of the group consisting of R³², R³³, and R³⁴;
R³², R³³, and R³⁴ are independently selected from the group consisting of hydrido, amnidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ethylamino, methylthio, ethylthio, isopropylthio, trifluoromethyl, pentafluoroethyl, 2,2,2-trfluoroethyl, trifluoromethoxy, 1,1,2,2-tetrafluoroethoxy, fluoro, chloro, bromo, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, metboxycarbonyl, ethoxycarbonyl, amidocarbonyl, N-methylamidocarbonyl, N,Nediethylamidocarbony), and cyano;
A is selected from the group consisting of a bond, NH, and N(CH₃);
X⁰ is selected from the group consisting of hydrido, hydroxy, amino, aminomethyl, 1-aminoethyl, methyl, ethyl, trifluoromethyl, pentafluoroethyl, 2,2,2-tzifluoroethyl, methoxy, hydroxymethyl, 1-hydroxyethyl, 2-hydroxyethyl, methylthio, ethylthio, tuifuoromethoxy, 1,1,2,2-etrafluoroethoxy, fluoro, chloro, and bromo;
R¹ is selected from the group consisting of hydnido, hydroxy, amino, methyl, ethyl, propyl, n-butyl, 2,2,2-trifluoroethyl, 3,3,3-tnifluoropropyl, 2,2,3,3,3-pentafluoropropyl, 2-aminoethyl, 3-aminopropyl, 2-hydroxyetliyl, 3-hydroxypropyl, and 2,3-dihydroxypropyl;
R² is Z⁰-Q^{b};
Z⁰ is a bond or CH₂;
Q^{b} is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, 5 isoxazolyl, 2-pyridyl, 3-pyridyl, 4-pyridyl, 2-pyrazinyl, 2-pyrimidinyl, 4-pyrimidinyl, pyrimidinyl, 3-pyridazinyl, 4-pyridazinyl, and 1,3,5-triazin-2-yl, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, amidino, guanidino, carboxy, methyl, ethyl, propyl, isopropyl, methoxy, ethoxy, isopropoxy, propoxy, hydroxy, amino, N-methylamino, N,N-dimethylamino, N-ethylamino, methylthio, ethylthio, isopropylthio, trifluoromethyl, pentafluoroethyl, 2,2,2-tifluoroethyl, 2,2,3,3,3-pentafluoropropyl, tfifluoromeffioxy, 1,1,2,2-tetrafluoroethoxy, fluoro, chloro, bromo, methanesulfonamido, anidosulfonyl, N-methylamidosulfonyl, N,N-dimethyylamidosulfonyl, hydroxymethyl, 1-hydroxyethyl, 2-hydroxyethyl, 2,2,2-tiuorlc-hydroxyethyl, amidocarbonyl, N-methylamidocarbonyl, N,N-dimethylamidocarbonyl, and cyano;
R¹⁰ and R¹² are independently selected from the group consisting of hydrido, amidino, guanidino, carboxy, carboxymethyl, methyl, ethyl, propyl, isopropyl, methoxy, ethoxy, isopropoxy, propoxy, hydroxy, amino, metboxyamino, ethoxyamino, acetamido, trfluoroacetamido, aminomethyl, 1-aminoethyl, 2-aminoethyl, N-methylamino, dimethylamino, N-ethylamino, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, aridocarbonyl, N-methylamidocarbonyl, N,N-dimethylamidocarbonyl, N-benzylamidocarbonyl, N-(2-chlorobenzyl)amidocarbonyl, N-3-fluorobenzyl)amidocarbonyl, N-(2-trifluoromethylbenzyl)amidocatbonyl, N-(1-phenylethyl)amidocarbonyl, N-(1-methyl-1-phenylethyl)aridocarbonyl, N-benzylamidosulfonyl, N-(2-chlorobenzyl)amidosulfonyl, N-isopropylamidocarbonyl, N-cyclobutylamidocarbonyl, N-cyclopentylamidocarbonyl, fluoro, chloro, bromo, cyano, cyclobutoxy, cyclohexoxy, cyclohexylmethoxy, 4-trifluoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, 5-bromopyrid-2-ylmethylamino, 4-butoxyphenamiino, 3-chlorobenzyl, 4-chlorophenoxy, 4-chloro-3-ethylphenoxy, 4-chloro-3-ethylbenzylamino, 4-chloro-3-ethylphenylaiino, 3-chlorobenzyloxy, 4-chlorobenzyloxy, 4-chlorobenzylsulfonyl, 4-chlorophenylamino, 4-chlorophenylsulfonyl, S-chloropyrid-3-yloxy, 2-cyanopyrid-3-yloxy, 2,3Xifluorobenzyloxy, 2,4-difluorobenzyloxy, 3,4-difluorobenzyloxy, 2,5-difluorobenzyloxy, 3,5-difluorophenoxy, 3,5-difluorobenzyloxy, 4-difluoromethoxybenzyloxy, 2,3-difluorophenoxy, 2,4-difluorophenoxy, 2,5-difluorophenoxy, 3,5-dimethylphenoxy, 3,Wdimethylphenoxy, 3,4-direthylbenzyloxy, 3,5-dimethylbenzyloxy, 4-ethoxyphenoxy, 4-ethylbenzyloxy, 3-ethylphenoxy, 4-ethylaminophenoxy, 3-ethyl-5-methylphenoxy, 4-fluorobenzyloxy, 2-fluoro-3-trifluoromethylbenzyloxy, 3-fluoro-5-tffluoromethylbenzyloxy, 4-fluoro-2-tifluoromethylbenzyloxy, 4-fluoro-3-trifluoromethylbenzy1-oxy, 2-fluorophenoxy, 4-fluorophenoxy, 2-fluoro-3-trifluoromethylphenoxy, 2-fluorobenzyloxy, 4-fluorophenylanuno, 2-fluoroftrifluoromethylphenoxy, 4-isopropylbenzyloxy, 3-isopropylphenoxy, 4-isopropylphenoxy, 4 isopmpyl-3-methylphenoxy, 4-isopropylbenzyloxy, 3-isopropylphenoxy, 4-isopropylphenoxy, 4-isopropyl-3-methylphenoxy, phenylamino, 1-phenylethoxy, 2-phenylethoxy, 2-phenylethyl, 2-phenylethylamino, phenylsulfonyl, 3-trifluoromethoxybenzyloxy, 4-trifluoromethoxybenzyloxy, 3-trifluoromethoxyphenoxy, 4-trifluoromethoxyphenoxy, 3-trifluoromethylbenzyloxy, 4-trifluoromethylbenzyloxy, 2,4-bis-trifluoromethylbenzyloxy, 3-trifluoromethylbenzyl, 3,5-bis-trifluoromethylbenzyloxy, 4-trifluoromethylpbenoxy, 3-trifluoromethylphenoxy, 3-trifluoromethylthiobenzyloxy, 4-trifluoromethylthiobenzyloxy, 2,3,4-trifluorophenoxy, 2,3,5-trifluorophenoxy, 3-pentafluoroethylphenoxy, 3-(1,1,2,2-tetafluoroethoxy)plhenoxy, and 3-trifluoromethytihiophenoxy;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine, 2-Q^{b}-5-Q^{s}-3-R¹⁶-4 R¹⁸ pyridine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
3-Q^{b}-5-Q^{s}-4-R¹⁶-2-R¹⁹-furan, 2,6-5-Q^{s}-3-R¹⁶-4-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ imidazole,
3-Q^{b}-5-Q^{s}-4-R¹⁶ isoxazole, 5-Q^{b}-3-Q^{s}-4-R¹⁶ isoxazole,
2-Q^{b}-5Q^{s}-4-R¹⁶ pyrazole, 4-Q^{b}-2-Q^{s}-5-R¹⁹ thiazole, and
2-Q^{b}-5-Q^{s}-4-R¹⁷ thiazole;
R¹⁶, R¹⁷, R¹⁸, and R¹⁹ are independently selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2Arifluoroethyl, 2,2,3,3-pentafluoropropyl, tnfluoromethoxy, 1,1,2,2-tetrafluoroethoxy, fluoro, chloro, bromo, hydroxymethyl, 1-hydroxyethyl, 2-hydroxyethyl, and cyano;
R¹⁶ or R¹⁹ is optionally selected from the group consisting of NR²⁰R²¹,N(R²⁶)C(NR²⁵)N(R²³)(R²⁴), and C(NR²⁵)NR²³R²⁴, with the proviso that R¹⁶, R¹⁹, and Q are not simultaneously hydrido;
Q^{b} is selected from the group consisting of NR²⁰R²¹, hydrido, C(NR²⁵)NR²³R²⁴, and N(R²⁶)C(NR²⁵)N(R²³)(R²⁴), with the proviso that no more than one of R²⁰, R²¹, R²³, and R²⁴ can be hydroxy, when any two of the group consisting of R²⁰, R²¹, R²³, and R²⁴ are bonded to the same atom and with the further proviso that said Q group is bonded directly to a carbon atom;
R²⁰, R²¹, R²³, R²⁴, R²⁵, and R²⁶ are independently selected from the group consisting of hydrido, methyl, ethyl, propyl, butyl, isopropyl, and hydroxy;
Q^{s} is selected from the group consisting of a bond, CH₂, and CH₂CH₂.

26. Compound of claim 24 of the Formula: 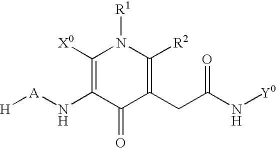
or a pharmaceutically acceptable salt thereof, wherein;
A is selected from the group consisting of CH₂N(CH₃), CH₂N(CH₂CH₃), CH₂CH₂N(CH₃), and CH₂CH₂N(CH₂CH₃);
X^{o} is selected from the group consisting of hydrido, hydroxy, amino, aminomethyl, 1-aminoethyl, methyl, ethyl, tWifluoromethyl, pentafluoroethyl, 2,2,2-trifluoroethyl, methoxy, hydroxymethyl, 1-hydtoxyethyl, 2-hydroxyethyl, methylthio, ethylthio, trifluoromethoxy,1,1,2,2-tetmfluoroethoxy, fluoro, chloro, and bromo;
R¹ is selected from the group consisting of hydrido, hydroxy, amino, methyl, ethyl, propyl, n-butyl, 2,2,5-trifluoroethyl, 3,3,3-trifluoropropyl, 2,2,3,3,3-pentafluoropropyl, 2-aminoethyl, 3-aminopropyl, 2-hydroxyethyl, 3-hydroxypropyl, and 2,3-dihydroxypropyl;
R² is Z⁰-Q^{b};
Z⁰ is a bond or CH₂;
Q is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pymzolyl, 4-pyrazolyl, 2-tWiazolyl, 3-isoxazolyl, 5 isoxazolyl, 2-pyridyl, 3-pyridyl, 4-pyridyl, 2-pyrazinyl, 2-pyrimidinyl, 4-pyridinyl, 5-pyrimidinyl, 3-pyridazinyl, 4-pyndazinyl, and 1,3,5-triazin-2,5-yl, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, amidino, guanidino, carboxy, methyl, ethyl, propyl, isopropyl, methoxy, ethoxy, isopropoxy, propoxy, hydroxy, amino, N-methylamino, N,N-dimethylamino, N-ethylamino, methylthio, ethylthio, isopropylthio, trifluoromethyl, pentafluoroethyl, 2,2,2-trifluoroethyl, 2,2,3,3,3-pentafluoropropyl, trifluoromnethoxy, 1,1,2,2-tetrafluoroethoxy, fluoro, chloro, bromo, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, amidocarbonyl, N-methylamidocarbonyl, N,N-dimethylamidocarbonyl, and cyano;
R¹⁰ and R¹⁰ are independently selected from the group consisting of hydrido, amidino, guanidino, carboxy, carboxymethyl, methyl, ethyl, propyl, isopropyl, methoxy, ethoxy, isopropoxy, propoxy, hydroxy, amino, methoxyamino, ethoxyamino, acetamido, trifluoroacetamiido, aminomethyl, 1-aminoethyl, 2-aminoethyl, N-methylamino, dimethylamino, N-ethylamino, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trifluorol-hydroxyethyl, methoxycarbonyl, ethoxycarbonyl, amidocarbonyl, N-methylamidocarbonyl, N,N-dimethylamidocarbonyl, N-benzylamidocarbonyl, N-(2-chlorobenzyl)amidocarbonyl, N-(3-fluorobenzyl)amidocarbonyl, N-(2-trifluoromethylbenzyl)amidocarbonyl, N-(1-phenylethyl)amidocarbonyl, N-(1-methyl-1-phenylethyl)amidocarbonyl, N-benzylamidosulfonyl, N-(2-chlorobenzyl)amidosulfonyl, N-isopropylamidocarbonyl, N-cyclobutylamidocarbonyl, N-cyclopentylamidocarbonyl, fluoro, chloro, bromo, cyano, cyclobutoxy, cyclohexoxy, cyclohexylmethoxy, 4-rifluoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, 5-brornopyrid-2-ylmethylamino, 4-butoxyphenamino, 3-chlorobenzyl, 4-chlorophenoxy, 4-ch1-oro-3-ethylphenoxy, 4-chloro-3-ethylbenzylamino, 4-chloro-3-ethylphenylamino, 3-ch1-orobenzyloxy, 4-chlorobenzyloxy, 4-chlorobenzylsulfonyl, 4-chlorophenylamino, 4-chlorophenylsulfonyl, 5-chloropyrid-3-yloxy, 2-cyanopyrid-3-yloxy, 2,3-difluorobenzyloxy, 2,4-difluorobenzyloxy, 3,4-difluorobenzyloxy, 2,5-difuorobenzyloxy, 3,5-difluorophenoxy, 3,5-difluorobeizyloxy, 4-difluoromethoxybenzyloxy, 2,3-difluorophenoxy, 2,4-difluorophenoxy, 2,5-difluorophenoxy, 3,5-dimetlylphenoxy, 3,4-dimethylphenoxy, 3,4-dimethylbenzyloxy, 3,5-dimethylbenzyloxy, 4-ethoxyphenoxy, 4-ethylbenzyloxy, 3-ethylphenoxy, 4-ethylaminophenoxy, 3-ethyl-5-methylphenoxy, 4-fluorobenzyloxy, 2-fluoro-3-trifluoromethylbenzyloxy, 3-fluoro-5-trifiuoromethylbenzyloxy, 4-fluoro-2-tnifluoromethylbenzyloxy, 4-fluoro-3-trifluoromethylbenzyloxy, 2-fluorophenoxy, 4-fluorophenoxy, 2-fluoro-3-trifluoromethylphenoxy, 2-fluorobenzyloxy, 4-fluorophenylamino, 2-fluoro trifluoromethylphenoxy, 4-isopropylbenzyloxy, 3-isopropylphenoxy, 4-isopropylphenoxy, 4-isopropyl-3-methylphenoxy, 4-isopropylbenzyloxy, 3-isopropylphenoxy, 4-isopropylphenoxy, 4-isopropyl-3-methylphenoxy, phenylamino, 1-phenylethoxy, 2-phenylethoxy, 2-phenylethyl, 2-phenylethylamino, phenylsuffonyl, 3-trifluoromethoxybenzyloxy, 4-trifluoromethoxybenzyloxy, 3-trifluoromethoxyphenoxy, 4-trifluoromethoxyphenoxy, 3-tnfluoromethylbenzyloxy, 4-trifluoromethylbenzyloxy, 2,4-bis-trifluoromethylbenzyloxy, 3-trifluoromethylbenzyl, 3,5-bis-trifuoromethylbenzyloxy, 4-t-ifluoromethylphenoxy, 3-trifluoromethylphenoxy, 3-tnfluoromethylthiobenzyloxy, 4-trifluoromethylthiobenzyloxy, 2,3,4-trifluorophenoxy, 2,3,5-triuorophenoxy, 3-pentafluoroethylphenoxy, 3-1,1,2,2-tetrafluoroethoxy)phenoxy, and 3-trifluoromethylthiophenoxy;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine, 2-Q^{b}-5-Q^{s}-3-R¹⁶-4 R¹⁸ pyridine,
3-Q^{b}-6-Q^{s}-2-R¹⁸-R¹⁸-2 R¹⁹ pyridazine,
3-Q^{b}-5-Q^{s}-4R¹⁶-2-R¹⁹ thiophene, 2-Q^{b}-5Q^{s}-3-R¹⁶-4-R¹⁹-thiophene,
3-Q^{b}-5-Q^{s}-4-R¹⁶-2-R¹⁹-furan, 2,6-5-Q^{s}-3-R¹⁶-4-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ imidazole,
3-Q^{b}-5-Q^{s}-4-R¹⁶ isoxazole, 5-Q^{b}-3-Q^{s}-4-R¹⁶ isoxazole,
3-Q^{b}-5-Q^{s}-4-R¹⁶ isoxazole, 5-Q^{b}-3-Q^{s}-4-R¹⁶ isoxazole,
2-Q^{b}-5Q^{s}-4-R¹⁶ pyrazole, 4-Q^{b}-2-Q^{s}-5-R¹⁹ thiazole, and
2-Q^{b}-5-Q^{s}-4-R¹⁷ thiazole;
R¹⁶, R¹⁷, R¹⁸, and R¹⁹ are independently selected from the group consisting of hydrido, methyl, ethyl, isopropyl, propyl, amidino, guanidino, methoxy, ethoxy, isopropoxy, propoxy, hydroxy, amino, aminomethyl, 1-aminoethyl, 2-aminoethyl, N-methylamino, dimethylamino, N-ethylarunio, methylthio, ethylthio, isopropylthio, trifluoromethylthio, methylsulfmyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, hydroxymethyl, 1-hydroxyethyl, 2-hydroxyethyl, and cyano;
Q^{b} is selected from the group consisting of NR²⁰R²¹, C(NR²⁵)NR²³R²⁴, and N(R²⁶)C(NR²⁵)N(R²³)(R²⁴),with the proviso that no more than one of R²⁰, R²¹, R²³, and R²⁴ can be hydroxy, when any two of the group consisting of R²⁰, R²¹, R²³, and R²⁴ are bonded to the same atom, and with the further proviso that said Q group is bonded directly to a carbon atom;
R²⁰, R²¹, R²³, R²⁴, R²⁵, and R²⁶ are independently selected from the group consisting of hydrido, methyl, ethyl, propyl, butyl, isopropyl, and hydroxy;
Q^{s} is selected from the group consisting of a bond, CE², and CH₂CH₂.

27. Compound of claim 26 or a pharmaceutically acceptable salt thereof, wherein;
A is selected from the group consisting of CH₂N(CH₃), CH₂N(CH₂CH₃), CH₂CH₂N(CH₃), and CH₂CH₂N(CH₂CH₃);
X^{o} is selected from the group consisting of hydrido, hydroxyl-aminoethyl, methyl, ethyl, trifluoromethyl, pentafluoroethyl, 2,2,2-trifluoroethyl, hydroxymethyl, 1-hydroxyethyl, 2-hydroxyethyl, fluoro, chloro, and bromo;
R¹ is selected from the group consisting of hydrido, hydroxy, amino, methyl, ethyl, 2,2,2-trifluoroethyl, 3,3,3-trifluoropropyl, 2-aminoethyl, and 2-hydroxyethyl;
R² is Z⁰-Q^{b};
Z⁰ is a bond or CH_{2;}
Q^{b} is selected from the group consisting of 3-amidocarbonyl-5-aminophenyl, 3-amino-5-(N-benzylamidocarbonyl)phenyl, 3-amino-5-benzylphenyl, 3-amino-5(2-phenylethyl)phenyl, 3-amio-benzylaminophenyl, 3-amino-5-(2-phenylethylamino)phenyl, 3-amino-5-benzyloxyphenyl, 3-amino-5-2-phenylethoxy)phenyl, 3-amino-5-(N-2-chlorobenzyl)amidocarbonyl)phenyl, 3-amino-5-(N-3-fluorobenzyl)amidocarbonyt)phenyl, 3-anon5-(N-2-tWifluoromethylbenzyl)amidocarbonyl)phenyl, 3-amino-5-(N-(1-phenylethyl)amidocarbonyl)phenyl, 3-amino-5-(N-(1-methyl-1-phenylethyl)amidocarbonyl)pbenyl, 3-amino-5-(N-benzylamidosulfonyl)phenyl, 3-amino-5-(N-(2-chlorobenzyl)amidosulfonyl)phenyl, 3-amino-5-(N-ethylaridocarbonyl)phenyl, 3-amino-5-(N-isopropylamidocarbonyl)phenyl, 3-ano-5-(N-propylamidocarbonyl)phenyl, 3-amino-5-(N-isobutylamidocarbonyl)phenyl, 3-amino-5-(N-(2-butyl)amidocarbonyl)phenyl, 3-amino-5-(N-cyclobutylamidocarbonyl)phenyl, 3-amino-5-N-cyclopentylamidocarbonyl)phenyl, 3-amino-5-(N-cyclohexylamidocarbonyl)phenyl, 3-amino-2-fluorophenyl, 3-amino-Shydroxymethylphenyl, 5-amino-3-methoxycarbonylphenyl, 3-amidinophenyl, 3-amino-2-methylphenyl, 5-amino-2-methylthiophenyl, 3-aminophenyl, 3-carboxyphenyl, 3-carboxy-5-hydroxyphenyl, 3-amino-5-carboxyphenyl, 3-chlorophenyl, 2-chlorophenyl, 3-cyanophenyl, 3,5-diaminophenyl, 3-dimethylaminophenyl, 2-fluorophenyl, 3-fluorophenyl, 2-hydroxyphenyl, 3-hydroxyphenyl, 3-methanesulfonylaminophenyl, 2-methoxyphenyl, 3-methoxyphenyl, 3-methoxyaminophenyl, 3-methoxycarbonylphenyl, 2-methylaminophenyl, 3-methylaminophenyl, 2-methylphenyl, 3-methylphenyl, 4-methylphenyl, phenyl, 3-trifluoroacetamidophenyl, 3-trifluoromethylphenyl, 2-trifluoromethylphenyl, 5-amino-2-thienyl, S-amino-3-thienyl, 3-bromo-2-thienyl, 3-pynidyl, 4-pyridyl, 2-thienyl, and 3-lhienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
R¹⁶ and R¹⁹ are independently selected from the group consisting of hydrido, amidino, amino, aminomethyl, methoxy, methylamino, hydroxy, hydroxymethyl, fluoro, chloro, and cyano;
R¹⁷ and R¹⁸ are independently selected from the group consisting of hydrido, fluoro, chloro, hydroxy, hydroxymethyl, amino, carboxy, and cyano;
Q^{b} is C(NR²⁵NR²³R²⁴;
R²³, R²⁴, and R²⁵ are independently selected from the group consisting of hydrido and methyl;
Q^{s} is CH_{2.}

28. A compound as recited in claim 27 or a pharmaceutically acceptable salt thereof where said compound is selected from the group consisting of:
2-[3-[N-[4[aminoiminomethylphenyl]methyl]-2-[3-aminophenyl]-5-[N,N-dimethylhydrazino]pyridonyl]]acetamide;
2-[3-[N-[[4-aminoiminomethylphenyl]methyl]-2-[3-aminophenyl]-5-[N-ethyl-N-methylhydrazinolXpyridonyl]]acetamide;
2-[3-[N-[[4-aminoiminomethylphenyl]methyl]-2-[3-aminophenyl]-5-[N,N-dimethylhydrazino]-N-methylpyridonyl]]acetamide;
2-[3-[N-[[4-aminoiminomethylphenyl]methyl]-2-[3-aminophenyl]-5-[N-ethyl-N-methylhydrazinol-N-methyltpyidonyl]]acetamide;
2-[3-[N-[[4-aminoiminomethylphenyl]methyl]-2-[3,5-diaminophenyl]-5-[N,N-dimethylhydmzino]lpyridonyl]]acetamide;
2-[3-[N-[[4-aminoiminomethylphenyl]methyl]-2-[3,5-&aminophenyl]-5-[N-ethyl-N-methylhydrazino]-pyridonyl]]acetamide;
2-[3-[N-[[4-aminoiminomethylphenyl]methyl]-2-[3,5-diaminophenyl]-5[N,N-dimethylhydrazino]-N-methyl4-pyridonyl]]acetamide;
2-[3-[N-[[4-aminoimiinomethylphenyl]methyl]-2-[3,5-diaminophenyl]-5-[N-ethyl-N-methylhydrazino]-N-methyl4-pyridonyl]]acetamide;
2-[3-[N-[[aininoiminomethylphenyl]methyl]-2-13-aminocarboxyphenyl-5-[N,N-dimethylhydrazino]4-pyridonyl]]acetamide;
2-[3-[N-[[Waminoiminomethylphenyl]methyl]-2-[3-amino-5-carboxyphenyl]-5-[N-ethyl-N-methylhydrazino]4-pyridonyl]]acetamide;
2-[3-[N-[[4-aminoiminomethylphenyl)methyl]-2-[3-amino-5 carboxyphenyl]-5-[N,N-dimethylhydrazinol-N-methylpyridonyl]]acetamide;
2-[3-[N-[[4-aminoiminomethylphenyl]methyl]-2-[3-amino-5 carboxyphenyl]-5[-ethyl-N-methylhydrazino]-N-methyl-4-pyridonyl]]acetamide;
2-[3-[N-[[4-aininoiminomethylphenyl]methyl]-2-[3-amino-5-(N-benzylamidocarbonyl)phenyl]-5-[N,N-dimethylhydrazino]4-pyridonyl]]acetamide;
2-[3-[N-[[4-aminoiminomethylphenyl]methyl]-2-[3-amino-5-(N-benzyladocarbonyl)phenyl]-5-[N-ethyl-N-methylhydraziao]A pyiidonyl]]acetamide;
2-[3-[N-[[Laminoiminomethylphenyl]methyl]-2-3-amino-5-(N-benzylamidocarbonyl)phenyl]-5-[N,N-dimethylhydrazino-]-4-pyridonyl]]acetamide;
2-[3-[N-[[4-minoiminomethylphenyl]methyl]-2[3-amino-(N-benzylamidocarbonyl)phenyl]-5-[N-ethyl-N-methylhydrazino]-N-methyl-4-pyridonyl]]acetamide.

29. Compound of claim 22 of the formula: 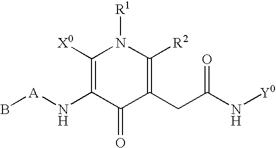
or a pharmaceutically acceptable salt thereof, wherein;
B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, cyano, and b;
A is a bond or (CH(R¹⁵))pa (W⁷)ᵣᵣ wherein rr is 0 or 1, pa is an integer selected from 0 through 3, and W⁷ is (R⁷)NC(O) or N(R⁷);
R⁷ is selected from the group consisting of hydrido, hydroxy and alkyl;
R¹⁵ is selected from the group consisting of hydrido, halo, alkyl, and haloalyl;
X⁰ is selected from the group consisting of hydrido, amino, alkyl, halo, haloalkyl, haloalkoxy, aminoalkyl, hydroxy, alkoxy, hydroxyalkyl, and alkylthio;
R¹ is independently selected from the group consisting of hydrido, hydroxy, amino, hydroxyalkyl, alkyl, aminoalkyl, and haloalkyl;
R² is Z⁰-Q^{b};
Z⁰ is selected from the group consisting of a bond, CH₂, CH₂CH₂, W⁰—(CH(R⁴²))ₚ wherein p is 0 or 1 and W° is selected, from the group consisting of O, S, and N(R⁴¹);
R⁴¹ and R⁴² are independently hydrido or alkyl;
Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independendy selected from the group consisting of hydrido, hydroxy, amino, amidino, guanidino, alkylamino, alkylthio, alkylsulfonamido, alkylsulfinyl, alkylsulfonyl, amidosulfonyl, alkyl, alkoxy, halo, haloalkyl, haloalkoxy, hydroxyalkyl, hydroxyhaloalkyl, carboxy, carboxamido, and cyano;
R¹⁰ and R¹² are independently selected from the group consisting of hydrido, acetamido, baloacetamido, amidino, guanidino, alkyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkoxyamino, alkylamino, arylamino, aralkylamino, heteroarylamino, heteroaralkylamino, heterocyclylamino, heterocyclylalkylamino, alkylsulfonamido, amidosulfonyl, arylsulfinyl, aralkylsulfinyl, cycloalkylsulfinyl, heteroarylsulfinyl, arylsulfonyl, aralkylsulfonyl, cycloalkylsulfonyli heteroarylsulfonyl, hydroxyalkyl, hydroxyhaloalkyl, aminoalkyl, carboalkoxy, carboxy, carboxyalkyl, carboxamido, halo, haloalkyl, and cyano;
Y⁰ is phenyl or a heteroaryl of 5 or 6 ring members, wherein one carbon of said phenyl or said heteroaryl is substituted by Q^{s}, a carbon two or thrie atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally NR²⁰R²¹ or C(NR²⁵)NR²³R²⁴, with the proviso that R¹⁶, R¹⁹, and Q are not simultaneously hydrido;
Q^{b} is selected from the group consisting of NR²⁰R²¹, hydrido, and C(NR²⁵)NR²³R²⁴, with the proviso that no more than one of R²⁰ and R²¹ is hydroxy at the same time and with the further proviso that no more than one of R²³ and R²⁴ is hydroxy at the same time;
R²⁰, R²¹, R²³, R²⁴, and R²⁵ are independently selected from the group consisting of hydrido, alkyl, and hydroxy;
Q^{s} is selected from the group consisting of a bond, CH₂, and CH₂CH₂.

30. Compound of claim 29 or a pharmaceutically acceptable salt thereof, wherein;
B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, Sisoxazolyl, 2,5-pyridyl, 3-pyridyl, 4-pyridyl, 2-pyrazinyl, 2-pyrimidinyl, 4-pyrimidinyl, 5-pytimidinyl, 3-pyridazinyl, 4-pyridazinyl, and 1,3,5-triazin-2-yl, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independently selected from the group consistng of hydrido, amidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ethylamino, methylthio, ethylthio, isopropylthio, trifluoromethyl, pentafluoroethyl, 2,2,2-trifluoroethyl, 2,2,3,3,3-pentafluoropropyl, tiiuoroniethoxy, 1,1,2,2-etraluoroethoxy, fluoro, chloro, bromo, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, cyano, and Q^{b};
A is selected from the group consisting of a bond, NH, N(CH₃), N(OH), CH₂, CH₃CH, CF₃CH, NHC(O), N(CH₃)C(O), C(O)NH,
C(O)N(CH₃), CH₂CH₂, CH₂CH₂CH₂, CH₃CHCH₂, and CF₃CHCH₂;
X^{o} is selected from the group consisting of hydrido, hydroxy, amino, aminomethyl, 1-aminoethyl, methyl, ethyl, trifluoromethyl, pentafluoroethyl, 2,2,2-trifluoroethyl, methoxy, hydroxymethyl, 1-hydroxyethyl, 2-hydroxyethyl, methylthio, ethylthio, ifuoromethoxy,1,1,2,2-tetafluoroethoxy, fluoro, chloro, and bromo;
R¹ is selected from the group consisting of hydrido, hydroxy, amino, methyl, ethyl, propyl, n-butyl, 2,2,2-trifluoroethyl, 3,3,3-triuoropropyl, 2,2,3,3,3-pentafluoropropyl, 2-mnoethyl, 3-aminopropyl, 2-hydroxyethyl, 3-hydroxypropyl, and 2,3-dihydroxypropyl;
R² is Z⁰-Q^{b};
Z⁰ is selected from the group consisting of a bond, CH₂, CH₂CH₂, O, S, NH, N(CH₃), OCH₂, SCH₂, N(H)CH₂, and N(CH₃)CH₂;
Q is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, 5 isoxazolyl, 2-pyridyl, 3-pyridyl, 4-pyridyl, 2-pyrazinyl, 2-pyrimidinyl, 4-pyrimidinyl, Spyrimidinyl, 3-pyridazinyl, 4-pyridazinyl, and 1,3,5-triazin-2-yl, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, amidino, guanidino, carboxy, methyl, ethyl, propyl, isopropyl, methoxy, ethoxy, isopropoxy, propoxy, hydroxy, amino, N-methylamino, N,N-dimethylamino, N-ethylamino, methylthio, ethylthio, isopropylthio, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromno, methanesulfonamido, amidosulfonyl, N-methylamidosulfonyl, N,N-dimethylamidosulfonyl, hydroxymethyl, 1-hydroxyethyl, 2-hydroxyethyl, 2,2,2-ttifluoro-1-hydroxyethyl, amidocarbonyl, N-methylamidocarbonyl, N,N-imethylamidocarbonyl, and cyano;
R¹⁰ and R¹² are independently selected from the group consisting of hydrido, amidino, guanidino, carboxy, carboxymethyl, methyl, ethyl, propyl, isopropyl, methoxy, ethoxy, isopropoxy, propoxy, hydroxy, amino, methoxyamino, ethoxyamino, acetamido, trifluoroacetamido, aminomethyl, 1-aminoethyl, 2-aminoethyl, N-methylamino, dimethylamino, N-ethylamino, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, anidocarbonyl, N-methylanudocarbonyl, N₁Ndimethylamidocarbonyl, N-benzylamidocarbonyl, N-(2-chlorobenzyl)amidocarbonyl, N-(3-fluorobenzyl)nnudocarbonyl, N-(2-trifluoromethylbenzyl)amidocarbonyl, N-(1-phenylethyl)amidocarbonyl, N-(1-methyl-1-phenylethyl)anidocarbonyl, N-benzylanidosulfonyl, N-(2-chlorobenzyl)amidosulfonyl, N-ethylanidocarbonyl, N-isopropylamidocarbonyl, N-propylaniudocarbonyl, N-isobutylaniidocarbonyl, N-(2-butyl)amidocarbonyl, N-cyclobutylanidocarbonyl, N-cyclopentylamidocarbonyl, N-cyclohexylaniidocarbonyl, fluoro, chloro, bromo, cyano, cyclobutoxy, cyclohexoxy, cyclohexylmethoxy, 4-trifluoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, 5-bromopyrid-2-ylmethylamino, 4-butoxyphenanuno, 3-chlorobenzyl, 4-chlorophenoxy, 4-chloro-3-ethylphenoxy, 4-chloro-3-ethylbenzylamino, 4-chloro-3-ethylphenylamino, 3-chlorobenzyloxy, 4-chlorobenzyloxy, 4-chlorobenzylsulfonyl, 4-chlorophenylamino, 4-chlorophenylsulfonyl, 5-chloropyrid-3-yloxy, 2-cyanopyrid-3-yloxy, 2,3-difluorobenzyloxy, 2,4-difluorobenzyloxy, 3,4-difluorobenzyloxy, 2,5-difluorobenzyloxy, 3,5-difluorophenoxy, 3,5-difluorobenzyloxy, 4-difluoromethoxybenzyloxy, 2,3-difluorophenoxy, 2,4-difluorophenoxy, 2,5Mifluorophenoxy, ,5-dimethylphenoxy, 3,4-dimethylphenoxy, 3,4-dimethylbenzyloxy, 3,5-dimethylbenzyloxy, 4-ethoxyphenoxy, 4-ethylbenzyloxy, 3-ethylphenoxy, 4-ethylaminophenoxy, 3-ethyl-5-methylphenoxy, 4-fluorobenzyloxy, 2-fluoro-3-trifluoromethylbenzyloxy, 3-fluoro-5-trifluoromethylbenzyloxy, 4-fluoro-2-trifluoromethylbenzyloxy, 4-fluoro-3-trifluoromethylbenzyloxy, 2-fluorophenoxy, 4-fluorophenoxy, 2-fluoro-3-trifluoromethylphenoxy, 2-fluorobenzyloxy, 4-fluorophenylamino, 2-fluoro-trifluoromethylphenoxy, 4-isopropylbenzyloxy, 3-isopropylphenoxy, 4-isopropylphenoxy, 4-isopropyl-3-methylphenoxy, 4-isopropylbenzyloxy, 3-isopropylphenoxy, 4-isopropylphenoxy, 4-isopropyl-3-methylphenoxy, phenylamino, 1-phenylethoxy, 2-phenylethoxy, 2-phenylethyl, 2-phenylethylamino, phenylsulfonyl, 3-trifluoromethoxybenzyloxy, 4-trifluoromethoxybenzyloxy, 3-trifluoromethoxyphenoxy, 4-trifluoromethoxyphenoxy, 3-tiifiuoromethylbenzyloxy, 4-trifluoromethylbenzyloxy, 2,4-bis-tnfiuoromethylbenzyloxy, 3-trifuoromethylbedzyl, 3,5-bis-trifluoromethylbenzyloxy, 4-trifuoromethylphenoxy, 3-trifluoromethylphenoxy, 3-trifluoromethylthiobenzyloxy, 4-trifluoromethylthiobenzyloxy, 2,3,4-trifluorophenoxy, 2,3,5-tffluorophenoxy, 3-pentafluoroethylphenoxy, 3-(1,1,2,2-tetrafluoroethoxy)phenoxy, and 3-trifluoromethylthiophenoxy;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine, 2-Q^{b}-5-Q^{s}-3-R¹⁶-4 R¹⁸ pyridine,
3-Q^{b}-6-Q^{s}-2-R¹⁶-5-R¹⁸-4-R¹⁹ pyridine,
2-Q^{b}-5-Q^{s}-4-R¹⁷-6-R¹⁸ pyrimidine, 5-Q^{b}-2-Q²-4-R¹⁶-6-R¹⁹ pyrimidine,
3-Q^{b}-5-Q^{s}-4R¹⁶-2-R¹⁹ thiophene, 2-Q^{b}-5Q^{s}-3-R¹⁶-4-R¹⁹-thiophene,
3-Q^{b}-5-Q^{s}-4-R¹⁶-2-R¹⁹-furan, 2,6-5-Q^{s}-3-R¹⁶-4-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ imidazole,
3-Q^{b}-5-Q^{s}-4-R¹⁶ isoxazole, 5-Q^{b}-3-Q^{s}-4-R¹⁶ isoxazole,
2-Q^{b}-5Q^{s}-4-R¹⁶ pyrazole, 4-Q^{b}-2-Q^{s}-5-R¹⁹ thiazole, and
2-Q^{b}-5-Q^{s}-4-R¹⁷ thiazole;
R¹⁶, R¹⁷, R¹⁸, and R¹⁹ are independently selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylamino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1, 1,2,2-tetrafluoroethoxy, fluoro, chloro, bromo, hydroxymethyl, 1-hydroxyethyl, 2-hydroxyethyl, and cyano;
R¹⁶ or R¹⁹ is optionally C(NR²⁵)NR²³R²⁴ with the proviso that R R¹⁹, and Q are not simultaneously hydndo;
Q^{b} is C(NR²⁵)NR²³R²⁴ or hydrido, with the proviso that no more than one of R²³ and R²⁵ is hydroxy at the same time;
R²³, R²⁴, and R²⁵ are independently selected from the group consisting of hydrido, methyl, ethyl, and hydroxy;
Q^{s} is selected from the group consisting of a bond, CH₂ and CH₂CH₂.

31. Compound of claim 30 or a pharmaceutically acceptable salt thereof, wherein;
B is selected from the group consisting of 2-aminophenyl, 3-aminophenyl, 3-amidinophenyl, 4-amidinophenyl, 3-crboxypbenyl, 3-carboxy-5-hydroxyphenyl, 3-chlorophenyl, 4-chlorophenyl, 3,4-dichlorophenyl, 2-fluorophenyl, 3-fluorophenyl, 3,4-difluorophenyl, 3-hydroxyphenyl, 4-hydroxyphenyl, 3-methoxyaminophenyl, 3-methoxyphenyl, 4-methoxyphenyl, 3-methylphenyl, 4-methylphenyl, phenyl, 3-triuoromethylphenyl, 2-imidazoyl, 2-pyridyl, 3-pyridyl, 5-chloro-3-trifluoromethyl-2-pyridyl, 4-pyridyl, 2-thienyl, 3-thienyl, and 3-trifluoromethyl-2-pyridyl;
A is selected from the group consisting of CH₂, CH₃C1, CF₃CH, NHC(O), CH₂CH₂, and CH₂CH₂CH₂;
X^{o} is selected from the group consisting of hydrido, hydroxy, laminoethyl, methyl, ethyl, tiifluoromethyl, pentafluoroethyl, 2,2,2-trifluoroethyl, hydroxymethyl, 1-hydroxyepthyl, 2-hydroxyethyl, fluoro, chloro, and bromo;
R¹ is selected from the group consisting of hydrido, hydroxy, amino, methyl, ethyl, propyl, 2,2,2-trifluoroethyl, 3,3,3-trifluoropropyl, 2,2,3,3,3-pentafluoropropyl, 2-aminoethyl, and 2-hydroxyethyl;
R² is Z⁰-Q^{b};
Z⁰ is selected from the group consisting of a bond, CH₂, O, S, NH, N(CH₃), OCH₂, and SCH_{2;}
Q is selected from the group consisting of 3-amidocarbonyl-5-aminophenyl, 3-amino-(N-benzylamidocarbonyl)phenyl, 3-amin-5-benzylphenyl, 3-amino-5(2-phenylethyl)phenyl, 3-amino-5-benzylaminophenyl, 3-amino-5(2-phenylethylamino)phenyl, 3-amino-5-benzyloxyphenyl, 3-amino-5-(2-phenylethoxy)phenyl, 3-aminoN-(2-chlorobenzyl)amidocarbonyl)phenyl, 3-amino-5-(N-(3-fluorobenzyl)amidocarbonyl)phenyl, 3-amino-5-(N-2-triuoromethylbenzyl)amidocarbonyl)phenyl, 3-amino-5-(N-(1-phenylethyl)awidocarbonyl)phenyl, 3-amino-5-(N-(1-methyl-1-phenylethyl)amidocarbonyl)phenyl, 3-amino-5-(N-benzylamidosulfonyl)phenyl, 3-amino-5-(N-(2-chlorobenzyl)amidosulfonyl)phenyl, 3-amino-5-(N-ethylamidocarbonyl)phenyl, 3-amino-5-(N-isopropylamidocarbonyl)phenyl, 3-amino-5-(N-propylamidocarbonyl)phenyl, 3-amino-5-(N-isobutylamidocarbonyl)phenyl, 3-amino-54N-(2-butyl)amidocarbonyl)phenyl, 3-amino-5-(N-cyclobutylamidocarbonyl)phenyl, 3-amino-5-(N-cyclopentylamidocarbonyl)phenyl, 3-amino-5-N-cyclohexylamidocarbonyl)phenyl, 5-amino-2-fluorophenyl, 3-amino-5-hydroxymethylphenyl, 5-amino-3-methoxycarbonylphenyl, 3-amidinophenyl, 3-amino-2-methylphenyl, 5-amino-2-methylthiophenyl, 3-aminophenyl, 3-amino-S(4-trifluoromethylbenzylamino)phenyl, 3-amino-5-(4-trifluoromethylbenzyloxy)phenyl, 3-carboxyphenyl, 3-carboxy-5-hydroxyphenyl, 3-amino-5-carboxyphenyl, 3-chlorophenyl, 2-chlorophenyl, 3-cyanophenyl, 3,5-diamiophenyl, 3-dimethylamtnophenyl, 2-fluorophenyl, 3-fluorophenyl, 2-hydroxyphenyl, 3-hydroxyphenyl, 3-methanesulfonylaminophenyl, 2-methoxyphenyl, 3-methoxyphenyl,
3-methoxyaminophenyl, 3-methoxycarbonylphenyl, 2-methylaminophenyl, 3-methylaininophenyl, 2-methylphenyl, 3-methylphenyl, 4-methylphenyl, phenyl, 3-trifluoroacetamidophenyl, 3-trifluoromethylphenyl, 2-trifluoromethylphenyl, 5-amino-2-thienyl, 5-amino-3-thienyl, 3-bromo-2-thienyl, 3-pyridyl, 4-pyridyl, 2-thienyl, and 3-thienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
R¹⁶ and R¹⁹ are independently selected from the group consisting of hydrido, ainidino, amo, aminomethyl, methoxy, methylamino, hydroxy, hydroxymethyl, fluoro, chloro, and cyano;
R¹⁶ or R¹⁹ is optionally C(NR²⁵)NR²³R²⁴ with the proviso that R R¹⁹, and Q are not simultaneously hydrido;
R¹⁷ and R¹⁸ are independently selected from the group consisting of hydrido, fluoro, chloro, hydroxy, hydroxymethyl, amino, carboxy, and cyano;
Q^{b} is C(NR²⁵)NR²³R²⁴ or hydrido;
R²³, R²⁴, and R²⁵ are independentdy hydrido or methyl;
Q^{s} is CH₂.

32. Compound of claim 29 of the Formula: 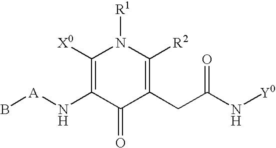
or a pharmaceutically acceptable salt thereof, wherein;
B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independently selected from the group consisting of hychido, acetan-ido, haloacetaindo, amidino, guanidino, alkoxy, hydroxy, amino, alkoxyamino, alkylamino, alkylthio, amidosulfonyl, alky, halo, haloalkyl, haloalkoxy, hydroxyalkyl, carboalkoxy, carboxy, carboxamido, cyano, and Q^{b};
A is a bond or (CH(R¹⁵))ₚₐ—(W⁷)ᵣᵣ wherein rr is 0 or 1, pa is an integer selected from 0 through 3, and W⁷ is N(R⁷);
R⁷ is hydrido or alkyl;
R¹⁵ is selected from the group consisting of hydrido, halo, alkyl, and haloalkyl;
X⁰ is selected from the group consisting of hydrido, alkyl, halo, haloalkyl, aminoalkyl, and hydroxyalkyl;
R¹ is selected from the group consisting of hydrido, alkyl, hydroxyalkyl, and haloalkyl;
R² is Z⁰-Q^{b};
Z⁰ is a bond;
Q is phenyl or a heteroaiyl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independendy selected from the group consisting of hydrido, hydroxy, amino, amidino, guanidino, alkylamino, alkylthio, alkoxy, alkylsulfinyl, alkylsulfonyl, amidosulfonyl, alkyl, halo, haloalkyl, haloalkoxy, hydroxyalkyl, carboxy, carboxamido, and cyano;
R¹⁰ and R¹² are independently selected from the group consisting of hydrido, acetamido, haloacetamido, amidino, guanidino, alkyl, alkoxy, alkoxyamino, hydroxy, amino, alkylamino, alkylsulfonamido, atnidosulfonyl, hydroxyalkyl, aminoalkyl, halo, haloalkyl, carboalkoxy, carboxy, carboxamido, carboxyalkyl, and cyano;
Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally NR²⁰R²¹ or C(NR²⁵)NR²³R²⁴ with the proviso that R¹⁶, R¹⁹, and Q^{b} are not simultaneously hydrido;
Q^{b} is selected from the group consisting of NR²⁰R²¹, hydrido, and C(NR²⁵)NR²³R²⁴;
R²⁰, R²¹, R²³, R²⁴, and R²⁵ are independently hydrido or alkyl;
Q^{b} is CH₂.

33. Compound of claim 32 or a pharmaceutically acceptable salt thereof, wherein;
B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, and Sisoxazolyl, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independently selected from the group consisting of hydrido, amidino, guanidino, methyl, ethyl, methoxy, ethoxy, hydroxy, amino, N-methylamino, dimethylamino, methoxyamino, methylthio, ethylthio, tnifluoromethyl, pentafluoroethyl, 2,2,2-trifluoroethyl, fluoro, chloro, bromo, amidosulfonyl, N-methylamidosulfonyl, hydroxymethyl, amidocarbonyl, carboxy, cyano, and Q^{b};
A is selected from the group consisting of a bond, NH, N(CH₃), CH₂, CH₃CH, and CH₂CH₂;
X⁰ is selected from the group consisting of hydrido, aminomethyl, methyl, ethyl, trifuoromethyl, 2,2,2-trifluoroethyl, hydroxymethyl, 2-hydroxyethyl, chloro, and fluoro;
R¹ is selected from the group consisting of hydrido, methyl, ethyl, propyl, 2,2,2-trifluoroethyl, 3,3,3-triuoropropyl, 2,2,3,3,3-pentafluoropropyl, and 2-hydroxyethyl;
R² is selected from the group consisting of phenyl, 2-thienyl, 2-furyl, 2-pyrrolyl, 2-imidazolyl, 2-thiazolyl, 3-isoxazolyl, 2-pyridyl, and 3-pyridyl, wherein a carbon adjacent to the carbon at the point of attachment of said phenyl or heteroaryl ring to the 4-pyridone ring is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, methyl, ethyl, methoxy, ethoxy, hydroxy, amino, N-methylamino, N,N-dimethylamino, methylthio, trifluoromethyl, pentafluoroethyl, 2,22-trifluoroethyl, fluoro, chloro, bromo, amidosulfonyl, N-methylamidosulfonyl, N,N-dimethylamidosulfonyl, hydroxymethyl, 1-hydroxyethyl, amidocarbonyl, N-methylamidocarbonyl, carboxy, and cyano;
R¹⁰ and R¹² are independently selected from the group consisting of hydrido, amidino, amidocarbonyl, N-methylamidocarbonyl, N-benzylanidocarbonyl, N-(2,5-chlorobenzyl)amidocarbonyl, N-(3-fluorobenzyl)amidocarbonyl, N-2-bifiuoromethylbenzyl)amidocarbonyl, N-(1-phenylethyl)amidocarbonyl, N-(1-methyl-1-phenylethyl)amidocarbonyl, N-benzylamidosulfonyl, N-2-chlorobenzyl)amidosulfonyl, N-ethylamidocarbonyl, N-isopropylamidocarbonyl, N-propylamidocarbonyl, N-isobutylamidocarbonyl, N-(2-butyl)amidocarbonyl, N-cyclobutylaniidocarbonyl, N-cyclopentylamidocarbonyl, N-eyclohexylamidocarbonyl, guanidino, methyl, ethyl, methoxy, ethoxy, hydroxy, hydroxymethyl, 1-hydroxyethyl, 2-hydroxyethyl, carboxy, carboxymethyl, amino, acetamido, trifluoromethyl, pentafluoroethyl, 2,2,2-trifluoroethyl, trifluoroacetamido, aminomethyl, N-methylamino, dimethylamino, methoxyamino, amidosulfonyl, N-methylamidosulfonyl, N,N-dimethylamidosulfonyl, methanesulfonamido, methoxycarbonyl, fluoro, chloro, bromo, and cyano;
Y⁰ is selected from the group consisting of:
1-Q^{b}-4-Q^{s}-2-R¹⁶-3-R¹⁷-5-R¹⁸-6-R¹⁹ benzene,
2-Q^{b}-6-Q^{s}-2-R¹⁷-5-R¹⁸-4-R¹⁹ pyridine, 2-Q^{b}-5-Q^{s}-3-R¹⁶-4 R¹⁷ thiophene,
3-Q^{b}-6-Q^{s}-2-R¹⁶-5-R¹⁸-4-R¹⁹ pyridine, 2-Q^{b}-5-Q^{s}-3-R¹⁶-4 R¹⁹ thiophene,
3-Q^{b}-5-Q^{s}-4-R¹⁶-2-R¹⁹ furan, 2-Q^{b}-5-Q^{s}-3-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ thiazole,
R¹⁶, R¹⁷, R¹⁸, and R¹⁹ are independendy selected from the group consisting of hydrido, methyl, ethyl, amidino, guanidino, methoxy, hydroxy, amino, aminomethyl, 1-aminoethyl, 2-raminoethyl, N-methylamino, dimethylamino, methylthio, ethylthio, trifiuoromethylthio, methylsulfinyl, methylsulfonyl, trifluoromethyl, pentafluoroethyl, 2,2,2-trifuoroethyl, trifluoromethoxy, fluoro, chloro, hydroxymethyl, carboxy, and cyano;
Q^{b} is NR²⁰R²¹ or C(NR²⁵)NR²³R²⁴;
R²⁰, R²¹, R²³, R²⁴, and R²⁵ are independently selected from the group consisting of hydrido, methyl, and ethyl;
Q^{s} is CH₂.

34. Compound of claim 33 or a pharmaceutically acceptable salt thereof, wherein;
B is selected from the group consisting of 2-aminophenyl, 3-aminophenyl, 3-amidinophenyl, 4-amidinophenyl, 3-carboxyphenyl, 3-carboxy-5-hydroxyphenyl, 3-chlorophenyl, 4-chlorophenyl, 3,4-dichlorophenyl, 2-fluorophenyl, 3-fluorophenyl, 3,4-difluorophenyl, 3-hydroxyphenyl, 4-hydroxyphenyl, 3-methoxyaminophenyl, 3-methoxyphenyl, 4-methoxyphenyl, 3-methylphenyl, 4-methylphenyl, phenyl, 3-trifluoromethylphenyl, 2-imidazoyl, 2-pyridyl, 3-pyridyl, 5-chloro-3-trifluoromethyl-2-pyridyl, 4-pyridyl, 2-thienyl, 3-thienyl, and 3-trifluoromethyl-2-pyridyl;
A is CH₂ or CH₂CH₂;
X^{o} is selected from the group consisting of hydrido, methyl, ethyl, trifluoromethyl, 2,2,2-trifluoroethyl, hydroxymethyl, 2-hydroxyethyl, chloro, and fluoro;
R¹ is selected from the group consisting of hydrido, methyl, ethyl, and 2,2,2-trifluoroethyl;
R² is selected from the group consisting of 3-amidocarbonyl-5-aminophenyl, 3-amidocarbonyl-5-aminophenyl, 3-amino-5-(N-benzylamidocarbonyl)phenyl, 3-amino-5-(N-(2-chlorobenzyl)anudocarbonyl)phenyl, 3-amino-5-(3-fluorobenzyl)amidocarbonyl)phenyl, 3-amino-S-(2-triuoromethylbenzyl)amidocarbonyl)phenyl, 3-amino-5-(1-phenylethyl)amidocarbonyl)phenyl, 3-amino-5-(N-(1-methyl-1-phenylethyl)amidocarbonyl)phenyl, 3-amino-5-(N-benzylamidosulfonyl)phenyl, 3-amino5-(N-(2-chlorobenzyl)amidosulfonyl)phenyl, 3-amino-5-(Nethylamidocarbonyl)phenyl, 3-amino-5-(N-isopropylamidocarbonyl)phenyl, 3-amino-5-(N-propylamidocarbonyl)phenyl, 3-amino-5-(N-isobutylamidocarbonyl)phenyl, 3-amino-5-(N-(2-butyl)amidocarbonyl)phenyl, 3-amino-5-N-cyclobutylamidocarbonyt)phenyl, 3-amino-5-(N-cyclopentylamidocarbonyl)phenyl, 3-amino-5-(N-cyclohexylamidocarbonyl)phenyl, S-amino-2-fluorophenyl, 3-amino-5-hydroxymethylphenyl, 5-amino-3-methoxycarbonylphenyl, 3-amidinophenyl, 3-amino-2-methylphenyl, 5-amino-2-methylthiophenyl, 3-aminophenyl, 3-carboxyphenyl, 3-carboxy-5-aminophenyl, 3-carboxy-5-hydroxyphenyl, 3-carboxymethyl-5-aminophenyl, 3-carboxymethyl-5-hydroxyphenyl, 3-carboxymethylphenyl, 3-chlorophenyl, 2-chlorophenyl, 3-cyanophenyl, 3,5-diaminophenyl, 34-methylaminophenyl, 2-fluorophenyl, 3-fluorophenyl, 2,5-difluorophenyl, 2-hydroxyphenyl, 3-hydroxyphenyl, 3-methanesulfonylaminophenyl, 2-methoxyphenyl, 3-methoxyphenyl, 3-methoxyaminophenyl, 3-methoxycarbonylphenyl, 2-methylaminophenyl, 3-methylaminophenyl, 2-methylphenyl, 3-methylphenyl, 4-methylphenyl, phenyl, 3-trifluoroacetamidophenyl, 3-trfluoromethylphenyl, 2-tlifluoromethylphenyl, 5-amino-2-thienyl, 5-amino-3-thienyl, 3-bromo-2-thienyl, 3-pyridyl, 4-pyridyl, 2-thienyl, and 3-thienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
R¹⁶ and R¹⁹ are independently selected from the group consisting of hydrido, amidino, amino, aminomethyl, methoxy, methylamino, hydroxy, hydroxymethyl, fluoro, chloro, and cyano;
R¹⁷ and R¹⁸ are independently selected from the group consisting of hydrido, fluoro, chloro, hydroxy, hydroxymethyl, amino, carboxy, and cyano;
Q^{b} is C(NR²⁵)NR²³R²⁴;
R²³, R²⁴, and R²⁵ are independently hydrido or methyl;
Q^{s} is CH₂.

35. Compound of claim 34 or a pharmaceutically acceptable salt thereof, wherein;
B is selected from the group consisting of 3-aminophenyl, 3-amidinophenyl, 4-amidinophenyl, 3-chlorophenyl, 4-chlorophenyl, 3,4-dichlorophenyl, 2-fluorophenyl, 4-methylphenyl, phenyl, 2-imidazoyl, 3-pyridyl, 4-pyridyl, and 3-trifluoromethyl-2-pyridyl;
A is CH₂ or CH₂CH₂;
X^{o} is selected from the group consisting of hydrido, methyl, ethyl, trifluoromethyl, 2,2,5-triuoroethyl, chloro, and fluoro;
R¹ is selected from the group consisting of hydrido, methyl, ethyl, and 2,2,5-tifluoroethyl;
R² is selected from the group consisting of 3-amudocarbonyt-5-aminophenyl, 3-amino-5-N-benzylamidocarbonyl)phenyl, 3-amino-5-(N-2-chlorobenzyl)amidocarbonyl)phenyl, 3-amino-5-(N-(3-fluorobenzyl)amidocarbonyl)phenyl, 3-amino-5-N-(2-trifluoromethylbenzyl)amidocarbonyl)phenyl, 3-amini5-(N-(1-phenylethyl)amidocarbonyl)phenyl, 3-amino-5-(N-(1-methyl-1-phenylethyl)amidocarbonyl)phenyl,
3-amino-5-(N-benzylamidosulfonyl)phe-yl, 3-amino-5-(N-(2-chlorobenzyl)amidosulfonyl)phenyl, 3-amino-5-(N-ethylamidocarbonyl)phenyl, 3-amino-5-(N-isopropylamidocarbonyl)phenyl, 3-amino-5-(N-propylamidocarbonyl)phenyl, 3-amino-5-(N-isobutylamidocarbonyl)phenyl, 3-amino-5-(N-2-butyl)amidocarbonyl)phenyl, 3-amino-5-(N-cyclobutylamidocarbonyl)phenyl, 3-amino-5-(N-cyclopentylamidocarbonyl)phenyl, 3-amino-5-(N-cyclohexylamidocarbonyl)phenyl, 3-aminophenyl, 3-carboxy-5-aminophenyl, 3-chlorophenyl, 3,§diaminophenyl, 3-dimethylaminophenyl, 3-hydroxyphenyl, 3-methanesulfonylaminophenyl, 3-methylaminophenyl, 2-methylphenyl, 3-methylphenyl, phenyl, 3-trifluoroacetamidophenyl, 3-bromno-thienyl, 2-thienyl, and 3-thienyl;
Y⁰ is selected from the group consisting of 5-amidino-Sthienylmethyl, 4-aniidinobenzyl, 2-fluoro4-amidinobenzyl, and 3-fluoro4-amdinobenzyl.

36. Compound of claim 29 where said compound is selected from the group of the Formula: 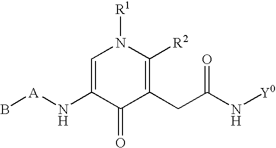
or a pharmaceutically acceptable salt thereof, wherein B, A, R¹, R², X^{o}, and Y⁰ are selected to form a compound selected from the group consisting of;
R² is 3-aminophenyl, B is phenyl, A is CH₂, Y⁰ is 4-amirdinobenzyl, and R¹ is methyl;
R² is 3-aminophenyl, B is 3-chlorophenyl, A is CH₂CH₂, Y⁰ is 4 amidinobenzyl, and R¹ is methyl;
R² is 3-aminophenyl, B is phenyl, A is CH₂, Y⁰ is 4-amidinobenzyl, and R¹ is hydrido;
R² is 3-aminophenyl, B is 2-iimudazoyl, A is CH₂CH₂CH₂, Y⁰ is 4 amidinobenzyl, and R¹ is methyl;
R² is 3-amidodarbonyl-iaminophenyl, B is 3-chlorophenyl, A is CH₂CH₂, is 4-amidinobenzyl, and R¹ is methyl;
R² is 3-amino-5-(N-benzylamidocarbonyl)phenyl, B is 3-chlorophenyl, A is CH₂CH₂, Y⁰ is 4-amidinobenzyl, and R¹ is methyl;
R² is 3-amino-5-(N-(2-chlorobenzyl)amidocarbonyl)phenyl, B is 3-chlorophenyl, A is CH₂CH₂, Y⁰ is 4-amidinobenzyl, and R¹ is methyl;
R² is 3-amino-5-(N-(2-chlorobenzyl)amidosulfonyl)phenyl, B is 3-chlorophenyl, A is CH₂CH₂, Y⁰ is 4-amidinobenzyl, and R¹ is methyl;
R² is 3-amino-S—(N-(2-trifluoromethylbenzyl)amidocarbonyl)-phenyl, B is 3-chlorophenyl, A is CH₂CH₂, Y⁰ is 4-amidinobenzyl, and R¹ is methyl;
R² is 3,5-iaminophenyl, B is 3-chlorophenyl, A is CH₂CH₂, Y⁰ is 4 amidinobenzyl, and R¹ is methyl;
R² is 3-amino-5-carboxyphenyl, B is 3-chlorophenyl, A is CH₂CH₂, Y⁰ is 4-amidinobenzyl, and R¹ is methyl;
R² is 3-amidocarbonyl-5-aminophenyl, B is 3-chlorophenyl, A is CH₂CH₂, is 4-amidinobenzyl, and R¹ is hydrido;
R² is 3-amino-5-(N-benzylamidocarbonyl)phenyl, B is 3-chlorophenyl, A is CH₂CH₂, Y⁰ is 4-amidinobenzyl, and R¹ is hydrido;
R² is 3-amino-5-(N-(2-chlorobenzyl)amidocarbonylphenyl, B is 3-chlorophenyl, A is CH₂CH_{2,5}-yo is 4-amidinobenzyl, and R¹ is hydrido;
R² is 3-amino-5-N-(2-chlorobenzyl)amidosulfonyl)phenyl, B is 3-chlorophenyl, A is CH₂CH₂, Y⁰ is 4-aamidinobenzyl, and R¹ is hydrido;
R² is 3-amino-5-(N-(2-trifluoromethylbenzyl)amidocarbonyl)-phenyl, B is 3-chlorophenyl, A is CH₂CH₂, Y⁰ is 4-amidinobenzyl, and R¹ is hydrido;
R² is 3,5-diaminophenyl, B is 3-chlorophenyl, A is CH₂CH_{2,5}, Y⁰ is 4 amidinobenzyl, and R¹ is hydrido; and
R² is 3-amino-5-caiboxyphenyl, B is 3-chlorophenyl, A is CH₂CH₂, Y⁰ is 4-amidinobenzyl, and R¹ is hydrido.

37. Compound of claim 22 of the Formula: 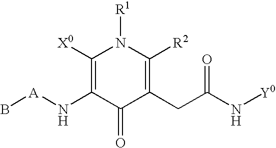
or a pharmaceutically acceptable salt thereof, wherein;
B is selected from the group consisting of hydrido, C2-C8 alkyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵ and R³⁶;
R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyallyl, hydroxyhaloalkyl, carboalkoxy, carboxy, carboxamido, cyano, and Q^{b};
A is a bond or (CH(R¹⁵))pa(W⁷)ᵣᵣ wherein rr is 0 or 1, pa is an integer selected from 0 through 3, and W⁷ is (R⁷)NC(O) or N(R⁷);
R⁷ is selected from the group consisting of hydrid, hydroxy and alkyl;
R¹⁵ is selected from the group consisting of hydrido, halo, xly and haloallyl;
X⁰ is selected from the group consisting of hydrido, halo, alkyl, halo, haloalkyl, haloalkoxy, aminoalkyl, hydroxy, alkoxy, hydroxyalkyl, and alkylthio;
R¹ is independently selected from the group consisting of hydrido, hydroxy, amino, hydroxyalkyl, alky, aminoalkyl, and haloaUkyl;
R² is Z⁰-Q^{b};
Z⁰ is selected from the group consisting of a bond, CH₂, CH₂CH₂, W⁰—(CH(R⁴²))ₚ wherein p is 0 or 1 and W is selected from the group consisting of O, S, and N(R⁴¹);
R⁴¹ and R⁴² are independently hydrido or alkyl;
Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, hydroxy, amino, amidino, guanidino, alkylamino, alkylthio, aliylsulfonamido, alkylsulfinyl, alkylsulfonyl, amidosuffonyl, alkyl, alkoxy, halo, haloalkyl, haloalkoxy, hydroxyalkyl, hydroxyhaloalkyl, carboxy, carboxamido, and cyano;
R¹⁰ and R¹² are independently selected from the group consisting of hydrido, acetamido, haloacetamido, aridino, guanidino, alkyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkoxyamino, alkylamino, arylamino, arkylamino, heteroarylamino, heteroaralkylamino, heterocyclylamino, heterocyclylalkylamino, alkylsulfonamido, amidosulfonyl, arylsulfinyl, aralkylsulfinyl, cycloalkylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;
Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally selected from the group consisting of NR²⁰R²¹,N(R²⁶)C(NR²⁵)N(R²³)(R²⁴), and C(NR²⁵)NR²³R²⁴, with the proviso that R¹⁶, R¹⁹, and Q^{b} are not simultaneously hydrido;
Q^{b} is selected from the group consisting of NR²⁰R²¹, hydrido, C(NR²⁵)NR²³R²⁴, and N(R²⁶)C(NR²⁵)N(R²³)(R²⁴), with the proviso that no more than one of R²⁰ and R²¹ is hydroxy at the same time and with the further proviso that no more than one of R²³ and R²⁴ is hydroxy at the same time;
R²⁰, R²¹, R²³, R²⁴, R²⁵ and R²⁶ are independently selected from the group consisting of hydrido, alkyl, and hydroxy;
Q^{s} is selected from the group consisting of a bond, CH₂, and CH₂CH₂.

38. Compound of claim 37 or a pharmaceutically acceptable salt thereof, wherein;
B is selected from the group consisting of hydrido, ethyl, 2-propynyl, 2-propenyl, propyl, isopropyl, butyl, 2-butenyl, 3-butenyl, 2-butynyl, sec-butyl, tert-butyl, isobutyl, 2-methylpropenyl, 1-pentyl, 2-pentenyl, 3-pentenyl, 4-pentenyl, 2-pentynyl, 3-pentynyl, 2-pentyl, 1-methyl-2-butenyl, 1-methyl-3-butenyl, 1-methyl-2-butynyl, 3-pentyl, 1-ethyl-2-propenyl, 2-methylbutyl, 2-methyl-2-butenyl, 2-methyl-3-butenyl, 2-methyl-3-butynyl, 3-methylbutyl, 3-methyl-2-butenyl, 3-methyl-3-butenyl, 1-hexyl, 2-hexenyl, 3-hexenyl, 4-hexenyl, 5-hexenyl, 2-hexynyl, 3-hexynyl, 4-hexynyl, 2-hexyl, 1-methyl-2-pentenyl, 1-methyl-3-pentenyl, 1-methylpentenyl, 1-methyl-2-pentynyl, 1-methyl-3-pentynyl, 3-hexyl, 1-ethyl-2-butenyl, 1-ethyl-3-butenyl, 1-propyl-2-propenyl, 1-ethyl-2-butynyl, 1-heptyl, 2-heptenyl, 3-heptenyl, 4-heptenyl, Sheptenyl, 6-heptenyl, 2-heptynyl, 3-heptynyl, 4-heptynyl, Sheptynyl, 2-heptyl, 1-methyl-2-hexenyl, 1-methyl-3-hexenyl, 1-methyl 1-hexenyl, 1-methyl-Shexenyl, 1-methyl-2-hexynyl,
1-methyl-3-hexynyl, 1-methyl4-hexynyl, 3-heptyl, 1-ethyl-2-pentenyl, 1-ethyl-3-pentenyl, i-ethyltpentenyl, 1-butyl-2-propenyl, 1-ethyl-2-pentynyl, 1-ethyl-3-pentynyl, 2,2,2-trifluoroethyl, 2,2-difluoropropyl, 4-trifluoromethyl-5,5,5-trifluoropentyl, 4-trifluoromethylpentyl, 5,5,6,6,6-pentafluorohexyl, and 3,3,3-trifluoropropyl, wherein each member of group B is optionally substituted at any carbon up to and including 5 atoms from the point of attachment of B to A with one or more of the group consisting of R³², R³³, R³⁴, R³⁵, and R³⁶;
R³², R³³, R³⁴, R³⁵, and R³⁶ are independently selected from the group consisting of hydrido, amidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ethylamino, methylthio, ethylthio, isopropylthio, trifluoromethyl, pentafluoroethyl, 2,2,2-trifluoroethyl, 2,2,3,3,3-pentafluoropropyl, trifluoromethoxy, 1,1,2,2-tetrafluoroethoxy, fluoro, chloro, bromo, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, cyano, and Q^{b};
A is selected from the group consisting of bond, NH, N(CH₃), N(OH), CH₂, CH₃CH, CF₃CH, NHC(O), N(CH₃)C(O), C(O)NH, C(O)N(CH₃),
CH₂CH₂, CH₂CH₂CH₂, CH₃CHCH₂, and CF₃CHCH₂;
X^{o} is selected from the group consisting of hydrido, hydroxy, amino, aminomethyl, 1-aminoethyl, methyl, ethyl, trifluoromethyl, pentafluoroethyl, 2,2,2-trifluoroethyl, methoxy, hydroxymethyl, 1-hydroxyethyl, 2-hydroxyethyl, methylthio, ethylthio, uffluoromethoxy,1,1,2,2-tetfluoroethoxy, fluoro, chloro, and bromo;
R¹ is selected from the group consisting of hydrido, hydroxy, amino, methyl, ethyl, propyl, n-butyl, 2,2,2-trifluoroethyl, 3,3,3-trifluoropropyl, 2,2,3,3,3-pentafluoropropyl, 2-aminoethyl, 3-aminopropyl, 2-hydroxyethyl, 3-hydroxypropyl, and 2,3-dihydroxypropyl;
R² is Z⁰-Q^{b};
Z⁰ is selected from the group consisting of a bond, CH₂, CH₂CH₂, O, S, NH, N(CH₃), OCH₂, SCH₂, N(H)CH₂, and N(CH₃)CH₂;
Q is selected from the group consisting of phenyl, 2-thienyl, 3-thienyl, 2-furyl, 3-fury1, 2-pyrrolyl, 3-pyrrolyl, 2-imidazolyl, 4-imidazolyl, 3-pyrazolyl, 4-pyrazolyl, 2-thiazolyl, 3-isoxazolyl, Sisoxazolyl, 2-pyridyl, 3-pyridyl, 4-pyridyl, 2-pyrazinyl, 2-pyrimidinyl, 4-pyrinidinyl, 5-pyrmidinyl, 3-pyridazinyl, 4-pyridaunyl, and 1,3,5-tiazin-2-yl, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, amidino, guanidino, carboxy, methyl, ethyl, propyl, isopropyl, methoxy, ethoxy, isopropoxy, propoxy, hydroxy, amino, N-methylamino, N,N-dimethylamino, N-ethylamino, methylthio, ethylthio, isopropylthio, trifluoromethyl, pentafluoroethyl, 2,2,2-tnfluoroethyl, 2,2,3,3,3-pentafluoropropyl, tifluoromethoxy, 1,1,2,2-tetrafluoroethoxy, fluoro, chloro, bromo, methanesulfonamido, amidosulfonyl, N-methylamidosilfonyl, N,N-dimethylanidosulfonyl, hydroxymethyl, 1-hydroxyethyl, 2-hydroxyethyl, 2,2,2-trifluoro-1-hydroxyethyl, amidocarbonyl, N-methylanudocarbonyl, N,N-dimethylamidocarbonyl, and cyano;
R¹⁰ and R¹² are independently selected from the group consisting of hydrido, anidino, guanidino, carboxy, carboxymethyl, methyl, ethyl, propyl, isopropyl, methoxy, ethoxy, isopropoxy, propoxy, hydroxy, amino, nethoxyamino, ethoxyamino, acetamido, trifluoroacetamido, aminomethyl,
1-amiinoethyl, Z-aminoethyl, N-methylamino, diinethylamino, N-ethylamino, methanesulfonanido, amidosulfonyl, N-methylaniudosulfonyl, N,N-dimnethylamidosulfonyl, hydroxymethyl, 1-hydroxyethyl, 2-hydroxyethyl, 2,2,2-tnifluoro-1-hydroxyethyl, methoxycarbonyl, ethoxycarbonyl, amidocarbonyl, N-methylamidocarbonyl, N,N dimethylamidocarbonyl, N-benzylamidocarbonyl, N-(2-chlorobenzyl)amidocarbonyl, N-(3-fluorobenzyl)amidocarbonyl, N-(2-trifluoromethylbenzyl)amidocarbonyl, N-(1-phenylethyl)anidocarbonyl, N-(1-methyl-1-phenylethyl)amidocarbonyl, N-benzylamidosulfonyl, N-(2-chlorobenzyl)amidosulfonyl, N-ethylamidocarbonyl, N-isopropylamidocarbonyl, N-propylamidocarbonyl, N-isobutylamidocarbonyl, N-(2-butyl)amidocarbonyl, N-cyclobutylamidocarbonyl, N-cyclopentylamidocarbonyl, N-cyclohexylamidocarbonyl, fluoro, chloro, bromo, cyano, cyclobutoxy, cyclohexoxy, cyclohexylmethoxy, 4-trifluoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, Sbromopyiid-2-ylmethylamino, 4-butoxyphenamino, 3-chlorobenzyl, 4-chlorophenoxy, 4-chloro-3-ethylphenoxy, 4-chloro-3-ethylbenzylamino, 4-chloro-3-ethylphenylamino, 3-chlorobenzyloxy, 4-chlorobenzyloxy, 4-chlorobenzylsulfonyl, 4-chlorophenylamino, 4-chlorophenylsulfonyl, 5-chloropyrid-3-yloxy, 2-cyanopyrid-3-yloxy, 2,3-difluorobenzyloxy, 2,4-difluorobenzyloxy, 3,4-difluorobenzyloxy, 2,5-difluorobenzyloxy, 3,5-difluorophenoxy, 3,5-difluorobenzyloxy, 4-difluoromethoxybenzyloxy, 2,3-difluorophenoxy, 2,4-difluorophenoxy, 2,5-difluorophenoxy, 3,5-dimethylphenoxy, 3,4-dimethylphenoxy, 3,4-dimethylbenzyloxy, 3,5-dimethylbenzyloxy, 4-ethoxyphenoxy, 4-ethylbenzyloxy, 3-ethylphenoxy, 4-ethylaminophenoxy, 3-ethyl-5-methylphenoxy, 4-fluorobenzyloxy, 2-fluoro-3-trifluoromethylbenzyloxy, 3-fluoro-5-trifluoromethylbenzyloxy, 4-fluoro2-trifluoromethyl be nzyloxy, 4-fluoro-3-trifluoromethylbenzyloxy, 2-fluorophenoxy, 4-fluorophenoxy, 2-fluoro-3-tiifluoromethylphenoxy, 2-fluorobenzyloxy, 4-fluorophenylatnino, 2-fluoro4-trifluoromethylphenoxy, 4-isopropylbenzyloxy, 3-isopropylphenoxy, 4-isopropylphenoxy, 4-isopropyl-3-methylphenoxy, 4-isopropylbenzyloxy, 3-isopropylphenoxy, 4-isopropylphenoxy, 4-isopropyl-3-methylphenoxy, phenylamino,
1-phenylethoxy, 2-phenylethoxy, 2-phenylethyl, 2-phenylethylamino, phenylsulfonyl, 3-trifluoromethoxybenzyloxy, 4-tifluoromethoxybenzyloxy, 3-trifluoromethoxyphenoxy, 4-trifluoromethoxyphenoxy, 3-trifuoromethylbenzyloxy, 4-trifluoromethylbenzyloxy, 2,4-bis-trfluoromethylbenzyloxy, 3-trifluoromethylbenzyl, 3,5-bis-trifluoromethylbenzyloxy, 4-trifluoromethylphenoxy, 3-trifluoromethylphenoxy, 3-trifluoromethylthiobenzyloxy, 4-trifluoromethylthiobenzyloxy, 2,3,4-trifluoropheDoxy, 2,3,5-trifluorophenoxy, 3-pentafluoroethylphenoxy, 3-(1,1,2,2-tetrafluoroethoxy)phenoxy, and 3-trifluoromethylthiophenoxy;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine, 2-Q^{b}-5-Q^{s}-3-R¹⁶-4 R¹⁸ pyridine,
3-Q^{b}-6-Q^{s}-2-R¹⁶-5-R¹⁸-4-R¹⁹ pyridine,
2-Q^{b}-5-Q^{s}-4-R¹⁷-6-R¹⁸ pyrimidine, 5-Q^{b}-2-Q²-4-R¹⁶-6-R¹⁹ pyrimidine,
3-Q^{b}-5-Q^{s}-4R¹⁶-2-R¹⁹ thiophene, 2-Q^{b}-5Q^{s}-3-R¹⁶-4-R¹⁹-thiophene,
3-Q^{b}-5-Q^{s}-4-R¹⁶-2-R¹⁹-furan, 2,6-5-Q^{s}-3-R¹⁶-4-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ imidazole,
3-Q^{b}-5-Q^{s}-4-R¹⁶ isoxazole, 5-Q^{b}-3-Q^{s}-4-R¹⁶ isoxazole,
2-Q^{b}-5Q^{s}-4-R¹⁶ pyrazole, 4-Q^{b}-2-Q^{s}-5-R¹⁹ thiazole, and
2-Q^{b}-5-Q^{s}-4-R¹⁷ thiazole;
R¹⁶, R¹⁷, R¹⁸, and R¹⁹ are independenfly selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylamino, N-ethylamino, methylthio, ethytltijo, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-tetrfluoroethoxy, fluoro, chloro, bromo, hydroxymethyl, 1-hydroxyethyl, 2-hydroxyethyl, and cyano;
R¹⁶ or R¹⁹ is optionally selected from the group consisting of NR²⁰R²¹, C(NR²⁵)NR²³R²⁴, and N(R²⁶)C(NR²⁵)N(R²³)(R²⁴), with the proviso that R¹⁶, R¹⁹, and Q^{b} are not simultaneously hydrido;
Q^{b} is selected from the group consisting of NR²⁰R²¹, hydrido, C(NR²⁵)NR²³R²⁴, and N(R²⁶)C(NR²⁵)N(R²³)(R²⁴), with the proviso that no more than one of R²⁰ and R²¹ is hydroxy at the same time and with the further proviso that no more than one of R²³ and R²⁴ is hydroxy at the same time;
R²⁰, R²¹, R²³, R²⁴, R²⁵, and R²⁶ are independently selected from the group consisting of hydrido, methyl, ethyl, propyl, butyl, isopropyl, and hydroxy;
Q^{s} is selected from the group consisting of a bond, CH₂, and CH₂CH₂.

39. Compound of Caim 38 or a pharmaceutically acceptable salt thereof, wherein;
B is selected from the group consisting of hydrido, ethyl, 2-propenyl, 2-propynyl, propyl, isopropyl, butyl, 2-butyl, (R)-2-butyl, (S)-2-butyl, tert-butyl, isobutyl, 1-pentyl, 3-pentyl, 2-methylbutyl, 2,2,2-trifluoroethyl, 6-amidocarbonylhexyl, 4-methyl-2-pentyl, 3-hydroxypropyl, 1-methoxy-2-propyl, 2-methoxyethyl, 2-methyl-2-butyl, 3-methyl-2-butyl, 2-imcthylaminopropyl, 2-cyanoethyl, 6-hydroxyhexyl, 2-hydroxyethyl, 2-amidinoethyl, 2-guanidinoethyl, 3-guanidinopropyl, 4-guanidinobutyl, 3-hydroxypropyl, 4-hydroxybutyl, 6-cyanohexyl, 2-dimethylaminoethyl, 3-methylbutyl, 2-methylbutyl, (S)-2-methylbutyl, 3-aminopropyl, 2-hexyl, and 4-aminobutyl;
A is selected from the group consisting of a bond, CH₂, NHC(O), CH₂CH₂, CH₂CH₂CH₂, and CH₃CHCH₂;
X^{o} is selected from the group consisting of hydrido, hydroxy, 1-aminoethyl, methyl, ethyl, trifluoromethyl, pentafluoroethyl, 2,2,2-trifluoroethyl, hydroxymethyl, 1-hydroxyethyl, 2-hydroxyethyl, fluoro, chloro, and bromo;
R¹ is selected from the group consisting of hydrido, hydroxy, amino, methyl, ethyl, propyl, 2,2,2-trifluoroethyl, 3,3,3-trifluoropropyl, 2,2,3,3,3-pentafluoropropyl, 2-aminoethyl, and 2-hydroxyethyl;
R² is Z⁰-Q^{b};
Z⁰ is selected from the group consisting of a bond, CH₂, O, S, NH, N(CH₃), OCH₂, and SCH_{2;}
Q^{b} is selected from the group consisting of 3-amidocarbonyl-5-amnnophenyl, 3-amino-5-N-benzylamidocarbonyl)phenyl, 3-amino-5-benzylphenyl, 3-amino-5(2-phenylethyl)phenyl, 3-amino-5-benzylaminophenyl, 3-amino-5(2-phenylethylamino)phenyl, 3-amino-5-benzyloxyphenyl, 3-amino-5-(2-phenylethoxy)phenyl, 3-amino-S—(N-(2-chlorobenzyl)amidocarbonyl)phenyl, 3-amino-(N-3-fluorobenzyl)amidocarbonyl)phenyl, 3-amino-5-(N-2-trfluoromethylbenzyl)amidocarbonyl)phenyl, 3-amino-5-(N-1-phenylethyl)amidocarbonyl)phenyl, 3-amino-5-(N-(1-methyl-1-phenylethyl)amidocarbonyl)phenyl, 3-amino-5-(N-benzyla]idosulfonyl)phenyl, 3-amino-S—(N-(2-chlorobenzyl)amidosulfonyl)phenyl, 3-amnin5-i(N-ethylamidocarbonyl)phenyl, 3-amino-5-(N-isopropylamidocaTbonyl)phenyl, 3-amino-5-N-propylamidocarbonyl)phenyl, 3-amino-54N-isobutylamidocarbonyl)phenyl, 3-amino-5-(N-2-butyl)amidocarbonyl)phenyl, 3-amino-5-(N-cyclobutylamidocarbonyl)phenyl, 3-amino-5-(N-cyclopentylamidocarbonyl)phenyly, 3-amino-5-(N-cyclohexylamidocarbonyl)phenyl, 5-amino-2-fluorophenyl, 3-amino-5-hydroxymethylphenyl, 5-amino-3-methoxycarbonylphenyl, 3-amidinophenyl, 3-amino-2-methylphenyl, 5-amino-2-methylthiophenyl, 3-aminophenyl, 3-amino-5(4-trifluoromethylbenzylamino)phenyl, 3-amino-5(4-trifluoromethylbenzyloxy)phenyl, 3-carboxyphenyl, 3-carboxy-5-hydroxyphenyl, 3-aino-5-carboxyphenyl, 3-chlorophenyl, 2-chlorophenyl, 3-cyanophenyl, 3,5-diaminophenyl, 3-dimethylaminophenyl, 2-fluorophenyl, 3-fluorophenyl, 2-hydroxyphenyl, 3-hydroxyphenyl, 3-methanesulfonylaminophenyl, 2-methoxyphenyl, 3-methoxyphenyl, 3-methoxyaminophenyl, 3-methoxycabonylphenyl, 2-methylaminophenyl, 3-methylaminophenyl, 2-methylphenyl, 3-methylphenyl, 4-methylphenyl, phenyl, 3-trifluoroacetamidophenyl, 3-trifluoromethylphenyl, 2-trifluoromethylphenyl, 5-amino-2-thienyl, 5-amino-3-thienyl, 3-bromo-2-thienyl, 3-pyridyl, 4-pyridyl, 2-thienyl, and 3-thienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
R¹⁶ and R¹⁹ are independently selected from the group consisting of 20 hydrido, aindino, amino, aminomethyl, methoxy, methylamino, hydroxy, hydroxymethyl, fluoro, chloro, and cyano;
R¹⁶ or R¹⁹ is optionally C(NR²⁵)NR²³R²⁴ with the proviso that R¹⁶, R¹⁹, and Q^{b} are not simultaneously hydrido;
R¹⁷ and R¹⁸ are independently selected from the group consisting of hydrido, fluoro, chloro, hydroxy, hydroxymethyl, amino, carboxy, and cyano;
Q^{b} is C(NR²⁵)NR²³R²⁴ or hydrido;
R²³, R²⁴, and R²⁵ are independently hydrido or methyl;
Q^{b} is CH₂.

40. Compound of claim 37 of the Formula: 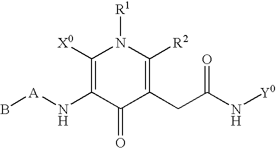
or a pharmaceutically acceptable salt thereof, wherein;
B is selected from the group consisting of hydrido, C2-C8 alkyl, C3-C8 alkenyl, C3-C8 alkynyl, and C2-C8 haloalkyl, wherein each member of group B is optionally substituted at any carbon up to and including 6 atoms from the point of attachment of B to A with one or more of the group consisting of R³², R³³, R³⁴, R³⁵, and R³⁶;
R³², R³³, R³⁴, R³⁵, and R³⁶ are independently selected from the group consisting of hydrido, acetamido, haloacetamido, amidino, guanidino, alkoxy, hydroxy, amino, alkoxyamino, alkylamino, alkylthio, amidosulfonyl, alkyl, halo, haloalkyl, haloalkoxy, hydroxyalkyl, carboalkoxy, carboxy, carboxamido, cyano, and Q^{b};
A is a bond or (CH(R¹⁵))ₚₐ—(W⁷)ᵣᵣ wherein rr is 0 or 1, pa is an integer selected from 0 through 3, and W is N(R⁷);
R⁷ is hydrido or alkyl;
R¹⁵ is selected from the group consisting of hydrido, halo, alkyl, and haloalkyl;
X⁰ is selected from the group consisting of hydrido, alkyl, halo, haloalkyl, aminoalkyl, and hydroxyalkyl;
R¹ is selected from the group consisting of hydrido, alkyl, hydroxyalkyl, and haloalkyl;
R² is Z⁰-Q^{b};
Z⁰ is a bond;
Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, hydroxy, amino, amidino, guanidino, alkylamino, alkylthio, alkoxy, alkylsulfinyl, alkylsulfonyl, amidosulfonyl, alkyl, halo, haloalkyl, haloalkoxy, hydroxyalkyl, carboxy, carboxamido, and cyano;
R¹⁰ and R¹² are independently selected from the group consisting of hydrido, acetamido, haloacetarido, amidino, guanidino, alkyl, alkoxy, alkoxyamino, hydroxy, amino, alkylamino, alkylsulfonamido, aridosulfonyl, hydroxyalkyl, aminoalkyl, halo, haloalkyl, carboalkoxy, carboxy, carboxamido, carboxyalkyl, and cyano;
Y⁰ is phenyl or a heteroaryl of 5 or 6 ring members, wherein one carbon of said phenyl or said heteroaryl is substituted by Q^{s}, a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} isoptionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloallylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally selected from the group consisting of NR²⁰R²¹,N(R²⁶)C(NR²⁵)N(R²³)(R²⁴), and C(NR²⁵:)NR²³R²⁴, with the proviso that R¹⁶, R¹⁹, and Q^{b} are not simultaneously hydrido;
Q^{b} is selected from the group consisting of NR²⁰R²¹, hydrido, N(R²⁶)C(NR²⁵)N(R²³)(R²⁴), and C(NR²⁵)NR²³R²⁴;
R²⁰, R²¹, R²³, R²⁴, R²⁵, and R²⁶ are independently hydrido or alkyl;
Q^{s} is CH_{2.}

41. Compound of claim 40 or a pharmaceutically acceptable salt thereof, wherein;
B is selected from the group consisting of hydrido, ethyl, 2-propenyl, 2-propynyl, propyl, isopropyl, butyl, 2-butenyl, 2-butynyl, sec-butyl, tert-butyl, isobutyl, 2-methylpropenyl, 1-pentyl, 2-pentenyl, 3-pentenyl, 2-pentynyl, 3-pentynyl, 2-pentyl, 3-pentyl, 2-methylbutyl, 2-methyl-2-butenyl, 3-methylbutyl, 3-methyl-2-butenyl, 1-hexyl, 2-hexenyl, 3-hexenyl, 4-hexenyl, 2-hexynyl, 3-hexynyl, 4-hexynyl, 2-hexyl, 1-methyl-2-pentenyl, 1-methyl-3-pentenyl, 1-methyl-2-pentynyl, 1-methyl-3-pentynyl, 3-hexyl, 1-ethyl-2-butenyl, 1-heptyl, 2-heptenyl, 3-heptenyl, 4-heptenyl, 5-heptenyl, 2-heptynyl, 3-heptynyl, 4-heptynyl, 5-heptynyl, 2-heptyl, 1-methyl-2-hexenyl, 1-methyl-3-hexenyl, 1-methylhexenyl, 1-methyl-2-hexynyl, 1-methyl-3-hexynyl, 1-methylhexynyl, 3-heptyl, 1-ethyl-2-pentenyl, 1-ethyl-3-pentenyl, 1-ethyl-2-pentynyl, 1-ethyl-3-pentynyl, 2,2,2-trifluoroethyl, 2,2-difluoropropyl, 4-triuoromethyl-5,5,5-trifluoropentyl, 4-trifluoromethylpentyl, 5,5,6,6,5-pentafluorohexyl, and 3,3,3-trifluoropropyl, wherein each member of group B is optionally substituted at any carbon up to and including 5 atoms from the point of attachment of B to A with one or more of the group consisting of R³², R³³, R³⁴, R³⁵, and R³⁶;
R³², R³³, R³⁴, R³⁵, and R³⁶ are independently selected from the group consisting of hydrido, amidino, guanidino, methyl, ethyl, methoxy, ethoxy, hydroxy, amino, N-methylamino, dimethylamino, methoxyamino, methylthio, ethylthio, trifluoromethyl, pentafluoroethyl, 2,2,2-rifluoroethyl, fluoro, chloro, bromo, amidosulfonyl, N-methylamidosulfonyl, hydroxymethyl, amidocarbonyl, carboxy, cyano, and Q^{b};
A is selected from the group consisting of a bond, NH, N(CH₃), CH₂, CH₃CH, and CH₂CH₂;
A is optionally selected from the group consisting of CH₂N(CH₃), CH₂N(CH₂CH₃), CH₂CH₂N(CH₃), and CH₂CH₂N(CH₂CH₃) with the proviso that B is hydrido;
X^{o} is selected from the group consisting of hydrido, aminomethyl, methyl, ethyl, trfluoromethyl, 2,2,2-trifluoroethyl, hydroxymethyl, 2-hydroxyethyl, chloro, and fluoro;
R¹ is selected from the group consisting of hydrido, methyl, ethyl, propyl, 2,2,2-trifluoroethyl, 3,3,3-trifluoropropyl, 2,2,3,3,3-pentafluoropropyl, and 2-hydroxyethyl;
R² is selected from the group consisting of phenyl, 2-thienyl, 2-furyl, 2-pyrrolyl, 2-imidazolyl, 2-thiazolyl, 3-isoxazolyl, 2-pyridyl, and 3-pyridyl, wherein a carbon adjacent to the carbon at the point of attachment of said phenyl or heteroaryl ring to the 4-pyridone ring is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
R⁹, R¹¹, and R¹³ are independently selected from the group consisting of hydrido, methyl, ethyl, methoxy, ethoxy, hydroxy, amino, N-methylamino, N,N-dimethylamino, methylthio, trifluoromethyl, pentafluoroethyl, 2,2,2-tWifluoroethyl, fluoro, chloro, bromo, amidosulfonyl, N-methylamidosulfonyl, N,N-dimethylamidosulfonyl, hydroxymethyl, 1-hydroxyethyl, amidocarbonyl, N-methylamidocarbonyl, carboxy, and cyano;
R¹⁰ and R¹² are independently selected from the group consisting of hydrido, amidino, amidocarbonyl, N-methylamidocarbonyl, N-benzylamidocarbonyl, N-(2,5-chlorobenzyl)amidocarbonyl, N-(3-fluorobenzyl)amidocarbonyl, N-(2-trifluoromethylbenzyl)amidocarbonyl, N-(1-phenylethyl)amidocarbonyl, N-(1-methyl-1-phenylethyl)amidocarbonyl, N-benzylamidosulfonyl, N-(2-chlorobenzyl)amidosulfonyl, N-ethylamidocarbonyl, N-isopropylamidocarbonyl, N-propylamidocarbonyl, N-isobutylamidocarbonyl, N-(2-butyl)amidocarbonyl, N-cyclobutylamidocarbonyl, N-yclopentylamidocarbonyl, N-cyclohexylamidocarbonyl, guanidino, methyl, ethyl, methoxy, ethoxy, hydroxy, hydroxymethyl, 1-hydroxyethyl, 2-hydroxyethyl, carboxy, cabxymethyl, amino, acetamido, trfluoromethyl, pentafluoroethyl, 2,2,2-trifluoroethyl, trifluoroacetamido, aminomethyl, N-methylamino, dimethylamino, methoxyamino, amidosulfonyl, N-methylamidosulfonyl, N,N-dimethylamidosulfonyl, methanesulfonamido, methoxycarbonyl, fluoro, chloro, bromo, and cyano;
Y⁰ is selected from the group consisting of:
1-Q^{b}-4-Q^{s}-2-R¹⁶-3-R¹⁷-5-R¹⁸-6-R¹⁹ benzene,
2-Q^{b}-6-Q^{s}-2-R¹⁷-5-R¹⁸-4-R¹⁹ pyridine, 2-Q^{b}-5-Q^{s}-3-R¹⁶-4 R¹⁷ thiophene,
3-Q^{b}-6-Q^{s}-2-R¹⁶-5-R¹⁸-4-R¹⁹ pyridine, 2-Q^{b}-5-Q^{s}-3-R¹⁶-4 R¹⁹ thiophene,
3-Q^{b}-5-Q^{s}-4-R¹⁶-2-R¹⁹ furan, 2-Q^{b}-5-Q^{s}-3-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ thiazole,
R¹⁶, R¹⁷, R¹⁸, and R¹⁹ are independently selected from the group consisting of hydrido, methyl, ethyl, amidino, guanidino, methoxy, hydroxy, amino, aminomethyl, 1-aminoethyl, 2-aminoethyl, N-methylamino, dimethylamino, methylthio, ethylthio, trifluoromethylthio, methylsulfinyl, methylsulfonyl, trifluoromethyl, pentafluoroethyl, 2,2,2-trifluoroethyl, trifluoromethoxy, fluoro, chloro, hydroxymethyl, carboxy, and cyano;
Q^{b} is selected from the group consisting of NR²⁰R²¹, C(NR²⁵)NR²³R²⁴, and N(R²⁶)C(NR²⁵)N(R²³)(R²⁴);
R²⁰, R²¹, R²³, R²⁴, R²⁵, and R²⁶ are independently selected from the group consisting of hydrido, methyl, and ethyl;
Q^{s} is CH₂.

42. Compound of claim 41 or a pharmaceutically acceptable salt thereof, wherein;
B is selected from the group consisting of hydrido, ethyl, 2-propenyl, 2-propynyl, propyl, isopropyl, butyl, 2-butyl, (R)-2-butyl, (S)-2-butyl, tert-butyl, isobutyl, 1-pentyl, 3-pentyl, 2-methylbutyl, 2,2,2-trifluoroethyl, 6-amidocarbonylhexyl, 4-methyl-2-pentyl, 3-hydroxypropyl, 1-methoxy-2-propyl, 2-methoxyethyl, 2-methyl-2-butyl, 3-methyl-2-butyl, 2-dimethylaminopropyl, 2-yanoethyl, 6-hydroxyhexyl, 2-hydroxyethyl, 2-amidinoethyl, 2-guanidinoethyl, 3-guanidinopropyl, 4-guanidinobutyl, 3-hydroxypropyl, 4-hydroxybutyl, 6-cyanohexyl, 2-dimethylaminoethyl, 3-methylbutyl, 2-methylbutyl, (S)-2-methylbutyl, 3-aminopropyl, 2-hexyl, and 4-aminobutyl;
A is selected from the group consisting of a bond, CH₂, CH₃CH, and CH₂CH₂;
X^{o} is selected from the group consisting of hydrido, methyl, ethyl, trifluoromethyl, 2,2,2-tifluoroethyl, hydroxymethyl, 2-hydroxyethyl, chloro, and fluoro;
R¹ is selected from the group consisting of hydrido, methyl, ethyl, and Z2,2-trifluoroethyl;
R² is selected from the group consisting of 3-amidocarbonyl-5-aminophenyl, 3-amidocarbonyl-5-aminophenyl, 3-amino-5S(N-benzylamidocarbonyl)phenyl, 3-amino-54N-2-chlorobenzyl)amidocarbonyl)phenyl, 3-amino-5-(N-(3-fluorobenzyl)amidocarbonyl)phenyl, 3-amino-5-(N-2-trifluoromethylbenzyl)amidocarbonyl)phenyl, 3-amino-5-(N-(1-phenylethyl)amidocarbonyl)phenyl, 3-amino-5-(N-(1-methyl-1-phenylethyl)amidocarbonyl)phenyl, 3-ammino-5-(N-benzylamidosulfonyl)phenyl, 3-amino-5-(N-(2-chlorobenzyl)amidosulfonyl)phenyl, 3-amino-(N-ethylamidocarbonyl)phenyl, 3-amino-5-(N-isopropylamidocarbonyl)phenyl, 3-amino-5-(N-propylamidocarbonyl)phenyl, 3-amino-5-(N-isobutylamidocarbonyl)phenyl, 3-amino-5-(N-2-butyl)amidocarbonyl)phenyl, 3-amino-5-(N-cyclobutylamidocarbonyl)phenyl, 3-amino-5-(N-cyclopentylamidocarbonyl)phenyl, 3-amino-(Ncyclohexylamidocarbonyl)phenyl, Samino-2-fluorophenyl, 3-amino-5-hydroxymethylphenyl, 5-amino-3-methoxycarbonylphenyl, 3-amidinophenyl, 3-amino-2-methylphenyl, 5-amino-2-methylthiophenyl, 3-aminophenyl, 3-carboxyphenyl, 3-carboxy-5-aminophenyl, 3-carboxy-5-hydroxyphenyl, 3-carboxymethyl-5-aminophenyl, 3-carboxymethyl-5-hydroxyphenyl, 3-carboxymethylphenyl, 3-chlorophenyl, 2-chlorophenyl, 3-cyanophenyl, 3,5-diaminophenyl, 3-dimethylaminophenyl, 2-fluorophenyl, 3-fluorophenyl, 2,5-difluorophenyl, 2-hydroxyphenyl, 3-hydroxyphenyl, 3-methanesulfonylaminophenyl, 2-methoxyphenyl, 3-methoxyphenyl, 3-methoxyaminophenyl, 3-methoxycarbonylphenyl, 2-methylaminophenyl, 3-methylaminophenyl, 2-methylphenyl, 3-methylphenyl, 4-methylphenyl, phenyl, 3-trifluoroacetamidophenyl, 3-trifluoromethylphenyl, 2-trifluoromethylphenyl, 5-amino-2-thienyl, 5-amino-3-thienyl, 3-bromo-2-thienyl, 3-pyridyl, 4-pyridyl, 2-thienyl, and 3-thienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-6-Q^{s}-2-R¹⁶-5-R¹⁸-4-R¹⁹ pyridine, 2-Q^{b}-5-Q^{s}-3-R¹⁶-4 R¹⁷ thiophene,
R¹⁶ and R¹⁹ are independently selected from the group consisting of hydrido, amidino, amino, aminomethyl, methoxy, methylamino, hydroxy, hydroxymethyl, fluoro, chloro, and cyano;
R¹⁷ and R¹⁸ are independently selected from the group consisting of hydrido, fluoro, chloro, hydroxy, hydroxymethyl, amino, carboxy, and cyano;
Q^{b} is C(NR²⁵)NR²³R²⁴ R²³, R²⁴, and R²⁵ are independently hydrido or methyl;
Q^{b} is CH₂.

43. Compound of claim 42 or a pharmaceutically acceptable salt thereof, wherein;
B is selected from the group consisting of hydrido, ethyl, 2-propenyl, 2-propynyl, propyl, isopropyl, butyl, 2-butyl, (R)-2-butyl, (S)₂-butyl, tert-butyl, isobutyl, 1-pentyl, 3-pentyl, 2-methylbutyl, 2,2,2-trifluoroethyl, 6-amidocarbonylhexyl, 4-methyl-2-pentyl, 3-hydroxypropyl, 1-methoxy-2-propyl, 2-methoxyethyl, 2-methyl-2-butyl, 3-methyl-2-butyl, 2-dimethylaminopropyl, 2-cyanoethyl, 6-hydroxyhexyl, 2-hydroxyethyl, 2-amnidinoethyl, 2-guanidinoethyl, 3-guanidinopropyl, 4-guanidinobutyl, 3-hydroxypropyl, 4-hydroxybutyl, 6-cyanohexyl, 2-dimethylaminoethyl, 3-methylbutyl, 2-methylbutyl, (S)-2-methylbutyl, 3-ariinopropyl, 2-hexyl, and 4-aminobutyl;
A is selected from the group consisting of a bond, CH₂, CH₃CH, and CH₂CH₂;
X^{o} is selected from the group consisting of hydrido, methyl, ethyl, trifluoromethyl, 2,2,2-trifluoroethyl, chloro, and fluoro;
R¹ is selected from the group consisting of hydrido, methyl, ethyl, and 2,2,5-trifluoroethyl;
R² is selected from the group consisting of 3-amidocarbonyl-5-minophenyl, 3-amino-5-(N-benzylamidocarbonyl)phenyl, 3-amino-5-(N-(2-chlorobenzyl)amidocarbonyl)phenyl, 3-amino 5-(N-(3-fluorobenzyl)amidocarbonyl)phenyl, 3-amino-5-(N-(2-trifluoromethylbenzyl)amidocarbonyl)phenyl, 3-amino-5-(N-(1-phenylethyl)amidocarbonyl)phenyl, 3-amino-5-(N-(1-methyl-1-phenylethyl)awidocarbonyl)phenyl, 3-amino-5-(N-benzylamidosulfonyl)phenyl, 3-amino-5-(N-(2-chlorobenzyl)amidosulfonyl)phenyl, 3-amino-(N-ethylamidocarbonyl)phenyl, 3-amino-5-(N-isopropylamidocarbonyl)phenyl, 3-amino-5-(N-propylamidocarbonyl)phenyl, 3-amino-(N-isobutylamidocarbonyl)phenyl, 3-amino-5-(N-2-butyl)amidocarbonyl)phenyl, 3-amino-(N-cyclobutylamidocarbonyl)phenyl, 3-amino-5-(N-cyclopentylamidocarbonyl)phenyl, 3-amino-5-(N-cyclohexylamidocarbonyl)phenyl, 3-aminophenyl, 3-carboxy-5-aminophenyl, 3-chlorophenyl, 3,5-diaminophenyl, 3-dimethylaminophenyl, 3-hydroxyphenyl, 3-methanesulfonylaminophenyl, 3-methylaminophenyl, 2-methylphenyl, 3-methylphenyl, phenyl, 3-trifluoroacetamidophenyl, 3-bromo-2-thienyl, 2-thienyl, and 3-thienyl;
Y⁰ is selected from the group consisting of 5-amidino-2-thienylmethyl, 4-amidinobenzyl, 2-fluoro4-amidinobenzyl, and 3-fluoro-4-amidinobenzyl.

44. Compound of claim 37 where said compound is selected from the group of the Formula: 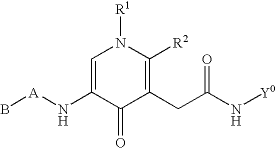
or a pharmaceutically acceptable salt thereof, wherein B, A, R¹, R², X^{o}, and Y⁰ are selected to form a compound selected from the group consisting of;
R² is 3-aminophenyl, B is 2,2,2-trifluoroethyl, A is a bond, YN is 4 amidinobenzyl, and R¹ is methyl;
R² is 3-aminophenyl, B is (S)-2-butyl, A is a bond, Y⁰ is 4-amidinobenzyl, and R¹ is methyl;
R² is 5-amiao-2-fluorophenyl, B is isopropyl, A is a bond, Y⁰ is 4 ainidinobenzyl, and R¹ is methyl;
R² is 2-methyl-3-acicophenyl, B is isopropyl, A is a bond, Y⁰ is 4 amidinobenzyl, and R¹ is methyl;
R² is 3-aminophenyl, B is ethyl, A is a bond, Y⁰ is 4-amidinobenzyl₁ and R¹ is methyl;
R² is 3-aminophenyl, B is ethyl, A is a bond, Y⁰ is 4-amidino-2-fluorobenzyl, and R¹ is methyl;
R² is 3-aminophenyl, B is 2-propenyl, A is a bond, Y⁰ is 4-amidinobenzyl, and R¹ is methyl;
R² is 3-aminophenyl, B is isopropyl, A is a bond, Y⁰ is 4-amidino-2-fluorobenzyl, and R¹ is methyl;
R² is 3-aminophenyl, B is isopropyl, A is a bond, Y⁰ is 4-amidinobenzyl, and R¹ is methyl;
R² is 3-aminophenyl, B is 2-butyl, A is a bond, Y⁰ is 4-amidinobenzyl, and R¹ is methyl;
R² is 3-aminophenyl, B is (R)-2-butyl, A is a bond, Y⁰ is 4-amidinobenzyl, and R¹ is methyl;
R² is 3-aminophenyl, B is 2-propynyl, A is a bond, Y⁰ is 4-amidinobenzyl, and R¹ is methyl;
R² is 3-ninophenyl, B is 3-pentyl, A is a bond, Y⁰ is 4&nidinobenzyl, and R¹ is hydrido;
R² is 3-aminophenyl, B is hydrido, A is CH₂, Y⁰ is 4-amidinobenzyl, and R¹ is methyl;
R² is 3-aminophenyl, B is ethyl, A is CH₂, Y⁰ is 4-amidinobenzyl, and R¹ is methyl;
R² is 3-aminophenyl, B is 2-methypropyl, A is a bond, Y⁰ is 4 amidinobenzyl, and R¹ is methyl;
R² is 3-aminophenyl, B is 2-propyl, A is CH₃CH, Y⁰ is 4-amidinobenzyl, and R¹ is methyl;
R² is 3-aminophenyl, B is propyl, A is a bond, Y⁰ is 4-amidino-2-fluorobenzyl, and R¹ is methyl;
R² is 3-aminophenyl, B is 6-amidocarbonylhexyl, A is a bond, Y⁰ is 4 amidinobenzyl, and R¹ is methyl;
R² is 3-aminophenyl, B is tert-butyl, A is a bond, Y⁰ is 4-amidinobenzyl, and R¹ is hydrido;
R² is 3-aminophenyl, B is tert-butyl, A is a bond, Y⁰ is 4-amidinobenzyl, and R¹ is methyl;
R² is 3-aminophenyl, B is 3-hydroxypropyl, A is a bond, Y⁰ is 4 amidinobenzyl, and R¹ is methyl;
R² is 3-aminophenyl, B is 2-methylpropyl, A is a bond, Y⁰ is 4-aamidino-2-fluorobenzyl, and R¹ is methyl;
R² is 3-aminophenyl, B is butyl, A is a bond, Y⁰ is 4-amidinobenzyl, and R¹ is methyl;
R² is 3-amiophenyl, B is 1-methoxy-2-propyl, A is a bond, Y⁰ is 4 amidinobenzyl, and R¹ is methyl;
R² is 3-aminophenyl, B is 2-methoxyethyl, A is a bond, Y⁰ is 4 amdinobenzyl, and R¹ is methyl;
R² is 3-aminophenyl, B is 2-propyl, A is a bond, Y⁰ is 5-amidino-2-thienylmethyl, and R¹ is methyl;
R² is amino-2-methylthiophenyl, B is 2-propyl, A is a bond, Y⁰ is 4 amidinobenzyl, and R¹ is methyl;
R² is 3-amino-5-carboxyphenyl, B is isopropyl, A is a bond, Y⁰ is 4 amidinobenzyl, and R¹ is methyl;
R² is 3-amino-carbomedioxyphenyl, B is isopropyl, A is a bond, Y⁰ is 4 amidinobenzyl, and R¹ is methyl;
R² is 3-aminophenyl, B is isopropyl, A is a bond, Y⁰ is 4-amidinobenzyl, and R¹ is ethyl;
R² is 3-amino-5-carboxamidophenyl, B is isopropyl, A is a bond, Y⁰ is 4 amnidinobenzyl, and R¹ is methyl;
R² is 3-amino-5-(N-benzyl-N-methylamidocarbonyl)phenyl, B is isopropyl, A is a bond, Y⁰ is 4-amidinobenzyl, and R¹ is methyl;
R² is 3-amino-5-(N-(1-phenylethyl)amidocarbonyl)phenyl, B is isopropyl, A is a bond, Y⁰ is 4-amidinobenzyl, and R¹ is methyl;
R² is 3-araino-5-(N-(2-phenyl-2-propyl)amidocarbonyl)phenyl, B is isopropyl, A is a bond, Y⁰ is 4-amidinobenzyl, and R¹ is methyl;
R² is 3-amino-5-(N-(2,4-dichlorobenzyl)amidocarbonyl)phenyl, B is isopropyl, A is a bond, Y⁰ is 4-amidinobenzyl, and R¹ is methyl;
R² is 3-amino-5-(N-(4-bromobenzyl)amidocarbonyl)phenyl, B is isopropyl, A is a bond, Y⁰ is 4-amidinobenzyl, and R¹ is methyl;
R² is 3-amino-5-(N-benzylamidocarbonyl)phenyl, B is isopropyl, A is a bond, Y⁰ is 4-amidinobenzyl, and R¹ is methyl;
R² is 3-amino-5-(N-(2-chlorobenzyl)amidocarbonyl)phenyl, B is isopropyl, A is a bond, Y⁰ is 4-amidinobenzyl, and R¹ is methyl;
R² is 3-amino-5-N-(2-trifluoromethylbenzyl)amidocarbonyl)phenyl, B is isopropyl, A is a bond, Y⁰ is 4-amaidinobenzyl, and R¹ is methyl;
R² is 3-amino-5-N-(3-fluorobenzyl)amidocarbonyl)phenyl, B is isopropyl, A is a bond, Y, is 4-amidinobenzyl, and R¹ is methyl;
R² is 3-amino-5-(N-(3-trifluoromethylbenzyl)amidocarbonyl)phenyl, B is isopropyl, A is a bond, Y⁰ is 4-amidinobenzyl, and R¹ is methyl;
R² is 3-amino-5-(N-isobutylamidocarbonyl)phenyl, B is isopropyl, A is a bond, Y⁰ is 4-amidinobenzyl, and R¹ is methyl;
R² is 3-amino(N-cyclobutylamidocarbonyl)phenyl, B is isopropyl, A is a bond, Y⁰ is 4-amidinobenzyl, and R¹ is methyl;
R² is 3-amino-5-(N-cycopentylamidocarbonyl)phenyl, B is isopropyl, A is a bond, Y⁰ is 4-amidinobenzyl, and R¹ is methyl;
R² is 3-amino-5-(N-ycloheptylamidocarbonyl)phenyl, B is isopropyl, A is a bond, Y⁰ is 4-amidinobenzyl, and R¹ is methyl;
R² is 3-amino-5-(N-(2-pyridylmethyl)amidocarbonyl)phenyl, B is isopropyl, A is a bond, Y⁰ is 4-amidinobenzyl, and R¹ is methyl;
R² is 3-amino-5-(N-3-pyridylmethyl)amidocarbonyl)phenyl, B is isopropyl, A is a bond, Y⁰ is 4-amidinobenzyl, and R¹ is methyl;
R² is 3-amino-5-N-(2-4-methoxyphenyl)ethyl)amidocarbonyl)phenyl, B is isopropyl, A is a bond, Y⁰ is 4-amidinobenzyl, and R¹ is methyl;
R² is 3-amino-5-(N-(3-phenylpropyl)amidocarbonyl)phenyl, B is isopropyl, A is a bond, Y⁰ is 4-amidinobenzyl, and R¹ is methyl;
R² is 3-amino-5-(N-(2,2-diphenylethyl)amidocarbonyl)phenyl, B is isopropyl, A is a bond, Y⁰ is 4-amidinobenzyl, and R¹ is methyl;
R² is 3-amino-5-(N-(2-naphthylmethyl)amidocarbonyl)phenyl, B is isopropyl, A is a bond, Y⁰ is 4-amidinobenzyl, and R¹ is methyl;
R² is 3-amino-5-N-(1,2,3,4-tetrahydronaphth-2-ylmethyl)amidocarbonyl)phenyl, B is isopropyl, A is a bond, Y⁰ is 4-amidinobenzyl, and R¹ is methyl;
R² is 3-aminophenyl, B is 2-propyl, A is a bond, Y⁰ is 4-amidino3-fluorobenzyl, and R¹ is hydrido;
R² is 3-carboxyphenyl, B is 2-propyl, A is a bond, Y⁰ is 4-amidinobenzyl, and R¹ is hydrido;
R² is 3-aminophenyl, B is 2-propyl, A is a bond, Y⁰ is 4-amidino-3-fluorobenzyl, and R¹ is methyl;
R² is 3,5-diaminophenyl, B is 2,2,2-trifluoroethyl, A is a bond, Y⁰ is 4 amidinobenzyl, and R¹ is methyl;
R² is 3,5-diaminophenyl, B is (S)-2-butyl, A is a bond, Y⁰ is 4 amidinobenzyl, and R¹ is methyl;
R² is 3,5-diaminophenyl, B is isopropyl, A is a bond, Y⁰ is 4 amidinobenzyl, and R¹ is methyl; R² is 3,5-dianiophenyl, B is isopropyl, A is a bond, Y⁰ is 4-amidino-2-fluorobenzylbenzyl, and R¹ is methyl;
R² is 3,5-iaminophenyl, B is ethyl, A is a bond, Y⁰ is 4-amidinobenzyl, and R¹ is methyl;
R² is 3,5-diaminophenyl, B is ethyl, A is a bond, Y⁰ is 4-amidino-2-fluorobenzyl, and R¹ is methyl;
R² is 3-amino-5-carboxyphenyl, B is 2,2,2-trifluoroethyl, A is a bond, Y⁰ is 4-amidinobenzyl, and R¹ is methyl;
R² is 3-amino-5-carboxyphenyl, B is (S)₂-butyl, A is a bond, Y⁰ is 4-amidinobenzyl, and R¹ is methyl;
R² is 3-amino-5-carboxyphenyl, B is isopropyl, A is a bond, Y⁰ is 4 amidino-2-fluorobenzylbenzyl, and R¹ is methyl;
R² is 3-amino-5-carboxyphenyl, B is ethyl, A is a bond, Y⁰ is 4 amidinobenzyl, and R¹ is methyl;
R² is 3-amino-5-carboxyphenyl, B is ethyl, A is a bond, Y⁰ is 4-amidino-2-fluorobenzyl, and R¹ is methyl;
R² is 3-amino-5-(N-benzylamidocarbonyl)phenyl, B is 2,2,2-trifluoroethyl, A is a bond, Y⁰ is 4-amidinobenzyl, and R¹ is methyl;
R² is 3-amino-5-(N-benzylamidocarbonyl)phenyl, B is (S)-2-butyl, A is a bond, Y⁰ is 4-amidinobenzyl, and R¹ is methyl;
R² is 3-amino-5-(N-benzylamidocarbonyl)phenyl, B is isopropyl, A is a bond, Y⁰ is 4-amidino-2-fluorobenzylbenzyl, and R¹ is methyl;
R² is 3-amino-5-(N-benzylamidocarbonyl)phenyl, B is ethyl, A is a bond, Y⁰ is 4-amidinobenzyl, and R¹ is methyl;
R² is 3-amino-5-(N-benzylamidocarbonyl)phenyl, B is ethyl, A is a bond, Y⁰ is 4-amidino-2-fluorobenzyl, and R¹ is methyl; and R² is 3,5-diaminophenyl, B is isopropyl, A is a bond, Y⁰ is 4 amidinobenzylbenzyl, and R¹ is hydrido.

45. Compound of claim 22 of the Formula: 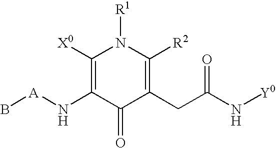
or a pharmaceutically acceptable salt thereof, wherein;
B is a C3-C7 cycloalkyl or a C4-C6 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, a ring carbon or nitrogen adjacent to the R position and two atoms from the point of attachment is optionally substituted with R¹², a zng carbon or nitrogen three atoms from the point of attachment and adjacent to the R position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;
R⁹, R¹¹, and R¹³ are independently selected from the group consisting of hydrido, hydroxy, amino, amiidino, guanidino, alkylamino, alkylthio, alkylsulfonamido, alkylsulfinyl, alkylsulfonyl, amidosulfonyl, alkyl, alkoxy, halo, haloalkyl, haloalkoxy, hydroxyalkyl, hydroxyhaloalkyl, carboxy, carboxamido, and cyano;
R¹⁰ and R¹² are independently selected from the group consisting of hydndo, acetamido, haloacetamido, amidino, guanidino, alkyl, aryl, aralkyl, cycloalkyl, cycloalkylalkyl, heteroaryl, heterocyclyl, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkoxyamino, alkylamino, arylamino, aralkylamino, heteroarylamino, heteroaralkylamino, heterocyclylamino, heterocyclylalkylamino, alkylsulfonamido, amidosulfonyl, arylsulfinyl, arikylsulfmnyl, cycloalkylsulfinyl, heteroarylsulfinyl, arylsulfonyl, aralkylsulfonyl, cycloalkylsulfonyl, heteroarylsulfonyl, hydroxyalkyl, hydroxyhaloalkyl, aminoalkyl, carboalkoxy, carboxy, carboxyalkyl, carboxamido, halo, haloalkyl, and cyano;
R³³ and R³⁴ are independently selected from the group consisting of hydtido, acetamido, haloacetanido, amidino, guanidino, alkoxy, hydroxy, amino, alkoxyamino, alkylamino, alkylthio, amidosulfonyl, alkyl, halo, haloalkyl, haloalkoxy, hydroxyalkyl, hydroxyhaloalkyl, carboalkoxy, carboxy, carboxamido, and cyano;
R³³ is optionally Q^{b};
A is a bond or (CH(R¹⁵))ₚₐ—(W⁷)ᵣᵣ wherein rr is 0 or 1, pa is an integer selected from 0 through 3, and W is (R⁷)NC(O) or N(R⁷);
R⁷ is selected from the group consisng of hydrido, hydroxy and alkyl;
R¹⁵ is selected from the group consisting of hydrido, halo, alkyl, and haloallyl;
X⁰ is selected from the group consisting of hydrido, amino, alkyl, halo, haloalkyl, haloalkoxy, aminoalkyl, hydroxy, alkoxy, hydroxyalkyl, and alkylthio;
R¹ is independently selected from the group consisting of hydrido, hydroxy, amino, hydroxyalkyl, alkyl, aminoalkyl, and haloalkyl;
R² is Z⁰-Q^{b};
Z2 is selected from the group consisting of a bond, CH₂, CH₂CH₂, W⁰—(CH(R:))ₚ wherein p is 0 or 1 and W⁰ is selected from the group consisting of O, S, and N(R⁴¹);
R⁴¹ and R⁴² are independently hydrido or alkyl;
Q is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
Y⁰ is phenyl or a heteroaryl of 5 or 6 ring members, wherein one carbon of said phenyl or said heteroaryl is substituted by Q^{s} a carbon two or three atoms from the point of attachment of Q^{s} to said phenyl or said heteroaryl is substituted by Q^{b}, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkyylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally NR²⁰ or and C(NR²⁵)NR²³R²⁴, with the proviso that R¹⁶, R¹⁹, and Q^{b} are not simultaneously hydrido;
Q^{b} is selected from the group consisting of NR²⁰R²¹, hydrido, and C(NR²⁵)NR²³R²⁴, with the proviso that no more than one of R²⁰ and R²¹ is hydroxy at the same time and with the further proviso that no more than one of R²³ and R²⁴ is hydroxy at the same time;
R²⁰, R²¹, R²³, R²⁴, and R²⁵ are independently selected from the group consisting of hydrido, alkyl, and hydroxy;
Q^{s} is selected from the group consisting of a bond, CH₂, and CH₂CH₂.

46. Compound of claim 45 or a pharmaceutically acceptable salt thereof, wherein;
B is selected-from the group consisting of cyclopropyl, cyclobutyl, oxetan-3-yl, azetidin-1-yl, azetidin-2-yl, azetidin-3-yl, thiaetan-3-yl, cyclopentyl, cyclohexyl, norbornyl, 7-oxabicyclo[2.2.1]heptan-2-yl, bicyclo[3.1.0-]hexanyl, cycloheptyl, 2-inorpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyfTolidinyl, 2-dioxanyl, 4H-2-pyranyl, 4H-3-pyranyl, 4H pyranyl, 4H-pyranone-2-yl, 4H-pyran4-one-3-yl, 2-tetrahydrofuranyl, 3-tetrahydrofuranyl, 2-tetrahydropyranyl, 3-tetrahydropyranyl, 4-tetrahydropyranyl, 2-tetrahydrothienyl, and 3-tetrahydrothienyl, wherein each ring carbon is optionally substituted with R³³, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, and a ring carbon or nitrogen adjacent to the R¹³ position and two atoms from the point of attachment is optionally substituted with R¹²;
R⁹, R¹¹, and R¹³ are independently selected from the group consisting of hydrido, amidino, guanidino, carboxy, methyl, ethyl, propyl, isopropyl, methoxy, ethoxy, isopropoxy, propoxy, hydroxy, amino, N-methylamino, N,N-dimethylamino, N-ethylamino, methylthio, ethylttio, isopropylthio, trifluoromethyl, pentafluoroethyl, 2,2,2-trifluoroethyl, 2,2,3,3,3-pentafluoropropyl, trifluoromethoxy, 1,1,2,2-teltafluoroethoxy, fluoro, chloro, bromo, methanesulfonamido, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, amidocarbonyl, N-methylamidocarbonyl, N,N-dimethylamidocarbonyl, and cyano;
R¹⁰ and R¹² are independently selected from the group consisting of hydrido, amidino, guanidino, carboxy, carboxymethyl, methyl, ethyl, propyl, isopropyl, methoxy, ethoxy, isopropoxy, propoxy, hydroxy, amino, methoxyamino, ethoxyamino, acetamido, trifluoroacetamido, aminomethyl, 1-aminoethyl, 2-aminoethyl, N-methylamino, dimethylamino, N-ethylamino, niethanesulfonamido, amidosulfonyl, N-methylamidosulfonyl, NjNdimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, N-benzylamidocarbonyl, N-(2-chlorobenzyl)amidocarbonyl, N-(3-fluorobenzyl)amidocarbonyl, N-(2-trifluoromethylbenzyl)aniidocarbonyl, N-(1-phenylethyl)amidocarbonyl, N-(1-methyl-1-phenylethyl)amidocarbonyl, N-benzylamidosulfonyl, N-(2-chlorobenzyl)amidosulfonyl, N-ethylamidocarbonyl, N-isopropylamidocarbonyl, N-propylamidocarbonyl, N-isobutylanidocarbonyl, N-(2-butyl)amidocarbonyl, N-cyclobutylarmidocarbonyl, N-cyclopentylamidocarbonyl, N-cyclohexylamidocarbonyl, fluoro, chloro, bromo, cyano, cyclobutoxy, cyclohexoxy, cyclohexylmethoxy, 4-trifluoromethycyclohexylmethoxy, cyclopentoxy, benzyl, benzyloxy, 4-bromo-3-fluorophenoxy, 3-bromobenzyloxy, 4-bromobenzyloxy, 4-bromobenzylamino, 5-bromopyrid-2-ylmethylamino, 4-butoxyphenamino, 3-chlorobenzyl, 4-chlorophenoxy, 4-chloro-3-ethylphenoxy, 4-chloro-3-ethylbenzylamino-4-chloro-3-ethylphenylamino, 3-chlorobenzyloxy, 4-chlorobenzyloxy, 4-chlorobenzylsulfonyl, 4-ch1-orophenylamino, 4-chlorophenylsulfonyl, 5-chloropyrid-3-yloxy, 2-cyanopyrid-3-yloxy, 2,3-difluorobenzyloxy, 2,4-difluorobenzyloxy, 3,4-difluorobenzyloxy, 2,5-difluorobenzyloxy, 3,5-difluorophenoxy, 3,5-difluorobenzyloxy, 4-difluoromethoxybenzyloxy, 2,3-difluorophenoxy, 2,4-difluorophenoxy, 2,5-difluorophenoxy, 3,5-dimethylphenoxy, 3,4-dimethylphenoxy, 3,4-dimethylbenzyloxy, 3,5-dimethylbenzyloxy, 4-ethoxyphenoxy, 4-ethylbenzyloxy, 3-ethylphenoxy, 4-ethylaminophenoxy, 3-ethyl-5-methylphenoxy, 4-fluorobenzyloxy, 2-fluoro-3-trifluoromethylbenzyloxy, 3-fluoro-5-trifluoromethylbenzyloxy, 4-fluoro-2-tzifluoromethylbenzyloxy, 4-fluoro-3-trifluoromethylbenzyloxy, 2-fluoroplbenoxy, 4-fluorophenoxy, 2-fluoro-3-trifluoromethylphenoxy, 2-fluorobenzyloxy, 4-fluorophenylamino, 2-fluorottrifluoromethylphenoxy, 4-isopropylbenzyloxy, 3-isopropylphenoxy, 4-isopropylphenoxy, 4-isopropyl-3-methylphenoxy, 4-isopropylbenzyloxy, 3-isopropylphenoxy, 4-isopropylphenoxy, 4-isopropyl-3-methylphenoxy, phenylamino, 1-phenylethoxy, 2-phenylethoxy, 2-phenylethyl, 2-phenylethylamino, phenylsulfonyl, 3-trifluoromethoxybenzyloxy, 4-trifluoromethoxybenzyloxy, 3-trifluoromethoxyphenoxy, 4-trifluoromethoxyphenoxy, 3-tifluoromethylbenzyloxy, 4-trifluoromethylbenzyloxy, 2,4-bis-trifluoromethylbenzyloxy, 3-txifiuoromethylbenzyl, 3,5-bis-tWhluoromethylbenzyloxy, 4-trifuoromethylphenoxy, 3-trifluoromethylphenoxy, 3-tfifluoromethylthiobenzyloxy, 4-triuoromethylthiobenzyloxy, 2,3,4-trifluorophenoxy, 2,3,5-trifluorophenoxy, 3-pentafluoroethylphenoxy, 3-(1,1,2,2-tetrafluoroethoxy)phenoxy, and 3-trifluoromethylthiophenoxy;
R³³ is selected from the group consisting of hydrido, amidino, guanidino, carboxy, methoxy, ethoxy, isopropoxy, propoxy, hydroxy, amino, methoxyamino, ethoxyamino, acetamido, trifluoroacetamido, N-methylamino, dimethylamino, N-ethylamino, methylthio, ethylthio, isopropylthio, trfluoromethyl, pentafluoroethyl, 2,2,2-triuoroethyl, 2,2,3,3,3-pentafluoropropyl, trifluoromethoxy, 1,1,2,2-tetrfluoroethoxy, fluoro, chloro, bromo, amidosulfonyl, N-methylamidosulfonyl, N,N-dimethylamidosulfonyl, hydroxymethyl, 1-hydroxyethyl, 2-hydroxyethyl, 2,2,2-trifluoro-1-hydroxyethyl, methoxycarbonyl, ethoxycarbonyl, amidocarbonyl, N-methylamidocarbonyl, N,N-dimethylamidocarbonyl, cyano, and Q^{b};
A is selected from the group consisting of a bond, NH, N(CH₃), N(OH), CH₂, CH₃CH, CF₃CH, NHC(O), N(CH₃)C(O), C(O)NH,
C(O)N(CH₃), CH₂CH₂, CH₂CH₂CH₂, CH₃CHCH₂, and CF,3CHCH₂;
X⁰ is selected from the group consisting of hydnido, hydroxy, amino, aminomethyl, 1-aminoethyl, methyl, ethyl, trifluoromethyl, pentafluoroethyl, 2,2,2-trifuoroethyl, methoxy, hydroxymethyl, 1-hydroxyethyl, 2-hydroxyethyl, methylthio, ethylthio, tiifiuoromethoxy,1,1,2,2-tetmfluoroethoxy, fluoro, chloro, and bromo;
R¹ is selected from the group consisting of hydrido, hydroxy, amino, methyl, ethyl, propyl, n-butyl, 2,2,2-trifluoroethyl, 3,3,3-trifluoropropyl, 2,2,3,3,3-pentafluoropropyl, 2-aminoethyl, 3-aminopropyl, 2-hydroxyethyl, 3-hydroxypropyl, and 2,3-dihydroxypropyl;
R² isZ⁰-Q^{b};
Z⁰ is selected from the group consisting of a bond, CH₂, CH₂CH₂, 0, 8, NH, N(CH₃), OCH₂, SCH₂, N(H)CH₂, and N(CH₃)CH₂;
Q^{b} is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, 5-isoxazolyl, 2-pyridyl, 3-pyridyl, 4-pyridyl, 2-pyrazinyl, 2-pyrinidinyl, 4-pyrimidinyl, 5-pyrimidinyl, 3-pyridazinyl, 4-pyridazinyl, and 1,3,5-triazin-2-yl, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine, 2-Q^{b}-5-Q^{s}-3-R¹⁶-4 R¹⁸ pyridine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
3-Q^{b}-5-Q^{s}-4-R¹⁶-2-R¹⁹-furan, 2,6-5-Q^{s}-3-R¹⁶-4-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ imidazole,
3-Q^{b}-5-Q^{s}-4-R¹⁶ isoxazole, 5-Q^{b}-3-Q^{s}-4-R¹⁶ isoxazole,
2-Q^{b}-5Q^{s}-4-R¹⁶ pyrazole, 4-Q^{b}-2-Q^{s}-5-R¹⁹ thiazole, and
2-Q^{b}-5-Q^{s}-4-R¹⁷ thiazole;
2-Q^{b}-5-Q^{s}-4-R¹⁷ thiazole;
R¹⁶, R¹⁷, R¹⁸, and R¹⁹ are independently selected from the group consisting of hydrido, methyl, ethyl, isopropyl, propyl, carboxy, amidino, guanidino, methoxy, ethoxy, isopropoxy, propoxy, hydroxy, amino, aminomethyl, 1-aminoethyl, 2-aminoethyl, N-methylamino, dimethylainino, N-ethylamino, methylthio, ethylthio, isopropylthio, trifluoromethylthio, methylsulfinyl, ethylsulfinyl, methylsulfonyl, ethylsulfonyl, trifluoromethyl, pentafluoroethyl, 2,2,2-trifluoroethyl, 2,2,3,3,3-pentafluoropropyl, trifluoromethoxy, 1,1,2,2-etrafluoroethoxy, fluoro, chloro, bromo, hydroxymethyl, 1-hydroxyethyl, 2-hydroxyethyl, and cyano;
R¹⁶ or R¹⁹ is optionally C(NR²⁵)NR²³R²⁴ with the proviso that R R¹⁹, and Q^{b} are not simultaneously hydrido;
Q^{b} is C(NR²⁵)NR²³R²⁴ or hydrido, with the proviso that no more than one of R²³ and R²⁴ is hydroxy at the same time;
R²³, R²⁴, and R²⁵ are independently selected from the group consisting of hydrido, methyl, ethyl, and hydroxy;
Q^{s} is selected from the group consisting of a bond, CH₂ and CH₂CH₂.

47. Compound of claim 46 or a pharmaceutically acceptable salt thereof, wherein;
B is selected from the group consisting of cyclopropyl, cyclobutyl, cyclopentyl, cyclohexyl, cycloheptyl, oxalan-2-yl, 2-(2R)-bicyclo[2.2.1]-heptyl, 1-pyrrolidinyl, 1-piperidinyl, oxetan-3-yl, azetidin-1-yl, azetidin-2-yl, azetidin-3-yl, 7-oxabicyclo[2.2.1]heptan-2-yl, bicyclo[3.1.0]hexanmyl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 4H-2-pyranyl, 4H-3-pyranyl, 4Hpyranyl, 4H-pyran-one-2-yl, 4H-pyranfone-3-yl, 2-tetrahydrofuranyl, 3-tetrahydrofuranyl, 2-tetrahydropyranyl, 3-tetrahydropyranyl, 4-tetrahydropyranyl, 2-tetrahydrothienyl, and 3-tetahydrothienyl;
A is selected from the group consisting of a bond, CH₂, NHC(O), CH₂CH₂, and CH₂CH₂CH₂;
X^{o} is selected from the group consisting of hydrido, hydroxy, 1-aminoethyl, methyl, ethyl, trifluoromethyl, pentafluoroethyl, 2,2,2-trifluoroethyl, hydroxymethyl, 1-hydroxyethyl, 2-hydroxyethyl, fluoro, chloro, and bromo;
R¹ is selected from the group consisting of hydrido, hydroxy, amino, methyl, ethyl, propyl, 2,2,2-trifluoroethyl, 3,3,3-trifluoropropyl, 2,2,3,3,3-pentafluoropropyl, 2-aminoethyl, and 2-hydroxyethyl;
R² isZ⁰-Q^{b};
Z⁰ is selected from the group consisting of a bond, CH₂, O. S, NH, N(CH₃), OCH₂, and SCH_{2;}
Q^{b} is selected from the group consisting of 3-amidocarbonyl-5-aminophenyl, 3-amino-5-(N-benzylaridocarbonyl)phenyl, 3-amino-5-benzylphenyl, 3-amino-5(2-phenylethyl)phenyl, 3-amino-5-benzylaminophenyl, 3-amino-5-(2-phenylethylamino)phenyl, 3-amino-5-benzyloxyphenyl, 3-amino-5-(2-phenylethoxy)phenyl, 3-amino-5-(N-(2-chlorobenzyl)amidocarbonyl)phenyl, 3-amino-5-(N-(3-fuorobenzyl)amidocarbonyl)phenyl, 3-amino-5-(N-2-trifluoromethylbenzyl)amidocarbonyl)phenyl, 3-amino-5-(N-(1-phenyletbyl)amidocarbonyl)phenyl, 3-amino-5-(N-1-methyl-1-phenylethyl)amidocarbonyt)phenyl, 3-amino-5-(N-benzylam-idosulfonyl)phenyl, 3-amino—(N42-chlorobenzyl)amidosulfonyl)phenyl, 3-amino-5-(N-ethylamidocarbonyl)phenyl, 3-amino-5-(N-isopropylamidocarbonyl)phenyl, 3-amino-5N-propylamidocarbonyl)phenyl, 3-amino-5-(N-isobutylamidocarbonyl)phenyl, 3-amino-5-(N-(2-butyl)amidocarbonyl)phenyl, 3-amino-5-(N-cyclobutylamidocarbonyl)phenyl, 3-amino-5-(N-cyclopentylamidocarbonyl)phenyl, 3-amino-5-(N-cyclohexylamidocarbonyl)phenyl, 5-amino-2-fluorophenyl,
3-amino5-hydroxymethylphenyl, 5-ar-ino-3-methoxycarbonylphenyl, 3-amidinophenyl, 3-amino-2-methylphenyl, 5-amino-2-methylthiophenyl, 3-aminophenyl, 3-amino-5(4-trifluoromethylbenzylamino)phenyl, 3-amino-S-(4-trifluoromethylbenzyloxy)phenyl, 3-carboxyphenyl, 3-carboxy-5-hydroxyphenyl, 3-anuno-5-carboxyphenyl, 3-chlorophenyl, 2-chlorophenyl, 3-cyanophenyl, 3,5-iaminophenyl, 3-dimethylaminophenyl, 2-fluorophenyl, 3-fluorophenyl, 2-hydroxyphenyl, 3-hydroxyphenyl, 3-methanesulfonylaminophenyl, 2-methoxyphenyl, 3-methoxyphenyl, 3-methoxyaminophenyl, 3-methoxycarbonylphenyl, 2-methylaminophenyl, 3-methylaminophenyl, 2-methylphenyl, 3-methylphenyl, 4-methylphenyl, phenyl, 3-trifluoroacetamidophenyl, 3-trifluoromethylphenyl, 2-triuoromethylphenyl, 5-amino-2,5-thienyl, 5-amino-3-thienyl, 3-bromo-2-thienyl, 3-pyridyl, 4-pyridyl, 2-thienyl, and 3-thienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
R¹⁶ and R¹⁹ are independently selected from the group consisting of 20 hydrido, amidino, amino, aminomethyl, methoxy, methylamino, hydroxy, hydroxymethyl, fluoro, chiloro, and cyano;
R¹⁶ or R¹⁹ is optionally C(NR²⁵)NR²³R²⁴ with the proviso that R R¹⁶, and Q^{b} are not simultaneously hydrido;
R¹⁷ and R¹⁸ are independently selected from the group consisting of hydrido, fluoro, chloro, hydroxy, hydroxymethyl, amino, carboxy, and cyano;
Q^{b} is C(NR²⁵)NR²³R²⁴ or hydrido;
R²³, R²⁴, and R²⁵ are independently hydrido or methyl;
Q^{s} is CH₂.

48. Compound of claim 45 of the Formula: 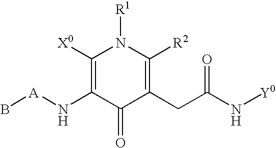
or a pharmaceutically acceptable salt thereof, wherein;
B is a C3-C7 cycloalkyl or a C4-C6 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;
R⁹, R¹¹, and R¹³ are independently selected from the group consisting of hydrido, hydroxy, amino, amidino, guanidino, alkylamino, alkylthio, alkoxy, alkylsulfinyl, alkylsulfonyl, amidosulfonyl, alkyl, halo, haloalkyl, haloalkoxy, hydroxyalkyl, carboxy, carboxamido, and cyano;
R¹⁰ and R¹² are independently selected from the group consisting of hydrido, acetamido, haloacetamido, anudino, guanidino, alkyl, alkoxy, alkoxyamino, hydroxy, amino, alkylamino, alkylsulfonamido, amidosulfonyl, hydroxyalkyl, armnoalkyl, halo, haloalkyl, carboalkoxy, carboxy, carboxamido, carboxyalkyl, and cyano;
R³³ and R³⁴ are independently selected from the group consisting of hyduido, acetamido, baloacetamido, amidino, guanidino, alkoxy, hydroxy, amino, alkoxyamino, aIlcylamino, alkylithio, amidosulfonyl, alkyl, halo, haloalkyl, haloalkoxy, hydroxyalkyl, carboalkoxy, carboxy, carboxamudo, and cyano;
R³³ is optionally Q^{b};
A is a bond or (CH(R¹⁵))ₚₐ—(W⁷)ᵣᵣ wherein rr is 0 or 1, pa is an integer selected from 0 through 3, and W is N(R⁷);
R⁷ is hydrido or alkyl;
R¹⁵ is selected from the group consisting of hydrido, halo, alkyl, and haloalkyl;
X⁰ is selected from the group consisting of hydrido, alkyl, halo, haloalkyl, aminoalkyl, and hydroxyalkyl;
R¹ is selected from the group consisting of hydrido, alkyl, hydroxyalkyl, and haloalkyl;
R² is Z⁰-Q^{b};
Z⁰ is a bond;
Q^{b} is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
Y⁰ is phenyl or a heteroaryl of 5 or 6 ring members, wherein one carbon of said phenyl or said heteroaryl is substituted by Qs, a carbon two or three atoms from the point of attachment of Q^{s} to said phenyl or said heteroaryl is substituted by Qb, a carbon adjacent to the point of attachment of Q^{s} is optionally substituted by R¹⁷, another carbon adjacent to the point of attachment of Q^{s} is optionally substituted by R¹⁸, a carbon adjacent to Q^{b} is optionally substituted by R¹⁶, and another carbon adjacent to Q^{b} is optionally substituted by R¹⁹;
R¹⁶, R¹⁷, R¹⁸, and R¹⁹ are independently selected from the group consisting of hydrido, amidino, guanidino, carboxy, haloalkylthio, alkoxy, hydroxy, amino, alkylamino, alkylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally NR²⁰R²¹ or C(NR²⁵)NR²³R²⁴ with the proviso that R¹⁶, R¹⁹, and Q^{b} are not simultaneously hydrido;
Q^{b} is selected from the group consisting of NR²⁰R²¹, hydrido, and C(NR²⁵)NR²³R²⁴;
R²⁰, R²¹, R²³, R²⁴ and R²⁵ are independently hydrido or alkyl;
Q^{s} is CH₂.

49. Compound of claim 48 or a pharmaceutically acceptable salt thereof, wherein;
B is selected from the group consisting of cyclopropyl, cyclobutyl, cyclopentyl, cyclohexyl, cycloheptyl, oxalan-2-yl, 2-2R)bicyclo[2.2.1]-heptyl, oxetan-3-yl, azetidin-1-yl, azetidin-2-yl, azetidin-3-yl, bicyclo[3.1.0]bexan-yl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 2-tetrahydrofuranyl, 3-tetrahydrofuranyl, 2-tetrahydropyranyl, 3-tetrahydropyranyl, 4-tetrahydropyranyl, 2-tetrahydrotbienyl, and 3-tetrahydrothienyl, wherein each ring carbon is optionally substituted with R³³, ring carbons and a nitrogen adjacent to the carbon atom at the point of attachment are optionally substituted with R⁹ or R⁹, a ring carbon or nitrogen adjacent to the R⁹ position and two atoms from the point of attachment are opfionally substituted with R¹⁰, and a ring carbon or nitrogen atom adjacent to the R¹³ position and two atoms from the point of attachment is optionally substituted with R¹²;
R⁹, R¹¹, and R¹³ are independently selected from the group consisting of hydrido, methyl, ethyl, methoxy, ethoxy, hydroxy, amino, N-methylamino, N,N-dimethylamino, methylthio, trifluoromethyl, pentafluoroethyl, 2,2,2-trifluoroethyl, fluoro, chloro, bromo, amidosulfonyl, N-methylamidosulfonyl, N,N-dimethylamidosulfonyl, hydroxymethyl, 1-hydroxyethyl, amidocarbonyl, N-methylamidocarbonyl, carboxy, and cyano;
R¹⁰ and R¹² are independently selected from the group consisting of hydnido, amidino, amidocarbonyl, N-methylamidocarbonyl, N-benzylamidocarbonyl, N-2-chlorobenzyl)amidocarbonyl, N-(3-fluorobenzyl)amidocarbonyl, N-(2-trifluoromethylbenzyl)amidocarbonyl, N-(1-phenylethyl)amidocarbonyl, N-(1-methyl-1-phenylethyl)amidocarbonyl, N-benzylaniidosulfonyl, N-(2-chlorobenzyl)amidosulfonyl, N-ethylamidocarbonyl, N-isopropylamidocarbonyl, N-propylamidocarbonyl, N-isobutylamidocarbonyl, N-(2,5-butyl)amidocarbonyl, N-yclobutylamidocarbonyl, N-cyclopentylamidocarbonyl, N-cyclohexylamidocarbonyl, guanidino, methyl, ethyl, methoxy, ethoxy, hydroxy, hydroxymethyl, 1-hydroxyethyl, 2-hydroxyethyl, carboxy, carboxymethyl, amino, acetamido, tiifluoromethyl, pentafluoroethyl, 2,2,2-trifuoroethyl, trfluoroacetamido, aminomethyl, N-methylamino, dimethylamino, methoxyamino, amidosulfonyl, N-methylamidosulfonyl, N,N-dimethylamidosulfonyl, methanesulfonamido, methoxycarbonyl, fluoro, chloro, bromo, and cyano;
R³³ is selected from the group consisting of hydrido, amidino, guanidino, methyl, ethyl, methoxy, ethoxy, hydroxy, carboxy, amino, N-methylamino, dimethylamino, methoxyamino, methylthio, ethylthio, trifluoromethyl, pentafluoroethyl, 2,2,2-trifluoroethyl, fluoro, chloro, bromo, amidosulfonyl, N-methylamidosulfonyl, hydroxymethyl, amidocarbonyl, cyano, and Q^{b};
A is selected from the group consisting of a bond, NH, N(CH₃), CH₂, CH₃CH, CH₂CH₂, and CH₂CH₂CH₂;
X^{o} is selected from the group consisting of hydrido, aminomethyl, methyl, ethyl, trifluoromethyl, 2,2,2-trifluoroethyl, hydroxymethyl, 2-hydroxyethyl, chloro, and fluoro;
R¹ is selected from the group consisting of hydrido, methyl, ethyl, propyl, 2,2,2-trifluoroethyl, 3,3,3-trifluoropropyl, 2,2,3,3,3-pentafluoropropyl, and 2-hydroxyethyl;
R² is selected from the group consisting of phenyl, 2-thienyl, 2-furyl, 2-pyrrolyl, 2-imidazolyl, 2-thiazolyl, 3-isoxazolyl, 2-pyridyl, and 3-pyridyl, wherein a carbon adjacent to the carbon at the point of attachment of said phenyl or heteroaryl ring to the 4-pyridone ring is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
Y⁰ is selected from the group consisting of:
1-Q^{b}-4-Q^{s}-2-R¹⁶-3-R¹⁷-5-R¹⁸-6-R¹⁹ benzene,
2-Q^{b}-6-Q^{s}-2-R¹⁷-5-R¹⁸-4-R¹⁹ pyridine, 2-Q^{b}-5-Q^{s}-3-R¹⁶-4 R¹⁷ thiophene,
3-Q^{b}-6-Q^{s}-2-R¹⁶-5-R¹⁸-4-R¹⁹ pyridine, 2-Q^{b}-5-Q^{s}-3-R¹⁶-4 R¹⁹ thiophene,
3-Q^{b}-5-Q^{s}-4-R¹⁶-2-R¹⁹ furan, 2-Q^{b}-5-Q^{s}-3-R¹⁷ furan,
3-Q^{b}-5-Q^{s}-R¹⁶-2-R¹⁹ pyrrole, 2-Q^{s}-3-R¹⁶-4-R¹⁷ pyrrole,
4-Q^{b}-2-Q^{s}-R¹⁹ imidazole, 2-Q^{b}-4-Q^{s}-5-R¹⁷ thiazole,
R¹⁶, R¹⁷, R¹⁸ and R¹⁹ are independently selected from the group consisting of hydrido, methyl, ethyl, amidino, guanidino, methoxy, hydroxy, amino, aminomethyl, 1-aminoethyl, 2-aminoethyl, N-methylamino, dimethylamino, methylthio, ethylthio, trifiuoromethylthio, methylsulfinyl, methylsuifonyl, trifluoromethyl, pentafluoroethyl, 2,2,2-trifluoroethyl, trifluoromethoxy, fluoro, chloro, hydroxymethyl, carboxy, and cyano;
Q^{b} is NR²⁰OR²¹ or C(NR²⁵)NR²³R²⁴;
R²⁰, R²¹, R²³, R²⁴, and R²⁵ are independently selected from the group consisting of hydrido, methyl, and ethyl;
Q^{s} is CH₂.

50. Compound of claim 49 or a pharmaceutically acceptable salt thereof, wherein;
B is selected from the group consisting of cyclopropyl, cyclobutyl, cyclopentyl, cyclohexyl, oxalan-2-yl, 2-(2R)-bicyclo[2.2.1]-heptyl, oxetan-3-yl, azetidin-1-yl, azetidin-2-yl, azetidin-3-yl, 1-pyrrolidinyl and l-piperidinyl;
A is selected from the group consisting of a bond, CH₂, CH₂CH₂ and CH₂CH₂CH₂;
X^{o} is selected from the group consisting of hydrido, methyl, ethyl, tnifluoromethyl, 2,2,2-trifluoroethyl, hydroxyinethyl, 2-hydroxyethyl, chloro, and fluoro;
R¹ is selected from the group consisting of hydrido, methyl, ethyl, and 2,2,2-trifluoroethyl;
R² is selected from the group consisting of 3-anidocarbonyl-5-aminophenyl, 3-amidocarbonyl-5-aminophenyl, 3-amino-5-(N-benzylanudocarbonyl)phenyl, 3-amino-5-(N-(2-chlorobenzyl)amidocarbonyl)phenyl, 3-amino-5-N-(3-fluorobenzyl)amidocarbonyl)phenyl, 3-amino-5-(N-(2-trifluoromethylbenzyl)amidocarbonyl)phenyl, 3-amino-5-(N-(1-phenylethyl)amidocarbonyl)phenyl, 3-amino-5(N-(1-methyl-1-phenylethyl)amidocarbonyl)phenyl, 3-amino-5-(N-benzylamidosulfonyl)phenyl, 3-amino-5-(N-(2-chlorobenzyl)amidosulfonyl)phenyl, 3-amino-5-(N-ethylamidocarbonyl)phenyl, 3-amino—(N-isopropylamidocarbonyl)phenyl, 3-amino-5-(N-propylamidocarbonyl)phenyl, 3-amino-5-(N-isobutylamidocarbonyl)phenyl, 3-amino-5-(N-(2-butyl)amidocarbonyl)phenyl, 3-amino-5-(N-cyclobutylamidocarbonyl)phenyl, 3-amino-i(N-cyclopentylamidocarbonyl)phenyl, 3-amino-5-(N-cyclohexylamidocarbonyl)phenyl, Samno-2-fluorophenyl, 3-amino-5-hydroxymethylphenyl, 5-aniuno-3-methoxycarbonylphenyl, 3-amidinophenyl, 3-aoiin-2-methylphenyl, 5-amino-2-methylthiophenyl, 3-aminophenyl, 3-carboxyphenyl, 3-carboxy-5-aminophenyl, 3-carboxy-5-hydroxyphenyl, 3-carboxymethyl-5-a-inophenyl, 3-carboxymethyl-5-hydroxyphenyl, 3-carboxymethylphenyl, 3-chlorophenyl, 2-chlorophenyl, 3-cyanophenyl, 3,5-diaminophenyl, 3-dimethylaminophenyl, 2-fluorophenyl, 3-fluorophenyl, 2,5-difluorophenyl, 2-hydroxyphenyl, 3-hydroxyphenyl, 3-methanesulfonylaminophenyl, 2-methoxyphenyl,
3-methoxyphenyl, 3-methoxyaminophenyl, 3-methoxycarbonylphenyl, 2-methylaminophenyl, 3-methylaminophenyl, 2-methylphenyl, 3-methylphenyl, 4-methylphenyl, phenyl, 3-trifluoroacetamidophenyl, 3-trifiuoromethylphenyl, 2-tifluoromethylphenyl, 5-amino-2,5-thienyl, aminiio-3-thienyl, 3-bromo-2-tbienyl, 3-pyridyl, 4-pyridyl, 2-thienyl, and 3-thienyl;
Y⁰ is selected from the group consisting of:
1-Q^{b}-4-Q^{s}-2-R¹⁶-3-R¹⁷-5-R¹⁸-6-R¹⁹ benzene,
2-Q^{b}-5-Q^{s}-6-R¹⁷-4-R¹⁸-3-R¹⁹ pydine,
3-Q^{b}-6-Q^{s}-2-R¹⁶-5-R¹⁸-4-R¹⁹ pyridine,
3-Q^{b}-5-Q^{s}-4R¹⁶-2-R¹⁹ thiophene, 2-Q^{b}-5Q^{s}-3-R¹⁶-4-R¹⁹-thiophene,
R¹⁶ and R¹⁹ are independently selected from the group consisting of hydrido, amidino, amino, aminomethyl, methoxy, methylamino, hydroxy, hydroxymethyl, fluoro, chloro, and cyano;
R¹⁷ and R¹⁸ are independently selected from the group consisting of hydrido, fluoro, chloro, hydroxy, hydroxymethyl, amino, carboxy, and cyano;
Q^{b} is C(NR²⁵)NR²³R²⁴;
R²³, R²⁴, and R²⁵ are independently hydrido or methyl;
Q^{s} is CH₂.

51. Compound of claim 50 or a pharmaceutically acceptable salt thereof, wherein;
B is selected from the group consisting of cyclopropyl, cyclobutyl, cyclopentyl, cyclohexyl, oxalan-2-yl, 2-(2R)bicyclo[2.2.1-j-heptyl, oxetan-3-yl, azetidin-1-yl, azetidin-2-yl, azetidin-3-yl, and 1-piperidinyl;
A is selected from the group consisting of a bond, CH₂, CH₂CH₂ and CH₂CH₂CH₂;
X^{o} is selected from the group consisting of hydrido, methyl, ethyl, trifluoromethyl, 2,2,2-trifluoroethyl, chloro, and fluoro;
R¹ is selected from the group consisting of hydrido, methyl, ethyl, and 2,2,2-ifluoroethyl;
R² is selected from the group consisting of 3-amidocarbonyl-5-aminophenyl, 3-amino-i (N-benzylamidocarbonyl)phenyl, 3-amino-5-(N-(2-chlorobenzyl)amidocarbonyl)phenyl, 3-amino-5-(N-(3-fluorobenzyl)amidocarbonyl)phenyl, 3-amino-5-(N-(2-trifluoromethylbenzyl)amidocarbonyl)phenyl, 3-amino-5-(N-1-phenylethyl)amidocarbonyl)phenyl, 3-amino-5-(N-(1-meyl-1-phenylethyl)wmdocarbonyl)phenyl, 3-amino-5-(N-benzylaraidosulfonyl)phenyl, 3-atnino-5-(N-(2-chlorobenzyl)amidosulfonyl)phenyl, 3-amino-5-(N-ethylamidocarbonyl)phenyl, 3-amino-5-(N-isopropylamidocarbonyl)phenyl, 3-amino-5-(N-propylamidocarbonyl)phenyl, 3-amino-5-(N-isobutylamidocarbonyl)phenyl, 3-amino-5-N-(2-butyl)amidocarbonyl)phenyl, 3-amino-SN-cyclobutylamidocarbonyl)phenyl, 3-amino-5-(N-cyclopentylamidocarbonyl)phenyl, 3-amino-5-(N-cyclohexylamidocarbonyl)phenyl, 3-aminophenyl, 3-arboxy-5-aminophenyl, 3-chlorophenyl, 3,5-dianiophenyl, 3-dimethylaminophenyl, 3-hydroxyphenyl, 3-methanesulfonylaminophenyl, 3-methylaminophenyl, 2-methylphenyl, 3-methylphenyl, phenyl, 3-trifluoroacetainidophenyl, 3-bromo-2-thienyl, 2-thienyl, and 3-thienyl;
Y⁰ is selected from the group consisting of Samidin-thienylmethyl, 4-amidinobenzyl, 2-fluoroamidinobenzyl, and 3-fluoro4-amdinobenzy].

52. Compound of claim 45 where said compound is selected from the group of the Formula: 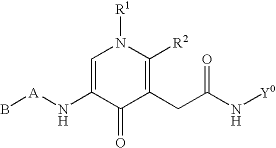
or a pharmaceutically acceptable salt thereof, wherein B, A, R¹, R², X^{o}, and Y⁰ are selected to form a compound selected from the group consisting of;
R² is 3-aminophenyl, B is cyclopropyl, A is a bond, Y⁰ is 4 amidinobenzyl, and R¹ is methyl;
R² is 3-aunophenyl, B is cyclobutyl, A is a bond, Y⁰ is 4-amidino-2-fluorobenzyl, and R¹ is methyl;
R² is 3-aminophenyl, B is cyclobutyl, A is a bond, Y⁰ is 4-amidinobenzyl, and R¹ is methyl;
R² is 3-aminophenyl, B is cyclopropyl, A is a bond, Y⁰ is 4-amidino-2-fluorobenzyl, and R¹ is methyl;
R² is 3-aminophenyl, B is cyclobutyl, A is a bond, Y⁰ is 4-amidinobenzyl, and R¹ is hydrido;
R² is 3-aminophenyl, B is cyclobutyl, A is a bond, Y⁰ is 4-amidino-3-fluorobenzyl, and R¹ is methyl;
R² is 3-aminophenyl, B is cyclopentyl, A is a bond, Y⁰ is 4-amidinobenzyl, and R¹ is methyl;
R² is 5-amino-2-thienyl, B is cyclobutyl, A is a bond, Y⁰ is 4 amidinobenzyl, and R¹ is methyl;
R² is 3-aminophenyl, B is cyclopropyl, A is CH₂, Y⁰ is 4-amidinobenzyl, and R¹ is methyl;
R² is 3-aminophenyl, B is 2-(2R)-bicyclo[2.2.1]-heptyl, A is a bond, Y⁰ is 4-amidinobenzyl, and R¹ is methyl;
R² is 3-aminophenyl, B is cyclopentyl, A is a bond, Y⁰ is 4-amidino-2-fluorobenzyl, and R¹ is methyl;
R² is 3-aminophenyl, B is cyclohexyl, A is CH₂CH₂, Y⁰ is 4 amidinobenzyl, and R¹ is hydrido;
R² is 3-aminophenyl, B is oxalan-2-yl, A is CH₂, Y⁰ is 4-amidinobenzyl, 10 and R¹ is methyl;
R² is 3-aminophenyl, B is 1-piperidinyl, A is CH₇CH₂, Y⁰ is 4 amidinobenzyl, and R¹ is methyl;
R² is 3-aminophenyl, B is 1-pyrrolidinyl, A is CH₂CH₂CH_{2,5}-e is 4 amidinobenzyl, and R¹ is methyl;
R² is 3-amino-5-carbomethoxyphenyl, B is cyclobutyl, A is a bond, Y⁰ is 4-amidinobenzyl, and R¹ is hydrido;
R² is 3-amino-5-arboxyphenyl, B is cyclobutyl, A is a bond, Y⁰ is 4 amidinobenzyl, and R¹ is hydrido;
R² is 3,5-diaminophenyl, B is cyclobutyl, A is a bond, Y⁰ is 4 amidinobenzyl, and R¹ is hydrido;
R² is 2-amino-carboxyfpyridyl, B is cyclobutyl, A is a bond, Y⁰ is 4-amidinobenzyl, and R¹ is hydrido;
R² is 3-amino-carbomethoxyphenyl, B is cyclobutyl, A is a bond, Y⁰ is 4-amidinobenzyl, and R¹ is methyl;
R² is 3-amino-carboxyphenyl, B is cyclobutyl, A is a bond, Y⁰ is 4 amidinobenzyl, and R¹ is methyl;
R² is 3,5-diaminophenyl, B is cyclopropyl, A is a bond, Y⁰ is 4 amidinobenzyl, and R¹ is methyl;
R² is 3,5-diaminophenyl, B is cyclobutyl, A is a bond, Y⁰ is 4-amidino-2-fluorobenzyl, and R¹ is methyl;
R² is 3,5-diaminophenyl, B is cyclopropyl, A is a bond, Y⁰ is 4-amidino-2-fluorobenzyl, and R¹ is methyl;
R² is 3,5-diaminophenyl, B is cyclobutyl, A is a bond, Y⁰ is 4 amidinobenzyl, and R¹ is hydrido;
R² is 3,5-diaminophenyl, B is cyclobutyl, A is a bond, is 4-amidino-3-fluorobenzyl, and R¹ is methyl;
R² is 3,5-diaminophenyl, B is cyclopentyl, A is a bond, Y⁰ is 4 amidinobenzyl, and R is methyl;
R² is 3-carboxy-5-aminophenyl, B is cyclopropyl, A is a bond, Y⁰ is 4 amidinobenzyl, and R¹ is methyl;
R² is 3-carboxy-5-aminophenyl, B is cyclobutyl, A is a bond, Y⁰ is 4 amidino-2-fluorobenzyl, and R¹ is methyl;
R² is 3-carboxy-5-aminophenyl, B is cyclopropyl, A is a bond, Y⁰ is 4 amidino-2-fluorobenzyl, and R¹ is methyl;
R² is 3-carboxy-5-aminophenyl, B is cyclobutyl, A is a bond, Y⁰ is 4 amidinobenzyl, and R¹ is hydrido;
R² is 3-carboxy-5-aminophenyl, B is cyclobutyl, A is a bond, Y⁰ is 4 amidino-3-fluorobenzyl, and R¹ is methyl;
R² is 3-carboxy-a5-inophenyl, B is cyclopentyl, A is a bond, Y⁰ is 4 amidinobenzyl, and R¹ is methyl;
R² is 3-amino-5-(N-benzylamidocarbonyl)phenyl, B is cyclopropyl, A is a bond, Y⁰ is 4-namidinobenzyl, and R¹ is methyl;
R² is 3-amino-5-N-benzylamidocarbonyl)phenyl, B is cyclobutyl, A is a bond, Y⁰ is 4-atnidino-2-fluorobenzyl, and R¹ is methyl;
R² is 3-amino-5-(N-benzylamidocarbonyl)phenyl, B is cyclobutyl, A is a bond, Y⁰ is 4-amidinobenzyl, and R¹ is methyl;
R² is 3-amino-5-N-benzylamidocarbonyl)phenyl, B is cyclopropyl, A is a bond, Y⁰ is 4-amidino2-fluorobenzyl, and R¹ is methyl;
R² is 3-amino-5-N-benzylanidocarbonyl)phenyl, B is cyclobutyl, A is a bond, Y⁰ is 4-amidinobenzyl, and R¹ is hydrido;
R² is 3-amino-5-N-benzylamidocarbonyl)phenyl, B is cyclobutyl, A is a bond, Y⁰ is 4-amidino-3-fluorobenzyl, and R¹ is methyl;
R² is 3-amino-5-N-benzylamidocarbonyl)phenyl, B is cyclopentyl, A is a bond, Y⁰ is 4-amidinobenzyl, and R¹ is methyl;
R² is 3-amino-5-(N-(2-chlorobenzyl)amidosulfonyl)phenyl, B is cyclopropyl, A is a bond, Y⁰ is 4-amidinobenzyl, and R¹ is methyl;
R² is 3-amino-5-(N-(2-chlorobenzyl)amidosulfonyl)phenyl, B is cyclobutyl, A is a bond, Y⁰ is 4-amidino-2-fluorobenzyl, and R¹ is methyl;
R² is 3-amino-5-(N-(2-chlorobenzyl)amidosulfonyl)phenyl, B is cyclobutyl, A is a bond, Y⁰ is 4-amidinobenzyl, and R¹ is methyl;
R² is 3-amino-5-(N-(2-chlorobenzyl)amidosulfonyl)phenyl, B is cyclopropyl, A is a bond, Y⁰ is 4-amidino-2-fluorobenzyl, and R¹ is methyl;
R² is 3-amino-5-(N-(2-chlorobenzyl)amidosulfonyl)phenyl, B is cyclobutyl, A is a bond, Y⁰ is 4-amidinobenzyl, and R¹ is hydrido;
R² is 3-amino-5-(N-(2-chlorobenzyl)amidosulfonyl)phenyl, B is cyclobutyl, A is a bond, Y⁰ is 4-amidino-3-fluorobenzyl, and R¹ is methyl;
R² is 3-amino-5-(N-(2-chlorobenzyl)amidosulfonyl)phenyl, B is cyclopentyl, A is a bond, Y⁰ is 4-amidinobenzyl, and R¹ is methyl;
R² is 3-amino-5-(N-(2-trifluoromethylbeuzYl)amidocarbonyl)-phenyl, B is cyclopropyl, A is a bond, Y⁰ is 4-amidinobenzyl, and R¹ is methyl;
R² is 3-amino-5-(N-(2-ffifluoromethylbenzyl)amidocarbonyl)-phenyl, B is cyclobutyl, A is a bond, Y” is 4-amidino-2-fluorobenzyl, and R¹ is methyl;
R² is 3-amno-5-(N-(2-f ifluoromethylbenzyl)adocarbonyl)-phenyl, Bis *cyclobutyl, A is a bond, Y⁰ is 4-amidinobenzyl, and R¹ is methyl;
R² is 3-amino-5-N-(2-ffifluoromethylbenzyl)umdocarbonyl)-phenyl, B is cyclopropyl, A is a bond, Y⁰ is 4-amidino2-fluorobenzyl, and R¹ is methyl;
R² is 3-ano-5-(N-(2-trifluoromethylbenzyl)adocarbonyl)-phenyl, B is cyclobutyl, A is a bond, Y⁰ is 4-aniidinobenzyl, and R¹ is hydrido;
R² is 3-amino-5-(N-(2-trifluoromethylbenzyl)aniidocarbonyl)-phenyl, B is cyclobutyl, A is a bond, Y⁰ is 4-amidino3-fluorobenzyl, and R¹ is methyl; and
R² is 3-amino-5-(N-(2-tuoromethylbenzyl)arridocarbonyl)phenyl, B is cyclopentyl, A is a bond, Y⁰ is 4-amidinobenzyl, and R¹ is methyl.

53. Compound of claim 22 of the Formula: 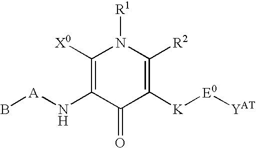
or a pharmaceutically acceptable salt thereof, wherein;
B is phenyl or a heteroaryl of 5 or 6 ring members, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independenty selected from the group consisting of hydrido, acetamido, haloacetamido, atidino, guanidino, alkylenedioxy, haloalkylthio, alkanoyloxy, alkoxy, hydroxy, amino, alkoxyamno, haloalkanoyl, nitro, alkylamino, nlkylthio, aryl, aralkyl, cycloalkyl, cycloalkylalkyl, heteroaryl, heterocyclyl, alkylsulfonamido, amidosulfonyl, alkyl, alkenyl, halo, haloalkyl, haloalkenyl, haloalkoxy, hydroxyalkyl, alkylamino, carboalkoxy, carboxy, carboxamido, cyano, and Q^{b};
B is optionally selected from the group consisting of hydrido, trialkylsilyl, C2-C8 alkyl, C3-C8 alkylenyl, C3-C8 alkenyl, C3-C8 alkynyl, and C2-C8 haloaflcyl, wherein each member of group B is optionally substituted at any carbon up to and including 6 atoms from the point of attachment of B to A with one or more of the group consisting of R³², R³³, R³⁴, R³⁵, and R³⁶;
B is optionally a C3-C12 cycloalkyl or a C4-C9 saturated heterocyclyl, wherein each ring carbon is optionally substituted with R³³, a ring carbon other than the ring carbon at the point of attachment of B to A is optionally substituted with oxo provided that no more than one ring carbon is substituted by oxo at the same time, ring carbons and a nitrogen adjacent to the carbon atom at the point of attachment are optionally substituted with R⁹ or R¹³, a ring carbon or nitrogen adjacent to the R position and two atoms from the point of attachment is optionally substituted with R¹⁰, a ring carbon or nitrogen adjacent to the R¹³ position and two atoms from the point of attachment is optionally substituted with R¹², a ring carbon or nitrogen three atoms from the point of attachment and adjacent to the R¹⁰ position is optionally substituted with R¹¹, a ring carbon or nitrogen three atoms from the point of attachment and adjacent to the R¹² position is optionally substituted with R³³, and a ring carbon or nitrogen four atoms from the point of attachment and adjacent to the R¹¹ and R³³ positions is optionally substituted with R³⁴;
R⁹, R¹⁰, R¹¹, R¹², and R¹³ are independently selected from the group consisting of hydrido, acetamido, haloacetamido, alkoxyamino, alkanoyl, haloalkanoyl, amino, guanidino, alkylenedioxy, haloalkylthio, alkoxy, cycloalkoxy, cycloalkylalkoxy, aralkoxy, aryloxy, heteroaryloxy, heteroaralkoxy,heterocyclyloxy, heterocyclylalkoxy, hydroxy, amino, alkylamino, N-alkyl-N-arylamino, arylamino, arakylamino, heteroarylamino, heteroaralkylamino, heterocyclylamino, heterocyclylalkylamino, alkylthio, alkylsulfinyl, arylsulfinyl, aralkylsulfimyl, cycloalkylsulfinyl, heteroarylsufinyl, alkylsulfamido, alkylsulfonyl, arylsulfonyl, aralkylsulfonyl, cycloalkylsulfonyl, heteroarylsulfonyl, amidosulfonyl, alkyl, aryl, aralkyl, cycloalkyl, cycloalkylalkyl, heteroaryl, heterocyclyl, halo, haloalkyl, haloalkoxy, hydroxyalkyl, hydroxyhaloalkyl, aminoalkyl, carboalkoxy, carboxy, carboxyalkyl, carboxamido, and cyano;
A is a bond or (CH(R¹⁵))ₚₐ—(W⁷)ᵣᵣ wherein rr is 0 or 1, pa is an integer selected from 0 through 3, and W is selected from the group consisting of O, S, C(O), (R⁷)NC(O), (R⁷)NC(S), and N(R⁷);
R⁷ is selected from the group consisdng of hydtido, hydroy and alkyl;
R¹⁵ is selected from the group consisting of hydrido, hydroxy, halo, allyl, and haloalkyl;
X⁰ is selected from the group consisting of hydrido, amino, alkyl, cyano, halo, haloalkyl, haloalkoxy, aminoalkyl, hydroxy, alkoxy, hydroxyalkyl, and alkylthio;
R¹ is selected from the group consisting of hydrido, alkyl, haloalkyl, amino, aminoalkyl, alkylamino, hydroxy, and hydroxyalkyl;
R² is Z⁰-Q^{b};
Z⁰ is selected from the group consisting of a bond, (CR⁴¹R⁴²)_{q} wherein q is 1 or 2, and (CH(R⁴¹))_{g}—WO-(CH(R⁴²))ₚ wherein g and p are integers independently selected from 0 through 3 and W⁰ is selected from the group consisting of O, S, C(O), S(O), N(R⁴¹), and ON(R⁴¹);
Z⁰ is optionally (CH(R⁴¹))e,W²(CH(R⁴²))ₕ wherein e and h are independently 0 or 1 and W²² is selected from the group consisting of CR⁴¹═CR⁴², 1,2,5-cyclopropyl, 1,2-cyclobutyl, 1,2-cyclohexyl, 1,3-cyclohexyl, 1,2-cyclopentyl, 1,3-cyclopentyl, 2,3-morpholinyl, 2,4-morpholinyl, 2,6-morpholinyl, 3,4-morpholinyl, 3,5-morpholinyl, 1,2-piperazinyl, 1,3-piperazinyl, 2,3-piperazinyl, 2,6-pipernzinyl, 1,2-pipejidinyl, 1,3-piperidinyl, 2,3-piperidinyl, 2,4-piperidinyl, 2,6-piperidinyl, 3,4-piperidinyl, 1,2-pyrrolidinyl,1,3-pyrrolidinyl, 2,3-pyrrolidinyl, 2,4-pyrrolidinyl, 2,5-pyrrolidinyl, 3,4-pyn-olidinyl, 2,3-tetrahydrofuranyl, 2,4-tetrahydrofuranyl, 2,5-tetrahydrofuranyl, and 3,4-tetrahydrcofuranyl, wherein Z⁰ is directly bonded to the 4-pyridone ring and W²² is optionally substituted with one or more substituents selected from the group consisting of R⁹, R¹⁰, R¹¹, R¹², and R¹³;
R⁴¹ and R⁴² are independently selected from the group consisting of hydrido, hydroxy, alkyl, and amino;
Q^{b} is phenyl or a heteroaryl of 5 or 6 ring members, wherein a carbon adjacent to the carbon at the point of attachment of said phenyl or heteroaryl ring to Z⁰ is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹, with the proviso that Q^{b} is other than a phenyl when Z⁰ is a bond;
Q^{b} is optionally hydrido with the proviso that Z⁰ is selected from other than a bond;
K is CHR wherein R is selected from the group consisting of hydrido, hydroxyalkyl, alkyl, alkoxyalkyl, alkylthioalkyl, and haloalkyl;
E⁰ is selected from the group consisting of a bond, C(O)N(H), (H)NC(O), (R⁷)NS(O)₂, and S(O)₂N(R⁷);
Y^{AT} is Q^{b}-Q^{s};
Q^{s} is (CR³⁷R³⁸)_{b} wherein b is an integer selected from 1 through 4, 37 is selected from the group consisting of hydrido, alkyl and haloalkyl, and R is selected from the group consisting of hydrido, alkyl,d haloalkyl, aroyl, and heteroaroyl with the proviso that there is at least one aroyl or heteroaroyl substituent, with the further proviso that no more than one aroyl or heteroaroyl is bonded to (CR³⁷R³⁸)_{b} at the same time, with the still further proviso that said aroyl and said heteroaroyl are optionally substituted with one or more substituents selected from the group consisting of R¹⁶, R¹⁷, R¹⁸, and R¹⁹, with another further proviso that said aroyl and said heteroaroyl are bonded to the CR³⁷R³⁸ that is directly bonded to e, with still another further proviso that no more than one alkyl or one haloalkyl is bonded to a CR³⁷R³⁸ at the same time, and with the additional proviso that said alkyl and haloalkyl are bonded to a carbon other than the one bonding said aroyl or said heteroaroyl;
R¹⁶, R¹⁷, R¹⁸, and R¹⁹ are independently selected from the group consisting of hydrido, amidino, guanidino, carboxy, haloalkylthio, alkoxy, hydroxy, amino, alkoxyamino, alkylamino, akylthio, alkylsulfinyl, alkylsulfonyl, alkanoyl, haloalkanoyl, alkyl, halo, haloalkyl, haloalkoxy, hydroxyalkyl, aminoalkyl, and cyano;
R¹⁶ or R¹⁹ is optionally selected from the group consisting of NR²⁰R²¹,N(R²⁶)C(NR²⁵)N(R²³)(R²⁴),andc(NR²⁵)NR²³R²⁴, with the proviso that R¹⁶, R¹⁹, and Q^{b} are not simultaneously hydrido;
Q^{b} is selected from the group consisting of NR²⁰R²¹, hydrido, N(R²⁶)C(NR²⁵)N(R²³)(R²⁴), and C(NR²⁵)NR²³R²⁴, with the proviso that no more than one of R²⁰ and R²¹ is selected from the group consisting of hydroxy, amino, alkylamino, and dialkylamino at the same time and with the further proviso that no more than one of R²³ and R²⁴ is selected from the group consisting of hydroxy, amino, alkylamino, and dialkylamino at the same time;
R²⁰, R²¹, R²³, R²⁴, R²⁵, and R²⁶ are independently selected from the group consisting of hydrido, alkyl, hydroxy, amino, alkylamino and dialkylamino.

54. Compound of claim 53 of the Formula: 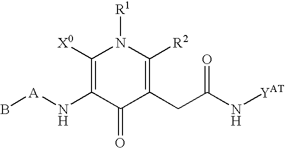
or a pharmaceutically acceptable salt thereof, wherein;
B is selected from the group consisting of phenyl, 2-thienyl, 3-thienyl, 2-furyl, 3-furyl, 2-pyrrolyl, 3-pyrrolyl, 2-imidazolyl, 4-imidazolyl, 3-pyrazolyl, 4-pyrazolyl, 2-thiazolyl, 3-isoxazolyl, and 5 isoxazolyl, wherein a carbon adjacent to the carbon at the point of attachment of said phenyl or heteroaryl ring to A is optionally substituted by R³², the other carbon adjacent to the carbon at the point of attachment is optionally substituted by R³⁶, a carbon adjacent to R³² and two atoms from the carbon at the point of attachment is optionally substituted by R³³, a carbon adjacent to R³⁶ and two atoms from the carbon at the point of attachment is optionally substituted by R³⁵, and any carbon adjacent to both R³³ and R³⁵ is optionally substituted by R³⁴;
R³², R³³, R³⁴, R³⁵, and R³⁶ are independently selected from the group consisting of hydrido, amidino, guanidino, methyl, ethyl, methoxy, ethoxy, hydroxy, amino, N-methylamino, dimethylamino, methoxyamino, methylthio, ethylthio, tifluoromethyl, pentafluoroethyl, 2,2,2-ttifluoroethyl, fluoro, chloro, bromo, amidosulfonyl, N-methylamidosulfonyl, hydroxymethyl, amidocarbonyl, carboxy, cyano, and Q^{b};
B is optionally selected from the group consisting of hydrido, ethyl, 2-propenyl, 2-propynyl, propyl, isopropyl, butyl, 2-butenyl, 2-butynyl, sec-butyl, tert-butyl, isobutyl, 2-methylpropenyl, 1-pentyl, 2-pentenyl, 3-pentenyl, 2-pentynyl, 3-pentynyl, 2-pentyl, 3-pentyl, 2-methylbutyl, 2-methyl-2-butenyl, 3-methylbutyl, 3-methyl-2-butenyl, 1-hexyl, 2-hexenyl, 3-hexenyl, 4-hexenyl, 2-hexynyl, 3-hexynyl, 4-hexynyl, 2-hexyl, 1-methyl-2-pentenyl, 1-methyl-3-pentenyl, 1-methyl-2-pentynyl, 1-methyl-3-pentynyl, 3-hexyl, 1-ethyl-2-butenyl, 1-heptyl, 2-heptenyl, 3-heptenyl, 4-heptenyl, 5-heptenyl, 2-heptynyl, 3-heptynyl, 4-heptynyl, 5-heptynyl, 2-heptyl, 1-methyl-2-hexenyl, 1-methyl-3-hexenyl, 1-methylhexenyl, 1-methyl-2-hexynyl, 1-methyl-3-hexynyl, 1-methyl-hexynyl, 3-heptyl, 1-ethyl-2-pentenyl, 1-thyl-3-pentenyl, 1-ethyl-2-pentynyl, 1-ethyl-3-pentynyl, 2,2,2-trifluoroethyl, 2,2-difluoropropyl, 4-trifluoromethyl-5,5,5-triuoropentyl, 4-trifiuoromethylpentyl, 5,5,6,6,6-pentafluorohexyl, and 3;3,3-trifluoropropyl, wherein each member of group B is optionally substituted at any carbon up to and including 5 atoms from the point of attachment of B to A with one or more of the group consisting of R³², R³³, R³⁴, R³⁵, and R³⁶;
B is optionally selected from the group consisting of cyclopropyl, cyclobutyl, cyclopentyl, cyclohexyl, cycloheptyl, oxalan-2-yl, 2-(2R)-bicyclo[2.2.1]-heptyl, oxetan-3-yl, azetidin-1-yl, azetidin-2-yl, azetidin-3-yl, bicyclo[3.1.0]hexanyl, 2-morpholinyl, 3-morpholinyl, 4-morpholinyl, 1-piperazinyl, 2-piperazinyl, 1-piperidinyl, 2-piperidinyl, 3-piperidinyl, 4-piperidinyl, 1-pyrrolidinyl, 2-pyrrolidinyl, 3-pyrrolidinyl, 2-dioxanyl, 2-tetrahydrofuranyl, 3-tetrahydrofuranyl, 2-tetrahydropyranyl, 3-tetrahydropyranyl, 4-tetrahydropyranyl, 2-tetrahydrothienyl, and 3-tetrahydrothienyl, wherein each ring carbon is optionally substituted with R³³, ring carbons and a nitrogen adjacent to the carbon atom at the point of attachment are optionally substituted with R⁹ or R¹³, a ring carbon or nitrogen adjacent to the R⁹ position and two atoms from the point of attachment is optionally substituted with R¹⁰, and a ring carbon or nitrogen adjacent to the R¹³ position and two atoms from the point of attachment is optionally substituted with R¹²;
R⁹, R¹¹, and R¹³ are independently selected from the group consisting of hydrido, methyl, ethyl, methoxy, ethoxy, hydroxy, amino, N-methylamino, N,N-dimethylamino, methylthio, trifluoromethyl, pentafluoroethyl, 2,2,2-bifluoroethyl, fluoro, chloro, bromo, amidosulfonyl, N-methylamidosulfonyl, N,N-dimethylamidosulfonyl, hydroxymethyl, 1-hydroxyethyl, amidocarbonyl, N-methylamidocarbonyl, carboxy, and cyano;
R¹⁰ and R¹² are independently selected from the group consisting of hydrido, amidino, amidocarbonyl, N-methylamidocarbonyl, N-benzylamidocarbonyl, N-(2-chlorobenzyl)amidocarbonyl, N-(3-fluorobenzyl)anridocarbonyl, N-(2-trifluoromethylbenzyl)amidocarbonyl, N-(1-phenylethyl)amidocarbonyl, N-(1-methyl-1-phenylethyl)amidocarbonyl, N-benzylamidosulfonyl, N-(2-eblorobenzyl)amidosulfonyl, N-ethylamidocarbonyl, Nisopropylamidocarbonyl, N-propylamidocarbonyl, N-isobutylamidocarbonyl, N-(2-butyl)amidocarbonyl, N-cyclobutylamidocarbonyl, N-cyclopentylamidocarbonyl, N-cyclohexylamidocarbonyl, guanidino, methyl, ethyl, methoxy, ethoxy, hydroxy, hydroxymethyl, 1-hydroxyethyl, 2-hydroxyethyl, carboxy, carboxymethyl, amino, acetamido, trifluoromethyl, pentafluoroethyl, 2,2,2-trifluoroethyl, trifluoroacetamido, anunomethyl, N-methylmo, dilnethylamino, methoxyamino, amidosulfonyl, N-methylamidosulfonyl, N,N-dimethylamidosulfonyl, methanesulfonamido, methoxycarbonyl, fluoro, chloro, bromo, and cyano;
A is selected from the group consisting of a bond, NH, N(CH₃), CH₂, CH₃CH, CH₂CH₂, and CH₂CH₂CH₂;
X^{o} is selected from the group consisting of hydrido, hydroxy, amino, amiinomethyl, 1-aminoethyl, methyl, ethyl, trifluoromethyl, pentafluoroethyl, 2,2,2-trifluoroethyl, methoxy, hydroxymethyl, 1-hydroxyethyl, 2-hydroxyethyl, methylthio, ethylthio, trifluoromethoxy, 1,1,2,2-tetrafluoroethoxy, fluoro, chloro, and bromo;
R¹ is selected from the group consisting of hydrido, hydroxy, amino, methyl, ethyl, propyl, n-butyl, 2,2,2-trilluoroethyl, 3,3,3-trifluoropropyl, 2,2,3,3,3-pentafluoropropyl, 2-aminoethyl, 3-aminopropyl, 2-hydroxyethyl, 3-hydroxypropyl, and 2,3-dihydroxypropyl;
R² is selected from the group consisting of phenyl, 2,5-thienyl, 2-furyl, 2-pyrrolyl, 2-imidazolyl, 2-thiazolyl, 3-isoxazolyl, 2-pyridyl, and 3-pyridyl, wherein a carbon adjacent to the carbon at the point of attachment of said phenyl or heteroaryl ring to the 4-pyridone ring is optionally substituted by R⁹, the other carbon adjacent to the carbon at the point of attachment is optionally substituted by R¹³, a carbon adjacent to R⁹ and two atoms from the carbon at the point of attachment is optionally substituted by R¹⁰, a carbon adjacent to R¹³ and two atoms from the carbon at the point of attachment is optionally substituted by R¹², and any carbon adjacent to both R¹⁰ and R¹² is optionally substituted by R¹¹;
Y^{AT} is Q^{b}-Q^{s};
Q^{s} is selected from the group consisting of:
C[R³⁷ (benzoyl)(CR³⁷R³⁸)_{b}],
C[R³⁷ (2-pyridylcarbonyl)(CR³⁷R³⁸)_{b}],
C[R³⁷(3-pyridylcarbonyl)(CR³⁷R³⁸)_{b}],
C[R³⁷(4-pyridylcarbonyl)(CR³⁷R³⁸)_{b}],
C[R³⁷(2-thienylcarbonyl)(CR³⁷R³⁸)_{b}],
C[R³⁷(3-thienylcarbonyl)(CR³⁷R³⁸)_{b}],
C[R³⁷(2-thiazolylcarbonyl)(CR³⁷R³⁸)_{b}],
C[R³⁷ (4-thiazolylcarbonyl)(CR³⁷R³⁸)_{b}], and
C[R³⁷(5-thiazolylcarbonyl)(CR³⁷R³⁸)_{b}], wherein b is an integer selected from 1 through 3, R³⁷-and R³⁸-are independently selected from the group consisting of hydrido, alkyl, and haloalkyl, with the proviso that said benzoyl and the heteroaroyls are optionally substituted with one or more substituents selected from the group consisting of R¹⁶, R¹⁷, R¹⁸, and R¹⁹ with the proviso that R¹⁷ and R¹⁸ are optionally substituted at a carbon selected from other than the meta and para carbons relative to the carbonyl of the benzoyl or heteroaroyl, with the further proviso that said benzoyl or said heteroaroyl are bonded to the carbon directly bonded to amide nitrogen of the 1-amidocarbonymethylene) group, and with the still further proviso that is no more than one alkyl or one haloalkyl is bonded to a C R³⁷R³⁸-at the same time;
R¹⁶, R¹⁷, R¹⁸, and R¹⁹ are independently selected from the group consisting of hydrido, methyl, ethyl, amidino, guanidino, methoxy, hydroxy, amino, aminomethyl, 1-amunoethyl, 2-aminoethyl, N-methylamino, dimethylamino, methylthio, ethylfhio, trifluoromethylthio, methylsulfinyl, methylsulfonyl, trifluoromethyl, pentafluoroethyl, 2,2,2-tnfluoroethyl, trifluoromethoxy, fluoro, chloro, hydroxymethyl, carboxy, and cyano;
Q^{b} i C(NR²⁵)NR²³R²⁴ or N(R²⁶)C(NR²⁵)N(R²³)(R²⁴);
R²³, R²⁴, R²⁵, and R²⁶ are independently selected from the group consisting of hydrido, methyl, and ethyl.

55. Compound of claim 54 or a pharmaceutically acceptable salt thereof, wherein;
B is selected from the group consisting of 2-aminophenyl, 3-aminophenyl, 3-amidinophenyl, 4-amidiiaophenyl, 3-carboxyphenyl, 3-carboxy-5-hydroxyphenyl, 3-chlorophenyl, 4-chlorophenyl, 3,4-dichlorophenyl, 2-fluorophenyl, 3-fluorophenyl, 3,4-difluorophenyl, 3-hydroxyphenyl, 4-hydroxyphenyl, 3-methoxyaminophenyl, 3-methoxyphenyl, 4-methoxyphenyl, 3-methylphenyl, 4-methylphenyl, phenyl, 3-trifluoromethylphenyl, 2-imidazoyl, 2-pyridyl, 3-pyridyl, schloro-3-trifluoromethyl-2-pyridyl, 4-pyridyl, 2-thienyl, 3-thienyl, and 3-trifluoromethyl-2-pyridyl;
B is optionally selected from the group consisting of hydrido, ethyl, 2-propenyl, 2-propynyl, propyl, isopropyl, butyl, 2-butyl, (R)-2-butyl, (S)-2-butyl, tert-butyl, isobutyl, 1-pentyl, 3-pentyl, 2-methylbutyl, 2,2,2-trifluoroethyl, 6-amidocazbonylhexyl, 4-methyl-2-pentyl, 3-hydroxypropyl, 1-methoxy-2-propyl, 2-methoxyethyl, 2-methyl-2-butyl, 3-methyl-2-butyl, 2-dimethylaminopropyl, 2-cyanoethyl, 6-hydroxyhexyl, 302-hydroxyethyl, 2-amidinoethyl, 2-guanidinoethyl, 3-guanidinopropyl, 4-guanidinobutyl, 3-hydroxypropyl, 4-hydroxybutyl, 6-cyanohexyl, 2-dimethylaminoethyl, 3-methylbutyl, 2-methylbutyl, (S)-2-methylbutyl, 3-aminopropyl, 2-hexyl, and 4-aminobutyl;
B is optionally selected from the group consisting of cyclopropyl, cyclobutyl, cyclopentyl, cyclohexyl, oxalan-2-yl, 2-(2R)-bicyclo[2.2.1]-heptyl, oxetan-3-yl, azetidin-1-yl, azetidin-2-yl, azetidin-3-yl, 1-pyrrolidinyl and 1-pipendinyl;
A is selected from the group consisting of a bond, CH₂, (G3CH, CH₂CH₂, and CH₂CH₂CH₂;
X^{o} is selected from the group consisting of hydrido, hydroxyl-aminoethyl, methyl, ethyl, trfluoromethyl, pentafluoroethyl, 2,2,2-trifluoroethyl, hydroxymethyl, 1-hydroxyethyl, 2-hydroxyethyl, fluoro, chloro, and bromo;
R¹ is selected from the group consisting of hydrido, hydroxy, amino, methyl, ethyl, 2,2,2-trifluoroethyl, 3,3,3-trifluoropropyl, 2-aminoethyl, and 2-hydroxyethyl;
R² is selected from the group consisting of 3-amidocarbonyl-5-aminophenyl, 3-amidocarbonyl-5-aminophenyl, 3-amino-5-(N-benzylamidocarbonyl)phenyl, 3-amino-5-(N-(2-chlorobenzyl)amidocarbonyl)phenyl, 3-amino-(N-(3-fluorobenzyl)amidocarbonyl)phenyl, 3-amino-(N-(2-trifluoromethylbenzyl)amidocarbonyl)phenyl, 3-amino-5-(N-1-phenylethyl)amidocarbonyl)phenyl, 3-amino-5-(N-(1-methyl-1-phenylethyl)amidocarbonyl)phenyl, 3-amino-5-(N-benzylamidosulfonyl)phenyl, 3-amiuno-5-N-(2-chlorobenzyl)amidosulfonyl)phenyl, 3-amino-5-(N-ethylamidocarbonyl)phenyl, 3-alino-5 (N-isopropylamidocarbonyl)phenyl, 3-amino-5-(N-propylamidocarbonyl)phenyl, 3-amino-(N-isobutylamidocarbonyl)phenyl, 3-amino-5-(2-butyl)amidocarbonyl)phenyl, 3-amino-5-(N-cyclobutylamidocarbonyl)phenyl, 3-amino-5-cyclopentylamidocarbonyl)phenyl, 3-amino-5-(N-cyclohexylamidocarbonyl)phenyl, 5-amino-2-fluorophenyl, 3-amino-5-hydroxymethylphenyl, 5-amino-3-methoxycarbonylphenyl, 3-amidinophenyl, 3-amino-2-methylphenyl, 5-amino-2-methylthiophenyl, 3-aminophenyl, 3-carboxyphenyl, 3-carboxy-5-aminophenyl, 3-carboxy-5-hydroxyphenyl, 3-carboxymethyl-5-aminophenyl, 3-carboxymethyl-5-hydroxyphenyl, 3-carboxymethylphenyl, 3-chlorophenyl, 2-chlorophenyl, 3-cyanophenyl, ,5-iaminophenyl, 3-dimethylaminophenyl, 2-fluorophenyl, 3-fluorophenyl, 2,5-difluorophenyl, 2-hydroxyphenyl, 3-hydroxyphenyl, 3-methanesulfonylaminophenyl, 2-methoxyphenyl, 3-methoxyphenyl, 3-methoxyaminophenyl, 3-methoxycarbonylphenyl, 2-methylaminophenyl, 3-methylaminophenyl, 2-methylphenyl, 3-methylphenyl, 4-methylphenyl, phenyl, 3-trifluoroacetamidophenyl, 3-trifluoromethylphenyl, 2-trifluoromethylphenyl, 5-amino-2-thienyl, Samino-3-thienyl, 3-bromo-2-thienyl, 3-pyridyl, 4-pyridyl, 2-thienyl, and 3-thienyl;
Y^{AT} is Q^{b}-Q^{s};
Q^{s} is selected from the group consisting of:
[CH(benzoyl)](CH₂)_{b}, [CH(2-pyridylcarbonyl)](CH₂)_{b},
[CH(3-pyridylcarbonyl)](CH₂)_{b}, [CH(4-pyridylcarbonyl)](CH₂)_{b},
[CH(2-thienylcarbonyl)](CH₂)_{b},[CH(3-thienylcarbonyl)](CH₂)_{b},
[CH(2-thiazolylcarbonyl)](CH₂)_{b}, [CH($thiazolylcarbonyl)](CH₂)_{b}, and [CH(5-thiazolylcarbonyl)](CH₂)_{b}, wherein b is an integer selected from 1 through 3, with the proviso that said benzoyl and said heteroaroyls are optionally substituted with one or more substituents selected from the group consisting of R¹⁶, R¹⁷, R¹⁸, and R¹⁹ with the proviso that R¹⁷ and R¹⁸ are optionally substituted at a carbon selected from other than the meta and para carbons relative to the carbonyl of the benzoyl or the heteroaroyl, and that said benzoyl or said heteroaroyl are bonded to the carbon directly bonded to amide nitrogen of the 1-(amidocarbonymethylene) group;
R¹⁶ and R¹⁹ are independently selected from the group consisting of hydrido, amidino, amino, aminomethyl, methoxy, methylamino, hydroxy, hydroxymethyl, fluoro, chloro, and cyano;
R¹⁷ and R¹⁸ are independently selected from the group consisting of hydrido, fluoro, chloro, hydroxy, hydroxymethyl, amino, carboxy, and cyano;
Q^{b} is N(R²⁶)C(NR²⁵)N(R²³)(R²⁴);
R²³, R²⁴, R²⁵, and R²⁶ are independently hydrido or methyl.

56. Compound of claim 55 or a pharmaceutically acceptable salt thereof, wherein;
B is selected from the group consisting of 3-anunophenyl, 3-amidinophenyl, 4-amidinophenyl, 3-chlorophenyl, 4-chlorophenyl, 3,4-dichlorophenyl, 2-fluorophenyl, 4-methylphenyl, phenyl, 2-imidazoyl, 3-pyridyl, 4-pyridyl, and 3-trifluoromethyl-2-pyridyl;
B is optionally selected from the group consisting of hydrido, ethyl, 2-propenyl, 2-propynyl, propyl, isopropyl, butyl, 2-butyl, (R)-2-butyl, (S)₂-butyl, tert-butyl, isobutyl, 1-pentyl, 3-pentyl, 2-methylbutyl, 2,2,2-trfluoroethyl, 6-amidocarbonylhexyl, 4-methyl-2-pentyl, 3-hydroxypropyl, 1-methoxy-2-propyl, 2-methoxycthyl, 2-methyl-2-butyl, 3-methyl-2-butyl, 2-dimethylaminopropyl, 2-cyanoethyl, 6-hydroxyhexyl, 2-hydroxyethyl, 2-anidinoethyl, 2-guanidinoethyl, 3-guanidinopropyl, 4-guanidinobutyl, 3-hydroxypropyl, 4-hydroxybutyl, 6-cyanohexyl, 2-dimethylaminoethyl, 3-methylbutyl, 2-methylbutyl, (S)-2-methylbutyl, 3-aminopropyl, 2-hexyl, and 4-aminobutyl;
B is optionally selected from the group consisting of cyclopropyl, cyclobutyl, cyclopentyl, cyclohexyl, oxalan-2-yl, 2-(2R)-bicyclo[2.2.1]-heptyl, oxetan-3-yl, azetidin-1-yl, azetidin-2-yl, azetidin-3-yl, and 1-piperidinyl;
A is selected from the group consisting of a bond, CH₂, CH₂CH₂ and CH₂CH₂CH₂;
X^{o} is selected from the group consisting of hydrido, aminomethyl, methyl, ethyl, trifluoromethyl, 2,2,2-tifluoroethyl, hydroxymethyl, 2-hydroxyethyl, chloro, and fluoro;
R¹ is selected from the group consisting of hydrido, methyl, ethyl, 2,2,2-trifluoroethyl, 3,3,3-bifluoropropyl, and 2-hydroxyethyl;
R² is selected from the group consisting of 3-amidocarLonyl-5-aminophenyl, 3-amino-i(N-benzylanidocarbonyl)phenyl, 3-amino-5-(N-(2-chlorobenzyl)amidocarbonyl)phenyl, 3-amino-5-(N-(3-fluorobenzyl)amidocarbonyl)phenyl, 3-amino-5-(N-(2-trifluoromethylbenzyl)amidocarbonyl)phenyl, 3-atnino-(N-(1-phenylethyl)amidocarbonyl)phenyl, 3-amino-1-(1-methyl-1-phenylethyl)amidocarbonyl)phenyl, 3-amino-5-(N-benzylamidosulfonyl)phenyl, 3-amino-5-(N-(2-chlorobenzyl)amidosulfonyl)phenyl, 3-amino-5-(N-ethylainidoarbonyl)phenyl, 3-amino-5-(N-isopropylamidocarbonyl)phenyl, 3-amino-5-(N-propylaniidocarbonyl)phenyl, 3-amino-5-(N-isobutylamidocarbonyl)phenyl, 3-amino-5-(N-(2-butyl)amidocarbonyl)phenyl, 3-amino-5-Ncyclobutylamidocarbonyl)phenyl, 3-amino-5-(N-cyclopentylamidocarbonyl)phenyl, 3-ainino-5-(N-cyclohexylamidocarbonyl)phenyl, 3-aminophenyl, 3-carboxy-5-aminophenyl, 3-chlorophenyl, 3,5-diaminophenyl, 3-imethylaminophenyl, 3-hydroxyphenyl, 3-methanesulfonylaminophenyl, 3-methylaminophenyl, 2-methylphenyl, 3-methylphenyl, phenyl, 3-trifluoroacetamidophenyl, 3-bromo-2-thienyl, 2-thienyl, and 3-thienyl;
Y^{AT} is selected from the group consisting of 5-guanidino-1-oxo-1-(2-thiazolyl)-2-pentyl, 5-guanidino-1-oxo-1-(4-thiazolyl)₂-pentyl, 5-guanidino-1-oxo-1-(5-thiazolyt)-2-pentyl, 5-guanidino-1-oxo-1-(4-amino-2-thiazolyl)-2-pentyl, and 5-guanidino-1-oxo-1-phenyl-2-pentyl.

57. A compound as recited in claim 53 where said compound is selected from the group of the Formula: 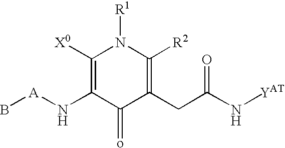
or a pharmaceutically acceptable salt thereof, wherein B, A, R¹, R², X°, and yA are selected to form a compound selected from the group consisting of;
R² is 3-aminophenyl, B is phenyl, A is CH₂CH₂, Y^{AT} is 5-guanidino-1-oxo-112-thiazolyl)-2-pentyl, R¹ is hydrido, and X⁰ is hydrido;
R² is 3,5-diauinophenyl, B is phenyl, A is CH₂CH₂, Y^{AT} is Sguanidino 1-oxo-112-thiazolyl)-2-pentyl, R¹ is hydrido, and X⁰ is hydrido;
R² is 3-carboxy-5-aminophenyl, B is phenyl, A is CH,2CH₂, Y^{AT} is 5-guanidino-1-oxo-1-(2-thiazolyl)₂-pentyl, R¹ is hydrido, and X⁰ is hydrido;
R² is 3-amino-5-(N-benzylamidocarbonyl)phenyl, B is phenyl, A is CH₂CH₂, Y^{AT} is 5-guanidino-1-oxo-1-(2-thiazolyl)-2-pentyl, R¹ is hydrido, and X⁰ is hydrido;
R² is 3,5-diaminophenyl, B is isopropyl, A is single bond, Y^{AT} is 5-guanidino-1-oxo-112-thiazolyl)-2-pentyl, R¹ is hydrido, and X⁰ is hydrido;
R² is 3-carboxy-5-aminophenyl, B is isopropyl, A is single bond, Y^{AT} is 5 guanidino-1-oxo-1-(2-thiazolyl)₂-pentyl, R¹ is hydrido, and X⁰ is hydrido;
R² is 3-amino-5-(N-benzylamidocarbonyl)phenyl, B is isopropyl, A is single bond, Y^{AT} is 5-guanidino-1-oxo-1-(2-thiazolyl)-2-pentyl, R¹ is hydrido, and X^{o} is hydrido;
R² is 3,5-diaminophenyl, B is cyclobutyl, A is single bond, Y^{AT} is 5-guanidino-1-oxo-1-(2-thiazolyl)-2-pentyl, R¹ is hydrido, and X⁰ is hydrido;
R² is 3-carboxy-5-aminophenyl, B is cyclobutyl, A is single bond, YA is 5 guanidino-1-oxo-1-(thiazolyl)₂-pentyl, R¹ is hydrido, and X⁰ is hydrido;
R² is 3-amino-5-N-benzylamidocarbonyl)phenyl, B is cyclobutyl, A is single bond, Y^{AT} is 5-guanidino1-oxo-1-(2-thiazolyl)₂-pentyl, R¹ is hydrido, and X⁰ is hydrido;
R² is 3-aminophenyl, B is phenyl, A is CH₂CH₂, Y^{AT} is S-guanidino-1-oxo-1-(2-thiazolyl)-2-pentyl, R¹ is methyl, and X⁰ is hydrido;
R² is 3,5-diaminophenyl, B is phenyl, A is CH₂CH₂, YT is S-guanidino-1-oxo-1-2-thiazolyl)-2-pentyl, R¹ is methyl, and X⁰ is hydrido;
R² is 3-carboxy-5-aminophenyl, B is phenyl, A is CH₂CH₂, Y^{AT} is S-guanidino-1-oxo-1-(2-thiazolyl)-2-pentyl, R¹ is methyl, and X⁰ is hydrido;
R² is 3-amino-5-(N-benzylamidocarbonyl)phenyl, B is phenyl, A is CH₂CH₂, YT is 5-guanidino-1-oxo-1-(2-thiazolyl)₂-pentyl, R¹ is methyl, and X⁰ is hydrido;
R² is 3,5-diaminophenyl, B is isopropyl, A is single bond, Y^{AT} is 5-guanidino-1-oxo-112-thiazolyl)-2-penty), R¹ is methyl, and X⁰ is hydrido;
R² is 3-carboxy-5-aminophenyl, B is isopropyl, A is single bond, Y^{AT} iS 5-guanidino-1-oxo-1-(2-thiazolyl)-2-pentyl, R¹ is methyl, and X⁰ is hydrido;
R² is 3-amino-5-N-benzylaniidocaibonyl)phenyl, B is isopropyl, A is single bond, y is 5-guanidino-1-oxo-1-(2-thiazolyl)-2-pentyl, R¹ is methyl, and X⁰ is hydrido;
R² is 3,5-diaminophenyl, B is cyclobutyl, A is single bond, Y^{AT} is 5-gmnidino-1-oxo-112-thiazolyl)-2-pentyl, R¹ is methyl, and X⁰ is hydrido; R² is 3-carboxy-5-aminophenyl, B is cyclobutyl, A is single bond, Y^{AT} is 5 guanidino-1-oxo-12-thiazolyl)-2,5-pentyl;R¹ is methyl, and X⁰ is hydrido; and
R² is 3-amino-5-(N-benzylamidocarbonyl)phenyl, B is cyclobutyl, A is single bond, Y⁰ is 5-guanidino-1-oxo-1-(2-thiazolyl)₂-pentyl, R¹ is methyl, and X⁰ is hydrido.

58. A composition for inhibiting thrombotic conditions in blood comprising a compound of claim 21 and a pharmaceutically acceptable carrier.

59. A method for inhibiting thrombotic conditions in blood comprising adding to blood a therapeutically effective amount of a composition of claim 58.

60. A method for inhibiting formation of blood platelet aggregates in blood comprising adding to blood a therapeutically effective amount of a composition of claim 58.

61. A method for inhibiting thrombus formation in blood comprising adding to blood a therapeutically effective amount of a composition of claim 58.

62. A method for treating or preventing venuous thromboembolism and pulmonary embolism in a mammal comprising administering to the manmal a therapeutically effective amount of a composition of claim 58.

63. A method for treating or preventing deep vein thrombosis in a mammal comprising administering to the mammal a therapeutically effective amount of a composition of claim 58.

64. A method for treating or preventing cardiogenic thromboembolism in a mammal comprising administering to the mammal a therapeutically effective amount of a composition of claim 58.

65. A method for treating or preventing thromboembolic stroke in humans and other mamals comprising administering to the mammal a therapeutically effective amount of a composition of claim 58.

66. A method for treating or preventing thrombosis associated with cancer and cancer chemotherapy in humans and other mammals comprising administering to the mamma a therapeutically effective amount of a composition of claim 58.

67. A method for treating or preventing unstable angina in humans and other manmals comprising administering to the mammal a therapeutically effective amount of a composition of claim 58.

68. A method for inhibiting thrombus formation in blood comprising adding to blood a therapeutically effective amount of a compound of claim 21 with a therapeutically effective amount of fibrinogen receptor antagonist.

69. The use of a compound of claim 21, or a pharmaceutically acceptable salt thereof, in the manufacture of medicament for inhibiting thrombus formation, treating thrombus formation, or preventing thrombus formation in a mammal.
